SEQUENCE LISTING (78 sequences)

SEQ ID NO: 1 - DNA, 499 bp, Artificial Sequence
  xemplary 499 base sequence of D1/D2 domain of H0 Metschnikowia
aaaccaacagggattgcctcagtaacggcgagtgaagcggcaaaagctcaaatttgaaatcccccgggaattgtaatttgaagagatttgggtccggccggcaggggttaagtccactggaaagtggcgccacagagggtgacagccccgtgaaccccttcaacgccttcatcccagatctccaagagtcgagttgtttgggaatgcagctctaagtgggtggtaaattccatctaaagctaaataccggcgagagaccgatagcgaacaagtacagtgatggaaagatgaaaagcactttgaaaagagagtgaaaaagtacgtgaaattgttgaaagggaagggcttgcaagcagacacttaactgggccagcatcggggcggcgggaaacaaaaccaccggggaatgtacctttcgaggattataaccccggtccttatttcctcgccaccccgaggcctgcaatctaaggatgctggcgtaatggttgcaagtcgc

SEQ ID NO: 2 - DNA, 499 bp, Artificial Sequence
  consensus D1/D2 domain sequence
aaaccaacagggattgcctcagtaacggcgagtgaagcggcaaaagctcaaatttgaaatcccccgggaattgtaatttgaagagatttgggtccggccggcaggggttaagtccactggaaagtggcgccacagagggtgacagccccgtgaaccccttcaacgccctcatcccagatctccaagagtcgagttgtttgggaatgcagctctaagtgggtggtaaattccatctaaagctaaataccggcgagagaccgatagcgaacaagtacagtgatggaaagatgaaaagcactttgaaaagagagtgaaaaagtacgtgaaattgttgaaagggaagggcttgcaagcagacacttaactgggccagcatcggggcggcgggaaacaaaaccaccggggaatgtacctttcgaggattataaccccggtctctatttcctyacyrccccgaggcctgcaatctaaggatgctggcgtaatggttgcaagtcgc

SEQ ID NO: 3 - DNA, 499 bp, Artificial Sequence
  D1/D2 domain sequences clone IH01-1 and H02-2
aaaccaacagggattgcctcagtaacggcgagtgaagcggcaaaagctcaaatttgaaatcccccgggaattgtaatttgaagagatttgggtccggccggcgggggttaagtccactggaaagtggcgccacagagggtgacagccccgtgagcccctctaacgcctctaccccaaatctccaagagtcgagttgtttgggaatgcagctctaagtgggtggtaaattccatctaaagctaaataccggcgagagaccgatagcgaacaagtacagtgatggaaagatgaaaagcactttgaaaagagagtgaaaaagtacgtgaaattgttgaaagggaagggcttgcaagcagacacttaactgggccagcatcggggcggcgggaaacaaaaccaccggggaatgtacctttcgaggattataaccccggtctcaatttcctcaccaccccgaggcctgcaatctaaggatgctggcgtaatggttgcaagtcgc

SEQ ID NO: 4 - DNA, 499 bp, Artificial Sequence
  D1/D2 domain sequences clone H01-2, H01-3 or H03-2
aaaccaacagggattgcctcagtaacggcgagtgaagcggcaaaagctcaaatttgaaatcccccgggaattgtaatttgaagagatttgggtccggccggcgggggttaagtccactggaaagtggcgccacagagggtgacagccccgtgaaccccttcaaagccttcatcccagatctccaagagtcgagttgtttgggaatgcagctctaagtgggtggtaaattccatctaaagctaaataccggcgagagaccgatagcgaacaagtacagtgatggaaagatgaaaagcactttgaaaagagagtgaaaaagtacgtgaaattgttgaaagggaagggcttgcaagcagacacttaactgggccagcatcggggcggcgggaaacaaaaccaccggggaatgtacctttcgaggattataaccccggtctctatttccatgttgccccgaggcctgcattctaaggatgctggcgtaatggttgcaagtcgc

SEQ ID NO: 5 - DNA, 499 bp, Artificial Sequence
  D1/D2 domain sequences clone H02-1
aaaccaacagggattgcctcagtaacggcgagtgaagcggcaaaagctcaaatttgaaatcccccgggaattgtaatttgaagagatttgggtccggccggcaggggttaagtccactggaaagtggcgccacagagggtgacagccccgtgagcccctctaaagcctctaccccagatctccaagagtcgagttgtttgggaatgcagctctaagtgggtggtaaattccatctaaagctaaataccggcgagagaccgatagcgaacaagtacagtgatggaaagatgaaaagcactttgaaaagagagtgaaaaagtacgtgaaattgttgaaagggaagggcttgcaagcagacacttaactgggccagcatcggggcggcgggaaacaaaaccaccggggaatgtacctttcgaggattatacccctggtctctatttccatgttgccccgaggcctgcaatctaaggatgctggcgtaatggttgcaagtcgc

SEQ ID NO: 6 - DNA, 499 bp, Artificial Sequence
  D1/D2 domain sequences clone H02-3
aaaccaacagggattgcctcagtaacggcgagtgaagcggcaaaagctcaaatttgaaatcccccgggaattgtaatttgaagagatttgggtccggccggcgggggttaagtccactggaaagtggcgccacagagggtgacagccccgtgagcccctctaacgcctctaccccaaatctccaagagtcgagttgtttgggaatgcagctctaagtgggtggtaaattccatctaaagctaaataccggcgagagaccgatagcgaacaagtacagtgatggaaagatgaaaagcactttgaaaagagagtgaaaaagtacgtgaaattgttgaaagggaagggcttgcaagcagacacttaactgggccagcatcggggcggcgggaaacaaaaccaccggggaatgtacctttcgaggattataaccccggtctctatttccatgttgccccgaggcctgcattctaaggatgctggcgtaatggttgcaagtcgc

SEQ ID NO: 7 - DNA, 499 bp, Artificial Sequence
  D1/D2 domain sequences clone H03-1
aaaccaacagggattgcctcagtaacggcgagtgaagcggcaaaagctcaaatttgaaatcccccgggaattgtaatttgaagagatttgggtccggccggcgggggttaagtccactggaaagtggcgccacagagggtgacagccccgtgagcccctctaacgcctctaccccagatctccaagagtcgagttgtttgggaatgcagctctaagtgggtggtaaattccatctaaagctaaataccggcgagagaccgatagcgaacaagtacagtgatggaaagatgaaaagcactttgaaaagagagtgaaaaagtacgtgaaattgttgaaagggaagggcttgcaagcagacacttaactgggccagcatcggggcggcgggaaacaaaaccaccggggaatgtacctttcgaggattataaccccggtctctatttccatgttgccccgaggcctgcattctaaggatgctggcgtaatggttgcaagtcgc

SEQ ID NO: 8 - DNA, 499 bp, Artificial Sequence
  D1/D2 domain sequences clone H1-1, H1-3
aaaccaacagggattgcctcagtaacggcgagtgaagcggcaaaagctcaaatttgaaatcccccgggaattgtaatttgaagagatttgggtccggccggcaggggttaagtccactggaaagtggcgccacagagggtgacagccccgtgaacccctttaacgccctcatcccagatctccaagagtcgagttgtttgggaatgcagctctaagtgggtggtaaattccatctaaagctaaataccggcgagagaccgatagcgaacaagtacagtgatggaaagatgaaaagcactttgaaaagagagtgaaaaagtacgtgaaattgttgaaagggaagggcttgcaagcagacacttaactgggccagcatcggggcggcgggaaacaaaaccaccggggaatgtacctctcgaggattataaccccggtctcaatttccttgttgccccgaggcctgcaatctaaggatgctggcgtaatggttgcaagtcgc

SEQ ID NO: 9 - DNA, 499 bp, Artificial Sequence
  D1/D2 domain sequences clone H1-2
aaaccaacagggattgcctcagtaacggcgagtgaagcggcaaaagctcaaatttgaaatcccccgggaattgtaatttgaagagatttgggtccggccggcaggggttaagtccactggaaagtggcgccacagagggtgacagccccgtgaaccccctcaacgccctcatcccagatctccaagagtcgagttgtttgggaatgcagctctaagtgggtggtaaattccatctaaagctaaataccggcgagagaccgatagcgaacaagtacagtgatggaaagatgaaaagcactttgaaaagagagtgaaaaagtacgtgaaattgttgaaagggaagggcttgcaagcagacacttaactgggccagcatcggggcggcgggaaacaaaaccaccggggaatgtacctttcgaggattatacccctggtctctatttccatgttgccccgaggcctgcaatctaaggatgctggcgtaatggttgcaagtcgc

SEQ ID NO: 10 - DNA, 499 bp, Artificial Sequence
  D1/D2 domain sequences clone H1-4
aaaccaacagggattgcctcagtaacggcgagtgaagcggcaaaagctcaaatttaaaatcccccgggaattgtaatttgaagagatttgggtccggccggcaggggttaagtccactggaaagtggcgccacagagggtgacagccccgtgaacccctttaacgccctcatcccagatctccaagagtcgagttgtttgggaatgcagctctaagtgggtggtaaattccatctaaagctaaataccggcgagagaccgatagcgaacaagtacagtgatggaaagatgaaaagcactttgaaaagagagtgaaaaagtacgtgaaatcgttgaaagggaagggcttgcaagcagacacttaactgggccagcatcggggcggcggggagcaaaaccaccggggaatgtacctttcgaggattataaccccggcccttactcccatactgccccgaggcctgcaatctaaggatgctggcgtaatggttgcaagtcgc

SEQ ID NO: 11 - DNA, 499 bp, Artificial Sequence
  D1/D2 domain sequences clone H1-5,  H2-5 or H2-7
aaaccaacagggattgcctcagtaacggcgagtgaagcggcaaaagctcaaatttgaaatcccccgggaattgtaatttgaagagatttgggtccggccggcaggggttaagtccactggaaagtggcgccacagagggtgacagccccgtgaacccctttaacgccctcatcccagatctccaagagtcgagttgtttgggaatgcagctctaagtgggtggtaaattccatctaaagctaaataccggcgagagaccgatagcgaacaagtacagtgatggaaagatgaaaagcactttgaaaagagagtgaaaaagtacgtgaaattgttgaaagggaagggcttgcaagcagacacttaactgggccagcatcggggcggcggggagcaaaaccaccggggaatgtacctttcgaggattataaccccggcccttactcccacaccaccccgaggcctgcaatctaaggatgctggcgtaatggttgcaagtcgc

SEQ ID NO: 12 - DNA, 499 bp, Artificial Sequence
  D1/D2 domain sequences clone H1-6
aaaccaacagggattgcctcagtaacggcgagtgaagcggcaaaagctcaaatttgaaatcccccgggaattgtaatttgaagagatttgggtccggccggcgggggttaagtccactggaaagtggcgccacagagggtgacagccccgtgaacccctttaacgccctcatcccagatctccaagagtcgagttgtttgggaatgcagctctaagtgggtggtaaattccatctaaagctaaataccggcgagagaccgatagcgaacaagtacagtgatggaaagatgaaaagcactttgaaaagagagtgaaaaagtacgtgaaattgttgaaagggaagggcttgcaagcagacacttaactgggccagcatcggggcggcgggaaacaaaaccaccggggaatgtacctctcgaggattataaccccggtctcaatttcctcaccaccccgaggcctgcaatctaaggatgctggcgtaatggttgcaagtcgc

SEQ ID NO: 13 - DNA, 499 bp, Artificial Sequence
  D1/D2 domain sequences clone H1-7
aaaccaacagggattgcctcagtaacggcgagtgaagcggcaaaagctcaaatttgaaatcccccgggaattgtaatttgaagagatttgggtccggccggcaggggttaagtccactggaaagtggcgccacagagggtgacagccccgtgagcccctctaaagcctctaccccaaatctccaagagtcgagttgtttgggaatgcagctctaagtgggtggtaaattccatctaaagctaaataccggcgagagaccgatagcgaacaagtacagtgatggaaagatgaaaagcactttgaaaagagagtgaaaaagtacgtgaaattgttgaaagggaagggcttgcaagcagacacttaactgggccagcatcggggcggcgggaaacaaaaccaccggggaatgtacctctcgaggattataaccccggtctcaatttcctcaccaccccgaggcctgcaatctaaggatgctggcgtaatggttgcaagtcgc

SEQ ID NO: 14 - DNA, 499 bp, Artificial Sequence
  D1/D2 domain sequences clone H1-8
aaaccaacagggattgcctcagtaacggcgagtgaagcggcaaaagctcaaatttgaaatcccccgggaattgtaatttgaagagatttgggtccggccggcaggggttaagtccactggaaagtggcgccacagagggtgacagccccgtaaaccccttcaaagccttcatcccagatctccaagagtcgagttgtttgggaatgcagctctaagtgggtggtaaattccatctaaagctaaataccggcgagagaccgatagcgaacaagtacagtgatggaaagatgaaaagcactttgaaaagagagtgaaaaagtacgtgaaattgttgaaagggaagggcttgcaagcagacacttaactgggccagcatcggggcggcgggaaacaaaaccaccggggaatgtacctttcgaggattataaccccggtccttactccctcaccatcccgaggcctgcaatctaaggatgctggcgtaatggttgcaagtcgc

SEQ ID NO: 15 - DNA, 499 bp, Artificial Sequence
  D1/D2 domain sequences clone H2-1
aaaccaacagggattgcctcagtaacggcgagtgaagcggcaaaagctcaaatttgaaatcccccgggaattgtaatttgaagagatttgggtccggccggcaggggttaagtccactggaaagtggcgccacagagggtgacagccccgtgaaccccttcaaagccttcatcccagatctccaagagtcgagttgtttgggaatgcagctctaagtgggtggtaaattccatctaaagctaaataccggcgagagaccgatagcgaacaagtacagtgatggaaagatgaaaagcactttgaaaagagagtgaaaaagtacgtgaaattgttgaaagggaagggcttgcaagcagacacttaactgggccagcatcggggcggcgggaaacaaaaccaccggggaatgtacctttcgaggattataaccccggtccttactcccacaccaccccgaggcctgcaatctaaggatgctggcgtaatggttgcaagtcgc

SEQ ID NO: 16 - DNA, 499 bp, Artificial Sequence
  D1/D2 domain sequences clone H2-2
aaaccaacagggattgcctcagtaacggcgagtgaagcggcaaaagctcaaatttgaaatcccccgggaattgtaatttgaagagatttgggttcggccggcaggggttaagtccactggaaagtggcgccacagagggtgacagccccgtgaaccccttcaacgccttcatcccagatctccaagagtcgagttgtttgggaatgcagctctaagtgggtggtaaattccatctaaagctaaataccggcgagagaccgatagcgaacaagtacagtgatggaaagatgaaaagcactttgaaaagagagtgaaaaagtacgtgaaattgttgaaagggaagggcttgcaagcagacacttaactgggccagcatcggggcggcgggaaacaaaaccaccggggaatgtacctctcgaggattataaccccggtctcaatttccttgttgccccgaggcctgcaatctaaggatgctggcgtaatggttgcaagtcgc

SEQ ID NO: 17 - DNA, 499 bp, Artificial Sequence
  D1/D2 domain sequences clone H2-3
aaaccaacagggattgcctcagtaacggcgagtgaagcggcaaaagctcaaatttgaaatcccccgggaattgtaatttgaagagatttgggtccggccggcaggggttaagtccactggaaagtggcgccacagagggtgacagccccgtgaaccccttcaacgccctcatcccagatctccaagagtcgagttgtttgggaatgcagctctaagtgggtggtaaattccatctaaagctaaataccggcgagagaccgatagcgaacaagtacagtgatggaaagatgaaaagcactttgaaaagagagtgaaaaagtacgtgaaattgttgaaagggaagggcttgcaagcagacacttaactgggccagcatcggggcggcgggaaacaaaaccaccggggaatgtacctttcgaggattataaccccggtccttactccctcaccatcccgaggcctgcaatctaaggatgctggcgtaatggttgcaagtcgc

SEQ ID NO: 18 - DNA, 499 bp, Artificial Sequence
  D1/D2 domain sequences clone H2-4
aaaccaacagggattgcctcagtaacggcgagtgaagcggcaaaagctcaaatttaaaatcccccgggaattgtaatttgaagagatttgggtccggccggcgggggttaagtccactggaaagtggcgccacagagggtgacagccccgtgaacccctttaacgccctcatcccagatctccaagagtcgagttgtttgggaatgcagctctaagtgggtggtaaattccatctaaagctaaataccggcgagagaccgatagcgaacaagtacagtgatggaaagatgaaaagcactttgaaaagagagtgaaaaagtacgtgaaattgttgaaagggaagggcttgcaagcagacacttaactgggccagcatcggggcggcggggagcaaaaccaccggggaatgtacctttcgaggattataaccccggcccttactcccacaccaccccgaggcctgcaatctaaggatgctggcgtaatggttgcaagtcgc

SEQ ID NO: 19 - DNA, 499 bp, Artificial Sequence
  D1/D2 domain sequences clone H2-6,  H3-7
aaaccaacagggattgcctcagtaacggcgagtgaagcggcaaaagctcaaatttgaaatcccccgggaattgtaatttgaagagatttgggtccggccggcgggggttaagtccactggaaagtggcgccacagagggtgacagccccgtgaaccccctcaacgccctcatcccagatctccaagagtcgagttgtttgggaatgcagctctaagtgggtggtaaattccatctaaagctaaataccggcgagagaccgatagcgaacaagtacagtgatggaaagatgaaaagcactttgaaaagagagtgaaaaagtacgtgaaattgttgaaagggaagggcttgcaagcagacacttaactgggccagcatcggggcggcgggaaacaaaaccaccggggaatgtacctttcgaggattataaccccggtctcaatttcctcaccaccccgaggcctgcaatctaaggatgctggcgtaatggttgcaagtcgc

SEQ ID NO: 20 - DNA, 499 bp, Artificial Sequence
  D1/D2 domain sequences clone H2-8
aaaccaacagggattgcctcagtaacggcgagtgaagcggcaaaagctcaaatttgaaatcccccgggaattgtaatttgaagagatttgggtccggccggcaggggttaagtccactggaaagtggcgccacagagggtgacagccccgtgaaccccctcaacgccctcatcccagatctccaagagtcgagttgtttgggaatgcagctctaagtgggtggtaaattccatctaaagctaaataccggcgagagaccgatagcgaacaagtacagtgatggaaagatgaaaagcactttgaaaagagagtgaaaaagtacgtgaaattgttgaaagggaagggcttgcaagcagacacttaactgggccagcatcggggcggcggggagcaaaaccaccggggaatgtacctttcgaggattataaccccggtccttttttccttgttgccccgaggcctgcaatctaaggatgctggcgtaatggttgcaagtcgc

SEQ ID NO: 21 - DNA, 499 bp, Artificial Sequence
  D1/D2 domain sequences clone H3-1,  H3-4,  H3-6
aaaccaacagggattgcctcagtaacggcgagtgaagcggcaaaagctcaaatttgaaatcccccgggaattgtaatttgaagagatttgggttcggccggcaggggttaagtccactggaaagtggcgccacagagggtgacagccccgtgaaccccttcaacgccctcatcccagatctccaagagtcgagttgtttgggaatgcagctctaagtgggtggtaaattccatctaaagctaaataccggcgagagaccgatagcgaacaagtacagtgatggaaagatgaaaagcactttgaaaagagagtgaaaaagtacgtgaaattgttgaaagggaagggcttgcaagcagacacttaactgggccagcatcggggcggcgggaaacaaaaccaccggggaatgtacctttcgaggattataaccccggtccttttttccttgttgccccgaggcctgcaatctaaggatgctggcgtaatggttgcaagtcgc

SEQ ID NO: 22 - DNA, 499 bp, Artificial Sequence
  D1/D2 domain sequences clone H3-2
aaaccaacagggattgcctcagtaacggcgagtgaagcggcaaaagctcaaatttgaaatcccccgggaattgtaatttgaagagatttgggtccggccggcaggggttaagtccactggaaagtggcgccacagagggtgacagccccgtgaaccccttcaacgccttcatcccagatctccaagagtcgagttgtttgggaatgcagctctaagtgggtggtaaattccatctaaagctaaataccggcgagagaccgatagcgaacaagtacagtgatggaaagatgaaaagcactttgaaaagagagtgaaaaagtacgtgaaattgttgaaagggaagggcttgcaagcagacacttaactgggccagcatcggggcggcgggaaacaaaaccaccggggaatgtacctctcgaggattataaccccggtctcaatttcctcaccaccccgaggcctgcaatctaaggatgctggcgtaatggttgcaagtcgc

SEQ ID NO: 23 - DNA, 499 bp, Artificial Sequence
  D1/D2 domain sequences clone H3-3
aaaccaacagggattgcctcagtaacggcgagtgaagcggcaaaagctcaaatttgaaatcccccgggaattgtaatttgaagagatttgggtccggccggcaggggttaagtccactggaaagtggcgccacagagggtgacagccccgtgaaccccttcaaagccttcatcccagatctccaagagtcgagttgtttgggaatgcagctctaagtgggtggtaaattccatctaaagctaaataccggcgagagaccgatagcgaacaagtacagtgatggaaagatgaaaagcactttgaaaagagagtgaaaaagtacgtgaaattgttgaaagggaagggcttgcaagcagacacttaactgggccagcatcggggcggcgggaaacaaaaccactggggaatgtacctttcgaggattataaccccggtccttactccctcaccatcccgaggcctgcaatctaaggatgctggcgtaatggttgcaagtcgc

SEQ ID NO: 24 - DNA, 499 bp, Artificial Sequence
  D1/D2 domain sequences clone H3-5
aaaccaacagggattgcctcagtaacggcgagtgaagcggcaaaagctcaaatttgaaatcccccgggaattgtaatttgaagagatttgggtccggccggcaggggttaagtccactggaaagtggcgccacagagggtgacagccccgtgaaccccttcaaagcttttaccccagatctccaagagtcgagttgtttgggaatgcagctctaagtgggtggtaaattccatctaaagctaaataccggcgagagaccgatagcgaacaagtacagtgatggaaagatgaaaagcactttgaaaagagagtgaaaaagtacgtgaaattgttgaaagggaagggcttgcaagcagacacttaactgggccagcatcggggcggcgggaaacaaaaccaccggggaatgtacctttcgaggattataaccccggtctcaatttccttgttgccccgaggcctgcaatctaaggatgctggcgtaatggttgcaagtcgc

SEQ ID NO: 25 - DNA, 499 bp, Artificial Sequence
  D1/D2 domain sequences clone H3-8
aaaccaacagggattgcctcagtaacggcgagtgaagcggcaaaagctcaaatttgaaatcccccgggaattgtaatttgaagagatttgggttcggccggcaggggttaagtccactggaaagtggcgccacagagggtgacagccccgtgaaccccttcaacgccctcatcccagatctccaagagtcgagttgtttgggaatgcagctctaagtgggtggtaaattccatctaaagctaaataccggcgagagaccgatagcgaacaagtacagtgatggaaagatgaaaagcactttgaaaagagagtgaaaaagtacgtgaaattgttgaaagggaagggcttgcaagcagacacttaactgggccagcatcggggcggcgggaaacaaaaccaccggggaatgtacctttcgaggattataaccccggtccttactccctcaccatcccgaggcctgcaatctaaggatgctggcgtaatggttgcaagtcgc

SEQ ID NO: 26 - DNA, 24 bp, Artificial Sequence
  primer NL1
gcatatcaataagcggaggaaaag

SEQ ID NO: 27 - DNA, 19 bp, Artificial Sequence
  primer NL4
ggtccgtgtttcaagacgg

SEQ ID NO: 28 - DNA, 17 bp, Artificial Sequence
  primer LR0R
acccgctgaacttaagc

SEQ ID NO: 29 - DNA, 17 bp, Artificial Sequence
  primer LR5
tcctgagggaaacttcg

SEQ ID NO: 30 - DNA, 22 bp, Artificial Sequence
  primer LS266
gcattcccaaacaactcgactc

SEQ ID NO: 31 - DNA, 20 bp, Artificial Sequence
  primer V9G
ttacgtccctgccctttgta

SEQ ID NO: 32 - DNA, 760 bp, Artificial Sequence
  D1/D2 domain sequence from PCR amplification
gattgcctcagtaacggcgagtgaagcggcaaaagctcaaatttgaaatcccccgggaattgtaatttgaagagatttgggtccggccggcgggggttaagtccactggaaagtggcgccacagagggtgacagccccgtgaacccctttaaagccttcatcccagatctccaagagtcgagttgtttgggaatgcagctctaagtgggtggtaaattccatctaaagctaaataccggcgagagaccgatagcgaacaagtacagtgatggaaagatgaaaagcactttgaaaagagagtgaaaaagtacgtgaaattgttgaaagggaagggcttgcaagcagacacttaactgggccagcatcggggcggcgggaaacaaaaccaccggggaatgtacctttcgaggattataaccccggtctctatttccatgctgccccgaggcctgcaatctaaggatgctggcgtaatggttgcaagtcgcccgtcttgaaacacggaccaaggagtctaacaatcatgcaagtgtttgggcccaaaacccatacgcgcaatgaaagtaaccggagcgaaccttctggtgcagctccagccacaccgagacccaaatcccggtgtgagcaagcatggctgttgggacccgaaagatggtgaactatacctggatagggtgaagccagaggaaactctggtggaggctcgtagcggttctgacgtgcaaatcgatcgtcgaatctgggtataggggcgaaagac

SEQ ID NO: 33 - DNA, 640 bp, Artificial Sequence
  ITS sequence from PCR amplification
cttagtgaggcctctggattgaatctagggccggggcgacccggccgtgggttgagaaactggtcaaacttggtcatttagaggaagtaaaagtcgtaacaaggtttccgtaggtgaacctgcggaaggatcattaaaaatattattacacacttttaggaaaaacctctgaaccttttttttcatatacacttttaaaaaactttcaacaacggatctcttggttctcgcatcgatgaagaacgcagcgaattgcgatacgtaatatgacttgcagacgtgaatcattgaatctttgaacgcacattgcgccccggggtattccccagggcatgcgtgggtgagcgatatttactctcaaacctccggtttggtcctgcttcggcctaatatcaacggcgctagaataagttttagccccattctttttcctcaccctcgtaagactacccgctgaacttaagcatatcaataagcggaggaaaagaaaccaacagggattgcctcagtaacggcgagtgaagcggcaaaagctcaaatttgaaatcccccgggaattgtaatttgaagagatttgggtccggccggcgggggttaagtccactggaaagtggcgccacagagggtgacagccccgtga

SEQ ID NO: 34 - DNA, 19 bp, Artificial Sequence
  primer ITS1
tccgtaggtgaacctgcgg

SEQ ID NO: 35 - protein, 360 aa, Metschnikowia
  Act1 protein from H0 Metschnikowia sp.
MCKAGFAGDDAPRAVFPSIVGRPRHQGIMVGMGQKDSYVGDEAQSKRGILTLRYPIEHGIVNNWDDMEKIWHHTFYNELRVAPEEHPVLLTEAPMNPKSNREKMTQIMFETFNVPAFYVSIQAVLSLYSSGRTTGIVLDSGDGVTHLVPIYAGFSMPHGILRLNLAGRDLTDYLMKILSERGYTFSTTAEREIVRDIKEKLCYVALDFEQEMQTSSQSSAIEKSYELPDGQVITIGNERFRAAEALFRPTDLGLEAVGIDQTTYNSIIKCDVDVRKELYGNIVMSGGTTLFPGIAERMQKEITALAPSSMKVKIIAPPERKYSVWIGGSILASLSTFQQMWISKQEYDESGPTIVHHKCF

SEQ ID NO: 36 - protein, 491 aa, Metschnikowia
  Aro8 protein from H0 Metschnikowia sp
MTKPLAKDLQHHLSTEAKSRKGSALKGAFKYYNQPGMTFLGGGLPLSDYFPFDKITADVPSAPFPNGCGARVTESDKTVIEVHKRKQDNSDSGYADVELARSLQYGYTEGHTELVQFLRDHTDTIHRVPYEDWDVITNVGNTQAWDAVLRTFTSRGDVILVEDHTFSSAMETAHAHGVTTYPVVMDTEGIVPSALEKLLDNWVGAKPRMLYTICTGQNPTGSCLSGERRREVYSLAQKHDLIIIEDEPYYFLQMEPYTRDLALRSSKHVHGHEEFIKALVPSFISMDVDGRVLRLDSVSKTIAPGARLGWVVGQKRLLERFLRLHETSIQNASGFTQSLLNGLFQRWGQKGYLDWLIGIRAEYTHKRDVAIDALYKYFPQEVVTILPPVAGMFFVVNLDASKHPKFEELGSDPLAVENSLYEAGLAHGCLMIPGSWFKADGETTPPQAPVPVDESLKNSIFFRGTYAAVPLDELEVGLKKFGEAVKAEFGL

SEQ ID NO: 37 - protein, 615 aa, Metschnikowia
  Aro10 protein from H0 Metschnikowia
MAPIITRASSEETTPQITDDQIPLGEYLFLRICQANPKLRSVFGIPGDFSLALLEHLYTKSVAKKVEFVGFCNELNAAYAADGYAKHIDGLSVLLTTFGVGELSTLNAIAGAFTEYAPVLHIVGTTSTKQAEQSRAAGTRDVRNIHHLVQNKNPLCAPNHDVYKPMVESLSVCQESLDMNGDLNLEKIDNVLRMVTNERRPGYIFIPSDVSDIMVSAGRLNQPLTFSELTDESALKNMASRILAKLYNSKHPSVLGDALADRFGGQTALDNLVEKLPSNFVKLFSTLLARNIDETLPNYIGVYSGKLSSDKIVIDELERNTDFLLTLGHANNEINSGVYSTDFSAITEYVEVHPDYILIDGEYVLIKNAETGKRLFSIVDLLTKLVSDFDASKMIHNNHAVNNIRARRETKQFSSLDTVSPGVITQNKLVDFFNDYLRPNDILLCDTCSFLFGVFELKFPRGVKFIAQTLYESIGYALPATFGAARAERDLGTNRRVVLIQGDGSAQMTIQEWSTYLRYDISSPEIFLLNNEGYTVERMIKGPTRSYNDIQDTWKWTEFFKIFGDEDCEKHEAEKVNTTNELEALTRRKTSEKIRLYELKLSKLDIVDKFRILRE

SEQ ID NO: 38 - protein, 370 aa, Metschnikowia
  Gpd1 protein from H0 Metschnikowia
MTATAPFKIESPFRIAIIGSGNWGTAVAKLVAENTAEKPEIFQKQVNMWVFEEDINGRKLTEIINTDHENVKYMPEVKLPENLVANPDIEATVKDADLLIFNIPHQFLPRVCKQLVGKVSPTARAISCLKGLEVDASGCKLLSQSITDTLGIYCGVLSGANIANEVARGRWSETSIAYNRPTDFRGEGKDICEFVLKEAFHRRYFHVRVIKDVIGASIAGALKNVVAIAAGFVEGEGWGDNAKSAIMRIGLKETIHFASYWEKFGIQGLSAPEPTTFTEESAGVADLITTCSGGRNVKVARYMIEKNVDAWEAEKALLNGQSSQGIITAKEVHELLVNYKLQEEFPLFEATYAVIYENADVNTWPTILAE

SEQ ID NO: 39 - protein, 544 aa, Metschnikowia
  Gxf1 protein from H0 Metschnikowia
MSQDELHTKSGVETPINDSLLEEKHDVTPLAALPEKSFKDYISISIFCLFVAFGGFVFGFDTGTISGFVNMSDFKTRFGEMNAQGEYYLSNVRTGLMVSIFNVGCAVGGIFLCKIADVYGRRIGLMFSMVVYVVGIIIQIASTTKWYQYFIGRLIAGLAVGTVSVISPLFISEVAPKQLRGTLVCCFQLCITLGIFLGYCTTYGTKTYTDSRQWRIPLGICFAWALFLVAGMLNMPESPRYLVEKSRIDDARKSIARSNKVSEEDPAVYTEVQLIQAGIDREALAGSATWMELVTGKPKIFRRVIMGVMLQSLQQLTGDNYFFYYGTTIFKAVGLQDSFQTSIILGIVNFASTFVGIYAIERMGRRLCLLTGSACMFVCFIIYSLIGTQHLYKNGFSNEPSNTYKPSGNAMIFITCLYIFFFASTWAGGVYCIVSESYPLRIRSKAMSVATAANWMWGFLISFFTPFITSAIHFYYGFVFTGCLAFSFFYVYFFVVETKGLSLEEVDILYASGTLPWKSSGWVPPTADEMAHNAFDNKPTDEQV

SEQ ID NO: 40 - protein, 548 aa, Metschnikowia
  Gxf2 protein from H0 Metschnikowia
MSAEQEQQVSGTSATIDGSASLKQEKTAEEEDAFKPKPATAYFFISFLCGLVAFGGYVFGFDTGTISGFVNMDDYLMRFGQQHADGTYYLSNVRTGLIVSIFNIGCAVGGLALSKVGDIWGRRIGIMVAMIIYMVGIIIQIASQDKWYQYFIGRLITGLGVGTTSVLSPLFISESAPKHLRGTLVCCFQLMVTLGIFLGYCTTYGTKNYTDSRQWRIPLGLCFAWALLLISGMVFMPESPRFLIERQRFDEAKASVAKSNQVSTEDPAVYTEVELIQAGIDREALAGSAGWKELITGKPKMLQRVILGMMLQSIQQLTGNNYFFYYGTTIFKAVGMSDSFQTSIVLGIVNFASTFVGIWAIERMGRRSCLLVGSACMSVCFLIYSILGSVNLYIDGYENTPSNTRKPTGNAMIFITCLFIFFFASTWAGGVYSIVSETYPLRIRSKGMAVATAANWMWGFLISFFTPFITSAIHFYYGFVFTGCLIFSFFYVFFFVRETKGLSLEEVDELYATDLPPWKTAGWTPPSAEDMAHTTGFAEAAKPTNKHV

SEQ ID NO: 41 - protein, 526 aa, Metschnikowia
  Gxs1 protein from H0 Metschnikowia
MGLESNKLIRKYINVGEKRAGSSGMGIFVGVFAALGGVLFGYDTGTISGVMAMPWVKEHFPKDRVAFSASESSLIVSILSAGTFFGAILAPLLTDTLGRRWCIIISSLVVFNLGAALQTAATDIPLLIVGRVIAGLGVGLISSTIPLYQSEALPKWIRGAVVSCYQWAITIGIFLAAVINQGTHKINSPASYRIPLGIQMAWGLILGVGMFFLPETPRFYISKGQNAKAAVSLARLRKLPQDHPELLEELEDIQAAYEFETVHGKSSWSQVFTNKNKQLKKLATGVCLQAFQQLTGVNFIFYFGTTFFNSVGLDGFTTSLATNIVNVGSTIPGILGVEIFGRRKVLLTGAAGMCLSQFIVAIVGVATDSKAANQVLIAFCCIFIAFFAATWGPTAWVVCGEIFPLRTRAKSIAMCAASNWLLNWAIAYATPYLVDSDKGNLGTNVFFIWGSCNFFCLVFAYFMIYETKGLSLEQVDELYEKVASARKSPGFVPSEHAFREHADVETAMPDNFNLKAEAISVEDASV

SEQ ID NO: 42 - protein, 628 aa, Metschnikowia
  Hgt19 protein from H0 Metschnikowia
MSEKPVVSHSIDTTSSTSSKQVYDGNSLLKTSNERDGERGNILSQYTEEQAMQMGRNYALKHNLDATLFGKAAAVARNPYEFNSMSFLTEEEKVALNTEQTKKWHIPRKLVEVIALGSMAAAVQGMDESVVNGATLFYPTAMGITDIKNADLIEGLINGAPYLCCAIMCWTSDYWNRKLGRKWTIFWTCAISAITCIWQGLVNLKWYHLFIARFCLGFGIGVKSATVPAYAAETTPAKIRGSLVMLWQFFTAVGIMLGYVASLAFYYIGDNGISGGLNWRLMLGSACLPAIVVLVQVPFVPESPRWLMGKERHAEAYDSLRQLRFSEIEAARDCFYQYVLLKEEGSYGTQPFFSRIKEMFTVRRNRNGALGAWIVMFMQQFCGINVIAYYSSSIFVESNLSEIKAMLASWGFGMINFLFAIPAFYTIDTFGRRNLLLTTFPLMAVFLLMAGFGFWIPFETNPHGRLAVITIGIYLFACVYSAGEGPVPFTYSAEAFPLYIRDLGMGFATATCWFFNFILAFSWPRMKNAFKPQGAFGWYAAWNIVGFFLVLWFLPETKGLTLEELDEVFDVPLRKHAHYRTKELVYNLRKYFLRQNPKPLPPLYAHQRMAVTNPEWLEKTEVTHEENI

SEQ ID NO: 43 - protein, 524 aa, Metschnikowia
  Hxt2.6 protein from H0 Metschnikowia
MSSTTDTLEKRDTEPFTSDAPVTVHDYIAEERPWWKVPHLRVLTWSVFVITLTSTNNGYDGSMLNGLQSLDIWQEDLGHPAGQKLGALANGVLFGNLAAVPFASYFCDRFGRRPVICFGQILTIVGAVLQGLSNSYGFFLGSRIVLGFGAMIATIPSPTLISEIAYPTHRETSTFAYNVCWYLGAIIASWVTYGTRDLQSKACWSIPSYLQAALPFFQVCMIWFVPESPRFLVAKGKIDQARAVLSKYHTGDSTDPRDVALVDFELHEIESALEQEKLNTRSSYFDFFKKRNFRKRGFLCVMVGVAMQLSGNGLVSYYLSKVLDSIGITETKRQLEINGCLMIYNFVICVSLMSVCRMFKRRVLFLTCFSGMTVCYTIWTILSALNEQRHFEDKGLANGVLAMIFFYYFFYNVGINGLPFLYITEILPYSHRAKGLNLFQFSQFLTQIYNGYVNPIAMDAISWKYYIVYCCILFVELVIVFFTFPETSGYTLEEVAQVFGDEAPGLHNRQLDVAKESLEHVEHV

SEQ ID NO: 44 - protein, 548 aa, Metschnikowia
  Hxt5 protein from H0 Metschnikowia
MSIFEGKDGKGVSSTESLSNDVRYDNMEKVDQDVLRHNFNFDKEFEELEIEAAQVNDKPSFVDRILSLEYKLHFENKNHMVWLLGAFAAAAGLLSGLDQSIISGASIGMNKALNLTEREASLVSSLMPLGAMAGSMIMTPLNEWFGRKSSLIISCIWYTIGSALCAGARDHHMMYAGRFILGVGVGIEGGCVGIYISESVPANVRGSIVSMYQFNIALGEVLGYAVAAIFYTVHGGWRFMVGSSLVFSTILFAGLFFLPESPRWLVHKGRNGMAYDVWKRLRDINDESAKLEFLEMRQAAYQERERRSQESLFSSWGELFTIARNRRALTYSVIMITLGQLTGVNAVMYYMSTLMGAIGFNEKDSVFMSLVGGGSLLIGTIPAILWMDRFGRRVWGYNLVGFFVGLVLVGVGYRFNPVTQKAASEGVYLTGLIVYFLFFGSYSTLTWVIPSESFDLRTRSLGMTICSTFLYLWSFTVTYNFTKMSAAFTYTGLTLGFYGGIAFLGLIYQVCFMPETKDKTLEEIDDIFNRSAFSIARENISNLKKGIW

SEQ ID NO: 45 - protein, 417 aa, Metschnikowia
  Pgk1 protein from H0 Metschnikowia
MSLSNKLSVKDLDLANKRVFIRVDFNVPLDGTTITNNQRIVAALPTIKYVLEQKPKAVILASHLGRPNGERVEKYSLAPVAKELQSLLSDQKVTFLNDSVGPEVEKAVNSASQGEVFLLENLRYHIEEEGSKKVDGNKVKASKEDVEKFRQGLTALADVYVNDAFGTAHRAHSSMVGLELPQKAAGFLMAKELEYFAKALENPTRPFLAILGGAKVSDKIQLIDNLLDKVDILIVGGGMAFTFKKVLDNMPIGTSLFDEAGSKNVENLIAKAKKNNVEIVLPVDFVTADDFNKDANTGVATQEEGIPDGWMGLDAGPKSRELFAEAVAKAKTIVWNGPPGVFEFEKFAQGTKSLLDAAVKSAEAGNTVIIGGGDTATVAKKFGVVEKLSHVSTGGGASLELLEGKELPGVVAISDKQ

SEQ ID NO: 46 - protein, 563 aa, Metschnikowia
  Qup2 protein from H0 Metschnikowia
MGFRNLKRRLSNVGDSMSVHSVKEEEDFSRVEIPDEIYNYKIVLVALTAASAAIIIGYDAGFIGGTVSLTAFKSEFGLDKMSATAASAIEANVVSVFQAGAYFGCLFFYPIGEIWGRKIGLLLSGFLLTFGAAISLISNSSRGLGAIYAGRVLTGLGIGGCSSLAPIYVSEIAPAAIRGKLVGCWEVSWQVGGIVGYWINYGVLQTLPISSQQWIIPFAVQLIPSGLFWGLCLLIPESPRFLVSKGKIDKARKNLAYLRGLSEDHPYSVFELENISKAIEENFEQTGRGFFDPLKALFFSKKMLYRLLLSTSMFMMQNGYGINAVTYYSPTIFKSLGVQGSNAGLLSTGIFGLLKGAASVFWVFFLVDTFGRRFCLCYLSLPCSICMWYIGAYIKIANPSAKLAAGDTATTPAGTAAKAMLYIWTIFYGITWNGTTWVICAEIFPQSVRTAAQAVNASSNWFWAFMIGHFTGQALENIGYGYYFLFAACSAIFPVVVWFVYPETKGVPLEAVEYLFEVRPWKAHSYALEKYQIEYNEGEFHQHKPEVLLQGSENSDTSEKSLA

SEQ ID NO: 47 - protein, 1718 aa, Metschnikowia
  Rpb1 protein from H0 Metschnikowia
MDQTTKKPRDGGLNDPRLGSIDRNFKCQTCGEDMAECPGHFGHIELAKPVFHIGFIAKIKKVCECVCMHCGKLLVDDANPLMAQAIRIRDPKKRFNAVWNVSKTKMVCEADTINEEGQVTAGRGGCGHTQPTVRRDGLKLWGTWKQNKTYDENEQPERRLLSPSEILSVFRHISPEDCHKLGFNEDYARPEWMLITVLPVPPPPVRPSIAFNDTARGEDDLTFKLADILKANINVQRLEIDGSPQHVISEFEALLQFHVATYMDNDIAGQPQALQKTGRPIKSIRARLKGKEGRLRGNLMGKRVDFSARTVISGDPNLDLDQVGVPISIARTLTYPEVVTPYNIHKLTEYVRNGPNEHPGAKYVIRDTGDRIDLMYNKRAGDIALQYGWKVERHLMDDDPVLFNRQPSLHKMSMMAHRVKVMPYSTFRLNLSVTSPYNADFDGDEMNLHVPQSPETRAEMSQICAVPLQIVSPQSNKPVMGIVQDTLCGIRKMTLRDNFIEYEQVMNMLYWIPNWDGVIPPPAVLKPKPLWSGKQLLSMAIPKGIHLQRFDDGRDMLSPKDSGMLIVDGEIIFGVVDKKTVGATGGGLIHTVMREKGPYVCAQLFSSIQKVVNYWLLHNGFSIGIGDTIADKDTMRDVTTTIQEAKQKVQEIIIDAQQNKLEPEPGMTLRESFEHNVSRILNQARDTAGRSAEMNLKDSNNVKQMVTSGSKGSFINISQMSACVGQQIVEGKRIPFGFGDRTLPHFTKDDYSPESKGFVENSYLRGLTPQEFFFHAMAGREGLIDTAVKTAETGYIQRRLVKALEDIMVHYDGTTRNSLGDIIQFVYGEDGIDATSVEKQSVDTIPGSDSSFEKRYRIDVLDPAKSIPESLLESGKQIKGDVAVQKVLDEEYDQLLKDRKFLREVVFPNGDYNWPLPVNLRRIIQNAQQIFHSGRQKASDLRLEEIVEGVQSLCTKLLVLRGKTELIKEAQENATLLFQCLLRSRLAARRVIEEFKLNKVSFEWVCGEIESQFQKSIVHPGEMVGVVAAQSIGEPATQMTLNTFHYAGVSSKNVTLGVPRLKEILNVAKNIKTPALTVYLEPEIAVDIEKAKVVQSAIEHTTLKNVTSSTEIYYDPDPRSTVIEEDYDTVEAYFAIPDEKVEETIDNQSPWLLRLELDRAKMLDKQLTMAQVAEKISQNFGEDLFVIWSDDTADKLIIRCRVIRDPKLEEEGEHEEDQILKRVEAHMLETISLRGIPGITRVFMMQHKMSTPDADGEFSQKQEWVLETDGVNLAEVITVPGVDASRTYSNNFIEILSVLGIEATRTALFKEILNVIAFDGSYVNYRHMALLVDVMTARGHLMAITRHGINRAETGALMRCSFEETVEILLDAGAAAELDDCRGISENVILGQMPPLGTGAFDVMVDEKMLQDASVSSDIGVAGQTDGGATPYRDYEMEDDKIQFEEGAGFSPIHTANVSDASGSLTSYGGQPSMVSPTSPFSFGATSPGYGGVTSPAYGATSPTYSPTSPTYSPTSPSYSPTSPSYSPTSPSYSPTSPSYSPTSPSYSPTSPSYSPTSPSYSPTSPSYSPTSPSYSPTSPSYSPTSPSYSPTSPSYSPTSPSYSPTSPSYSPTSPSYSPTSPSYSPTSPSYSPTSPSYSPTSPQYSPTSPSYSPTSPQYSPTSPSYSPTSPQYSPTSPSYSPTSPQYSPTSPQYSPGSPAYSPGSPSYSTEKKDEDKK

SEQ ID NO: 48 - protein, 1233 aa, Metschnikowia
  Rpb2 protein from H0 Metschnikowia
MSQEPVEDPYVYDEEDAHSITPEDCWTVISSFFQEKGLVSQQLDSFDEFIESNIQELVWEDSHLILDQPAQHTSEDQYENKRFEITFGKIYISKPTQTEGDGTTHPMFPQEARLRNLTYSSPLYVDMSKKKFLSDDRVRKGNELEWVEEKVDGEEAQSKVFLGKVPIMLRSKFCMLRDLGEHEFYELKECPYDMGGYFVINGSEKVLIAQERSAANIVQVFKKAAPSPISHVAEIRSALEKGSRLISSMQIKLYGRDDKGTTGRTIKATLPYIKEDIPIVIVFRALGVVPDGDILEHICYDANDWQMLEMLKPCVEEGFVIQEREVALDFIGRRGVLGIRREKRIQYAKDILQKELLPNITQEAGFESRKAFFLGYMVNRLLLCALERKEPDDRDHFGKKRLDLAGPLLASLFRLLFKKLTRDIYNYMQRCVENDKEFNLTLAVKSQTITDGLRYSLATGNWGEQRKAMSARAGVSQVLNRYTYSSTLSHLRRTNTPIGRDGKIAKPRQLHNTHWGLVCPAETPEGQACGLVKNLSLMTCISVGTSSEPILYFLEEWGMEPLEDYVPSNAPDCTRVFVNGVWVGTHREPAQLVDTMRRLRRKGDISPEVSIIRDIREMEFKIFTDAGRVYRPLFIVDDDPESETKGELMLQKEHVHKLLNSAYDEYDEDDSNAYTWSSLVNDGVVEYVDAEEEETIMIAMTPEDLEASKSALSETQQQDLQMEEQELDPAKRIKPTYTSSTHTFTHCEIHPSMILGVAASIIPFPDHNQSPRNTYQSAMGKQAMGVFLTNYAVRMDTMANILYYPQKPLATTRAMEHLKFRELPAGQNAVVAIACYSGYNQEDSMIMNQSSIDRGLFRSLFFRSYMDLEKRQGMKALETFEKPSRSDTLRLKHGTYEKLDDDGLIAPGVRVSGEDIIIGKTTPIPPDTEELGQRTQYHTKRDASTPLRSTESGIVDQVLLTTNGDGAKFVKVRMRTTKVPQIGDKFASRHGQKGTIGVTYRHEDMPFSAQGIVPDLIINPHAIPSRMTVAHLIECLLSKVSSLSGLEGDASPFTDVTAEAVSKLLREHGYQSRGFEVMYNGHTGKKMMAQVFFGPTYYQRLRHMVDDKIHARARGPVQVLTRQPVEGRSRDGGLRFGEMERDCMIAHGAAGFLKERLMEASDAFRVHVCGICGLMSVIANLKKNQFECRSCKNKTNIYQIHIPYAAKLLFQELMAMNISPRLYTERSGISVRV

SEQ ID NO: 49 - protein, 458 aa, Metschnikowia
  Tef1 protein from H0 Metschnikowia
MGKEKSHVNVVVIGHVDSGKSTTTGHLIYKCGGIDKRTIEKFEKEAAELGKGSFKYAWVLDKLKAERERGITIDIALWKFETPKYHVTVIDAPGHRDFIKNMITGTSQADCAILIIAGGVGEFEAGISKDGQTREHALLAYTLGVRQLIVAVNKMDSVKWDKNRFEEIIKETSNFVKKVGYNPKTVPFVPISGWNGDNMIEASTNCPWYKGWEKETKAGKSSGKTLLEAIDAIEPPTRPTDKALRLPLQDVYKIGGIGTVPVGRVETGVIKAGMVVTFAPAGVTTEVKSVEMHHEQLVEGLPGDNVGFNVKNVSVKEIRRGNVCGDSKQDPPKAAASFTAQVIVLNHPGQISSGYSPVLDCHTAHIACKFDTLLEKIDRRTGKSLESEPKFVKSGDAAIVKMVPTKPMCVEAFTDYPPLGRFAVRDMRQTVAVGVIKAVEKSDKAGKVTKAAQKAAKK

SEQ ID NO: 50 - protein, 248 aa, Metschnikowia
  Tpi1 protein from H0 Metschnikowia
MARQFFVGGNFKMNGTKESLTAIVDTLNKADLPENVEVVIAPPAPYLSLVVEANKQKTVEVAAQNVFSKASGAYTGEIAPQQLKDLGANWTLTGHSERRTIIKESDEFIAEKTKFALESGVSVILCIGETLEEKKAGITLEVCARQLDAVSKIVSDWTNVVIAYEPVWAIGTGLAATAQDAQDIHKEIRAHLSKTIGAEQAEAVRILYGGSVNGKNAVDFKDKADVDGFLVGGASLKPEFIDIIKSRL

SEQ ID NO: 51 - protein, 609 aa, Metschnikowia
  Xks1 protein from H0 Metschnikowia
MTYSSSSGLFLGFDLSTQQLKIIVTNENLKALGTYHVEFDAQFKEKYAIKKGVLSDEKTGEILSPVHMWLEAIDHVFGLMKKDNFPFGKVKGISGSGMQHGSVFWSKSASSSLKNMAEYSSLTEALADAFACDTSPNWQDHSTGKEIKDFEKVVGGPDKLAEITGSRAHYRFTGLQIRKLAVRSENDVYQKTDRISLVSSFVASVLLGRITTIEEADACGMNLYNVTESKLDEDLLAIAAGVHPKLDNKSKRETDEGVKELKRKIGEIKPVSYQTSGSIAPYFVEKYGFSPDSKIVSFTGDNLATIISLPLRKNDVLVSLGTSTTVLLVTESYAPSSQYHLFKHPTIKNAYMGMICYSNGALARERVRDAINEKYGVAGDSWDKFNEILDRSGDFNNKLGVYFPIGEIVPNAPAQTKRMEMNSHEDVKEIEKWDLENDVTSIVESQTVSCRVRAGPMLSGSGDSNEGTPENENRKVKTLIDDLHSKFGEIYTDGKPQSYESLTSRPRNIYFVGGASRNKSIIHKMASIMGATEGNFQVEIPNACALGGAYKASWSLECESRQKWVHFNDYLNEKYDFDDVDEFKVDDKWLNYIPAIGLLSKLESNLDQN

SEQ ID NO: 52 - protein, 318 aa, Metschnikowia
  Xyl1 protein from H0 Metschnikowia
MATIKLNSGYDMPQVGFGCWKVTNSTCADTIYNAIKVGYRLFDGAEDYGNEKEVGEGINRAIDEGLVARDELFVVSKLWNNFHHPDNVEKALDKTLGDLNVEYLDLFLIHFPIAFKFVPFEEKYPPGFYCGEGDKFIYEDVPLLDTWRALEKFVKKGKIRSIGISNFSGALIQDLLRGAEIPPAVLQIEHHPYLQQPRLIEYVQSKGIAITAYSSFGPQSFVELDHPKVKECVTLFEHEDIVSIAKAHDKSAGQVLLRWATQRGLAVIPKSNKTERLLSNLNVNDFDLSEAELEQIAKLDVGLRFNNPWDWDKIPIFH

SEQ ID NO: 53 - protein, 362 aa, Metschnikowia
  Xyl2 protein from H0 Metschnikowia
MPANPSLVLNKVNDITFENYEVPLLTDPNDVLVQVKKTGICGSDIHYYTHGRIGDFVLTKPMVLGHESAGVVVEVGKGVTDLKVGDKVAIEPGVPSRTSDEYKSGHYNLCPHMCFAATPNSNPDEPNPPGTLCKYYKSPADFLVKLPEHVSLELGAMVEPLTVGVHASRLGRVTFGDHVVVFGAGPVGILAAAVARKFGAASVTIVDIFDSKLELAKSIGAATHTFNSMTEGVLSEALPAGVRPDVVLECTGAEICVQQGVLALKAGGRHVQVGNAGSYLKFPITEFVTKELTLFGSFRYGYNDYKTSVAILDENYKNGKENALVDFEALITHRFPFKNAIEAYDAVRAGDGAVKCIIDGPE

SEQ ID NO: 54 - protein, 543 aa, Metschnikowia
  Xyt1 protein from H0 Metschnikowia
MGYEEKLVAPALKFKNFLDKTPNIHNVYVIAAISCTSGMMFGFDISSMSVFVDQQPYLKMFDNPSSVIQGFITASMSLGSFFGSLTSTFISEPFGRRASLFICGILWVIGAAVQSSSQNRAQLICGRIIAGWGIGFGSSVAPVYGSEMAPRKIRGTIGGIFQFSVTVGIFIMFLIGYGCSFIQGKASFRIPWGVQMVPGLILLIGLFFIPESPRWLAKQGYWEDAEIIVANVQAKGNRNDANVQIEMSEIKDQLMLDEHLKEFTYADLFTKKYRQRTITAIFAQIWQQLTGMNVMMYYIVYIFQMAGYSGNTNLVPSLIQYIINMAVTVPALFCLDLLGRRTILLAGAAFMMAWQFGVAGILATYSEPAYISDTVRITIPDDHKSAAKGVIACCYLFVCSFAFSWGVGIWVYCSEVWGDSQSRQRGAALATSANWIFNFAIAMFTPSSFKNITWKTYIIYATFCACMFIHVFFFFPETKGKRLEEIGQLWDEGVPAWRSAKWQPTVPLASDAELAHKMDVAHAEHADLLATHSPSSDEKTGTV

SEQ ID NO: 55 - protein, 323 aa, Metschnikowia
  Tal1 protein from  H0 Metschnikowia
MSNSLESLKATGTVIVTDTGEFDSIAKYTPQDATTNPSLILAASKKAEYAKVIDVAIKYAEDKGSNPKEKAAIALDRLLVEFGKEILSIVPGRVSTEVDARLSFDKDATVKKALEIIELYKSIGISKDRVLIKIASTWEGIQAAKELEAKHDIHCNLTLLFSFVQAVACAEAKVTLISPFVGRILDWYKASTGKEYDAESDPGVVSVRQIYNYYKKYGYNTIVMGASFRNTGEIKALAGCDYLTVAPKLLEELMNSSEEVPKVLDAASASSASEEKVSYIDDESEFRFLLNEDAMATEKLAQGIRGFAKDAQTLLAELENRFK

SEQ ID NO: 56 - protein, 677 aa, Metschnikowia
  Tkl1 protein from  H0 Metschnikowia
MSDIDQLAISTIRLLAVDAVAKANSGHPGAPLGLAPAAHAVWKEMKFNPKNPDWVNRDRFVLSNGHACALLYAMLHLYGFDMSLDDLKQFRQLNSKTPGHPEKFEIPGAEVTTGPLGQGISNAVGLAIAQKQFAATFNKDDFAISDSYTYAFLGDGCLMEGVASEASSLAGHLQLNNLIAFWDDNKISIDGSTEVAFTEDVLKRYEAYGWDTLTIEKGDTDLEGVAQAIKTAKASKKPTLIRLTTIIGYGSLQQGTHGVHGAPLKPDDIKQLKEKFGFDPTKSFVVPQEVYDYYGTLVKKNQELESEWNKTVESYIQKFPEEGAVLARRLKGELPEDWAKCLPTYTADDKPLATRKLSEMALIKILDVVPELIGGSADLTGSNLTRAPDMVDFQPPQTGLGNYAGRYIRYGVREHGMGAIMNGIAGFGAGFRNYGGTFLNFVSYAAGAVRLSALSHLPVIWVATHDSIGLGEDGPTHQPIETLAHFRATPNISVWRPADGNEVSAAYKSAIESTSTPHILALTRQNLPQLAGSSVEKASTGGYTVYQTTDKPAVIIVASGSEVAISIDAAKKLEGEGIKANVVSLVDFHTFDKQPLDYRLSVLPDGVPIMSVEVMSSFGWSKYSHEQFGLNRFGASGKAEDLYKFFDFTPEGVADRAAKTVQFYKGKDLLSPLNRAF

SEQ ID NO: 57 - DNA, 1083 bp, Metschnikowia
  ACT1 gene from H0 Metschnikowia
atgtgcaaagccggttttgccggtgacgacgcacctcgtgctgtgttcccatctatcgtgggtagaccaagacaccagggtatcatggtcggcatgggtcaaaaggactcttatgttggtgacgaggcccagtccaagagaggtattttgactttgagataccccattgagcatggtatcgtgaacaactgggacgacatggagaagatctggcatcacaccttctacaacgagttgagagtcgcccctgaggaacacccagtcttgttgaccgaggctccaatgaaccctaagtccaacagagagaagatgactcaaatcatgttcgagactttcaacgttccggctttctacgtttccatccaggccgtcttgtccttgtactcctccggtagaaccactggtattgttttagattctggtgacggtgttactcacttggttcctatctatgctggattctccatgcctcacggtattttgagattgaacttggctggtagagacttgaccgactacttgatgaagattttgtccgagcgtggttacactttctccaccactgccgagagagaaattgtccgtgacatcaaggagaaattgtgctacgtcgccttggactttgagcaggagatgcaaacgtcttctcaatcttccgctatcgagaaatcgtacgagttgccagatggacaagtcatcactattggtaacgagagatttagagctgccgaggccttgttccgtcctactgacttgggcttggaggctgttggtatcgaccaaaccacttacaactctatcatcaagtgtgacgtcgacgttagaaaggagttgtacggtaacattgttatgtccggtggtactactttattcccaggtattgctgagcgtatgcaaaaggagattaccgcgttggctccttcctccatgaaggtcaagattattgctccacctgagagaaagtactctgtatggattggtggctccatcttggcttccttgtccactttccaacagatgtggatctcgaagcaagagtacgacgagtctggaccaactatcgttcaccacaagtgtttttaa

SEQ ID NO: 58 - DNA, 1476 bp, Metschnikowia
  ARO8 gene from H0 Metschnikowia
atgactaaaccacttgctaaggatttgcagcaccacttgagcacggaggccaagtcacgcaagggcctggcgcttaagggcgcattcaagtactacaaccagcccgggatgacgtttctcggcggcggattgccccttctggactatttcccctttgataaaatcactgcggacgtgccgctggcgccgttcccaaacggatgtggtgcgagagtcaccgaatcagacaaaaccgtgattgaggtgcataagcggaaacaagacaacagtgacagcggctacgcggacgttgagttggcgcgtagtttgcagtacggatacacggagggacacactgagcttgtgcagttcttacgtgaccacaccgacacgatccaccgcgtgccatatgaagattgggacgtgatcaccaatgtgggcaacacgcaagcgtgggacgccgtgttgcggacgtttacgctgcgtggtgacgtgatcttggtggaagaccacaccttttcgctggccatggagaccgcgcacgcgcacggcgtcaccacttatcccgtggtgatggacaccgagggaatcgtgccatcggcgttggagaaactcttggacaactgggttggcgcaaagccgcgcatgctctacacgatctgcacgggacagaacccaactggatcgtgtctcagtggggaacgccgccgcgaggtgtatctgttggcacagaaacatgatttgatcatcatcgaggacgagccgtactacttcttgcagatggagccatatacacgtgatttggcgcttcgcctgctgaagcacgtgcacggccatgaggagttcatcaaggcgcttgttccctcgttcatctcgatggacgtggacggacgtgtgctccgactcgactccgtgtcgaagacgatcgctccaggcgcccgtttgggctgggtcgtggggcagaaacgcctcttggagcgattcttgcgtttgcacgaaacgtcgatccagaacgcttcgggtttcacgcagctgctcttgaacggcttgtttcaaagatggggccagaagggatacttggactggttgattggtatccgtgctgagtacactcacaagagggacgtggcaattgatgctttatacaagtacttcccgcaagaagtagtgacgattttgccgcccgtggccggtatgttctttgttgtcaacttggacgccagcaagcacccgaaatttgaggagttgggcagcgacccgttggctgtcgagaacagcctctacgaggctggcttggcgcacgggtgcttgatgattcctggctcgtggttcaaggctgacggcgagaccaccccgccacaagcgcctgtgcctgtggacgagctgttgaagaacagcattttctttaggggtacttacgcggcagtacccttggacgagttggaggttggcttgaagaagtttggcgaggctgtcaaggccgagtttggtttgtaa

SEQ ID NO: 59 - DNA, 1848 bp, Metschnikowia
  ARO10 gene from H0 Metschnikowia
atggcaccaatcatcaccagggcttcatccgaagaaacaacaccccaaattacagacgaccagatccctttgggggagtaccttttcctcagaatctgccaggcaaatccaaaacttcgctcggtgtttggcattcccggagacttcagtttggcgttattggagcatctctataccaagctggtggcgaaaaaagttgagtttgttggtttctgtaacgagctcaatgcggcatatgcagcagatggatatgcaaagcatattgacggcttgagtgtcttgcttacgacttttggggtgggagaactatccactttgaacgccatagccggcgcattcacagagtacgctccagtattgcatattgtcggcaccacatctacgaaacaggcggagcagtccagggcggcaggcacgagagatgtaagaaacatccatcacttggtgcagaacaaaaacccgctttgtgcgcccaatcacgatgtatataagcccatggtggaaagtttatctgtatgccaggaatccttggacatgaatggcgacttgaacttggaaaagatcgataacgtcttgagaatggtcacaaatgagaggagaccagggtacattttcattccgagcgatgtttccgatatcatggtttccgcaggcaggttgaatcagccgttgacctttagtgaattgacagatgagtctgcgttgaaaaacatggccctgagaattttggcaaaactttacaattcaaagcacccttctgtacttggcgatgcattagcagacaggtttggggggcaaactgctttggataaccttgttgaaaagttaccatcgaatttcgtcaagttgttttccacgcttttggccagaaacatcgacgagactttaccgaactatatcggggtctacagcggcaaattgtcctccgataagattgtcattgacgaattggagagaaacaccgactttttgttgaccctcggccatgctaacaatgagatcaattccggggtatactcaactgacttttctgcaatcaccgagtatgtggaggtgcatccagattacattctcattgatggcgagtacgttctcatcaaaaacgcagaaaccggaaagagattgttttcaattgttgatttgcttactaagcttgtctcagatttcgatgcatcgaagatgattcacaacaatcatgctgttaacaacattagagcgaggcgcgaaaccaagcagttttcgtcattggatacggtttcgcctggagtgatcacgcaaaacaagttggttgattttttcaatgactacttgcggccaaacgatatcttgttgtgcgatacatgcagttttctttttggtgtgttcgagcttaagttcccgaggggcgtcaagtttattgcacaaaccttatacgaatcgatcgggtatgcacttcccgcgacttttggcgctgcaagggccgaaagggatttgggcacgaacagaagagtggtgttgatacagggagatggttctgcccaaatgacaatccaggaatggtccacatatttgagatacgacattctgtcgccagaaatctttttgctcaacaacgagggctacacggttgaaaggatgatcaaagggcccactcggtcctataacgatattcaggacacttggaaatggacggaatttttcaagattttcggcgacgaagactgcgagaagcatgaggctgaaaaagtcaacaccacaaacgaattggaagctttgactaggcgcaaaacaagcgagaagatccgcttgtatgaactcaagttgagcaaattagacattgtggacaaatttcggatcttgcgtgaatag

SEQ ID NO: 60 - DNA, 1113 bp, Metschnikowia
  GPD1 gene from H0 Metschnikowia
atgaccgctactgctcctttcaagatcgaatcccccttcagaattgccatcatcggctccggtaactggggtaccgccgtggccaagcttgtggctgagaacaccgctgagaagccggaaatcttccagaaacaggtgaacatgtgggtgtttgaggaggacatcaacggccgcaaattgaccgagatcatcaacactgaccatgagaacgtcaagtacatgccagaggtgaagttgccagaaaacttggttgcaaacccagacattgaggccaccgtcaaggatgctgacctccttattttcaacatcccccaccagttcttgccaagagtgtgcaagcaattggttggcaaggtttcgcctaccgccagagccatttcctgtcttaagggcttggaggtggatgcctctggctgcaaattgttgtcgcagtccatcaccgacaccttgggcatctactgtggtgtcttgtccggtgccaacatcgccaacgaggtggctagaggccgctggtccgagacctccatcgcctacaacagacccaccgacttccgtggcgagggcaaggatatctgtgagtttgtgttgaaggaggccttccacagaagatacttccacgtgcgcgtgatcaaggacgttattggcgcctcgatcgccggtgcgttgaagaacgttgtggccattgccgccggcttcgtcgaaggtgagggctggggtgacaatgccaagtctgccatcatgagaatcggcctcaaggagaccattcactttgcctcgtactgggagaagtttggcatccagggtctttctgctcctgagcctaccaccttcaccgaggagtctgccggtgttgccgacttgatcaccacgtgttccggtggtagaaacgtcaaggttgccagatacatgattgagaagaatgtcgacgcttgggaggctgagaaggccttgttgaacggccagtcctcgcaaggtatcatcaccgccaaggaggtgcacgagttgttggtgaactacaagttgcaagaggagttcccattgttcgaggccacctacgctgtcatttacgagaacgccgatgtcaacacctggcctacgattttggccgagtaa

SEQ ID NO: 61 - DNA, 1635 bp, Metschnikowia
  GXF1 gene from H0 Metschnikowia
atgtctcaagacgaacttcatacaaagtctggtgttgaaacaccaatcaacgattcgcttctcgaggagaagcacgatgtcaccccactcgcggcattgcccgagaagtccttcaaggactacatttccatttccattttctgtttgtttgtggcatttggtggttttgttttcggtttcgacaccggtacgatttccggtttcgtcaacatgtccgacttcaagaccagatttggtgagatgaatgcccagggcgaatactacttgtccaatgttagaactggtttgatggtttctattttcaacgtcggttgcgccgttggtggtatcttcctttgtaagattgccgatgtttatggcagaagaattggtcttatgttttccatggtggtttatgtcgttggtatcattattcagattgcctccaccaccaaatggtaccaatacttcattggccgtcttattgctggcttggctgtgggtactgtttccgtcatctcgccacttttcatttccgaggttgctcctaaacagctcagaggtacgcttgtgtgctgcttccagttgtgtatcaccttgggtatctttttgggttactgcacgacctacggtacaaagacttacactgactccagacagtggagaatcccattgggtatctgtttcgcgtgggctttgtttttggtggccggtatgttgaacatgcccgagtctcctagatacttggttgagaaatcgagaatcgacgatgccagaaagtccattgccagatccaacaaggtttccgaggaagaccccgccgtgtacaccgaggtgcagcttatccaggctggtattgacagagaggcccttgccggcagcgccacatggatggagcttgtgactggtaagcccaaaatcttcagaagagtcatcatgggtgtcatgcttcagtccttgcaacaattgactggtgacaactactttttctactacggaaccacgattttcaaggctgttggcttgcaggactctttccagacgtcgattatcttgggtattgtcaactttgcctcgacttttgtcggtatttacgccattgagagaatgggcagaagattgtgtttgttgaccggatctgcgtgcatgtttgtgtgtttcatcatctactcgctcattggtacgcagcacttgtacaagaacggcttctctaacgaaccttccaacacatacaagccttccggtaacgccatgatcttcatcacgtgtctttacattttcttctttgcctcgacctgggccggtggtgtttactgtatcgtgtccgagtcttacccattgagaatcagatccaaggccatgtctgtcgccaccgccgccaactggatgtggggtttcttgatctcgttcttcacgcctttcatcacctccgccatccacttttactacggttttgttttcactggctgcttggcgttctccttcttctacgtctacttctttgtcgtggagaccaagggtctttccttggaggaggttgacattttgtacgcttccggtacgcttccatggaagtcctctggctgggtgcctcctaccgcggacgaaatggcccacaacgccttcgacaacaagccaactgacgaacaagtctaa

SEQ ID NO: 62 - DNA, 1647 bp, Metschnikowia
  GXF2 gene from H0 Metschnikowia
atgagtgccgaacaggaacaacaagtatcgggcacatctgccacgatagatgggctggcgtccttgaagcaagaaaaaaccgccgaggaggaagacgccttcaagcctaagcccgccacggcgtactttttcatttcgttcctctgtggcttggtcgcctttggcggctacgttttcggtttcgataccggcacgatttccgggtttgttaacatggacgactatttgatgagattcggccagcagcacgctgatggcacgtattacctttccaacgtgagaaccggtttgatcgtgtcgatcttcaacattggctgtgccgtcggtggtcttgcgctttcgaaagttggtgacatctggggcagaagaattggtattatggttgctatgatcatctacatggtgggaatcatcatccagatcgcttcacaggataaatggtaccagtacttcattggccgtttgatcaccgggttgggtgtcggcaccacgtccgtgctcagtcctcttttcatctccgagtcggctccgaagcatttgagaggcacccttgtgtgttgtttccagctcatggtcaccttgggtatctttttgggctactgcacgacctacggtaccaagaactacactgactcgcgccagtggcggattcccttgggtctttgctttgcatgggcgcttttgttgatctcgggaatggttttcatgcccgaatccccacgtttcttgattgaacgccagagattcgacgaggcgaaggcctccgtggccaaatcgaaccaggtctcgaccgaggaccccgccgtgtacactgaagtggagttgatccaggccggtattgaccgtgaggcattggccggatccgctggctggaaagagcttatcacgggcaagcccaagatgttgcagcgtgtgattttgggaatgatgctccagtcgatccagcagctcaccggtaacaactactttttctactacggtaccacgatcttcaaggccgtgggcatgtcggactcgttccagacctcgattgttttgggtattgtcaacttcgcctccacttttgtcggaatctgggccatcgagcgtatgggccgcagatcttgtttgcttgttggttccgcgtgcatgagtgtgtgtttcttgatctactccatcttgggttccgtcaacctttacatcgacggctacgagaacacgccttcgaacacgcgtaagcctaccggtaacgccatgatcttcatcacgtgtttgtttatcttcttcttcgcctccacctgggccggtggtgtgtacagtattgtttctgaaacatacccattgagaatccggtctaaaggtatggccgtggccaccgctgccaactggatgtggggtttcttgatttcgttcttcacgcctttcatcacctcggccatccacttctactacgggtttgtgttcacagggtgtcttattttctccttcttctacgtgttcttctttgttagggaaaccaagggtctctcgttggaagaggtggatgagttatatgccactgacctcccaccatggaagaccgcgggctggacgcctccttctgctgaggatatggcccacaccaccgggtttgccgaggccgcaaagcctacgaacaaacacgtttaa

SEQ ID NO: 63 - DNA, 1647 bp, Metschnikowia
  GXS1 gene from H0 Metschnikowia
atgagcatctttgaaggcaaagacgggaagggggtatcctccaccgagtcgctttccaatgacgtcagatatgacaacatggagaaagttgatcaggatgttcttagacacaacttcaactttgacaaagaattcgaggagctcgaaatcgaggcggcgcaagtcaacgacaaaccttcttttgtcgacaggattttatccctcgaatacaagcttcatttcgaaaacaagaaccacatggtgtggctcttgggcgctttcgcagccgccgcaggcttattgtctggcttggatcagtccattatttctggtgcatccattggaatgaacaaagcattgaacttgactgaacgtgaagcctcattggtgtcttcgcttatgcctttaggcgccatggcaggctccatgattatgacacctcttaatgagtggttcggaagaaaatcatcgttgattatttcttgtatttggtataccatcggatccgctttgtgcgctggcgccagagatcaccacatgatgtacgctggcagatttattcttggtgtcggtgtgggtatagaaggtgggtgtgtgggcatttacatttccgagtctgtcccagccaatgtgcgtggtagtatcgtgtcgatgtaccagttcaatattgctttgggtgaagttctagggtatgctgttgctgccattttctacactgttcatggtggatggaggttcatggtggggtcttctttagtattctctactatattgtttgccggattgtttttcttgcccgagtcacctcgttggttggtgcacaaaggcagaaacggaatggcatacgatgtgtggaagagattgagagacataaacgatgaaagcgcaaagttggaatttttggagatgagacaggctgcttatcaagagagagaaagacgctcgcaagagtctttgttctccagctggggcgaattattcaccatcgctagaaacagaagagcacttacttactctgtcataatgatcactttgggtcaattgactggtgtcaatgccgtcatgtactacatgtcgactttgatgggtgcaattggtttcaacgagaaagactctgtgttcatgtcccttgtgggaggcggttctttgcttataggtaccattcctgccattttgtggatggaccgtttcggcagaagagtttggggttataatcttgttggtttcttcgttggtttggtgctcgttggtgttggctaccgtttcaatcccgtcactcaaaaggcggcttcagaaggtgtgtacttgacgggtctcattgtctatttcttgttctttggttcctactcgaccttaacttgggtcattccatccgagtcttttgatttgagaacaagatctttgggtatgacaatctgttccactttcctttacttgtggtctttcaccgtcacctacaacttcaccaagatgtccgccgccttcacatacactgggttgacacttggtttctacggtggcattgcgttccttggtttgatttaccaggtctgcttcatgcccgagacgaaggacaagactttggaagaaattgacgatatcttcaatcgttctgcgttctctatcgcgcgcgagaacatctccaacttgaagaagggtatttggtaa

SEQ ID NO: 64 - DNA, 1887 bp, Metschnikowia
  HGT19 gene from H0 Metschnikowia
atgtcagaaaagcctgttgtgtcgcacagcatcgacacgacgctgtctacgtcatcgaaacaagtctatgacggtaactcgcttcttaagaccctgaatgagcgcgatggcgaacgcggcaatatcttgtcgcagtacactgaggaacaggccatgcaaatgggccgcaactatgcgttgaagcacaatttagatgcgacactctttggaaaggcggccgcggtcgcaagaaacccatacgagttcaattcgatgagttttttgaccgaagaggaaaaagtcgcgcttaacacggagcagaccaagaaatggcacatcccaagaaagttggtggaggtgattgcattggggtccatggccgctgcggtgcagggtatggatgagtcggtggtgaatggtgcaacgcttttctaccccacggcaatgggtatcacagatatcaagaatgccgatttgattgaaggtttgatcaacggtgcgccctatctttgctgcgccatcatgtgctggacatctgattactggaacaggaagttgggccgtaagtggaccattttctggacatgtgccatttctgcaatcacatgtatctggcaaggtctcgtcaatttgaaatggtaccatttgttcattgcgcgtttctgcttgggtttcggtatcggtgtcaagtctgccaccgtgcctgcgtatgctgccgaaaccaccccggccaaaatcagaggctcgttggtcatgctttggcagttcttcaccgctgtcggaatcatgcttggttacgtggcgtctttggcattctattacattggtgacaatggcatttctggcggcttgaactggagattgatgctaggatctgcatgtcttccagctatcgttgtgttagtccaagttccgtttgttccagaatcccctcgttggctcatgggtaaggaaagacacgctgaagcatatgattcgctccggcaattgcggttcagtgaaatcgaggcggcccgtgactgtttctaccagtacgtgttgttgaaagaggagggctcttatggaacgcagccattcttcagcagaatcaaggagatgttcaccgtgagaagaaacagaaatggtgcattgggcgcgtggatcgtcatgttcatgcagcagttctgtggaatcaacgtcattgcttactactcgtcgtcgatcttcgtggagtcgaatctttctgagatcaaggccatgttggcgtcttgggggttcggtatgatcaatttcttgtttgcaattccagcgttctacaccattgacacgtttggccgacgcaacttgttgctcactactttccctcttatggcggtattcttactcatggccggattcgggttctggatcccgttcgagacaaacccacacggccgtttggcggtgatcactattggtatctatttgtttgcatgtgtctactctgcgggcgagggaccagttcccttcacatactctgccgaagcattcccgttgtatatccgtgacttgggtatgggctttgccacggccacgtgttggttcttcaacttcattttggcattttcctggcctagaatgaagaatgcattcaagcctcaaggtgcctttggctggtatgccgcctggaacattgttggcttcttcttagtgttatggttcttgcccgagacaaagggcttgacgttggaggaattggacgaagtgtttgatgtgcctttgagaaaacacgcgcactaccgtaccaaagaattagtatacaacttgcgcaaatacttcttgaggcagaaccctaagccattgccgccactttatgcacaccaaagaatggctgttaccaacccagaatggttggaaaagaccgaggtcacgcacgaggagaatatctag

SEQ ID NO: 65 - DNA, 1575 bp, Metschnikowia
  HXT2.6 gene from H0 Metschnikowia
atgctgagcactaccgataccctcgaaaaaagggacaccgagcctttcacttcagatgctcctgtcacagtccatgactatatcgcagaggagcgtccgtggtggaaagtgccgcatttgcgtgtattgacttggtctgttttcgtgatcaccctcacctccaccaacaacgggtatgatggcctgatgttgaatggattgcaatccttggacatttggcaggaggatttgggtcaccctgcgggccagaaattgggtgccttggccaacggtgttttgtttggtaaccttgctgctgtgccttttgcttcgtatttctgcgatcgttttggtagaaggccggtcatttgtttcggacagatcttgacaattgttggtgctgtattacaaggtttgtccaacagctatggattttttttgggttcgagaattgtgttgggttttggtgctatgatagccactattccgctgccaacattgatttccgaaatcgcctaccctacgcatagagaaacttccactttcgcctacaacgtgtgctggtatttgggagccattatcgcctcctgggtcacatacggcaccagagatttacagagcaaggcttgctggtcaattccttcttatctccaggccgccttacctttctttcaagtgtgcatgatttggtttgtgccagagtctcccagattcctcgttgccaagggcaagatcgaccaagcaagggctgttttgtctaaataccatacaggagactcgactgaccccagagacgttgcgttggttgactttgagctccatgagattgagagtgcattggagcaggaaaaattgaacactcgctcgtcatactttgactttttcaagaagagaaactttagaaagagaggcttcttgtgtgtcatggtcggtgttgcaatgcagctttctggaaacggcttagtgtcctattacttgtcgaaagtgctagactcgattggaatcactgaaaccaagagacagctcgagatcaatggctgcttgatgatctataactttgtcatctgcgtctcgttgatgagtgtttgccgtatgttcaaaagaagagtattatttctcacgtgtttctcaggaatgacggtttgctacacgatatggacgattttgtcagcgcttaatgaacagagacactttgaggataaaggcttggccaatggcgtgttggcaatgatcttcttctactattttttctacaacgttggcatcaatggattgccattcctatacatcaccgagatcttgccttactcacacagagcaaaaggcttgaatttattccaattctcgcaatttctcacgcaaatctacaatggctatgtgaacccaatcgccatggacgcaatcagctggaagtattacattgtgtactgctgtattctcttcgtggagttggtgattgtgtttttcacgttcccagaaacttcgggatacactttggaggaggtcgcccaggtatttggtgatgaggctcccgggctccacaacagacaattggatgttgcgaaagaatcactcgagcatgttgagcatgtttga

SEQ ID NO: 66 - DNA, 1647 bp, Metschnikowia; identical to SEQ ID NO: 63
  HXT5 gene from H0 Metschnikowia

SEQ ID NO: 67 - DNA, 1254 bp, Metschnikowia
  PGK1 gene from H0 Metschnikowia
atgtctttatctaacaaattgtctgtgaaagacttggacctcgctaacaagagagtcttcatcagagtcgacttcaacgttcctcttgacggaaccaccatcaccaacaaccagagaattgttgctgctttgccaaccatcaaatacgtcttggagcagaagccaaaggccgtcatcttggcttcccacttgggcagaccaaacggtgagagagttgagaagtactcgttggctccagttgccaaggaattgcagtccttgttgtctgaccagaaggtcacattcttgaacgacagcgttggacctgaggtcgagaaggctgtcaacagcgcctctcagggcgaggtgttcttgttggagaacttgcgttaccacatcgaggaggaaggctccaagaaggtcgacggcaacaaggtcaaggcttccaaggaggatgtcgagaagttcagacaaggattgaccgccttggccgacgtctacgtcaacgacgctttcggtaccgcccacagagcccactcttctatggttggtcttgaattgcctcagaaggctgccggtttcttgatggccaaggagttggagtacttcgccaaggccttggagaaccctaccagaccattcttggccatcttgggtggtgccaaggtctccgacaagatccagttgatcgacaacttgttggacaaggtcgacatcttgattgttggtggtggtatggctttcaccttcaagaaggttttggacaacatgccaattggtacttctcttttcgacgaggccggctccaagaacgtcgagaacttgattgccaaggctaagaagaacaacgtcgagattgtcttgcccgttgactttgtcaccgctgacgacttcaacaaggatgccaacactggtgttgccacccaagaggagggtatcccagacggatggatgggtcttgatgccggtccaaagtccagagaactctttgctgaggctgttgctaaggccaagaccattgtctggaacggcccaccaggtgttttcgagtttgagaaattcgctcagggcaccaagtccttgttggacgctgccgtcaagtccgccgaggctggcaacaccgtcatcattggcggtggtgacactgccactgttgccaagaagttcggtgtcgttgagaagttgtctcacgtctccactggtggtggtgcctccttggagttgttggagggtaaggagttgccaggtgtcgttgccatttctgacaagcagtaa

SEQ ID NO: 68 - DNA, 1692 bp, Metschnikowia
  QUP2 gene from H0 Metschnikowia
atgggctttcgcaacttaaagcgcaggctctcaaatgttggcgactccatgtcagtgcactctgtgaaagaggaggaagacttctcccgcgtggaaatcccggatgaaatctacaactataagatcgtccttgtggctttaacagcggcgtcggctgccatcatcatcggctacgatgcaggcttcattggtggcacggtttcgttgacggcgttcaaactggaatttggcttggacaaaatgtctgcgacggcggcttctgctatcgaagccaacgttgtttccgtgttccaggccggcgcctactttgggtgtcttttcttctatccgattggcgagatttggggccgtaaaatcggtcttcttctttccggctttcttttgacgtttggtgctgctatttctttgatttcgaactcgtctcgtggccttggtgccatatatgctggaagagtactaacaggtttggggattggcggatgtctgagtttggccccaatctacgtttctgaaatcgcgcctgcagcaatcagaggcaagcttgtgggctgctgggaagtgtcatggcaggtgggcggcattgttggctactggatcaattacggagtcttgcagactcttccgattagctcacaacaatggatcatcccgtttgctgtacaattgatcccatcggggcttttctggggcctttgtcttttgattccagagctgccacgttttcttgtatcgaagggaaagatcgataaggcgcgcaaaaacttagcgtacttgcgtggacttagcgaggaccacccctattctgtttttgagttggagaacattagtaaggccattgaagagaacttcgagcaaacaggaaggggttttttcgacccattgaaagctttgtttttcagcaaaaaaatgctttaccgccttctcttgtccacgtcaatgttcatgatgcagaatggctatggaatcaatgctgtgacatactactcgcccacgatcttcaaatccttaggcgttcagggctcaaacgccggtttgctctcaacaggaattttcggtcttcttaaaggtgccgcttcggtgttctgggtctttttcttggttgacacattcggccgccggttttgtctttgctacctctctctcccctgctcgatctgcatgtggtatattggcgcatacatcaagattgccaacccttcagcgaagcttgctgcaggagacacagccaccaccccagcaggaactgcagcgaaagcgatgctttacatatggacgattttctacggcattacgtggaatggtacgacctgggtgatctgcgcggagattttcccccagtcggtgagaacagccgcgcaggccgtcaacgcttcttctaattggttctgggctttcatgatcggccacttcactggccaggcgctcgagaatattgggtacggatactacttcttgtttgcggcgtgctctgcaatcttccctgtggtagtctggtttgtgtaccccgaaacaaagggtgtgcctttggaggccgtggagtatttgttcgaggtgcgtccttggaaagcgcactcatatgctttggagaagtaccagattgagtacaacgagggtgaattccaccaacataagcccgaagtactcttacaagggtctgaaaactcggacacgagcgagaaaagcctcgcctga

SEQ ID NO: 69 - DNA, 5157 bp, Metschnikowia
  RPB1 gene from H0 Metschnikowia
atggaccagacaaccaagaaacccagagatggtggcttgaacgatccacgtttgggctccatcgaccgtaacttcaagtgtcaaacctgtggcgaagatatggctgaatgtccgggccattttggccacattgagttggccaagcccgtgtttcacatcggttttattgccaagatcaagaaagtgtgcgagtgtgtttgtatgcactgtggaaaacttcttgttgacgatgctaaccccttgatggctcaggccattcggatcagggatccgaagaagcgcttcaacgccgtgtggaacgtgtccaagaccaagatggtgtgtgaagcagacactatcaatgaagaaggccaggtcacagccgggagaggaggatgtggccacacgcagccaactgtgcgcagagacggcttgaagttgtggggtacttggaaacagaacaaaacttacgacgagaacgaacagccagaacgtcgtttgttaagtccatcagagattttgagcgttttcagacacatcagccccgaggactgtcataagttgggctttaacgaggactatgccagacctgagtggatgttgatcacggttttgcctgtcccaccaccaccagtgaggccttccattgcctttaacgatacggctagaggtgaggatgatttgacgttcaagttggctgacattctcaaagcaaatatcaacgtacagcgtcttgaaatcgacggttcgccacagcacgtcatcagtgagttcgaggctttgttacagtttcatgtggcgacttacatggataatgatatcgctggccagcctcaggcgcttcaaaagaccggtcgtcctatcaaatcgatcagagccagattgaagggtaaagaggggagattgagaggtaacttgatgggcaaacgtgtggacttttctgcgcgtactgttatttctggtgaccccaatctcgaccttgaccaggtcggtgtgcctatatccattgctaggactttgacttatcctgaggttgtcaccccatacaacattcacaaattgaccgagtatgttcgcaatggccctaatgagcaccctggtgcgaaatatgtcattcgtgacaccggtgaccgtattgatctaatgtacaacaaaagggcgggtgacattgccttgcagtatgggtggaaggttgaacgtcatttgatggacgacgatccagttttgtttaatcgtcaaccctccttgcataagatgtccatgatggcacatcgagtcaaagtcatgccctactccacattcagattgaatttgtccgtcacttctccttacaatgctgatttcgatggtgatgagatgaacttacatgttcctcagtcgcctgagaccagagccgagatgtctcaaatttgcgcggttccgcttcaaatcgtctctccacaatcgaacaaacctgtgatgggtattgtgcaagacacattgtgtggtatccgtaaaatgacattacgcgacaatttcattgaatatgagcaagtcatgaacatgttgtactggatccctaactgggatggtgtcattcctccgccggcggtactcaagcccaagccattgtggtcgggtaaacagttgttgtctatggccattcccaagggtattcacttgcagaggttcgatgacggaagggacatgctcagtccaaaagatctggggatgttgattgttgacggtgagatcatctttggtgttgttgacaaaaaaaccgtcggcgccactggaggcggattgatccacacggtcatgagagagaagggtccatacgtctgtgcgcagcttttcagctcgatccagaaggttgtcaattattggcttttgcataatggtttctctatcggtattggtgacacaattgccgacaaagacaccatgcgtgatgtgacaacgaccattcaagaggccaaacagaaggtccaggaaatcatcattgacgcccagcaaaacaagttggagcctgaacccggtatgactctcagagaatcgttcgagcataatgtttcccgtattctcaatcaagctcgtgatactgctggccgttccgctgaaatgaacttgaaggatctgaacaacgtgaaacagatggtcacatccggatcgaaaggttctttcatcaacatctctcaaatgtctgcctgtgtcggtcaacaaattgttgagggtaagcgtattcccttcggttttggtgatcgtacgttacctcattttaccaaggatgactactcgcctgaatcgaagggttttgttgagaactcgtacctcagaggcttgactccccaggagtttttctttcacgctatggcaggaagagaaggtcttattgatactgccgtcaagactgcagaaacaggttacatccagcgtcgtttagtcaaagctttggaagatattatggtgcattatgatggcacaaccagaaactctttaggcgacatcatccagtttgtttatggtgaggacggaattgatgctacatcggttgaaaagcaatcagttgatactatacccggttcagactcctcgtttgagaagcgctacagaattgacgttttggacccagctaaatccattcctgagtcgttgctagagtcaggcaagcaaatcaagggagatgtggcagttcagaaggtgttggatgaagagtacgaccaattgctcaaggatcgtaagttcttgagagaggttgttttccccaatggtgactacaactggccattacccgttaatttgcgtcgtattattcaaaatgctcagcagattttccacagtggccgtcaaaaagcttccgacttaagattggaagagatagtcgaaggcgtgcagtccctttgtaccaagcttcttgttctccgaggaaagacggagctcatcaaggaggcgcaggaaaatgcgactttgcttttccagtgcttgttgagatctaggttggctgctcgtcgtgtcattgaggagttcaagctcaataaggtctcttttgaatgggtatgtggtgaaatcgagtcccagtttcagaagtctattgtacacccaggtgagatggttggtgttgtcgctgcgcagtctatcggtgagcctgcgacgcagatgactttaaacaccttccattacgccggtgtctcttccaaaaacgttacccttggtgtccctcgtcttaaggaaattttgaatgtggcgaaaaacatcaaaacgccggctcttaccgtgtacttggagcccgagatcgctgttgacattgaaaaggccaaggttgttcaatcggctattgaacacaccacgttgaagaacgtgacctcgtccacagaaatctactacgatcctgatcctagaagcaccgtgattgaggaagattatgatactgttgaagcttactttgccattcccgacgagaaggtcgaggaaactatcgacaatcagtctccatggttgcttcgtcttgaattggacagagccaaaatgttggataagcaacttacgatggctcaagtggccgagaagatttcgcagaactttggagaagacttgttcgttatttggtctgatgacactgcagacaagttgatcatccgttgtcgtgttatccgcgatccaaaattggaagaggaaggcgagcacgaggaggaccaaattttgaagagagtggaggcccacatgttggagacaatctcattgcgtggtatccctggtatcacgagagtctttatgatgcaacataagatgagcacgccagatgcggatggtgaatttctgcaaaagcaagaatgggttttggaaactgatggtgtaaacttggccgaggtcatcactgttcctggcgtcgatgcatcccgaacctattccaacaacttcatcgagattctttctgtgctcggtattgaggcgactcgtactgctttgttcaaggaaattctcaatgtcattgcatttgacggttcatacgtcaactaccgtcatatggctttgcttgtggacgtcatgactgcacgtggtcatttgatggctatcacccgtcatggtattaacagagcggaaactggtgctttgatgcgttgttcttttgaagagacggttgagatcttgttggatgctggtgccgctgctgaactagatgactgccgtggtatctccgagaatgtcatattaggacaaatgccacctttgggtaccggtgcttttgatgtgatggtcgacgagaagatgttgcaggacgcaagtgtgagttctgatattggtgttgctggtcagactgacggaggtgcgacgccatatagagactatgagatggaggatgataagattcaatttgaggaaggtgcgggattctcgccaattcataccgcaaatgtatctgatgcctctgggtctttaacctcgtacggcgggcaaccatccatggtatcacctacctcgccattctcgtttggcgccacgtctcctgggtatggcggtgtgacctcgcctgcgtacggcgcaacttcgccaacgtactcaccaacgtcaccaacatactcgccaacttcgcccagttactcaccgacgtcaccaagttactcaccgacgtcaccaagttactcaccgacgtcaccaagttactcaccgacgtcaccaagttactcgccaacatcgccaagttattcgccaacttcaccaagttattcgccaacttcgccaagttactcgccaacttcgccaagttattcgcctacttcgccaagttattcgccaacttcgccaagttactcaccgacgtcaccaagttactcaccgacgtcaccaagttactcaccgacgtcaccaagttactcgcctacttcgccaagttactcgcctacttcgccaagttactcacctacttcgccaagttattcgcctacttcgcctagttactcacctacttcgccgcagtattcgccaacttcgcctagttactctccgacgtcgccgcagtattcgccaacttcgccaagctactcgcctacgtcaccgcaatacctgccaacgtcgccaagttactcgcccacttcgcctcaatactctccaacttcgcctcaatactcgccgggctcaccggcatattcaccaggctcaccactgtactctactgagaagaaggacgaggacaagaagtga

SEQ ID NO: 70 - DNA, 3702 bp, Metschnikowia
  RPB2 gene sequence from the H0 Metschnikowia
atgtcgcaggagccggtagaagacccttacgtctacgacgaggaggacgcgcacagcatcacgcccgaggactgctggacggtgattctgtcgtttttccaggaaaaaggccttgtctcacagcagttggactcgttcgacgagttcatcgagtcaaacatccaggagttggtgtgggaggactcgcacttgattctcgaccagccggcgcaacatacttccgaggaccagtatgaaaataagcggtttgaaatcacgtttggcaagatctatatttcgaagccaacgcagaccgagggcgacggaacaacgcacccgatgttcccacaggaggcacgcttgcgtaacttgacctacagctcgccgctttacgtggacatgctgaaaaagaagtttctttccgatgacagagtgagaaagggtaacgagctagaatgggtggaggagaaagtcgatggcgaggaggcccagctgaaggtgttcttgggtaaggtgccaatcatgctaaggtcgaagttttgcatgttgcgggacttgggcgagcacgagttctacgagttgaaagagtgcccttacgatatgggtggctatttcgtcatcaacggttccgaaaaagtcttgatcgcccaggagcgctcggcggctaacattgtccaggtgtttaagaaggcagcgccctcgcccatctcgcacgtggcggagatccgttccgcgcttgaaaagggttcccgtttgatctcctcgatgcagatcaaactatatggtcgtgacgacaagggcaccactggcagaacaatcaaggccacattgccctacatcaaggaagacatcccgattgtgattgtattcagagccctcggcgtggtccccgatggagacattttggaacacatttgttacgatgcaaacgattggcaaatgttagagatgttgaagccatgtgtggaggaaggtttcgtgatccaggagcgcgaagtcgcacttgactttatcggtagaagaggtgtcttgggtatcagaagggaaaagcgtatccagtacgcaaaggatattttacagaaagagttgttgcctaacatcacacaggaggccggtttcgagtcaagaaaggcattcttcttgggttacatggtcaaccgtttgttgttatgtgcattagaaagaaaggagcctgacgacagagatcattttggcaagaagagattggatttggccggacccttgttggcatccttgttccgtctcttattcaaaaagcttaccagggatatctataactacatgcagcggtgcgtggagaatgacaaggagtttaatctcacgttggcggtcaagtcacagaccatcactgatggtttgcggtactcgttggccacaggtaattggggtgaacaaagaaaggccatgagtgcacgtgccggtgtgtcgcaggtgttgaacagatacacatactcatcgacattgtcgcatttgagaagaacaaatactccaattggccgtgacggtaagatcgccaaacctagacagttgcacaacacccactggggtcttgtatgtcctgcagaaactcctgagggtcaggcgtgtggtttggtgaagaatttgtctttgatgacgtgtatatccgttggtacctcttccgagccgatcttgtatttcttggaagagtggggtatggaacccttggaggactatgttccttcgaacgcaccagactgcacaagagtctttgtcaacggtgtatgggttggcacacacagagaaccggcacagcttgtcgataccatgaggaggttgagaaggaagggcgatatctctcccgaggtgtcgatcatcagggacatcagagaaatggagttcaagatcttcaccgatgcaggccgtgtctaccgtccgttgttcatcgtggacgacgacccagagtccgaaaccaagggtgagttgatgttgcaaaaagagcacgtgcacaagttgttgaactcggcctacgatgaatatgacgaggatgactccaatgcgtacacatggtcgtcgttggtgaatgatggtgtggtagagtacgttgacgccgaggaggaggagacaatcatgatcgccatgaccccagaggatttggaggcttccaagagtgcgttgtcggagactcagcaacaggatcttcaaatggaggaacaagagcttgatcctgcaaagcgaatcaaaccaacttatacctcatccacacacaccttcacgcattgtgagattcatccttcgatgattttgggtgtcgccgcctctatcattccgttccccgaccataaccagtcgccgcgtaacacataccagtctgctatgggtaaacaagccatgggtgtatttttgactaactatgccgttagaatggacacaatggcaaatatcttatactacccacagaaacccttggccacaacaagagccatggagcacttgaagttccgtgagttgcctgctggtcagaatgcagtggtggccattgcttgttactccggctacaaccaagaagattccatgatcatgaaccagtcgtcgattgatagaggattgttccggtctttgtttttcagatcttacatggatctagagaagagacaaggtatgaaagccttggagacgtttgaaaagccatccagatctgacaccttgagattgaagcatggaacctacgaaaagttagatgacgatggtttgatcgcgcctggtgtcagggtcagtggtgaggatatcatcatcggtaaaaccacacctattccacctgacaccgaggagttgggtcagagaacccagtatcataccaagagagatgcctcgacgccattgagaagcacggagtctggtattgttgaccaggttcttttgaccacaaatggtgacggcgccaagttcgtcaaggtcagaatgagaacgacgaaggttccacaaatcggtgacaagtttgcctccagacacggacaaaagggtacaatcggtgtcacatatagacacgaggatatgcctttcagtgcacagggtattgtgcctgacttgatcataaacccgcatgctattccatctcgtatgacagtcgctcacttgatcgagtgtttgttgtcgaaagtctcttccttgtccggattggaaggtgacgcctcgccattcacggacgtcacagccgaggctgtttccaaattgttgagagagcacggataccaatctagaggtttcgaggtgatgtacaatggtcacaccggtaagaagatgatggcgcaagtgttctttggcccaacgtactaccagagattgaggcatatggtggatgacaagatccacgctagagccagaggtccagttcaagttttgaccaggcagcctgtggaaggtagatccagggatggtggattacgtttcggagagatggagagagattgtatgattgcgcacggagctgctggattcttaaaggaaagattgatggaggcttcggatgctttcagagttcacgtttgtggaatctgtggtttgatgtcggtgattgcaaacttgaagaagaaccagttcgagtgtcggtcgtgcaaaaacaagaccaacatttaccagatccacattccatacgcagccaaattgttgttccaggagttgatggccatgaacatttctcctagattgtacacggagagatcaggaatcagtgtgcgtgtctga

SEQ ID NO: 71 - DNA, 1377 bp, Metschnikowia
  TEF1 gene from H0 Metschnikowia
atgggtaaagaaaagtcgcacgtcaacgtcgttgtcattggacacgtcgattccggtaagtctactaccaccggtcacttgatctacaagtgtggtggtattgacaagagaactatcgagaagttcgagaaggaggccgccgagttgggtaagggttctttcaagtacgcttgggtgttggacaagttgaaggctgagagagagagaggtatcactatcgacattgccttgtggaagttcgagactcctaagtaccacgtcaccgtcattgacgccccaggtcacagagatttcatcaagaacatgatcactggtacttcccaggctgactgtgctatcttgatcatcgccggtggtgttggtgagttcgaggctggtatctccaaggatggccagaccagagagcacgctttgttggcttacaccttgggtgttagacaattgattgttgccgtcaacaagatggactccgtcaagtgggacaagaacagatttgaggagatcatcaaggagacctctaacttcgtcaagaaggttggttacaaccctaagactgtgccattcgtgccaatctctggttggaacggtgacaacatgattgaggcttccaccaactgcccatggtacaagggttgggagaaggagaccaaggccggtaagtcttccggtaagaccttgttggaggccattgacgccattgagccaccaaccagacctaccgacaaggccttgagattgcctttgcaggatgtctacaagatcggtggtatcggaacggtgccagtcggccgtgtcgagaccggtgtcatcaaggccggtatggtcgtcaccttcgccccagctggtgtcaccactgaggtcaagtccgtcgagatgcaccacgagcagttggttgagggtcttccaggtgacaacgttggtttcaacgtcaagaacgtctctgttaaggagatcagaagaggtaacgtctgtggtgactccaagcaggacccaccaaaggctgccgcttctttcaccgctcaggttattgtgttgaaccaccctggtcagatctcctctggttactctccagtgttggactgtcacaccgcccacattgcctgtaaattcgacaccttgttggagaagattgacagaagaactggtaagtccttggagtctgagcctaagttcgtcaagtctggtgacgccgccattgtcaagatggtgccaaccaagccaatgtgtgttgaggctttcaccgactacccacctttgggtagattcgccgtcagagacatgagacagactgttgctgtcggtgtcatcaaggccgtcgagaagtccgacaaggctggtaaggtcaccaaggctgctcagaaggctgccaagaagtaa

SEQ ID NO: 72 - DNA, 747 bp, Metschnikowia
  TPI1 gene from H0 Metschnikowia
atggctcgtcaatttttcgtcggaggtaacttcaaaatgaacggcactaaggagtcgctcaccgccattgtcgacaccttgaacaaggccgacttgcccgagaacgtcgaggtggtgattgctcccccagccccatacctttccctcgtggtcgaggccaacaagcagaagaccgtggaggtcgctgctcaaaacgtgttcagcaaggcctccggtgcctacacaggtgagattgctcctcagcaattgaaggacttgggcgccaactggaccttgaccggccactctgagagaagaacgatcatcaaggagtccgacgagttcatcgccgagaagaccaagtttgctttggagtctggtgttagcgtcatcttgtgtatcggtgagaccttggaggagaagaaggctggcatcacgcttgaggtgtgcgccagacaattggacgctgtgtccaagattgtttccgactggaccaacgtcgtcattgcttacgagcccgtctgggctattggtactggcttggccgccactgcccaggatgctcaggacatccacaaggagatcagagcccacttgtctaagaccattggcgctgaacaagccgaggccgtcagaatcttgtacggtggttccgtcaacggcaaaaacgctgttgacttcaaggacaaggctgatgttgacggattcttggttggcggtgcctccttgaagccagagttcattgacatcatcaagtctagattgtaa

SEQ ID NO: 73 - DNA, 1830 bp, Metschnikowia
  XKS1 gene from H0 Metschnikowia
atgacttatagttccagctctggcctctttttgggcttcgacttgtcgacgcagcagcttaaaatcattgtgacaaacgagaacttgaaggcgcttggtacctaccatgttgagtttgatgctcaattcaaagagaaatacgcgatcaaaaagggtgttttgtcagatgaaaaaacgggcgagattttatcacccgtgcacatgtggctagaggcaattgaccatgtctttgggttgatgaaaaaagacaatttccccttcggaaaagtgaaaggcataagcggttcagggatgcagcacggatcggtcttttggtcgaagtctgcttcttcatccttaaagaatatggccgaatattcctctttaacagaagccttggctgatgcctttgcgtgtgatacttctcccaactggcaggaccattcgacagggaaagaaatcaaagactttgagaaagtcgttggaggcccggacaaattggcggaaattacaggctcaagagctcactacaggttcactgggttgcagattcggaagttggcagtgagatctgagaatgacgtttaccagaaaaccgatagaatatctttggtgtcgagttttgttgcgtccgttcttttgggcaggatcaccacaattgaggaggcggacgcttgcggaatgaatttatacaatgtgaccgagtctaagcttgatgaagatttgttagcaatcgctgcaggggtgcatccaaagctcgataacaaatccaaaagggaaacagacgagggtgtcaaagaactaaagcgaaagattggtgagatcaaacccgtgagttatcagacttcgggctcaatcgcaccatattttgtcgagaaatacggcttctctccagattcgaagattgtttcgtttacgggtgataatcttgcgaccatcatctctttgcctttgagaaaaaacgacgtcttggtgtcactaggcacatccaccaccgtacttttggtgaccgagagctacgcgccttcttcgcagtatcatcttttcaagcatcctacaattaagaatgcttacatgggaatgatttgctacagtaatggcgcgctagcaagagaaagagttcgtgacgccatcaatgagaagtatggtgtggcaggggattcttgggacaagttcaatgagatcttggatcgctcaggcgacttcaacaataagttgggtgtttactttcccatcggtgaaattgtgcccaatgctccggcccagacaaagagaatggaaatgaactcgcatgaggatgtgaaagagatcgaaaagtgggatttggaaaacgatgtcacttctattgttgagtcacaaaccgttagttgccgagtgagagcgggcccaatgctttctggatcgggtgactcgaatgaaggaacgcccgaaaatgaaaataggaaagtcaaaacactcatcgacgatttacactctaagttcggcgaaatttacacagacgggaaacctcagagctacgagtctttgacttcgaggccgcggaacatctactttgtcggaggggcttcaagaaacaagagtatcatacacaagatggcttcgatcatgggtgctaccgaaggaaactttcaggttgagattccgaatgcgtgtgctcttggcggcgcctacaaggcaagctggagccttgagtgtgagagcagacaaaagtgggtgcacttcaatgattacctcaatgagaagtacgatttcgatgatgtggatgagttcaaagtggacgacaaatggctcaactatattccggcgattggcttgttgtcgaaattggaaagcaaccttgaccagaactaa

SEQ ID NO: 74 - DNA, 957 bp, Metschnikowia
  XYL1 gene from H0 Metschnikowia
atggctactatcaaattgaactctggatacgacatgccccaagtgggttttgggtgctggaaagtaactaacagtacatgtgctgatacgatctacaacgcgatcaaagttggctacagattatttgatggcgctgaagattacgggaacgagaaagaggtgggcgaaggaatcaacagggccattgacgaaggcttggtggcacgtgacgagttgttcgtggtgtccaagctctggaacaacttccatcatccagacaacgtcgagaaggcgttggacaagactttgggcgacttgaatgtcgagtacttggacttgttcttgatccatttcccaattgcgttcaaattcgtgccctttgaggagaaatacccgcccggcttctactgtggagaaggcgataagtttatctacgaggatgtgcctttgcttgacacgtggcgggcattggagaagtttgtgaagaagggtaagatcagatccatcggaatctcgaacttttccggcgcgttgatccaggacttgctcaggggcgccgagatcccccctgccgtgttgcagattgagcaccacccatacttgcagcagcccagattgattgagtatgtgcagtccaagggtattgccatcacagcctactcctcttttggcccacagtcgtttgtggagttggaccaccccaaggtcaaggagtgtgtcacgcttttcgagcacgaagacattgtttccatcgctaaagctcacgacaagtccgcgggccaggtattattgaggtgggccacgcaaaggggtcttgccgtgattccaaagtcaaacaaaaccgagcgtttgttgctgaatttgaatgtgaacgattttgatctctctgaagcagaattggagcaaatcgcaaagttggacgtgggcttgcgcttcaacaacccttgggactgggacaagattccaatcttccattaa

SEQ ID NO: 75 - DNA, 1089 bp, Metschnikowia
  XYL2 gene from H0 Metschnikowia
atgcctgctaacccatccttggttttgaacaaagtgaacgacatcacgttcgagaactacgaggttccgttactcacagaccccaacgatgtattggttcaggtgaaaaagactggaatctgtggatctgacatccactactacacccacggcagaattggcgacttcgtgttgacaaagccaatggttttgggccacgaatccgccggtgtggtcgtggaggtcggcaaaggtgtcactgacttgaaggttggtgataaggttgccattgagcccggagtgccttctcgcaccagtgacgagtacaagagtggccactacaacttgtgcccacacatgtgttttgccgccacgcccaactctaaccccgacgagccaaacccgccagggactttgtgcaaatattacaagtccccagcggacttcttggtgaaattgcctgagcacgtctcccttgagttgggcgctatggtcgagcctttgactgtcggtgtgcacgcctcgcgtttgggccgtgtcacttttggtgaccacgttgtggttttcggtgctggcccagtcggtatccttgcggctgccgtggccagaaagtttggcgctgccagcgtgactatcgtcgacatcttcgacagcaaattggaattggccaagtccattggcgcggccactcacacattcaactcaatgactgagggtgttctttcggaggctttgcccgcgggcgtgagacctgacgttgtattggagtgcactggagcagagatctgtgtgcagcaaggtgtacttgcgttgaaggctggtggccgccacgtgcaagttggaaatgccggctcctatctcaaattccccatcaccgaatttgttaccaaggagttgactctctttggatccttccgttacggttacaacgactacaagacgtcggtcgccatcttggacgagaattacaagaacgggaaggagaatgcgttggtggactttgaagccttgattactcaccgtttccccttcaagaatgccattgaggcttacgacgcggtgcgcgctggcgacggagctgtcaagtgtatcattgacggcccagagtaa

SEQ ID NO: 76 - DNA, 1632 bp, Metschnikowia
  XYT1 gene from H0 Metschnikowia
atgggttacgaggaaaagcttgtagcgcccgcgttgaaattcaaaaactttcttgacaaaacccccaatattcacaatgtctatgtcattgccgccatctcctgtacatcaggtatgatgtttggatttgatatctcgtcgatgtctgtctttgtcgaccagcagccatacttgaagatgtttgacaaccctagttccgtgattcaaggtttcattaccgcgctgatgagtttgggctcgtttttcggctcgctcacatccacgttcatctctgagccttttggtcgtcgtgcatcgttgttcatttgtggtattctttgggtaattggagcagcggttcaaagttcgtcgcagaacagggcccaattgatttgtgggcgtatcattgcaggatggggcattggctttgggtcatcggtggctcctgtttacgggtccgagatggctccgagaaagatcagaggcacgattggtggaatcttccagttctccgtcaccgtgggtatctttatcatgttcttgattgggtacggatgctctttcattcaaggaaaggcctctttccggatcccctggggtgtgcaaatggttcccggccttatcctcttgattggacttttctttattcctgaatctccccgttggttggccaaacagggctactgggaagacgccgaaatcattgtggccaatgtgcaggccaagggtaaccgtaacgacgccaacgtgcagattgaaatgtcggagattaaggatcaattgatgcttgacgagcacttgaaggagtttacgtacgctgaccttttcacgaagaagtaccgccagcgcacgatcacggcgatctttgcccagatctggcaacagttgaccggtatgaatgtgatgatgtactacattgtgtacattttccagatggcaggctacagcggcaacacgaacttggtgcccagtttgatccagtacatcatcaacatggcggtcacggtgccggcgcttttctgcttggatctcttgggccgtcgtaccattttgctcgcgggtgccgcgttcatgatggcgtggcaattcggcgtggcgggcattttggccacttactcagaaccggcatatatctctgacactgtgcgtatcacgatccccgacgaccacaagtctgctgcaaaaggtgtgattgcatgctgctatttgtttgtgtgctcgtttgcattctcgtggggtgtcggtatttgggtgtactgttccgaggtttggggtgactcccagtcgagacaaagaggcgccgctcttgcgacgtcggccaactggatcttcaacttcgccattgccatgttcacgccgtcctcattcaagaatatcacgtggaagacgtatatcatctacgccacgttctgtgcgtgcatgttcatacacgtgtttttctttttcccagaaacaaagggcaagcgtttggaggagataggccagctttgggacgaaggagtcccagcatggaggtcagccaagtggcagccaacagtgccgctcgcgtccgacgcagagcttgcacacaagatggatgttgcgcacgcggagcacgcggacttattggccacgcactcgccatcttcagacgagaagacgggcacggtctaa

SEQ ID NO: 77 - DNA, 972 bp, Metschnikowia
  TAL1 gene from H0 Metschnikowia
atgtctaactctttggaatccttgaaagctaccggcaccgtgatcgtcaccgacactggtgagttcgactcgattgccaagtacaccccacaagatgccaccaccaacccttcgttgattttagccgcctcgaaaaaggctgagtacgccaaggtgattgatgttgctattaaatacgccgaggacaagggcagcaaccctaaggagaaggccgccattgccttggacagattgttggtggagttcggtaaggaaatcttgctgattgtgcctggcagagtgtctaccgaggttgacgccagattgtcgtttgacaaggacgccaccgtcaagaaggcgcttgagatcatcgaattgtacaagtccattggcatctcgaaggacagagtgttgatcaagatcgcttccacctgggaaggtatccaggccgccaaggagttggaggccaagcacgacatccactgtaacttgacgcttttgttcagtttcgtgcaggcggtggcgtgtgccgaggccaaggtcactttgatctcgcctttcgtcggcagaatcttggactggtacaaggcctccaccggcaaggagtacgatgccgagtccgaccctggtgttgtgtctgtcagacagatctacaactactacaagaagtacggctacaacacgattgtcatgggcgcgtctttcagaaacactggcgagatcaaggccttggctggctgcgactacttgactgtggcccctaagttgttggaggagttgatgaactcttccgaggaggtgcctaaggtgttggacgctgcctcggccagctccgcgtctgaggagaaggtttcctacattgacgacgagagcgagttcagattcttgttgaacgaggacgccatggccaccgagaagttggcccagggtatcagaggctttgccaaggacgcccagaccttgttggccgagttggagaacagattcaagtag

SEQ ID NO: 78 - DNA, 2034 bp, Metschnikowia
  TKL1 gene from H0 Metschnikowia
atgtccgacatcgatcaattggctatttctaccatccgtttgttggcggtcgacgccgtggccaaggccaactctggtcaccccggtgccccattgggtctcgcccctgccgcccacgccgtttggaaggagatgaaattcaacccaaagaaccccgactgggtcaacagagaccgttttgtgttgtcgaacggtcacgcttgcgctttgttatacgccatgttgcacctttacggcttcgacatgtcgcttgacgacttgaagcagttccgtcagttgaactcgaaaacacccggacatcccgagaagtttgaaatcccaggtgccgaggtcaccacgggccccttgggtcagggtatctccaacgccgtgggtttggccattgcacagaagcaattcgctgccacgttcaacaaggacgatttcgccatctctgactcgtacacctacgccttcttgggtgacggatgtttgatggagggtgtcgcctcggaagcatcttctttggctggccacctccaattgaacaacttgattgcgttctgggacgacaacaagatctcgatcgatggatccactgaagtggccttcaccgaggacgtgttgaagcgttacgaggcttacggttgggacacgctcacgattgagaagggtgacactgacttggagggcgtcgctcaggcgatcaagactgccaaggcgctgaagaagcctactttgatccgtttgaccaccatcatcggctacggctcgctccagcagggtacccacggtgttcacggtgctccattgaagccagatgacatcaagcagttgaaggagaagtttggcttcgacccaaccaagtcgtttgtcgtgcctcaggaagtttacgactactacggcacactcgtaaagaagaaccaggagttggagtccgagtggaacaagaccgtcgagtcctacatccagaaattcccagaggagggcgctgtcttggcgcgcagactcaagggtgagttgcctgaggactgggccaagtgcttgcctacttacaccgctgatgacaagccgttggccacgagaaagttgtctgagatggctctcatcaagatcttggatgtcgttccagagcttattggtggctctgccgacttgaccggctcgaacttgacccgtgcccctgacatggttgacttccagccccctcagaccggcttgggtaactacgctggtagatacatccgttacggtgtgcgtgagcacggtatgggtgccatcatgaacggtatcgccggttttggtgctggtttccgtaactacggcggtaccttcttgaacttcgtctcgtacgccgccggtgctgtgcgtttgtcggctctttctcacttgcctgtgatctgggttgctacgcatgactcgattggtttgggtgaggacggtcctacccaccagcctattgagaccttggcccacttcagagctacccctaacatctctgtgtggagacctgctgacggtaacgaggtgtcagctgcttacaagtctgccattgagtctacctctaccccacacatcttggccttgaccagacagaacttgcctcaattggctggttcttctgtggagaaggcctctaccggtggttacaccgtgtaccagaccactgacaagcctgccgtcatcatcgtggcttctggttccgaggtggccatctctattgacgccgccaagaagttggagggtgagggcatcaaggccaacgttgtttccttggttgacttccacactttcgacaagcagcctttggactaccgtttatctgttttgccagatggcgtgccaatcatgtccgttgaggtgatgtcctcgttcggctggtccaagtattctcacgagcagttcggcttgaacagattcggtgcctccggcaaggccgaagacctttacaagttcttcgacttcacgccagaaggcgttgctgacagagccgccaagaccgtgcagttctacaagggcaaggacctcctttcgcctttgaacagagccttctaa

(19) United States
(12) Patent Application Publication
(10) Pub. No.: US 2018/0195093 A1
(43) Pub. Date: Jul. 12, 2018

(54) METSCHNIKOWIA SPECIES FOR BIOSYNTHESIS OF COMPOUNDS

(71) Applicant: Creatus Biosciences Inc., Vancouver (CA)

(72) Inventors: Hendrik Jurgens Jansen van Vuuren, Lions Bay (CA); Zongli Luo, Vancouver (CA); Allan George DeBono, Vancouver (CA); Andrew Taplin Ferguson, Vancouver (CA)

(21) Appl. No.: 15/849,223

(22) Filed: Dec. 20, 2017

Related U.S. Application Data
(60) Provisional application No. 62/437,610, filed on Dec. 21, 2016.

(51) Int. Cl. C12P 7/18; C07K 14/39; C12P 7/22; C12P 7/16; C12P 7/06; C12R 1/645

(52) U.S. Cl. CPC C12P 7/18; C07K 14/39; C12R 1/645; C12P 7/16; C12P 7/06; C12P 7/22

(57) ABSTRACT

Provided herein are Metschnikowia species that produce useful compounds from xylose when cultured, as well as methods to make and use these Metschnikowia species.

### CROSS-REFERENCE TO RELATED APPLICATION

This application claims the benefit of priority of U.S. Provisional Application No. 62/437,610, filed on Dec. 21, 2016, the content of which is herein incorporated by reference in its entirety.

### FIELD

The present invention relates to the field of molecular biology and microbiology. Provided herein are Metschnikowia species that produce useful compounds from xylose when cultured, as well as methods to make and use these Metschnikowia species.

### REFERENCE TO SEQUENCE LISTING

The instant application contains a Sequence Listing which has been submitted in ASCII format via EFS-Web and is hereby incorporated by reference in its entirety. Said ASCII copy, created on Dec. 19, 2017, is named 14305-008-999_Sequence_Listing.txt and is 188,107 bytes in size.

### BACKGROUND

Xylose is an abundant sugar present in lignocellulosic biomass, a renewable feedstock for producing bioderived chemicals. However, the use of lignocellulosic biomass and the production of bioderived chemicals are limited by the naturally low xylose uptake in microbial organisms. Therefore, a microbial organism that can use xylose to produce bioderived compounds, such as xylitol, represents an unmet need.

Xylitol is a five-carbon sugar alcohol widely used as a low-calorie, low-carbohydrate alternative to sugar (Drucker et al., Arch of Oral Biol. 24:965-970 (1979)). Xylitol is approximately as sweet as sucrose but has 33% fewer calories. Xylitol has been reported to not affect insulin levels of people with diabetes and individuals with hyperglycemia. The consumption of xylitol is also reportedly beneficial for dental health, reducing the incidence of caries. For example, xylitol in chewing gum is reported to inhibit growth of Streptoccocus mutans (Haresaku et al., Caries Res. 41:198-203 (2007)), and to reduce the incidence of acute middle ear infection (Azarpazhooh et al., Cochrane Database of Systematic Reviews 11:CD007095 (2011)). Moreover, xylitol has been reported to inhibit demineralization of healthy tooth enamel and to re-mineralize damaged tooth enamel (Steinberg et al., Clinical Preventive Dentistry 14:31-34 (1992); Maguire et al., British Dental J. 194:429-436 (2003); Grillaud et al., Arch of Pediatrics and Adolescent Medicine 12:1180-1186 (2005)).

Commercially, xylitol may be produced by chemical reduction of xylose, although this can present difficulties associated with separation and purification of xylose or xylitol from hydrolysates. Microbial systems for the production of xylitol have been described (Sirisansaneeyakul et al., J. Ferment. Bioeng. 80:565-570 (1995); Onishi et al., Agric. Biol. Chem. 30:1139-1144 (1966); Barbosa et al., J. Ind. Microbiol. 3:241-251 (1988); Gong et al., Biotechnol. Lett. 3:125-130 (1981); Vandeska et al., World J. Microbiol. Biotechnol. 11:213-218 (1995); Dahiya et al., Cabdirect.org 292-303 (1990); Gong et al., Biotechnol. Bioeng. 25:85-102 (1983)). For example, yeast from the genus Candida has been described as being useful for xylitol production. However, Candida spp. may be opportunistic pathogens, so the use of these organisms in processes related to food products are not desirable.

The Metschnikowia species, methods and compositions provided herein meet these needs and provide other related advantages.

### SUMMARY OF THE INVENTION

Provided herein is an isolated novel Metschnikowia species. This Metschnikowia species produces xylitol at specified rates and efficiencies that are distinct from other Metschnikowia species. For example, in some aspects, provided herein is a Metschnikowia species that produces at least 0.1 g/L/h of xylitol from xylose when cultured under aerobic conditions and at 30° C. for three days in liquid yeast extract peptone (YEP) medium including 4% xylose. In some aspects, provided herein is an isolated Metschnikowia species that produces at least 1 g/L of xylitol from xylose when cultured under aerobic conditions and at 30° C. for three days in liquid yeast nitrogen base (YNB) medium including 4% xylose. In some aspects, provided herein is an isolated Metschnikowia species that produces at least 1 g/L of xylitol from xylose when cultured under aerobic conditions and at 30° C. for two days in liquid yeast nitrogen base (YNB) medium including 2% xylose and 2% glucose.

Also provided herein is an isolated Metschnikowia species that produces a distinct combination of compounds. For example, in some aspects, provided herein is an isolated Metschnikowia species that produces about 0.11 g/L/h of xylitol, about 6.8E-05 g/L/h of n-butanol, about 2.5E-04 g/L/h of isobutanol, about 2.4E-04 g/L/h of isopropanol, about 2.64E-04 g/L/h of ethanol and about 3.73E-06 g/L/h of 2-phenylethyl alcohol when cultured under aerobic conditions for three days in liquid yeast extract peptone (YEP) medium including 4% xylose. In another aspect, provided herein is an isolated Metschnikowia species that produces compounds xylitol, n-butanol, isobutanol, isopropanol, ethanol and 2-phenylethyl alcohol at a concentration of about 8,000 mg/L xylitol, about 4.85 mg/L n-butanol, about 18.06 mg/L isobutanol, about 17.5 mg/L isopropanol, about 19.7 mg/L ethanol and about 0.269 mg/L 2-phenylethyl alcohol when cultured under aerobic conditions for three days in liquid yeast extract peptone (YEP) medium including 4% xylose. In yet another aspect, provided herein is an isolated Metschnikowia species that produces compounds xylitol, n-butanol, isobutanol, isopropanol, ethanol and 2-phenylethyl alcohol at a relative ratio of 99.26% xylitol, 0.061% n-butanol, 0.223% isobutanol, 0.217% isopropanol, 0.236% ethanol and 0.003% 2-phenylethyl alcohol when cultured under aerobic conditions for three days in liquid yeast extract peptone (YEP) medium including 4% xylose.

Still further provided herein is an isolated Metschnikowia species that has distinguishing genetic characteristics. For example, in some aspects, provided herein is an isolated Metschnikowia species having a D1/D2 domain sequence that includes: (1) a nucleic acid sequence that is at least 96.8% identical to SEQ ID NO: 1; (2) a nucleic acid sequence within the consensus sequence of SEQ ID NO: 2; or (3) a nucleic acid sequence including residues 1-153, 178 to 434 and 453 to 499 of SEQ ID NO: 2 with no more than 4 nucleotide substitutions therein, and at least one nucleic acid sequence encoding an amino acid sequence selected from SEQ ID NOS: 37, 40, 42, 44, 49, 51, 52, 55 and 56. In some aspects, provided herein is an isolated Metschnikowia species having a D1/D2 domain sequence that includes: (1) a nucleic acid sequence that is at least 96.8% identical to SEQ ID NO: 1; or (2) a nucleic acid sequence within the consensus sequence of SEQ ID NO: 2; or (3) a nucleic acid sequence including residues 1-153, 178 to 434 and 453 to 499 of SEQ ID NO: 2 with no more than 4 nucleotide substitutions therein, and at least one encoding nucleic acid sequence selected from SEQ ID NOS: 57-78. In a particular aspect, provided herein is an isolated Metschnikowia species having: (1) a nucleic acid sequence that is at least 97.1% identical to the D1/D2 domain consensus sequence of SEQ ID NO: 2; and (2) an encoding nucleic acid sequence of SEQ ID NO: 70.

Also provided herein is an isolated Metschnikowia species that has both distinguishing genetic characteristic and physiological characteristics. For example, in some aspects, provided herein is an isolated Metschnikowia species having: (1) a D1/D2 domain sequence that is at least 96.8% identical to SEQ ID NO: 1; and (2) an encoding nucleic acid sequence of SEQ ID NO: 68, and wherein said isolated Metschnikowia species grows to an OD₆₀₀ of about 25 within 41 hours of culturing in yeast extract peptone (YEP) medium including 2% xylose as the sole carbon source.

In a further aspect, the isolated Metschnikowia species provided herein have a specific D1/D2 domain sequence. For example, in some aspects, the D1/D2 domain sequence includes a nucleic acid sequence selected from SEQ ID NOS: 1 and 3-25. Additionally, in some aspects, the D1/D2 domain sequence of the isolated Metschnikowia species provided herein does not include the D1/D2 domain sequence of a Metschnikowia species selected from Metschnikowia andauensis, Metschnikowia chrysoperlae, Metschnikowia fructicola, Metschnikowia pulcherrima, Metschnikowia shanxiensis, Metschnikowia sinensis, and Metschnikowia zizyphicola.

In one aspect, provided herein is an isolated Metschnikowia species designated Accession No. 081116-01, deposited at the International Depositary Authority of Canada, an International Depositary Authority, on Nov. 8, 2016, under the terms of the Budapest Treaty.

Also provided herein is a recombinant version of the deposited Metschnikowia species. Thus, in some aspects, provided herein is an isolated Metschnikowia species designated Accession No. 081116-01, deposited at the International Depositary Authority of Canada, an International Depositary Authority, on Nov. 8, 2016, under the terms of the Budapest Treaty, wherein the Metschnikowia species further includes a metabolic pathway capable of producing a bioderived compound from xylose or a genetic modification, or both. The metabolic pathway of the Metschnikowia species, in some embodiments, includes at least one exogenous nucleic acid sequence encoding at least one enzyme of the metabolic pathway. The bioderived compound can be selected from any of the bioderived compounds described herein, including, but not limited to, phenyl-ethyl alcohol, 2-methyl-butanol, or 3-methyl-butanol.

Also provided herein are methods for producing a bioderived compound (e.g., xylitol, arabitol, ethanol, n-butanol, isobutanol, isopropanol, ethyl acetate, phenyl-ethyl alcohol, 2-methyl-butanol, or 3-methyl-butanol) using the isolated Metschnikowia species provided herein. Accordingly, in some aspects, provided herein is a method for producing xylitol including culturing the isolated Metschnikowia species provided herein under conditions and for a sufficient period of time to produce xylitol from xylose. Such Metschnikowia species can produce at least 0.1 g/L/h, at least 0.2 g/L/h, at least 0.3 g/L/h, at least 0.4 g/L/h, at least 0.50 g/L/h, at least 0.60 g/L/h, at least 0.70 g/L/h, at least 0.80 g/L/h, at least 0.90 g/L/h, at least 1.00 g/L/h, at least 1.50 g/L/h, at least 2.00 g/L/h, at least 2.50 g/L/h, at least 3.00 g/L/h, at least 3.50 g/L/h, at least 4.00 g/L/h, at least 5.00 g/L/h, at least 6.00 g/L/h, at least 7.00 g/L/h, at least 8.00 g/L/h, at least 9.00 g/L/h, or at least 10.00 g/L/h of xylitol from xylose.

The methods provided herein can include culturing the Metschnikowia species provided herein with xylose as a carbon source in combination with other co-substrates. Accordingly, in some aspects, the conditions include culturing the isolated Metschnikowia species in medium including xylose and a C3 carbon source, a C4 carbon source, a C5 carbon source, a C6 carbon source, or a combination thereof. The conditions can also include culturing the isolated Metschnikowia species in medium including xylose and a co-substrate selected from cellobiose, galactose, glucose, ethanol, acetate, arabinose, arabitol, sorbitol and glycerol, or a combination thereof. The culturing conditions can include aerobic culturing conditions, batch cultivation, fed-batch cultivation or continuous cultivation. The methods can also include separating the xylitol from other components in the culture.

In some aspects, provided herein is a bioderived compound (e.g. xylitol, arabitol, ethanol, n-butanol, isobutanol, isopropanol, ethyl acetate, phenyl-ethyl alcohol, 2-methyl-butanol, or 3-methyl-butanol) produced by a method described herein.

In some aspects, provided herein is a composition having the isolated Metschnikowia species described herein. Additionally or alternatively, also provided herein is a composition having the bioderived compound (e.g. xylitol, arabitol, ethanol, n-butanol, isobutanol, isopropanol, ethyl acetate, phenyl-ethyl alcohol, 2-methyl-butanol, or 3-methyl-butanol) described herein. In some embodiments, the composition is culture medium having xylose, and, in some embodiments, the composition is culture medium from which the isolated Metschnikowia species described herein has been removed. In some embodiments the composition includes impurities from the method used to produce the composition, which can include glycerol, arabitol, a C7 sugar alcohol, or a combination thereof. In a specific embodiment, the C7 sugar alcohol is volemitol or an isomer thereof. The composition can also include a specific amount of the impurities, such as when the amount of glycerol or arabitol, or both, is at least 10%, 20%, 30% or 40% greater than the amount of the respective glycerol or arabitol, or both, produced by a microbial organism other than the isolated Metschnikowia species described herein.

In another aspect, provided herein are isolated polypeptides and isolated nucleic acids, which correspond to the proteins and nucleic acids identified herein from the novel Metschnikowia species described herein. Accordingly, in some aspects, provided herein is an isolated polypeptide having an amino acid sequence selected from SEQ ID NOS: 37, 40, 42, 44, 49, 51, 52, 55 and 56. In some aspects, provided herein is an isolated nucleic acid having a nucleic acid sequence selected from SEQ ID NOS: 57-78. Still further provided is a vector having the isolated nucleic acid sequences described herein, as well as a host cell having such a vector.

### BRIEF DESCRIPTION OF THE DRAWINGS

FIG. 1 shows a sequence alignment between of all D1/D2 sequences identified from individual H0 Metschnikowia sp. clones. SEQ ID NOS: 2 and 3-25 are depicted.

FIG. 2 shows a neighbor-joining tree of all RPB2 sequences for the H0 Metschnikowia sp., members of the Metschnikowia pulcherrima clade and the outgroup species, Metschnikowia kunwiensis, which shows the distances between the different species.

FIG. 3 shows exemplary growth curves for the H0 Metschnikowia sp. as compared to members of the Metschnikowia pulcherrima clade.

FIG. 4 shows the production of xylitol from xylose for H0 Metschnikowia sp. and Saccharomyces cerevisiae M2 strain. YP+4% Xylose indicates yeast extract peptone medium having 4% xylose. YP+10% Xylose indicates yeast extract peptone medium having 10% xylose.

FIGS. 5A-5D show cell growth curves for H0 Metschnikowia sp. and Metschnikowia pulcherrima flavia (FL) strain cultured in different media. FIG. 5A is YNB medium with 4% glucose (YNBG). FIG. 5B is YNB medium with 4% xylose (YNBX). FIG. 5C is YNB medium with 2% glucose and 2% xylose (YNBGX). FIG. 5D is YPD medium with 4% xylose (YPDX).

FIGS. 6A and 6B show glycerol and ethanol produced by H0 Metschnikowia sp. and FL strain in YNBG, YNBGX and YPDX media.

FIGS. 7A-7D show arabitol levels produced during the growth of H0 Metschnikowia sp. and FL strain in YNBG (FIG. 7A), YNBX (FIG. 7B), YNBGX (FIG. 7C) and YPDX (FIG. 7D) media.

FIGS. 8A-8C show xylitol levels produced during the growth of H0 Metschnikowia sp. and FL strain in YNBX (FIG. 8A), YNBGX (FIG. 8B) and YPDX (FIG. 8C) media.

FIGS. 9A-9D show peak ratios production of various volatile compounds produced by H0 Metschnikowia sp. and FL strain in YNBG (FIG. 9A), YNBX (FIG. 9B), YNBGX (FIG. 9C) and YPDX (FIG. 9D) media.

### DETAILED DESCRIPTION

The compositions and methods provided herein are based, in part, on the discovery, isolation and characterization of a novel yeast species within the Metschnikowia genus. Isolation and characterization of this novel Metschnikowia species, referred to herein as “H0” or the “H0 Metschnikowia sp.,” has revealed numerous advantageous properties, novel genes and proteins, and valuable uses for the H0 Metschnikowia sp. and a recombinant H0 Metschnikowia sp. thereof. For example, some of the advantageous properties of the H0 Metschnikowia sp. include its ability to utilize glucose, xylose, and cellobiose as a carbon source for producing a bioderived compound, such as xylitol, arabitol, n-butanol, isobutanol, isopropanol, ethanol, or phenylethyl alcohol. Exemplary novel genes of the H0 Metschnikowia sp. include ACT1, ARO8, ARO10, GPD1, GXF1, GXF2, GXS1, HGT19, HXT2.6, HXT5, PGK1, QUP2, RPB1, RPB2, TEF1, TPI1, XKS1, XYL1, XYL2, XYT1, TAL1 and TKL1, as well as novel proteins for Aro10, Gxf2, Hgt19, Hxt5, Tef1, Xks1, Xyl1, Tal1 and Tkl1. Accordingly, the H0 Metschnikowia sp. can be used in a method for producing a bioderived compound, such as xylitol, arabitol, n-butanol, isobutanol, isopropanol, ethanol, or phenylethyl alcohol, by culturing the H0 Metschnikowia sp. in medium having xylose as the carbon source for production of the bioderived compound. Also provided herein are compositions having a bioderived compound produced by the methods that use the H0 Metschnikowia sp. or recombinant H0 Metschnikowia sp. to produce the bioderived compound. Still further provided herein are isolated polypeptides directed to the novel proteins of the H0 Metschnikowia sp. and isolated nucleic acids directed to the novel genes of the H0 Metschnikowia sp., as well as host cells including such nucleic acids.

As used herein, the term “aerobic” when used in reference to a culture or growth condition is intended to mean that free oxygen (O₂) is available in the culture or growth condition. This includes when the dissolved oxygen in the liquid medium is more than 50% of saturation.

As used herein, the term “anaerobic” when used in reference to a culture or growth condition is intended to mean that the culture or growth condition lacks free oxygen (O₂).

As used herein, the term “attenuate,” or grammatical equivalents thereof, is intended to mean to weaken, reduce or diminish the activity or amount of an enzyme or protein. Attenuation of the activity or amount of an enzyme or protein can mimic complete disruption if the attenuation causes the activity or amount to fall below a critical level required for a given pathway to function. However, the attenuation of the activity or amount of an enzyme or protein that mimics complete disruption for one pathway can still be sufficient for a separate pathway to continue to function. For example, attenuation of an endogenous enzyme or protein can be sufficient to mimic the complete disruption of the same enzyme or protein for production of a particular compound (e.g., xylitol), but the remaining activity or amount of enzyme or protein can still be sufficient to maintain other pathways or reactions, such as a pathway that is critical for the host Metschnikowia species to survive, reproduce or grow. Attenuation of an enzyme or protein can also be weakening, reducing or diminishing the activity or amount of the enzyme or protein in an amount that is sufficient to increase yield of xylitol, but does not necessarily mimic complete disruption of the enzyme or protein.

As used herein, the term “biobased” means a product that is composed, in whole or in part, of a bioderived compound. A biobased or bioderived product is in contrast to a petroleum derived product, wherein such a product is derived from or synthesized from petroleum or a petrochemical feedstock.

As used herein, the term “bioderived” means derived from or synthesized by a biological organism and can be considered a renewable resource since it can be generated by a biological organism. Such a biological organism, in particular the Metschnikowia species disclosed herein, can utilize feedstock or biomass, such as, sugars (e.g., xylose, cellobiose, glucose, fructose, galactose (e.g., galactose from marine plant biomass), and sucrose), carbohydrates obtained from an agricultural, plant, bacterial, or animal source, and glycerol (e.g., crude glycerol byproduct from biodiesel manufacturing).

As used herein, the term “carbon source” refers to any carbon containing molecule used by an organism for the synthesis of its organic molecules, including, but not limited to the bioderived compounds described herein. This includes molecules with different amounts of carbon atoms. Specific examples include a C3 carbon source, a C4 carbon source, a C5 carbon source and a C6 carbon source. A “C3 carbon source” refers to a carbon source containing three carbon atoms, such as glycerol. A “C4 carbon source” refers to a carbon source containing four carbon atoms, such as erythrose or threose. A “C5 carbon source” refers to a carbon source containing five carbon atoms, such as xylose, arabinose, arabitol, ribose or lyxose. A “C6 carbon source” refers to a carbon source containing six carbon atoms, such as glucose, galactose, mannose, allose, altrose, gulose, or idose.

As used herein, the term “D1/D2 domain” is a 450-600 nucleotide domain at the 5′ end of a large subunit of (26S) rDNA found in most yeast. Most yeast species can be identified from sequence divergence of the D1/D2 domain. Conspecific strains of yeast generally have less than a 1% divergence in the nucleotide sequence for the D1/D2 domain, whereas biological species are separated by a greater than 1% divergence for this domain. However, in rare instances, such as for the species Clavispora lusitaniae (Lachance et al., FEMS Yeast Res. 2003; 4:253-8), Metschnikowia andauensis and Metschnikowia fructicola (Sipiczki et al., PLoS One. 2013; 8:e67384), and the unique Metschnikowia species described herein, a greater than 1% difference for the D1/D2 domain can be found within the same species. For example, the unique Metschnikowia species described herein has a divergence of up to 3.8% in the D1/D2 domain. Methods of assaying the nucleotide sequence of the D1/D2 domain are well known in the art. One exemplary method for assaying the D1/D2 domain for a Metschnikowia species, as described in more detail herein, includes amplifying a 499 nucleotide sequence by PCR using the primer pair NL1 (5′-GCATATCAATAAGCGGAGGAAAAG-3′; SEQ ID NO: 26) and NL4 (5′-GGTCCGTGTTTCAAGACGG -3′; SEQ ID NO: 27).

The term “encode” or a grammatical equivalent thereof as it is applied to a nucleic acid sequence refers to a sequence of nucleic acids that code for amino acids of a peptide, polypeptide or protein upon translation if the nucleic acids are RNA or transcription and translation if the nucleic acids are DNA. Accordingly, the term “encoding nucleic acid sequence,” refers to a sequence of nucleic acids that code for amino acids upon transcription and/or translation. Such a sequence would include, for example, a genomic DNA sequence that corresponds to an exon of a eukaryotic gene or cDNA of a eukaryotic gene. Such sequences are in contrast to the enhancer, promoters and introns of the same gene, which do not, under normal conditions, code for any amino acids.

The term “exogenous” as it is used herein is intended to mean that the referenced molecule or the referenced activity is introduced into the Metschnikowia species described herein. The molecule can be introduced, for example, by introduction of an encoding nucleic acid into the host Metschnikowia species' genetic material, such as by integration into a host chromosome or as non-chromosomal genetic material such as a plasmid. Alternatively or additionally, the molecule introduced can be or include, for example, a non-coding nucleic acid that modulates (e.g., increases, decreases or makes constitutive) the expression of an encoding nucleic acid, such as a promoter or enhancer. Therefore, the term as it is used in reference to expression of an encoding nucleic acid refers to introduction of the encoding nucleic acid in an expressible form into the host Metschnikowia species and/or introduction of a nucleic acid that increases expression (e.g., overexpresses) of an encoding nucleic acid of the host Metschnikowia species. When used in reference to a biosynthetic activity, the term refers to an activity that is introduced into the host Metschnikowia species. The source can be, for example, a homologous or heterologous encoding nucleic acid that expresses the referenced activity following introduction into the Metschnikowia species. Therefore, the term “endogenous” refers to a referenced molecule or activity that is present in the host Metschnikowia species. Similarly, the term when used in reference to expression of an encoding nucleic acid refers to expression of an encoding nucleic acid contained within the microbial organism. The term “heterologous” refers to a molecule or activity derived from a source other than the referenced Metschnikowia species, whereas “homologous” refers to a molecule or activity derived from the host Metschnikowia species. Accordingly, exogenous expression of an encoding nucleic acid disclosed herein can utilize either or both a heterologous or homologous encoding nucleic acid.

It is understood that when more than one exogenous nucleic acid is included in a Metschnikowia species that the more than one exogenous nucleic acid refers to the referenced encoding nucleic acid or biosynthetic activity, as discussed above. It is also understood that a microbial organism can have one or multiple copies of the same exogenous nucleic acid. It is further understood, as disclosed herein, that such more than one exogenous nucleic acid can be introduced into the host Metschnikowia species on separate nucleic acid molecules, on polycistronic nucleic acid molecules, or a combination thereof, and still be considered as more than one exogenous nucleic acid. For example, as disclosed herein a microbial organism can be engineered to express two or more exogenous nucleic acids encoding a desired pathway enzyme or protein. In the case where two exogenous nucleic acids encoding a desired activity are introduced into a host Metschnikowia species, it is understood that the two exogenous nucleic acids can be introduced as a single nucleic acid, for example, on a single plasmid, on separate plasmids, can be integrated into the host chromosome at a single site or multiple sites, and still be considered as two exogenous nucleic acids. Similarly, it is understood that more than two exogenous nucleic acids can be introduced into a host organism in any desired combination, for example, on a single plasmid, on separate plasmids, can be integrated into the host chromosome at a single site or multiple sites, and still be considered as two or more exogenous nucleic acids, for example three exogenous nucleic acids. Thus, the number of referenced exogenous nucleic acids or biosynthetic activities refers to the number of encoding nucleic acids or the number of biosynthetic activities, not the number of separate nucleic acids introduced into the host organism.

As used herein, the term “genetic modification,” “gene disruption,” or grammatical equivalents thereof, is intended to mean a genetic alteration that renders the encoded gene product functionally inactive, or active but attenuated. The genetic alteration can be, for example, deletion of the entire gene, deletion of a regulatory sequence required for transcription or translation, deletion of a portion of the gene that results in a truncated gene product, or by any of the various mutation strategies that inactivate or attenuate the encoded gene product well known in the art. One particularly useful method of gene disruption is complete gene deletion because it reduces or eliminates the occurrence of genetic reversions in the Metschnikowia species provided herein. A gene disruption also includes a null mutation, which refers to a mutation within a gene or a region containing a gene that results in the gene not being transcribed into RNA and/or translated into a functional gene product. Such a null mutation can arise from many types of mutations including, for example, inactivating point mutations, deletion of a portion of a gene, entire gene deletions, or deletion of chromosomal segments.

As used herein, the term “inactivate,” or grammatical equivalents thereof, is intended to mean to stop the activity of the enzyme or protein. Such inactivation can be accomplished by deletion of the entire nucleic acid sequence encoding the enzyme or protein. Inactivation can also be accomplished by deletion of a portion of the nucleic acid sequence encoding the enzyme or protein such that the resulting enzyme or protein encoded by the nucleic acid sequence does not have the activity of the full length enzyme or protein. Additionally, inactivation of an enzyme or protein can be accomplished by substitutions or insertions, including in combination with deletions, into the nucleic acid sequence encoding the enzyme or protein. Insertions can include heterologous nucleic acids, such as those described herein.

As used herein, the term “isolated” when used in reference to a Metschnikowia species described herein is intended to mean an organism that is substantially free of at least one component as the referenced microbial organism is found in nature. The term includes a Metschnikowia species that is removed from some or all components as it is found in its natural environment. The term also includes a microbial organism that is removed from some or all components as the microbial organism is found in non-naturally occurring environments. Therefore, an isolated Metschnikowia species is partly or completely separated from other substances as it is found in nature or as it is grown, stored or subsisted in non-naturally occurring environments. Specific examples of isolated Metschnikowia species include a partially pure microbial organism, a substantially pure microbial organism and a microbial organism cultured in a medium that is non-naturally occurring.

As used herein, the term “medium,” “culture medium,” “growth medium” or grammatical equivalents thereof refers to a liquid or solid (e.g., gelatinous) substance containing nutrients that supports the growth of a cell, including any microbial organism such as the Metschnikowia species described herein. Nutrients that support growth include: a substrate that supplies carbon, such as, but are not limited to, xylose, cellobiose, galactose, glucose, ethanol, acetate, arabitol, sorbitol and glycerol; salts that provide essential elements including magnesium, nitrogen, phosphorus, and sulfur; a source for amino acids, such as peptone or tryptone; and a source for vitamin content, such as yeast extract. Specific examples of medium useful in the methods and in characterizing the Metschnikowia species described herein include yeast extract peptone (YEP) medium and yeast nitrogen base (YNB) medium having a carbon source such as, but not limited to xylose, glucose, cellobiose, galactose, or glycerol, or a combination thereof. The formulations of YEP and YNB medium are well known in the art. For example, YEP medium having 4% xylose includes, but is not limited to, yeast extract 1.0 g, peptone 2.0 g, xylose 4.0 g, and 100 ml water. As another example, YNB medium having 2% glucose and 2% xylose includes, but is not limited to, biotin 2 calcium pantothenate 400 folic acid 2 μg, inositol 2000 μg, niacin 400 μg, p-aminobenzoic acid 200 μg, pyridoxine hydrochloride 400 μg, riboflavin 200 μg, thiamine hydrochloride 400 μg, boric acid 500 μg, copper sulfate 40 μg, potassium iodide 100 μg, ferric chloride 200 μg, manganese sulfate 400 μg, sodium molybdate 200 μg, zinc sulfate 400 μg, potassium phosphate monobasic 1 g, magnesium sulfate 500 mg, sodium chloride 100 mg, calcium chloride 100 mg, 20 g glucose, 20 g, xylose and 1 L water. The amount of the carbon source in the medium can be readily determined by a person skilled in the art. When more than one substrate that supplies carbon is present in the medium, these are referred to as “co-substrates.” Medium can also include substances other than nutrients needed for growth, such as a substance that only allows select cells to grow (e.g., antibiotic or antifungal), which are generally found in selective medium, or a substance that allows for differentiation of one microbial organism over another when grown on the same medium, which are generally found in differential or indicator medium. Such substances are well known to a person skilled in the art.

As used herein, the term “Metschnikowia species” refers to any species of yeast that falls within the Metschnikowia genus. Exemplary Metschnikowia species include, but are not limited to, Metschnikowia pulcherrima, Metschnikowia fructicola, Metschnikowia chrysoperlae, Metschnikowia reukaufii, Metschnikowia andauensis, Metschnikowia shanxiensis, Metschnikowia sinensis, Metschnikowia zizyphicola, Metschnikowia bicuspidata, Metschnikowia lunata, Metschnikowia zobellii, Metschnikowia australis, Metschnikowia agaveae, Metschnikowia gruessii, Metschnikowia hawaiiensis, Metschnikowia krissii, Metschnikowia sp. strain NS-O-85, Metschnikowia sp. strain NS-O-89 and the unique Metschnikowia species described herein, Metschnikowia sp. H0, alternatively known as “H0 Metschnikowia sp.” The Metschnikowia species described herein, i.e., the “H0 Metschnikowia sp.”, is a newly discovered species, which is designated Accession No. 081116-01, and was deposited at International Depositary Authority of Canada (“IDAC”), an International Depositary Authority, at the address of 1015 Arlington Street, Winnipeg, Manitoba, Canada R3E 3R2, on Nov. 8, 2016, under the terms of the Budapest Treaty. The proposed scientific name for the H0 Metschnikowia sp. is Metschnikowia vinificola (vinifi: from vinifera (species of wine grape vine); cola: from Latin word “incola” meaning inhabitant). Thus, the species name of vinificola (inhabitant of vinifera) refers to the isolation of the type strain from wine grapes.

Additionally, a Metschnikowia species referred to herein can include a “non-naturally occurring” or “recombinant” Metschnikowia species. Such an organism is intended to mean a Metschnikowia species that has at least one genetic alteration not normally found in the naturally occurring Metschnikowia species, including wild-type strains of the referenced species. Genetic alterations include, for example, modifications introducing expressible nucleic acids encoding metabolic polypeptides, other nucleic acid additions, nucleic acid deletions and/or other gene disruption of the microbial organism's genetic material. Such modifications include, for example, coding regions and functional fragments thereof, for heterologous, homologous or both heterologous and homologous polypeptides for the referenced species. Additional modifications include, for example, non-coding regulatory regions in which the modifications alter expression of a gene or operon. Exemplary metabolic polypeptides include enzymes or proteins within a metabolic pathway described herein.

A metabolic modification refers to a biochemical reaction that is altered from its naturally occurring state. Therefore, the Metschnikowia species described herein can have genetic modifications to one or more nucleic acid sequence encoding metabolic polypeptides, or functional fragments thereof, which alter the biochemical reaction that the metabolic polypeptide catalyzes, including catabolic or anabolic reactions and basal metabolism. Exemplary metabolic modifications are disclosed herein.

As used herein, the term “metabolic pathway” refers to one or more metabolic polypeptides (e.g., proteins or enzymes) that catalyze the conversion of a substrate compound to a product compound and/or produce a co-substrate for the conversion of a substrate compound to a product compound. Such a product compound can be one of the bioderived compounds described herein, or an intermediate compound that can lead to the bioderived compound upon further conversion by other proteins or enzymes of the metabolic pathway. Accordingly, a metabolic pathway can be comprised of a series of metabolic polypeptides (e.g., two, three, four, five, six, seven, eight, nine, ten or more) that act upon a substrate compound to convert it to a given product compound through a series of intermediate compounds. The metabolic polypeptides of a metabolic pathway can be encoded by an exogenous nucleic acid as described herein or produced naturally by the Metschnikowia species.

As used herein, the term “overexpression” or grammatical equivalents thereof, is intended to mean the expression of a gene product (e.g., ribonucleic acids (RNA), protein or enzyme) in an amount that is greater than is normal for a host Metschnikowia species, or at a time or location within the host Metschnikowia species that is different from that of wild-type expression.

As used herein, the terms “sequence identity” or “sequence homology,” when used in reference to a nucleic acid sequence or an amino acid sequence, refers to the similarity between two or more nucleic acid molecules or between two or more polypeptides. Identity can be determined by comparing a position in each sequence, which may be aligned for purposes of comparison. When a position in the compared sequence is occupied by the same base or amino acid, then the molecules are identical at that position. A degree of identity between sequences is a function of the number of matching or homologous positions shared by the sequences. The alignment of two sequences to determine their percent sequence identity can be done using software programs known in the art, such as, for example, those described in Ausubel et al., Current Protocols in Molecular Biology, John Wiley and Sons, Baltimore, Md. (1999). Preferably, default parameters are used for the alignment. One alignment program well known in the art that can be used is BLAST set to default parameters. In particular, programs are BLASTN and BLASTP, using the following default parameters: Genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+SwissProtein+SPupdate+PIR. Details of these programs can be found at the National Center for Biotechnology Information.

As used herein, the term “substantially anaerobic” when used in reference to a culture or growth condition is intended to mean that the amount of dissolved oxygen in a liquid medium is less than about 10% of saturation. The term also is intended to include sealed chambers maintained with an atmosphere of less than about 1% oxygen that include liquid or solid medium.

As used herein, the term “sugar alcohol” refers to an alcohol produced by the reduction of an aldehyde or ketone of a sugar. Thus a “C7 sugar alcohol” refers to an alcohol produced by the reduction of an aldehyde or ketone of a sugar having seven carbon atoms, such as volemitol or an isomer thereof.

As used herein, the term “xylitol” refers to a pentose sugar alcohol having the chemical formula of C₅H₁₂O₅, a Molar mass of 152.15 g/mol, and one IUPAC name of (2R,3r,4S)-pentane-1,2,3,4,5-pentol [(2S,4R)-pentane-1,2,3,4,5-pentol]. Xylitol is commonly used as a low-calorie, low-carbohydrate alternative to sugar, which does not affect insulin levels of people with diabetes and individuals with hyperglycemia.

As used herein, the term “xylose” refers to a five carbon monosaccharide with a formyl functional group having the chemical formula of C₅H₁₀O₅, a Molar mass of 150.13 g/mol, and one IUPAC name of (3R,4S,5R)-oxane-2,3,4,5-tetrol. Xylose is also known in the art as D-xylose, D-xylopyranose, xyloside, d-(+)-xylose, xylopyranose, wood sugar, xylomed and D-xylopentose.

Provided herein are novel isolated Metschnikowia species that produce xylitol, and other bioderived compounds, from xylose when cultured in medium having xylose. Accordingly, in some embodiments, provided herein an isolated Metschnikowia species that produces at least 0.1 g/L/h of xylitol from xylose when cultured. Also provided herein is an isolated Metschnikowia species that produces at least 1 g/L of xylose to xylitol when cultured.

As can be understood by a person skilled in the art, the amount of xylitol from xylose produced by the isolated Metschnikowia species provided herein can vary depending on the culturing conditions and/or the metabolic modifications made to the Metschnikowia species as described herein. Accordingly, in some embodiments, the amount of xylitol produced by the isolated Metschnikowia species is at least 0.2 g/L/h of xylitol from xylose. In some embodiments, the amount of xylitol produced by the isolated Metschnikowia species is at least 0.3 g/L/h of xylitol from xylose. In some embodiments, the amount of xylitol produced by the isolated Metschnikowia species is at least 0.4 g/L/h of xylitol from xylose. In some embodiments, the amount of xylitol produced by the isolated Metschnikowia species is at least 0.50 g/L/h of xylitol from xylose. In some embodiments, the amount of xylitol produced by the isolated Metschnikowia species is at least 0.60 g/L/h of xylitol from xylose. In some embodiments, the amount of xylitol produced by the isolated Metschnikowia species is at least 0.70 g/L/h of xylitol from xylose. In some embodiments, the amount of xylitol produced by the isolated Metschnikowia species is at least 0.80 g/L/h of xylitol from xylose. In some embodiments, the amount of xylitol produced by the isolated Metschnikowia species is at least 0.90 g/L/h of xylitol from xylose. In some embodiments, the amount of xylitol produced by the isolated Metschnikowia species is at least 1.00 g/L/h of xylitol from xylose. In some embodiments, the amount of xylitol produced by the isolated Metschnikowia species is at least 1.50 g/L/h of xylitol from xylose. In some embodiments, the amount of xylitol produced by the isolated Metschnikowia species is at least 2.00 g/L/h of xylitol from xylose. In some embodiments, the amount of xylitol produced by the isolated Metschnikowia species is at least 2.50 g/L/h of xylitol from xylose. In some embodiments, the amount of xylitol produced by the isolated Metschnikowia species is at least 3.00 g/L/h of xylitol from xylose. In some embodiments, the amount of xylitol produced by the isolated Metschnikowia species is at least 3.50 g/L/h of xylitol from xylose. In some embodiments, the amount of xylitol produced by the isolated Metschnikowia species is at least 4.00 g/L/h of xylitol from xylose. In some embodiments, the amount of xylitol produced by the isolated Metschnikowia species is at least 5.00 g/L/h of xylitol from xylose. In some embodiments, the amount of xylitol produced by the isolated Metschnikowia species is at least 6.00 g/L/h of xylitol from xylose. In some embodiments, the amount of xylitol produced by the isolated Metschnikowia species is at least 7.00 g/L/h of xylitol from xylose. In some embodiments, the amount of xylitol produced by the isolated Metschnikowia species is at least 8.00 g/L/h of xylitol from xylose. In some embodiments, the amount of xylitol produced by the isolated Metschnikowia species is at least 9.00 g/L/h of xylitol from xylose. In some embodiments, the amount of xylitol produced by the isolated Metschnikowia species is or at least 10.00 g/L/h of xylitol from xylose.

In some embodiments, the conversion efficiency of the isolated Metschnikowia species provided herein to convert xylose to xylitol is at least 0.01 g xylitol per 1 g xylose. The conversion efficiency can be at least 0.02 g xylitol per 1 g xylose. The conversion efficiency can be at least 0.03 g xylitol per 1 g xylose. The conversion efficiency can be at least 0.04 g xylitol per 1 g xylose. The conversion efficiency can be at least 0.05 g xylitol per 1 g xylose. The conversion efficiency can be at least 0.06 g xylitol per 1 g xylose. The conversion efficiency can be at least 0.07 g xylitol per 1 g xylose. The conversion efficiency can be at least 0.08 g xylitol per 1 g xylose. The conversion efficiency can be at least 0.09 g xylitol per 1 g xylose. The conversion efficiency can be at least 0.1 g xylitol per 1 g xylose. The conversion efficiency can be at least 0.15 g xylitol per 1 g xylose. The conversion efficiency can be at least 0.2 g xylitol per 1 g xylose. The conversion efficiency can be at least 0.25 g xylitol per 1 g xylose. The conversion efficiency can be at least 0.3 g xylitol per 1 g xylose. The conversion efficiency can be at least 0.35 g xylitol per 1 g xylose. The conversion efficiency can be at least 0.4 g xylitol per 1 g xylose. The conversion efficiency can be at least 0.45 g xylitol per 1 g xylose. The conversion efficiency can be at least 0.5 g xylitol per 1 g xylose. The conversion efficiency can be at least 0.55 g xylitol per 1 g xylose. The conversion efficiency can be at least 0.6 g xylitol per 1 g xylose. The conversion efficiency can be at least 0.65 g xylitol per 1 g xylose. The conversion efficiency can be at least 0.7 g xylitol per 1 g xylose. The conversion efficiency can be at least 0.75 g xylitol per 1 g xylose. The conversion efficiency can be at least 0.8 g xylitol per 1 g xylose. The conversion efficiency can be at least 0.85 g xylitol per 1 g xylose. The conversion efficiency can be at least 0.9 g xylitol per 1 g xylose. The conversion efficiency can be at least 0.95 g xylitol per 1 g xylose. The conversion efficiency can be at least 1 g xylitol per 1 g xylose.

In some embodiments, the concentration of xylitol produced in the culture medium by the isolated Metschnikowia species is at least 1 g/L. In some embodiments, the concentration of xylitol produced in the culture medium by the isolated Metschnikowia species is at least 2 g/L. In some embodiments, the concentration of xylitol produced in the culture medium by the isolated Metschnikowia species is at least 3 g/L. In some embodiments, the concentration of xylitol produced in the culture medium by the isolated Metschnikowia species is at least 4 g/L. In some embodiments, the concentration of xylitol produced in the culture medium by the isolated Metschnikowia species is at least 5 g/L. In some embodiments, the concentration of xylitol produced in the culture medium by the isolated Metschnikowia species is at least 10 g/L. In some embodiments, the concentration of xylitol produced in the culture medium by the isolated Metschnikowia species is at least 20 g/L. In some embodiments, the concentration of xylitol produced in the culture medium by the isolated Metschnikowia species is at least 30 g/L. In some embodiments, the concentration of xylitol produced in the culture medium by the isolated Metschnikowia species is at least 40 g/L. In some embodiments, the concentration of xylitol produced in the culture medium by the isolated Metschnikowia species is at least 50 g/L. In some embodiments, the concentration of xylitol produced in the culture medium by the isolated Metschnikowia species is at least 60 g/L. In some embodiments, the concentration of xylitol produced in the culture medium by the isolated Metschnikowia species is at least 70 g/L. In some embodiments, the concentration of xylitol produced in the culture medium by the isolated Metschnikowia species is at least 80 g/L. In some embodiments, the concentration of xylitol produced in the culture medium by the isolated Metschnikowia species is at least 90 g/L. In some embodiments, the concentration of xylitol produced in the culture medium by the isolated Metschnikowia species is at least 100 g/L. In some embodiments, the concentration of xylitol produced in the culture medium by the isolated Metschnikowia species is at least 150 g/L. In some embodiments, the concentration of xylitol produced in the culture medium by the isolated Metschnikowia species is at least 200 g/L. In some embodiments, the concentration of xylitol produced in the culture medium by the isolated Metschnikowia species is at least 250 g/L. In some embodiments, the concentration of xylitol produced in the culture medium by the isolated Metschnikowia species is at least 300 g/L.

Also provided herein is an isolated Metschnikowia species that produces a combination of bioderived compounds described herein, each at a specific rate. For example, an isolated Metschnikowia species provided herein can produce about 0.11 g/L/h of xylitol and one or more of the following compounds: about 6.8E-05 g/L/h of n-butanol, about 2.5E-04 g/L/h of isobutanol, about 2.4E-04 g/L/h of isopropanol, about 2.64E-04 g/L/h of ethanol or about 3.73E-06 g/L/h of 2-phenylethyl alcohol. In some embodiments, an isolated Metschnikowia species provided herein can produce about 6.8E-05 g/L/h of n-butanol. In some embodiments, an isolated Metschnikowia species provided herein can produce about 2.5E-04 g/L/h of isobutanol. In some embodiments, an isolated Metschnikowia species provided herein can produce about 2.4E-04 g/L/h of isopropanol. In some embodiments, an isolated Metschnikowia species provided herein can produce about 2.64E-04 g/L/h of ethanol. In some embodiments, an isolated Metschnikowia species provided herein can produce about 3.73E-06 g/L/h of 2-phenylethyl alcohol. When an isolated Metschnikowia species described herein produces a combination of bioderived compounds at specific rates, then the ratio of these compounds can be determined. Accordingly, in some embodiments, an isolated Metschnikowia species described herein produces compounds xylitol, n-butanol, isobutanol, isopropanol, ethanol and 2-phenylethyl alcohol at a concentration of about 8,000 mg/L xylitol, about 4.85 mg/L n-butanol, about 18.06 mg/L isobutanol, about 17.5 mg/L isopropanol, about 19.7 mg/L ethanol and about 0.269 mg/L 2-phenylethyl alcohol.

Culturing conditions that can yield the rate of xylitol from xylose described herein include conditions that vary the amount of aeration of the medium, the temperature of the medium, the amount of time the culture is grown for and the composition of the medium. In some embodiments, the culturing of the isolated Metschnikowia species occurs under aerobic conditions. In some embodiments, the culturing of the isolated Metschnikowia species occurs under substantially anaerobic conditions. In some embodiments, the temperature of the medium ranges from 20° C. to 35° C., or alternatively 26° C. to 35° C., or alternatively 28° C. to 32° C., or alternatively at about 30° C. In some embodiments, the culture is grown for 1 day. In some embodiment, the culture is grown for 2 days. In some embodiments, the culture is grown for 3 days. In some embodiments, the culture is grown for 4 days. In some embodiments, the culture is grown for 5 days. In some embodiments, the culture is grown for 6 days. In some embodiments, the culture is grown for 7 or more days. The composition of the medium can be any medium well known in the art for culturing yeast, especially species within the genus of Metschnikowia. Exemplary medium include, but are not limited to, yeast extract peptone (YEP) medium or yeast nitrogen base (YNB) medium. Additionally, the carbon source in the medium used by the isolated Metschnikowia species can include xylose as the only carbon source, as well as xylose in combination with other carbon sources described herein. The amount of the carbon source in the medium can range from 1% to 20% (e.g., 1% to 20% xylose), or alternatively 2% to 14% (e.g., 2% to 14% xylose), or alternatively 4% to 10% (e.g., 4% to 10% xylose). In some embodiments, the amount of the carbon source is 4% (e.g., 4% xylose).

In some embodiments, xylose is not the only carbon source. For example, in some embodiments, the medium includes xylose and a C3 carbon source, a C4 carbon source, a C5 carbon source, a C6 carbon source, or a combination thereof. Accordingly, in some embodiments, the medium includes xylose and a C3 carbon source (e.g., glycerol). In some embodiments, the medium includes xylose and a C4 carbon source (e.g., erythrose or threose). In some embodiments, the medium includes xylose and a C5 carbon source (e.g., arabitol, ribose or lyxose). In some embodiments, the medium includes xylose and a C6 carbon source (e.g., glucose, galactose, mannose, allose, altrose, gulose, and idose). Alternatively or additionally, in some embodiments, the medium includes xylose and cellobiose, galactose, glucose, arabitol, sorbitol and glycerol, or a combination thereof. In a specific embodiment, the medium includes xylose and glucose. The amount of the two or more carbon sources in the medium can range independently from 1% to 20% (e.g., 1% to 20% xylose and 1% to 20% glucose), or alternatively 2% to 14% (e.g., 2% to 14% xylose and 2% to 14% glucose), or alternatively 4% to 10% (e.g., 4% to 10% xylose and 4% to 10%). In a specific embodiment, the amount of each of the carbon sources is 2% (e.g., 2% xylose and 2% glucose)

Based on the conditions described herein, in a specific embodiment, provided herein is an isolated Metschnikowia species that produces at least 0.1 g/L/h of xylitol from xylose when cultured under aerobic conditions and at 30° C. for three days in liquid yeast extract peptone (YEP) medium comprising 4% xylose. In another specific embodiment, provided herein is an isolated Metschnikowia species that converts at least 0.1% (w/v) xylose to xylitol when cultured under aerobic conditions and at 30° C. for three days in liquid yeast nitrogen base (YNB) medium comprising 4% xylose. In yet another specific embodiment, provided herein is an isolated Metschnikowia species that converts at least 0.1% (w/v) xylose to xylitol when cultured under aerobic conditions and at 30° C. for two days in liquid yeast nitrogen base (YNB) medium comprising 2% xylose and 2% glucose. In still another specific embodiment, an isolated Metschnikowia species provided herein can produce about 0.11 g/L/h of xylitol, about 6.8E-05 g/L/h of n-butanol, about 2.5E-04 g/L/h of isobutanol, about 2.4E-04 g/L/h of isopropanol, about 2.64E-04 g/L/h of ethanol and about 3.73E-06 g/L/h of 2-phenylethyl alcohol when cultured under aerobic conditions for three days in liquid yeast extract peptone (YEP) medium comprising 4% xylose. In still another specific embodiment, an isolated Metschnikowia species provided herein can produce compounds xylitol, n-butanol, isobutanol, isopropanol, ethanol and 2-phenylethyl alcohol at a concentration of about 8,000 mg/L xylitol, about 4.85 mg/L n-butanol, about 18.06 mg/L isobutanol, about 17.5 mg/L isopropanol, about 19.7 mg/L ethanol and about 0.269 mg/L 2-phenylethyl alcohol when cultured under aerobic conditions for three days in liquid yeast extract peptone (YEP) medium comprising 4% xylose. In still another specific embodiment, an isolated Metschnikowia species provided herein can produce compounds xylitol, n-butanol, isobutanol, isopropanol, ethanol and 2-phenylethyl alcohol at a relative ratio of 99.26% xylitol, 0.061% n-butanol, 0.223% isobutanol, 0.217% isopropanol, 0.236% ethanol and 0.003% 2-phenylethyl alcohol when cultured under aerobic conditions for three days in liquid yeast extract peptone (YEP) medium comprising 4% xylose.

Suitable purification and/or assays to test for the production of a bioderived compound produced by a Metschnikowia species described herein, including assays to test for production of xylitol, n-butanol, isobutanol, isopropanol, ethanol or 2-phenylethyl alcohol, can be performed using well known methods (see also Examples). Suitable replicates, such as triplicate cultures, can be grown for each Metschnikowia species to be tested. Compound and byproduct formation in the Metschnikowia species can be monitored. The final product, intermediates, and other compounds can be analyzed by methods such as HPLC (High Performance Liquid Chromatography), GC-MS (Gas Chromatography-Mass Spectroscopy) and LC-MS (Liquid Chromatography-Mass Spectroscopy) or other suitable analytical methods using routine procedures well known in the art. The release of compound in the fermentation broth can also be tested with the culture supernatant. Byproducts and residual carbon sources can be quantified by HPLC using, for example, a cation-exchange column, a refractive index detector, and a UV detector (Lin et al., Biotechnol. Bioeng. 90:775-779 (2005)), or other suitable assay and detection methods well known in the art. The individual enzyme or protein activities from a metabolic pathway can also be assayed using methods well known in the art.

An isolated Metschnikowia species provided herein, in addition to or as an alternative to the above production characteristic, can be identified by genetic characteristic. For example, in some embodiments, an isolated Metschnikowia species described herein has a D1/D2 domain sequence that includes SEQ ID NO: 1. In some embodiments, an isolated Metschnikowia species described herein has a D1/D2 domain sequence with a nucleic acid sequence that is at least 96.8%, at least 96.9%, at least 97%, at least 97.1%, at least 97.2%, at least 97.3%, at least 97.4%, at least 97.5%, at least 97.5%, at least 97.6%, at least 97.7%, at least 97.8%, at least 97.9%, at least 98%, at least 98.1%, at least 98.2%, at least 98.3%, at least 98.4%, at least 98.5%, at least 98.6%, at least 98.7%, at least 98.8%, at least 98.9%, at least 99%, at least 99.1%, at least 99.2%, at least 99.3%, at least 99.4%, at least 99.5%, at least 99.6%, at least 99.7%, at least 99.8%, or at least 99.9% identical to SEQ ID NO: 1. In some embodiments, an isolated Metschnikowia species described herein has a D1/D2 domain sequence that includes a nucleic acid sequence within the consensus sequence of SEQ ID NO: 2. In some embodiments, an isolated Metschnikowia species described herein has a D1/D2 domain sequence that is at least 97.1,% at least 97.2%, at least 97.3%, at least 97.4%, at least 97.5%, at least 97.6%, at least 97.7%, at least 97.8%, at least 97.9%, at least 98.0%, at least 98.1%, at least 98.2%, at least 98.3%, at least 98.4%, at least 98.5%, at least 98.6%, at least 98.7%, at least 98.8%, at least 98.9%, at least 99.0%, at least 99.1%, at least 99.2%, at least 99.3%, at least 99.4%, at least 99.5%, at least 99.6%, at least 99.7%, at least 99.8%, at least 99.9% identical to the D1/D2 domain consensus sequence of SEQ ID NO: 2. In some embodiments, an isolated Metschnikowia species described herein has a D1/D2 domain sequence that includes a nucleic acid sequence comprising residues 1-153, 178 to 434 and 453 to 499 of SEQ ID NO: 2 with no more than 1, 2, 3 or 4 nucleotide substitutions therein.

In addition or alternatively to the sequence of the D1/D2 domain, an isolated Metschnikowia species described herein can be identified by the presence of a nucleic acid sequence that is unique to H0 Metschnikowia sp. Accordingly, in some embodiments, an isolated Metschnikowia species described herein has at least one nucleic acid sequence encoding an amino acid sequence selected from Aro10 (SEQ ID NO: 37), Gxf2 (SEQ ID NO: 40), Hgt19 (SEQ ID NO: 42), Hxt5 (SEQ ID NO: 44), Tef1 (SEQ ID NO: 46), Xks1 (SEQ ID NO: 51), Xyl1 (SEQ ID NO: 52), Tal1 (SEQ ID NO: 55) and Tkl1 (SEQ ID NO: 56). In some embodiments, an isolated Metschnikowia species described herein includes a nucleic acid sequence encoding the amino acid sequence the Aro10 protein (SEQ ID NO: 37). In some embodiments, an isolated Metschnikowia species described herein includes a nucleic acid sequence encoding the amino acid sequence the Gxf2 protein (SEQ ID NO: 40). In some embodiments, an isolated Metschnikowia species described herein includes a nucleic acid sequence encoding the amino acid sequence the Hgt19 protein (SEQ ID NO: 42). In some embodiments, an isolated Metschnikowia species described herein includes a nucleic acid sequence encoding the amino acid sequence the Hxt5 protein (SEQ ID NO: 44). In some embodiments, an isolated Metschnikowia species described herein includes a nucleic acid sequence encoding the amino acid sequence the Tef1 protein (SEQ ID NO: 49). In some embodiments, an isolated Metschnikowia species described herein includes a nucleic acid sequence encoding the amino acid sequence the Xks1 protein (SEQ ID NO: 51). In some embodiments, an isolated Metschnikowia species described herein includes a nucleic acid sequence encoding the amino acid sequence the Xyl1 protein (SEQ ID NO: 52). In some embodiments, an isolated Metschnikowia species described herein includes a nucleic acid sequence encoding the amino acid sequence the Tal1 protein (SEQ ID NO: 55). In some embodiments, an isolated Metschnikowia species described herein includes a nucleic acid sequence encoding the amino acid sequence the Tkl1 protein (SEQ ID NO: 56).

In some embodiments, an isolated Metschnikowia species described herein has at least one encoding nucleic acid sequence selected from ACT1 (SEQ ID NO: 57), ARO8 (SEQ ID NO: 58), ARO10 (SEQ ID NO: 59), GPD1 (SEQ ID NO: 60), GXF1 (SEQ ID NO: 61), GXF2 (SEQ ID NO: 62), GXS1 (SEQ ID NO: 63), HXT19 (SEQ ID NO: 64), HXT2.6 (SEQ ID NO: 65), HXT5 (SEQ ID NO: 66), PGK1 (SEQ ID NO: 67), QUP2 (SEQ ID NO: 68), RPB1 (SEQ ID NO: 69), RPB2 (SEQ ID NO: 70), TEF1 (SEQ ID NO: 71), TPI1 (SEQ ID NO: 72), XKS1 (SEQ ID NO: 73), XYL1 (SEQ ID NO: 74), XYL2 (SEQ ID NO: 75), XYT1 (SEQ ID NO: 76), TAL1 (SEQ ID NO: 77) and TKL1 (SEQ ID NO: 78). In some embodiments, an isolated Metschnikowia species described herein includes an encoding nucleic acid sequence of ACT1 (SEQ ID NO: 57). In some embodiments, an isolated Metschnikowia species described herein includes an encoding nucleic acid sequence of ARO8 (SEQ ID NO: 58). In some embodiments, an isolated Metschnikowia species described herein includes an encoding nucleic acid sequence of ARO10 (SEQ ID NO: 59). In some embodiments, an isolated Metschnikowia species described herein includes an encoding nucleic acid sequence of GPD1 (SEQ ID NO: 60). In some embodiments, an isolated Metschnikowia species described herein includes an encoding nucleic acid sequence of GXF1 (SEQ ID NO: 61). In some embodiments, an isolated Metschnikowia species described herein includes an encoding nucleic acid sequence of GXF2 (SEQ ID NO: 62). In some embodiments, an isolated Metschnikowia species described herein includes an encoding nucleic acid sequence of GXS1 (SEQ ID NO: 63). In some embodiments, an isolated Metschnikowia species described herein includes an encoding nucleic acid sequence of HXT19 (SEQ ID NO: 64). In some embodiments, an isolated Metschnikowia species described herein includes an encoding nucleic acid sequence of HXT2.6 (SEQ ID NO: 65). In some embodiments, an isolated Metschnikowia species described herein includes an encoding nucleic acid sequence of HXT5 (SEQ ID NO: 66). In some embodiments, an isolated Metschnikowia species described herein includes an encoding nucleic acid sequence of PGK1 (SEQ ID NO: 67). In some embodiments, an isolated Metschnikowia species described herein includes an encoding nucleic acid sequence of QUP2 (SEQ ID NO: 68). In some embodiments, an isolated Metschnikowia species described herein includes an encoding nucleic acid sequence of RPB1 (SEQ ID NO: 69). In some embodiments, an isolated Metschnikowia species described herein includes an encoding nucleic acid sequence of RPB2 (SEQ ID NO: 70). In some embodiments, an isolated Metschnikowia species described herein includes an encoding nucleic acid sequence of TEF1 (SEQ ID NO: 71). In some embodiments, an isolated Metschnikowia species described herein includes an encoding nucleic acid sequence of TPI1 (SEQ ID NO: 72). In some embodiments, an isolated Metschnikowia species described herein includes an encoding nucleic acid sequence of XKS1 (SEQ ID NO: 73). In some embodiments, an isolated Metschnikowia species described herein includes an encoding nucleic acid sequence of XYL1 (SEQ ID NO: 74). In some embodiments, an isolated Metschnikowia species described herein includes an encoding nucleic acid sequence of XYL2 (SEQ ID NO: 75). In some embodiments, an isolated Metschnikowia species described herein includes an encoding nucleic acid sequence of XYT1 (SEQ ID NO: 76). In some embodiments, an isolated Metschnikowia species described herein includes an encoding nucleic acid sequence of TAL1 (SEQ ID NO: 77). In some embodiments, an isolated Metschnikowia species described herein includes an encoding nucleic acid sequence of TKL1 (SEQ ID NO: 78).

In addition or alternatively to the sequence of the D1/D2 domain and the unique protein and encoding nucleic acids of H0 Metschnikowia sp., an isolated Metschnikowia species described herein can be identified by certain physiological characteristics. For example, in some embodiments, an isolated Metschnikowia species described herein grows to an OD₆₀₀ of about 25 within 41 hours of culturing in yeast extract peptone (YEP) medium comprising 2% xylose as the sole carbon source. Other identifying characteristics include: cells that are globose to oval in shape; multilateral budding; abundant spherical chlamydospore-like ‘pulcherrima’ cells when grown in YPD broth for 7 days at 30° C.; slow growth at 4° C., normal growth at 20° C. to 33° C., and/or no growth at 37° C. on YPD agar; secretion of pink pigment into medium; and the assimilation D-glucose, D-galactose, D-xylose, sucrose, glycerol, ethanol, succinate and cellobiose.

In certain specific embodiments, an isolated Metschnikowia species described herein includes a nucleic acid sequence that is at least 96.8% identical to SEQ ID NO: 1 and at least one nucleic acid sequence encoding an amino acid sequence selected from SEQ ID NOS: 37, 40, 42, 44, 49, 51, 52, 55 and 56.

In certain specific embodiments, an isolated Metschnikowia species described herein includes a nucleic acid sequence within the consensus sequence of SEQ ID NO: 2 and at least one nucleic acid sequence encoding an amino acid sequence selected from SEQ ID NOS: 37, 40, 42, 44, 49, 51, 52, 55 and 56.

In certain specific embodiments, an isolated Metschnikowia species described herein includes a nucleic acid sequence comprising residues 1-153, 178 to 434 and 453 to 499 of SEQ ID NO: 2 with no more than 4 nucleotide substitutions therein, and at least one nucleic acid sequence encoding an amino acid sequence selected from SEQ ID NOS: 37, 40, 42, 44, 49, 51, 52, 55 and 56.

In certain specific embodiments, an isolated Metschnikowia species described herein includes a D1/D2 domain sequence that includes a nucleic acid sequence that is at least 96.8% identical to SEQ ID NO: 1 and at least one encoding nucleic acid sequence selected from SEQ ID NOS: 57-78.

In certain specific embodiments, an isolated Metschnikowia species described herein includes a D1/D2 domain sequence that includes a nucleic acid sequence within the consensus sequence of SEQ ID NO: 2 and at least one encoding nucleic acid sequence selected from SEQ ID NOS: 57-78.

In certain specific embodiments, an isolated Metschnikowia species described herein includes a D1/D2 domain sequence that includes a nucleic acid sequence comprising residues 1-153, 178 to 434 and 453 to 499 of SEQ ID NO: 2 with no more than 4 nucleotide substitutions therein, and at least one encoding nucleic acid sequence selected from SEQ ID NOS: 57-78.

In certain specific embodiments, an isolated Metschnikowia species described herein includes: a D1/D2 domain sequence that is at least 96.8% identical to SEQ ID NO: 1; and an encoding nucleic acid sequence of SEQ ID NO: 70, and wherein the isolated Metschnikowia species grows to an OD₆₀₀ of about 25 within 41 hours of culturing in yeast extract peptone (YEP) medium comprising 2% xylose as the sole carbon source.

In certain specific embodiments, an isolated Metschnikowia species described herein includes a nucleic acid sequence that is at least 97.1% identical to the D1/D2 domain consensus sequence of SEQ ID NO: 2; and an encoding nucleic acid sequence of SEQ ID NO: 70.

Also provided herein is an isolated Metschnikowia species having one of the specific D1/D2 domain sequence described herein. For example, in some embodiments, an isolated Metschnikowia species described herein includes a nucleic acid sequence selected from one of SEQ ID NOS: 1 and 3-25. Accordingly, in some embodiments, the isolated Metschnikowia species includes a nucleic acid sequence of SEQ ID NO: 1. In some embodiments, the isolated Metschnikowia species includes a nucleic acid sequence of SEQ ID NO: 3. In some embodiments, the isolated Metschnikowia species includes a nucleic acid sequence of SEQ ID NO: 4. In some embodiments, the isolated Metschnikowia species includes a nucleic acid sequence of SEQ ID NO: 5. In some embodiments, the isolated Metschnikowia species includes a nucleic acid sequence of SEQ ID NO: 6. In some embodiments, the isolated Metschnikowia species includes a nucleic acid sequence of SEQ ID NO: 7. In some embodiments, the isolated Metschnikowia species includes a nucleic acid sequence of SEQ ID NO: 8. In some embodiments, the isolated Metschnikowia species includes a nucleic acid sequence of SEQ ID NO: 9. In some embodiments, the isolated Metschnikowia species includes a nucleic acid sequence of SEQ ID NO: 10. In some embodiments, the isolated Metschnikowia species includes a nucleic acid sequence of SEQ ID NO: 11. In some embodiments, the isolated Metschnikowia species includes a nucleic acid sequence of SEQ ID NO: 12. In some embodiments, the isolated Metschnikowia species includes a nucleic acid sequence of SEQ ID NO: 13. In some embodiments, the isolated Metschnikowia species includes a nucleic acid sequence of SEQ ID NO: 14. In some embodiments, the isolated Metschnikowia species includes a nucleic acid sequence of SEQ ID NO: 15. In some embodiments, the isolated Metschnikowia species includes a nucleic acid sequence of SEQ ID NO: 16. In some embodiments, the isolated Metschnikowia species includes a nucleic acid sequence of SEQ ID NO: 17. In some embodiments, the isolated Metschnikowia species includes a nucleic acid sequence of SEQ ID NO: 18. In some embodiments, the isolated Metschnikowia species includes a nucleic acid sequence of SEQ ID NO: 19. In some embodiments, the isolated Metschnikowia species includes a nucleic acid sequence of SEQ ID NO: 20. In some embodiments, the isolated Metschnikowia species includes a nucleic acid sequence of SEQ ID NO: 21. In some embodiments, the isolated Metschnikowia species includes a nucleic acid sequence of SEQ ID NO: 22. In some embodiments, the isolated Metschnikowia species includes a nucleic acid sequence of SEQ ID NO: 23. In some embodiments, the isolated Metschnikowia species includes a nucleic acid sequence of SEQ ID NO: 24. In some embodiments, the isolated Metschnikowia species includes a nucleic acid sequence of SEQ ID NO: 25.

In certain specific embodiments, an isolated Metschnikowia species described herein includes a D1/D2 domain that does not comprise the D1/D2 domain of a known Metschnikowia species. For example, such domains that are not included are the D1/D2 domains of, but not limited to, a species within the Metschnikowia pulcherrima clade, such as Metschnikowia andauensis, Metschnikowia chrysoperlae, Metschnikowia fructicola, Metschnikowia pulcherrima, Metschnikowia shanxiensis, Metschnikowia sinensis, and Metschnikowia zizyphicola.

In some embodiments, provided herein is an isolated Metschnikowia species designated Accession No. 081116-01, deposited at the International Depositary Authority of Canada, an International Depositary Authority, on Nov. 8, 2016, under the terms of the Budapest Treaty. The isolated Metschnikowia species designated Accession No. 081116-01 is referred to herein as “H0” or the “H0 Metschnikowia sp.” The International Depositary Authority of Canada is located at 1015 Arlington Street, Winnipeg, Manitoba, Canada R3E 3R2.

Also provided herein is a recombinant Metschnikowia species. Accordingly, in some embodiments, provided herein is an isolated Metschnikowia species designated Accession No. 081116-01, deposited at the International Depositary Authority of Canada, an International Depositary Authority, on Nov. 8, 2016, under the terms of the Budapest Treaty, wherein the Metschnikowia species further includes a metabolic pathway capable of producing a bioderived compound from xylose or a genetic modification, or both. In a specific embodiment, the metabolic pathway comprises at least one exogenous nucleic acid sequence encoding at least one enzyme of the metabolic pathway.

As described herein, the recombinant Metschnikowia species provided can be modified to include a metabolic pathway capable of producing a bioderived compound from xylose. When that modification includes the introduction of a heterologous exogenous nucleic acid sequence encoding at least one enzyme of the metabolic pathway, the coding sequence of enzyme can be modified in accordance with the codon usage of the host. The standard genetic code is well known in the art, as reviewed in, for example, Osawa et al., Microbiol Rev. 56(1):229-64 (1992). Yeast species, including but not limited to Saccharomyces cerevisiae, Candida azyma, Candida diversa, Candida magnoliae, Candida rugopelliculosa, Yarrowia lipolytica, and Zygoascus hellenicus, use the standard code. Certain yeast species use alternative codes. For example, “CUG,” standard codon for “Leu,” encodes “Ser” in “CUG” clade species such as Candida albicans, Candida cylindracea, Candida melibiosica, Candida parapsilosis, Candida rugose, Pichia stipitis, and Metschnikowia species. The DNA codon table for the H0 Metschnikowia sp. is provided below. The DNA codon CTG in a foreign gene from a non “CUG” clade species needs to be changed to TTG, CTT, CTC, TTA or CTA for a functional expression of a protein in the Metschnikowia species. Other codon optimization can result in increase of protein expression of a foreign gene in the Metschnikowia species. Methods of Codon optimization are well known in the art (e.g. Chung et al., BMC Syst Biol. 6:134 (2012); Chin et al., Bioinformatics 30(15):2210-12 (2014)), and various tools are available (e.g. DNA2.0 at https://www.dna20.com/services/genegps; and OPTIMIZER at http://genomes.urv.es/OPTIMIZER).

Codons for H0 Metschnikowia sp.

| Amino Acid | SLC | DNA codons | | | | | | |
|---|---|---|---|---|---|---|---|---|
| Isoleucine | I | ATT | ATC | ATA | | | | |
| Leucine | L | CTT | CTC | CTA | TTA | TTG | | |
| Valine | V | GTT | GTC | GTA | GTG | | | |
| Phenylalanine | F | TTT | TTC | | | | | |
| Methionine | M | ATG | | | | | | |
| Cysteine | C | TGT | TGC | | | | | |
| Alanine | A | GCT | GCC | GCA | GCG | | | |
| Glycine | G | GGT | GGC | GGA | GGG | | | |
| Proline | P | CCT | CCC | CCA | CCG | | | |
| Threonine | T | ACT | ACC | ACA | ACG | | | |
| Serine | S | TCT | TCC | TCA | TCG | AGT | AGC | CTG |
| Tyrosine | Y | TAT | TAC | | | | | |
| Tryptophan | W | TGG | | | | | | |
| Glutamine | O | CAA | CAG | | | | | |
| Asparagine | N | AAT | AAC | | | | | |
| Histidine | H | CAT | CAC | | | | | |
| Glutamic acid | E | GAA | GAG | | | | | |
| Aspartic acid | D | GAT | GAC | | | | | |
| Lysine | K | AAA | AAG | | | | | |
| Arginine | R | CGT | CGC | CGA | CGG | AGA | AGG | |
| Stop codons | Stop | TAA | TAG | TGA | | | | |

In some embodiments, the isolated Metschnikowia species provided herein can have one or more biosynthetic pathways to produce compounds such as xylitol, arabitol, ethanol, n-butanol, isobutanol, isopropanol, ethyl acetate, phenyl-ethyl alcohol, 2-methyl-butanol, or 3-methyl-butanol from xylose. The biosynthetic pathway can be an endogenous pathway or an exogenous pathway. The Metschnikowia species provided herein can further have expressible nucleic acids encoding one or more of the enzymes or proteins participating in one or more biosynthetic pathways for products such as xylitol, arabitol, ethanol, n-butanol, isobutanol, isopropanol, ethyl acetate, phenyl-ethyl alcohol, 2-methyl-butanol, and 3-methyl-butanol. The nucleic acids for some or all of a particular biosynthetic pathway can be expressed, depending upon what enzymes or proteins are endogenous to the Metschnikowia species. In some embodiments, the Metschnikowia species can have endogenous expression of all enzymes of a biosynthetic pathway to produce a compound from xylose and naturally produce the compound, which can be improved by further modifying or increasing expression of an enzyme or protein of the biosynthetic pathway (e.g., a xylose transporter). In some embodiments, the Metschnikowia species can be deficient in one or more enzymes or proteins for a desired biosynthetic pathway, then expressible nucleic acids for the deficient enzyme(s) or protein(s) are introduced into the Metschnikowia species for subsequent exogenous expression. Alternatively, if the Metschnikowia species exhibits endogenous expression of some pathway genes, but is deficient in others, then an encoding nucleic acid is needed for the deficient enzyme(s) or protein(s) to achieve biosynthesis of the desired compound. Thus, a recombinant Metschnikowia species can further include exogenous enzyme or protein activities to obtain a desired biosynthetic pathway or a desired biosynthetic pathway can be obtained by introducing one or more exogenous enzyme or protein activities that, together with one or more endogenous enzymes or proteins, produces a desired compound such as xylitol, arabitol, ethanol, n-butanol, isobutanol, isopropanol, ethyl acetate, phenyl-ethyl alcohol, 2-methyl-butanol, or 3-methyl-butanol from xylose.

The Metschnikowia species provided herein can contain stable genetic alterations, which refers to microorganisms that can be cultured for greater than five generations without loss of the alteration. Generally, stable genetic alterations include modifications that persist greater than 10 generations, particularly stable modifications will persist more than about 25 generations, and more particularly, stable genetic modifications will be greater than 50 generations, including indefinitely.

In the case of gene disruptions, a particularly useful stable genetic alteration is a gene deletion. The use of a gene deletion to introduce a stable genetic alteration is particularly useful to reduce the likelihood of a reversion to a phenotype prior to the genetic alteration. For example, stable growth-coupled production of a biochemical can be achieved, for example, by deletion of a gene encoding an enzyme catalyzing one or more reactions within a set of metabolic modifications. The stability of growth-coupled production of a biochemical can be further enhanced through multiple deletions, significantly reducing the likelihood of multiple compensatory reversions occurring for each disrupted activity.

Those skilled in the art will understand that the genetic alterations, including metabolic modifications exemplified herein, are described with reference to a suitable host organism such as a Metschnikowia species provided herein and their corresponding metabolic reactions or a suitable source organism for desired genetic material such as genes for a desired metabolic pathway. However, given the complete genome sequencing of a wide variety of organisms and the high level of skill in the area of genomics, those skilled in the art will readily be able to apply the teachings and guidance provided herein to essentially all other organisms. For example, the metabolic alterations exemplified herein can readily be applied to other species by incorporating the same or analogous encoding nucleic acid from species other than the referenced species. Such genetic alterations include, for example, genetic alterations of species homologs, in general, and in particular, orthologs, paralogs or nonorthologous gene displacements.

An ortholog is a gene or genes that are related by vertical descent and are responsible for substantially the same or identical functions in different organisms. For example, mouse epoxide hydrolase and human epoxide hydrolase can be considered orthologs for the biological function of hydrolysis of epoxides. Genes are related by vertical descent when, for example, they share sequence similarity of sufficient amount to indicate they are homologous, or related by evolution from a common ancestor. Genes can also be considered orthologs if they share three-dimensional structure but not necessarily sequence similarity, of a sufficient amount to indicate that they have evolved from a common ancestor to the extent that the primary sequence similarity is not identifiable. Genes that are orthologous can encode proteins with sequence similarity of about 25% to 100% amino acid sequence identity. Genes encoding proteins sharing an amino acid similarity less that 25% can also be considered to have arisen by vertical descent if their three-dimensional structure also shows similarities. Members of the serine protease family of enzymes, including tissue plasminogen activator and elastase, are considered to have arisen by vertical descent from a common ancestor.

Orthologs include genes or their encoded gene products that through, for example, evolution, have diverged in structure or overall activity. For example, where one species encodes a gene product exhibiting two functions and where such functions have been separated into distinct genes in a second species, the three genes and their corresponding products are considered to be orthologs. For the production of a biochemical compound, those skilled in the art will understand that the orthologous gene harboring the metabolic activity to be introduced or disrupted is to be chosen for construction of the Metschnikowia species provided herein. An example of orthologs exhibiting separable activities is where distinct activities have been separated into distinct gene products between two or more species or within a single species. A specific example is the separation of elastase proteolysis and plasminogen proteolysis, two types of serine protease activity, into distinct molecules as plasminogen activator and elastase. A second example is the separation of mycoplasma 5′-3′ exonuclease and Drosophila DNA polymerase III activity. The DNA polymerase from the first species can be considered an ortholog to either or both of the exonuclease or the polymerase from the second species and vice versa.

In contrast, paralogs are homologs related by, for example, duplication followed by evolutionary divergence and have similar or common, but not identical functions. Paralogs can originate or derive from, for example, the same species or from a different species. For example, microsomal epoxide hydrolase (epoxide hydrolase I) and soluble epoxide hydrolase (epoxide hydrolase II) can be considered paralogs because they represent two distinct enzymes, co-evolved from a common ancestor, that catalyze distinct reactions and have distinct functions in the same species. Paralogs are proteins from the same species with significant sequence similarity to each other suggesting that they are homologous, or related through co-evolution from a common ancestor. Groups of paralogous protein families include HipA homologs, luciferase genes, peptidases, and others.

A nonorthologous gene displacement is a nonorthologous gene from one species that can substitute for a referenced gene function in a different species. Substitution includes, for example, being able to perform substantially the same or a similar function in the species of origin compared to the referenced function in the different species. Although generally, a nonorthologous gene displacement will be identifiable as structurally related to a known gene encoding the referenced function, less structurally related but functionally similar genes and their corresponding gene products nevertheless will still fall within the meaning of the term as it is used herein. Functional similarity requires, for example, at least some structural similarity in the active site or binding region of a nonorthologous gene product compared to a gene encoding the function sought to be substituted. Therefore, a nonorthologous gene includes, for example, a paralog or an unrelated gene.

Therefore, in identifying and constructing the Metschnikowia species provided herein having biosynthetic capability, those skilled in the art will understand with applying the teaching and guidance provided herein to a particular species that the identification of metabolic modifications can include identification and inclusion or inactivation of orthologs. To the extent that paralogs and/or nonorthologous gene displacements are present in the referenced microorganism that encode an enzyme catalyzing a similar or substantially similar metabolic reaction, those skilled in the art also can utilize these evolutionally related genes. Similarly for a gene disruption, evolutionally related genes can also be disrupted or deleted in a host microbial organism to reduce or eliminate functional redundancy of enzymatic activities targeted for disruption.

Orthologs, paralogs and nonorthologous gene displacements can be determined by methods well known to those skilled in the art. For example, inspection of nucleic acid or amino acid sequences for two polypeptides will reveal sequence identity and similarities between the compared sequences. Based on such similarities, one skilled in the art can determine if the similarity is sufficiently high to indicate the proteins are related through evolution from a common ancestor. Algorithms well known to those skilled in the art, such as Align, BLAST, Clustal W and others compare and determine a raw sequence similarity or identity, and also determine the presence or significance of gaps in the sequence which can be assigned a weight or score. Such algorithms also are known in the art and are similarly applicable for determining nucleotide sequence similarity or identity. Parameters for sufficient similarity to determine relatedness are computed based on well known methods for calculating statistical similarity, or the chance of finding a similar match in a random polypeptide, and the significance of the match determined. A computer comparison of two or more sequences can, if desired, also be optimized visually by those skilled in the art. Related gene products or proteins can be expected to have a high similarity, for example, 25% to 100% sequence identity. Proteins that are unrelated can have an identity which is essentially the same as would be expected to occur by chance, if a database of sufficient size is scanned (about 5%). Sequences between 5% and 24% may or may not represent sufficient homology to conclude that the compared sequences are related. Additional statistical analysis to determine the significance of such matches given the size of the data set can be carried out to determine the relevance of these sequences.

Exemplary parameters for determining relatedness of two or more sequences using the BLAST algorithm, for example, can be as set forth below. Briefly, amino acid sequence alignments can be performed using BLASTP version 2.0.8 (Jan. 5, 1999) and the following parameters: Matrix: 0 BLOSUM62; gap open: 11; gap extension: 1; x_dropoff: 50; expect: 10.0; wordsize: 3; filter: on. Nucleic acid sequence alignments can be performed using BLASTN version 2.0.6 (Sep. 16, 1998) and the following parameters: Match: 1; mismatch: −2; gap open: 5; gap extension: 2; x_dropoff: 50; expect: 10.0; wordsize: 11; filter: off. Those skilled in the art will know what modifications can be made to the above parameters to either increase or decrease the stringency of the comparison, for example, and determine the relatedness of two or more sequences.

Microbial organisms having a biosynthesis pathway to produce xylitol from xylose are known in the art. In some embodiments, provided herein Metschnikowia species having a biosynthesis pathway for producing xylitol from xylose. Provided herein are also methods of producing a bioderived xylitol by culturing the Metschnikowia species provided herein having a xylitol biosynthesis pathway under conditions and for a sufficient period of time to produce xylitol.

Many yeast species (Candida spp., Debaryomyces hansenii, Pichia anomala, Kluyveromvces spp, Pachysolen tannophilus, Saccharomyces spp. and Schizosaccharomyces pombe) have been identified with the ability to convert xylose to xylitol (Sirisansaneeyakul et al., J. Ferment. Bioeng. 80:565-570 (1995); Onishi et al., Agric. Biol. Chem. 30:1139-1144 (1966); Barbosa et al., J. Ind. Microbiol. 3:241-251 (1988); Gong et al., Biotechnol. Lett. 3:125-130 (1981); Vandeska et al., World J. Microbiol. Biotechnol. 11:213-218 (1995); Dahiya et al., Cabdirect.org 292-303 (1990); Gong et al., Biotechnol. Bioeng. 25:85-102 (1983)). The ability to produce xylitol from xylulose has also been discovered in various yeast (Saccharomyces spp., D. hansenii, Pichia farinose, Hansenula spp., Endomycopsis chodatii, Candida spp. and Cryptococcus neoformans) (Onishi et al., Appl. Microbiol. 18:1031-1035 (1969)). The majority of research into the biological production of xylitol is with yeast, and novel yeast species capable of converting xylose to xylitol continue to be discovered (Kamat et al., J. App. Microbiol. 115: 1357-1367 (2013); Bura et al., J. Ind. Microbiol. Biotechnol. 39:1003-1011 (2012); Junyapate et al., Antonie Van Leeuwenhoek 105:471-480 (2014); Guaman-Burneo et al., Antonie Van Leeuwenhoek 108: 919-931 (2015); Cadete et al., Int. J. Syst. Evolv. Microbiol. 65:2968-2974 (2015)).

Saccharomyces cerevisiae is a yeast organism that is used in many food processes, but does not naturally utilize xylose efficiently. It has been engineered to produce xylitol from xylose by expressing xylose reductases from other yeast species such as Scheffersomyces stipitis (Pichia stipitis) and Candida shehatae (Hallborn et al., Bio/Technology 9:1090-1095; Hallborn et al., Appl. Microbiol. Biotechol. 42:326-333 (1994); Lee et al., Process Biochem. 35:1199-1203 (2000); Giovinden et al., Appl. Microbiol. Biotechnol. 55:76-80 (2001); Chung et al., Enzyme Microb. Technol. 30:809-816 (2002)).

Alternate pathways for xylitol production in S. cerevisiae have been explored. Expression of Scheffersomyces stipitis xylitol dehydrogenase and deletion of the xylulokinase gene in a transketolase-deficient strain of S. cerevisiae allowed conversion of glucose to xylitol through a multistep pathway (Toivari et al., Appl. Enviorn. Microbiol. 73:5471-5476 (2007)).

Expression of Neurospora crassa cellodextrin transporter and intracellular β-glucosidase allowed it to simultaneously utilize cellobiose and xylose during xylitol production (Oh et al., Metab. Eng. 15:226-234 (2013); Zha et al., PLoS One 8:e68317 (2013)). Furthermore, the overexpression of S. cerevisae ALD5, IDP2 or S. stipitis ZWF1 lead to increased NADPH levels, resulting in higher xylitol productivity (Oh et al., Metab. Eng. 15:226-234 (2013)).

Xylitol production can be improved by the use of both NADPH-preferring and NADH-preferring xylose reductases to decrease the limitation of NAD(P)H cofactors. This strategy was used in S. cerevisiae with the expression of wild-type NADPH-preferring and mutant NADH-preferring S. stipitis xylose reductase and S. cerevisiae ZWF1 and ACS1 (Jo et al., Biotechnol. 1 10:1935-1943 (2015)).

In order to decrease processing costs of xylitol production, S. stipitis xylose reductase, Aspergillus aculeatus β-glucosidase, Apsergillus oryzae β-xylosidase, and Trichoderma reesei endoxylanase were expressed in S. cerevisiae (Guirimand et al., Appl. Microbiol. Biotechnol. 100:3477-3487 (2016)). Expression of these fungal enzymes allowed direct degradation of hemicellulose without the addition of exogenous enzymes.

Candida tropicalis is pathogenic, but is also one of the natural producers of xylitol. Several patents and literature have described the application of yeast from genus Candida as the host strain for xylitol production from xylose; i.e. C. tropicalis ATCC 13803 (PCT/IN2009/000027 & KR100259470), C. tropicalis ATCC 9968 (PCT/FI1990/000015), C. tropicalis KFCC 10960 (KR100199819), C. tropicalis (NRRL 12968) (PCT/IN2013/000523), C. tropicalis ATCC 750 (West et al., World J. Mircrobiol. Biotechnol. 25:913-916 (2009)) and C. tropicalis ATCC 7349 (SAROTE et al., J. Ferment. and Bioeng. 80:565-570 (1995)). One strategy used to improve xylitol production in C. tropicalis was the expression of an NADH-preferring xylose reductase from C. parapsilosis, which allowed reduction of xylose with both NADPH and NADH (Lee et al., Appl. Enviorn. Microbiol. 69:6179-6188 (2003)). Deletion of xylitol dehydrogenase increases xylitol production by blocking xylitol catabolism, but a co-substate such as glucose or glycerol is needed to regenerate NADPH for xylose reductase activity (Ko et al., Appl. Environ. Microbiol. 72:4207-4213 (2006); Ko et al., Biotechnol. Lett. 28:1159-1162 (2006)). Further improvements for xylitol production were made by combining deletion of the xylitol dehydrogenase gene with expression of Neurospora crassa xylose reductase (Jeon et al., Bioprocess Biosyst. Eng. 35:191-198 (2012)). The xylose uptake and xylitol productivity of this strain was again further improved by expressing a xylose transporter from Arabidopsis thaliana (Jeon et al., Bioprocess Biosyst. Eng. 36:809-817 (2013)).

If glycerol is provided as a co-substrate, NADPH regeneration can be enhanced by expressing glucose-6-phosphate dehydrogenase and 6-phosphogluconate dehydrogenase in C. tropicalis (Ahmad et al., Bioprocess Biosyst. Eng. 35:199-204 (2012)). Xylitol production can also be enhanced by deleting glycerol kinase and expressing three NADPH-regenerating glycerol dehydrogenases from Scheffersomyces stipitis (Ahmad et al., Bioprocess Biosyst. Eng. 36:1279-1284 (2013)). One of the problems with producing xylitol from mixed sugar substrates is that the xylose reductase from C. tropicalis can convert arabinose to arabitol, a contaminant in xylitol production. To prevent this, the endogenous xylose reductase was deleted and a mutant xylose-specific xylose reductase from Neurospora crassa was expressed along with bacterial arabinose assimilation enzymes (Yoon et al., Biotechnol. Lett. 33:747-753 (2011); Nair et al., ChemBioChem 9:1213-1215 (2008)). This minimized arabitol formation while allowing arabinose assimilation for cell growth.

Kluyveromyces marxianus is a thermotolerant yeast often found in dairy products. It can be used for xylitol production due to its high growth rate, tolerance to temperatures up to 52° C., and ability to utilize various sugars. Expression of the Neurospora crassa xylose reductase alone or in conjunction with deletion of the xylitol dehydrogenase gene in K. marxianus led to xylitol production optimally at 42° C. (Zhang et al., Bioresour. Technol. 152:192-201 (2014)). Further improvements to xylitol production were made by testing the expression of various xylose transporters: K. marxianus aquaglyceroporin, Candida intermedia glucose/xylose facilitator, or C. intermedia glucose/xylose symporter (Zhang et al., Bioresour. Technol. 175:642-645 (2015)). The expression of the C. intermedia glucose/xylose facilitator was found to be effective at increasing xylitol yield and productivity, and notably, produced the highest reported final xylitol concentration. K. marxianus was also used in an evolutionary adaptation experiment that resulted in a strain with improved xylose utilization and xylitol production capabilities (Sharma et al., Bioprocess Biosyst. Eng. 39:835-843 (2016)).

Two other yeast species have been genetically engineered to explore xylitol production. Debaryomyces hansenii is another natural producer of xylitol that is osmotolerant and non-pathogenic. Xylitol production was enhanced in this species by deletion of the xylitol dehydrogenase gene (Pal et al., Bioresour. Technol. 147:449-455 (2013)). Pichia pastoris is a yeast commonly used for protein expression. It has been engineered to produce xylitol directly from glucose through the glucose-arabitol-xylulose-xylitol pathway (Cheng et al., Appl. Microbiol. Biotechnol. 98:3539-3552 (2014)). This was achieved by expressing xylitol dehydrogenase from Gluconobacter oxydans and the xylulose-forming arabitol dehydrogenase from Klebsiella pneumoniae.

In addition to filamentous fungi and yeast, a limited number of bacterial species (Corynebacterium sp. and Enterobacter liquefaciens) have been observed to produce xylitol from xylose (Yoshitake et al., Agric. Biol. Chem. 35:905-911 (1971); Yoshitake et al., Agric. Biol. Chem. 37:2261-2267 (1973); Yoshitake et al., Agric. Biol. Chem. 40:1493-1503 (1976); Rangaswamy et al., Appl. Microbiol. Biotechnol. 60:88-93 (2002)). Mycobacterium smegmatis has also been reported to be able to produce xylitol from xylulose (Izumori et al., J. Ferment. Technol. 66:33-36 (1988)). A subsequent screen of bacteria discovered that Gluconobacter spp. and Acetobacter xylinum are capable of converting arabitol to xylitol through the sequential conversion of arabitol to xylulose and xylulose to xylitol (Suzuki et al., Biosci. Biotechnol. Biochem. 66:2614-2620 (2002)).

Microalgae are an attractive platform for the production of renewable resources. Xylitol production in microalgae has been reported once, where expression of the xylose reductase from Neurospora crassa in Chlamydomonas reinhardtii allowed it to convert a small amount of xylose to xylitol (Pourmir et al., J. Biotechnol. 165:178-183 (2013)).

The extracts of various filamentous fungi (Penicillium spp., Aspergillus spp., Rhizopus nigricans, Gliocladium roseum, Byssochlamys fulva, Myrothecium verrucaria, Neurospora crassa, Rhodotorula glutinis and Torulopsis utilis) have been observed to contain an enzyme capable of converting xylose to xylitol (Chiang et al., Nature 188:79-81 (1960); Chiang et al., Biochem. Biophys. Res. Commun. 3:554-559 (1960); Chiang et al., Biochem. Biophys. Acta. 29:664-5 (1958)). Subsequent studies identified additional filamentous fungi (Petromyces albertensis, Penicillium spp. and Aspergillus niger) capable of converting xylose to xylitol with varying degrees of efficiency (Dahiya et al., Can. J. Microbiol. 37:14-18 (1991); Sampaio et al., Brazilian J. Microbiol. 34:325-328 (2003)).

Trichoderma reesei, a filamentous fungus that secretes celluloytic enzymes, produced more xylitol when the genes for xylitol dehydrogenase and L-arabinitol-4-dehydrogenase were deleted in order to block xylitol metabolism (Dashtban et al., Appl. Biochem. Biotecnol. 169:554-569(2013)). Xylitol production also increased in T. reesei when xylose reductase was overexpressed and xylulokinase was inhibited (Hong et al., Biomed Res. Int. 2014:169705 (2014)). Phanerochaete sordida, a white-rot fungus with ligninolytic activity, produced more xylitol when it expressed the xylose reductase gene from Phanerochaete chrysosporium (Hirabayashi et al., J. Biosci. Bioeng. 120:6-8 (2015)).

Bacteria metabolize xylose with xylose isomerases instead of with the xylose reductase-xylitol dehydrogenase pathway. Therefore, the use of bacterial hosts for xylitol production typically involves recombinant expression of xylose reductases. Xylose reductase from Candida tropicalis was expressed in Escherichia coli and was found to be functional for xylitol production from xylose (Suzuki et al., J. Biosci. Bioeng. 87:280-284 (1999)). A subsequent study expressed xylose reductases from Candida boidinii, Candida tenuis and Scheffersomyces stipitis in conjunction with a deletion of the endogenous xylulokinase gene (Cirino et al., Biotechnol. Bioeng. 95:1167-1176 (2006)). In order to improve xylitol production from mixtures of glucose and xylose, the cyclic AMP receptor protein was replaced with a mutant that circumvents glucose repression of xylose metabolism. Expressing the xylose transporters, XylE or XylFGH, has similar effects to replacing the cyclic AMP receptor protein with a mutant form (Khankal et al., J. Biotehnol. 134:246-252 (2008)).

Cofactor regeneration is also important for improving xylitol production in bacteria, which has been explored in E. coli through a large number of gene deletions and expression of cofactor regenerating pathways (Chin et al., Biotechnol. Bioeng. 102:209-220 (2009); Chin et al., iBiotechnol. Prog. 27:333-341 (2011); Iverson et al., World J. Microbiol. Biotechnol. 29:1225-1232 (2013); Iverson et al., BMC Syst. Biol. 10:31 (2016)). Another study aimed at improving xylitol production from mixtures of glucose and xylose disrupted the phosphoenolpyruvate-dependent glucose phosphotransferase system to eliminate catabolite repression (Su et al., Metab. Eng. 31:112-122 (2015)). Endogenous xylose metabolism was blocked in this strain by disrupting xylose isomerase, xylulose kinase, and the phosphoenolpyruvate-dependent fructose phosphotransferase system, and the Neurospora crassa xylose reductase was expressed to optimize xylitol production.

Lactococcus lactis is a well-characterized bacterium commonly used for dairy processes such as cheese production, and could be adopted for other food-related processes. L. lactis was able to produce xylitol from xylose when it expressed the S. stipitis xylose reductase and the Lactobacillus brevis xylose transporter (Nyyossola et al., J. Biotechnol. 118:55-56 (2005)).

Corynebacterium glutamicum is a bacterium with many industrial uses such as the production of MSG. It has been engineered to co-utilize xylose and glucose, which is an important trait for xylitol productivity (Sasaki et al., Appl. Microbiol. Biotechnol. 85:105-115 (2009)). To optimize xylitol production in C. glutamicum, it has been engineered to express a pentose transporter and a mutant xylose reductase from Candida tenuis in conjunction with disruptions of its lactate dehydrogenase, xylulokinase, and phosphoenolpyruvate-dependent fructose phosphotransferase genes (Sasaki et al., Appl. Microbiol. Biotechnol. 86:1057-1066 (2010)). Xylitol production in C. glutamicum was also achieved by expressing Scheffersomyces stipitis xylose reductase (Kim et al., Enzyme Microb. Technol. 46:366-371 (2010)). Expression of Rhodotorula mucilaginosa xylose reductase, E. coli 1-arabinose isomerase, Agrobacterium tumefaciens d-psicose 3 epimerase, Mycobacterium smegmatis 1-xylulose reductase, and a fusion pentose transporter allowed the production of xylitol from mixtures of xylose and arabinose without the formation of arabitol (Dhar et al., J. Biotechnol. 230:63-71 (2016)).

It is understood that the Metschnikowia species provided herein can be used as the host strain for production of xylitol. Further metabolic engineering can be used to adopt the Metschnikowia species to further increase xylitol production in these Metschnikowia species.

Microbial organisms having a biosynthesis pathway to produce arabitol from xylose are known in the art. In some embodiments, provided herein Metschnikowia species having a biosynthesis pathway for producing arabitol from xylose. Provided herein are also methods of producing a bioderived arabitol by culturing the Metschnikowia species provided herein having an arabitol biosynthesis pathway under conditions and for a sufficient period of time to produce arabitol.

Some yeast species have been identified that can produce arabitol from xylose. For example, the recently identified Zygocaccharomyces rouxxii NRRL 27,624 strain has been known to produce D-arabitol as the main metabolic product from glucose (Saha et al., 2007, J. Ind. Microbial. Biotechnol., 34:519-523). However, it also was identified as producing D-arabitol and xylitol from xylose and from a mixture of xylose and xylulose (Saha et al., 2007). Based on these results, the pathway for production of D-arabitol from xylose included a xylose reductase, a xylitol dehydrogenase and an arabitol dehydrogenase (Saha et al., 2007). Additionally, Candida maltosa has been shown to produce D-arabitol from D-xylulose by a xylulose reductase (Cheng et al., 2011, Microbial. Cell Factories, 10:5). Production of arabitol was also found to be improved by the addition of xylose with glycerol in the yeast species within the genus of Debaryomyces, Geotrichum and Metschnikowia (International Application Publication WO 2012/011962, published Jan. 26, 2012).

It is understood that the Metschnikowia species provided herein can be used as the host strain for production of arabitol. Further metabolic engineering can be used to adopt the Metschnikowia species to further increase arabitol production in these Metschnikowia species.

Microbial organisms having a biosynthesis pathway to produce ethanol from xylose are known in the art. In some embodiments, provided herein are Metschnikowia species having at least one exogenous nucleic acid encoding an enzyme of a biosynthesis pathway for producing ethanol from xylose. With enhanced xylose uptake the microbial organism can also have improved production of ethanol from xylose. Provided herein are also methods of producing a bioderived ethanol by culturing the Metschnikowia species provided herein having an ethanol biosynthesis pathway under conditions and for a sufficient period of time to produce ethanol.

Ethanol has a number of uses and is most commonly used as a fuel additive. As a fuel additive, ethanol is a low value product with much of the cost of its production attributed to the cost of raw materials. It would be desirable, therefore, to develop ethanologens and fermentation processes for the production of ethanol from readily available, inexpensive starting materials, such as lignocellulose. Fermentation of both glucose and xylose is currently regarded as a high priority for economical conversion of biomass into ethanol. Most microorganisms are able to ferment glucose but few have been reported to utilize xylose efficiently and even fewer ferment this pentose to ethanol.

A relatively small number of wild type microorganisms can ferment D-xylose. These microorganisms are generally not suitable for large-scale fermentation. This unfavorability may arise, for example, as a result of unfamiliarity with the microorganisms, difficulty obtaining the microorganisms, poor productivity and/or growth on pretreated lignocellulosics or unsatisfactory yield when grown on mixed sugars derived from biomass. (C. Abbas, “Lignocellulosics to ethanol: meeting ethanol demand in the future,” The Alcohol Textbook, 4^{th} Edition. (K. A. Jacques, T. P. Lyons and D. R. Kelsall, eds). Nottingham University Press, Nottingham, UK, 2003, pp. 41-57.; C. Abbas, “Emerging biorefineries and biotechnological applications of nonconventional yeast: now and in the future,” The Alcohol Textbook, 4^{th} Edition. (K. A. Jacques, T. P. Lyons and D. R. Kelsall, eds). Nottingham University Press, Nottingham, United Kingdom, 2003, pp. 171-191).

Yeasts are considered promising microorganisms for alcoholic fermentation of xylose (see Ryabova, supra). They have larger cells than bacteria, are resistant to viral infection, and tend to be more resistant to negative feedback from ethanol. Furthermore, yeast growth and metabolism have been extensively studied for a number of species.

A number of yeasts are known to naturally ferment D-xylose. These include, for example, Pichia stipitis, Candida shehatae, and Pachysolen tannophilus (see Ryabova, supra; Cite 2, C. Abbas 2003). The common brewer's yeast Saccharomyces cerevisiae is not known to ferment D-xylose naturally, but a number of strains of metabolically engineered S. cerevisiae that do ferment D-xylose have been reported.

Numerous studies have described the metabolism of D-xylose by recombinant S. cerevisiae (see, e.g., Matsushika et al., Applied Microbiology and Biotechnology 84, no. 1 (2009): 37-53; U.S. Pat. Pub. No. 2005/0153411A1 (Jul. 14, 2005); U.S. Pat. Pub. No. 2004/0231661A1 (Nov. 25, 2004); U.S. Pat. No. 4,368,268 (Jan. 11, 1983); U.S. Pat. No. 6,582,944 (Jun. 24, 2003); U.S. Pat. No. 7,226,735 (Jun. 5, 2007); U.S. Pat. Pub. No. 2004/0142456A1 (Jul. 22, 2004); Jeffries, T. W. & Jin, Y-S., Appl. Microbiol. Biotechnol. 63: 495-509 (2004); Jin, Y-S., Met. Eng. 6: 229-238 (2004); Pitkanen, J-Y., Helsinki Univ. of Tech., Dept. of Chem. Tech., Technical Biochemistry Report (January 2005); Porro, D. et al., App. & Env. Microbiol. 65(9): 4211-4215 (1999); Jin, Y-S., et al., App. & Env. Microbiol. 70(11): 6816-6825 (2004); Sybirna, K, et al., Curr. Genetics 47(3): 172-181 (2005); Toivari, M. H., et al., Metabolic Eng. 3:236-249 (2001).

D-Xylose metabolism in yeast proceeds along a pathway similar to that of glucose via pentose phosphate pathway. Carbon from D-xylose is processed to ethanol via the glycolytic cycle or to CO2 via respiratory TCA cycle. Fermentation to ethanol relies in part on the metabolism of pyruvate, which is a metabolite that may be used in either respiration or fermentation (see van Hoek, P., et al., Appl. & Enviro. Microbiol. 64(6); 2133-2140 (1998)). Pyruvate enters fermentation following decarboxylation of pyruvate to acetaldehyde by the enzyme pyruvate decarboxylase (E.C. 4.1.1.1). Pyruvate decarboxylase is a member of the family of biotin-dependent carboxylases. It catalyzes the decarboxylation of pyruvate to form oxaloacetate with ATP cleavage. The oxaloacetate can be used for synthesis of fat, glucose, and some amino acids or other derivatives. The enzyme is highly conserved and found in a variety of prokaryotes and eukaryotes.

Other microbial organisms capable of ethanol production from xylose are also known in the art. The thermotolerant methylotrophic yeast Hansenula polymorpha (also known as Pichia angusta) was reported to have optimum and maximum growth temperatures of 37° C. and 48° C., respectively, and can naturally ferment D-xylose under certain conditions. (U.S. Pat. No. 8,071,298; Voronovsky et al., FEMS Yeast Res. 5(11): 1055-62 (2005)). Additionally, three strains of Pichia stipitis and three of Candida shehatae were reported to ferment xylose when subjected to both aerobic and microaerophilic conditions. Of the strains considered, P. stipitis NRRL Y-7124 was able to utilize all but 7 g/L of 150 g/L xylose supplied aerobically to produce 52 g/L ethanol at a yield of 0.39 g per gram xylose (76% of theoretical yield) and at a rate comparable to the fastest shown by C. shehatae NRRL Y-12878. For all strains tested, fermentation results from aerobic cultures were more favorable than those from microaerophilic cultures. Slininger, P. J. et al., Biotechnol Lett (1985) 7: 431.

For example, Zymomonas mobilis, a bacterial ethanologen that grows on glucose, fructose, and sucrose, metabolizing these sugars to CO2 and ethanol via the Entner-Douderoff pathway. Though wild type strains cannot use xylose as a carbon source, recombinant strains of Z. mobilis that are able to grow on this sugar have been engineered (U.S. patent publication No. 20080187973, U.S. Pat. No. 5,514,583, U.S. Pat. No. 5,712,133, WO 95/28476, Feldmann et al. (1992) Appl Microbiol Biotechnol 38: 354-361, Zhang et al. (1995) Science 267:240-243).

The conversion of xylose to ethanol by recombinant Escherichia coli has been reported. The addition of small amounts of calcium, magnesium, and ferrous ions stimulated fermentation. Beall et al., Biotechnology and Bioengineering 38, no. 3 (1991): 296-303.

It is understood that the Metschnikowia species provided herein can be used as the host strain for production of ethanol. Further metabolic engineering can be used to adopt the Metschnikowia species to further increase ethanol production in these Metschnikowia species.

Microbial organisms having a biosynthesis pathway to produce n-butanol from xylose are known in the art. In some embodiments, provided herein are Metschnikowia species having at least one exogenous nucleic acid encoding an enzyme of a biosynthesis pathway for producing n-butanol from xylose. With enhanced xylose uptake the microbial organism can also have improved production of n-butanol from xylose. Provided herein are also methods of producing a bioderived n-butanol by culturing the Metschnikowia species provided herein having a n-butanol biosynthesis pathway under conditions and for a sufficient period of time to produce n-butanol.

Butanol offers a number of advantages as a fuel. Butanol is a four-carbon alcohol, a clear neutral liquid miscible with most solvents (alcohols, ether, aldehydes, ketones and hydrocarbons) and is sparingly soluble in water (water solubility 6.3% as compared to ethanol which is totally miscible). It has an octane rating comparable to gasoline, making it a valuable fuel for any internal combustion engine made for burning gasoline. Fuel testing also has proven that butanol does not phase separate in the presence of water, and has no negative impact on elastomer swelling. Butanol not only has a higher energy content that is closer to that of gasoline than ethanol, so it is less of a compromise on fuel economy, but it also can be easily added to conventional gasoline due to its low vapor pressure.

Butanol biosynthesis can be achieved through the acetone, butanol, and ethanol fermentation pathway (the “ABE pathway”). The products of this butanol fermentative production pathway using a solvent-producing species of the bacterium Clostridium acetobutylicum are six parts butanol, three parts acetone, and one part ethanol. Butanol-production pathway has been introduced to various host organisms. For instance, the pathway was expressed in Escherichia coli (Atsumi et al., Nature 451:86-89 (2008)) and Saccharomyces cerevisiae (Steen et al., Microb. Cell Fact 7:36 (2008)) for their high growth rates and the efficiency of genetic tools. Pseudomonas putida, Lactobacillus brevis and Bacillus subtilis were used for their potentially higher solvent tolerance (Nielsen et al., Metab. Eng. 11:262-273 (2009); Berezina et al., Appl. Microbiol. Biot. 87:635-646 (2010)).

An alternative to the use of food crops as starting material for butanol production is biomass, specifically lignocellulosic biomass. Clostridium spp. strains have been engineered to produce butanol for xylose, such as C. saccharoperbutylacetonicum (e.g., C. saccharoperbutylacetonicum strain ATCC 27021 or C. saccharoperbutylacetonicum strain ATCC 27022). See e.g. U.S. Pat. No. 8,900,841. Clostridium cellulolyticum was engineered to divert its native valine synthesis pathway for isobutanol production from crystalline cellulose (Higashide et al., Appl. Environ. Microb. 77:2727-2733 (2011)). Clostridium cellulovorans, which natively produces butyric acid as the main metabolic product, was introduced with an aldehyde/alcohol dehydrogenase (AdhE2) to convert precursor butyryl-CoA to 1-butanol from cellulose (Yang et al., Metab. Eng. 32:39-48 (2015)). 1-Butanol production from xylose was also demonstrated using Thermoanaerobacterium saccharolyticum (Bhandiwad et al., Metab. Eng. 21:17-25 (2014)).

To increase the cellulose decomposition rate and to reduce chance of contamination, thermophilic organisms were used. The first example of isobutanol production in thermophiles was demonstrated in Geobacillus thermoglucosidasius using cellobiose as substrate (Lin et al., Metab. Eng. 24:1-8 (2014)). In this work, thermostabilities of enzymes involved in isobutanol synthesis were investigated. The result of this study was applied to the direct conversion of cellulose to isobutanol in Clostridium thermocellum by expressing and optimizing the isobutanol biosynthesis pathway (Lin et al., Metab. Eng. 31:44-52 (2015)).

One of the most effective ethanol-producing yeasts, S. cerevisiae, has several advantages such as high ethanol production from hexoses and high tolerance to ethanol and other inhibitory compounds in the acid hydrolysates of lignocellulose biomass. Although standard strains of this yeast cannot utilize pentoses, such as xylose, a recombinant yeast strain can be provided that can ferment xylose and cellooligosaccharides by integrating genes for the intercellular expression of xylose assimilation pathways, such as xylose reductase and xylitol dehydrogenase from Pichia stipitis and a gene for displaying β-glucosidase from A. acleatus. See e.g. U.S. Patent Publication No. 20100129885; U.S. Patent Publication No. 20100261241;

It is understood that the Metschnikowia species provided herein can be used as the host strain for production of n-butanol. Further metabolic engineering can be used to adopt the Metschnikowia species to further increase n-butanol production in these Metschnikowia species.

Microbial organisms having a biosynthesis pathway to produce isobutanol from xylose are known in the art. In some embodiments, provided herein are Metschnikowia species having at least one exogenous nucleic acid encoding an enzyme of a biosynthesis pathway for producing isobutanol from xylose. With enhanced xylose uptake the microbial organism can also have improved production of isobutanol from xylose. Provided herein are also methods of producing a bioderived isobutanol by culturing the Metschnikowia species provided herein having a isobutanol biosynthesis pathway under conditions and for a sufficient period of time to produce isobutanol.

Isobutanol, also a biofuel candidate, has been produced in recombinant microorganisms expressing a heterologous, five-step metabolic pathway (See, e.g., WO/2007/050671, WO/2008/098227, and WO/2009/103533). The recombinant microorganism including a pathway for the production of isobutanol from five-carbon (pentose) sugars including xylose is also known in the art. (See e.g., WO 2012173659; WO 2011153144). The recombinant microorganism can be engineered to express a functional exogenous xylose isomerase. Exogenous xylose isomerases functional in yeast are known in the art. See, e.g., US2006/0234364. The exogenous xylose isomerase gene can be operatively linked to promoter and terminator sequences that are functional in the yeast cell.

For example, recombinant Saccharomyces cerevisiae was known to produce isobutanol from xylose. See e.g. US20130035515, Brat et al., FEMS yeast research 13.2 (2013): 241-244; Lee, Won-Heong et al. Bioprocess and biosystems engineering 35.9 (2012): 1467-1475; Simultaneous overexpression of an optimized, cytosolically localized valine biosynthesis pathway together with overexpression of xylose isomerase XylA from Clostridium phytofermentans, transaldolase Tal1 and xylulokinase Xks 1 enabled recombinant Saccharomyces cerevisiae cells to complement the valine auxotrophy of ilv2,3,5 triple deletion mutants for growth on D-xylose as the sole carbon source. Moreover, after additional overexpression of ketoacid decarboxylase Aro10 and alcohol dehydrogenase Adh2, the cells were able to ferment D-xylose directly to isobutanol.

It is understood that the Metschnikowia species provided herein can be used as the host strain for production of isobutanol. Further metabolic engineering can be used to adopt the Metschnikowia species to further increase isobutanol production in these Metschnikowia species.

Microbial organisms having a biosynthesis pathway to produce isopropanol from xylose are known in the art. In some embodiments, provided herein are Metschnikowia species having at least one exogenous nucleic acid encoding an enzyme of a biosynthesis pathway for producing isopropanol from xylose. With enhanced xylose uptake the microbial organism can also have improved production of isopropanol from xylose. Provided herein are also methods of producing a bioderived isopropanol by culturing the Metschnikowia species provided herein having an isopropanol biosynthesis pathway under conditions and for a sufficient period of time to produce isopropanol.

Polymerization of ethylene provides polyethylene, a type of plastic with a wide range of useful applications. Ethylene is traditionally produced by refined non-renewable fossil fuels, but dehydration of biologically-derived ethanol to ethylene offers an alternative route to ethylene from renewable carbon sources, i.e., ethanol from fermentation of fermentable sugars. Similarly, isopropanol and n-propanol can be dehydrated to propylene, which in turn can be polymerized to polypropylene. As with polyethylene, using biologically-derived propanol starting material (i.e., isopropanol or n-propanol) would result in “Green Polypropylene.” See e.g. WO 2009/049274, WO 2009/103026, WO 2009/131286, WO 2010/071697, WO 2011/031897, WO 2011/029166, WO 2011/022651 , WO 2012/058603.

Production of isoproponal has been observed in recombinant Lactobacillus host cells (e.g., Lactobacillus reuteri) engineered to have an isopropanol pathway and produce increased amounts of isopropanol. See e.g. WO2013178699 A1. Direct isopropanol production from cellobiose by engineered Escherichia coli using a synthetic pathway was also observed. See e.g. Soma et al., Journal of bioscience and bioengineering 114.1 (2012): 80-85.

It is understood that the Metschnikowia species provided herein can be used as the host strain for production of isopropanol. Further metabolic engineering can be used to adopt the Metschnikowia species to further increase isopropanol production in these Metschnikowia species.

Microbial organisms having a biosynthesis pathway to produce ethyl acetate from xylose are known in the art. In some embodiments, provided herein are Metschnikowia species having at least one exogenous nucleic acid encoding an enzyme of a biosynthesis pathway for producing ethyl acetate from xylose. With enhanced xylose uptake the microbial organism can also have improved production of ethyl acetate from xylose. Provided herein are also methods of producing a bioderived ethyl acetate by culturing the Metschnikowia species provided herein having an ethyl acetate biosynthesis pathway under conditions and for a sufficient period of time to produce ethyl acetate.

Ethyl acetate is an environmentally friendly solvent with many industrial applications. Microbial synthesis of ethyl acetate is desirable. The ability of yeasts for producing larger amounts of this ester is known for a long time and can be applied to large-scale ester production from renewable raw materials. Pichia anomala, Candida utilis, and Kluyveromyces marxianus are yeasts which convert sugar into ethyl acetate with a high yield. Loser et al., Appl Microbiol Biotechnol (2014) 98:5397-5415.

Synthesis of much ethyl acetate requires oxygen which is usually supplied by aeration. Ethyl acetate is highly volatile so that aeration results in its phase transfer and stripping. This stripping process cannot be avoided but requires adequate handling during experimentation and offers a chance for a cost-efficient process-integratedrecovery of the synthesized ester.

It is understood that the Metschnikowia species provided herein can be used as the host strain for production of ethyl acetate. Further metabolic engineering can be used to adopt the Metschnikowia species to further increase ethyl acetate production in these Metschnikowia species.

Microbial organisms having a biosynthesis pathway to produce phenyl-ethyl alcohol from xylose are known in the art. In some embodiments, provided herein are Metschnikowia species having at least one exogenous nucleic acid encoding an enzyme of a biosynthesis pathway for producing phenyl-ethyl alcohol from xylose. With enhanced xylose uptake the microbial organism can also have improved production of phenyl-ethyl alcohol from xylose. Provided herein are also methods of producing a bioderived phenyl-ethyl alcohol by culturing the Metschnikowia species provided herein having a phenyl-ethyl alcohol biosynthesis pathway under conditions and for a sufficient period of time to produce phenyl-ethyl alcohol.

Phenyl-ethyl alcohol a colorless, transparent, slightly viscous liquid that can be produced by microbial organisms. Phenyl-ethyl alcohol has been found in a number of natural essential oils, in food, spices and tobacco, and in undistilled alcoholic beverages, beers and wines. It prevents or retards bacterial growth, and thus protects cosmetics and personal care products from spoilage. Phenyl-ethyl alcohol also imparts a fragrance to a product.

It is understood that the Metschnikowia species provided herein can be used as the host strain for production of phenyl-ethyl alcohol. Further metabolic engineering can be used to adopt the Metschnikowia species to further increase phenyl-ethyl alcohol production in these Metschnikowia species.

Microbial organisms having a biosynthesis pathway to produce 2-methyl-butanol from xylose are known in the art. In some embodiments, provided herein are Metschnikowia species having at least one exogenous nucleic acid encoding an enzyme of a biosynthesis pathway for producing 2-methyl-butanol from xylose. With enhanced xylose uptake the microbial organism can also have improved production of 2-methyl-butanol from xylose. Provided herein are also methods of producing a bioderived 2-methyl-butanol by culturing the Metschnikowia species provided herein having a 2-methyl-butanol biosynthesis pathway under conditions and for a sufficient period of time to produce 2-methyl-butanol.

2-methyl-butanol can be used as a solvent and an intermediate in the manufacture of other chemicals. 2-methyl-butanol also has applications in fuel and lubricating oil additives, flotation aids, manufacture of corrosion inhibitors, pharmaceuticals, paint solvent, and extraction agent.

It is understood that the Metschnikowia species provided herein can be used as the host strain for production of 3-methyl butanol. Further metabolic engineering can be used to adopt the Metschnikowia species to further increase 2-methyl butanol production in these Metschnikowia species.

Microbial organisms having a biosynthesis pathway to produce 3-methyl-butanol from xylose are known in the art. In some embodiments, provided herein are Metschnikowia species having at least one exogenous nucleic acid encoding an enzyme of a biosynthesis pathway for producing 3-methyl-butanol from xylose. With enhanced xylose uptake the microbial organism also has improved production of 3-methyl-butanol from xylose. Provided herein are also methods of producing a bioderived 3-methyl-butanol by culturing the Metschnikowia species provided herein having a 3-methyl-butanol biosynthesis pathway under conditions and for a sufficient period of time to produce 3-methyl-butanol.

3-methyl-butanol (also known as isoamyl alcohol or isopentyl alcohol) is a clear, colorless alcohol. 3-methyl-butanol is a main ingredient in the production of banana oil, an ester found in nature and also produced as a flavouring in industry. It is also the main ingredient of Kovac's reagent, used for the bacterial diagnostic indole test. 3-methyl-butanol is also used as an antifoaming agent in the Chloroform:Isomyl Alcohol reagent.

It is understood that the Metschnikowia species provided herein can be used as the host strain for production of 3-methyl-butanol. Further metabolic engineering can be used to adopt the Metschnikowia species to further increase 3-methyl-butanol production in these Metschnikowia species.

Depending on the biosynthetic pathway constituents of a Metschnikowia species a for a particular compound, the Metschnikowia species provided herein can include at least one exogenously expressed biosynthetic pathway-encoding nucleic acid and up to all encoding nucleic acids for one or more biosynthetic pathways of the compound. The compound can be, for example, xylitol, arabitol, ethanol, n-butanol, isobutanol, isopropanol, ethyl acetate, phenyl-ethyl alcohol, 2-methyl-butanol, or 3-methyl-butanol. For example, ethanol biosynthesis can be established in a Metschnikowia species deficient in a pathway enzyme or protein that is required to produce ethanol from xylose through exogenous expression of the corresponding encoding nucleic acid. In other words, in a Metschnikowia species deficient in all enzymes or proteins of an ethanol pathway, exogenous expression of all enzyme or proteins in the pathway can be included, although it is understood that all enzymes or proteins of a pathway can be expressed even if the Metschnikowia species contains at least one of the pathway enzymes or proteins. For example, exogenous expression of all enzymes or proteins in a pathway for production of ethanol can be included in the H0 Metschnikowia sp. provided herein to enhance the production of ethanol from xylose, although the H0 Metschnikowia sp. has endogenous expression for all enzymes of the ethanol biosynthesis pathway from xylose.

Given the teachings and guidance provided herein, those skilled in the art will understand that the number of encoding nucleic acids to introduce in an expressible form will, at least, parallel the pathway deficiencies of the Metschnikowia species. Therefore, a Metschnikowia species of provided herein can have one, two, three, four, five, six, seven or eight up to all nucleic acids encoding the enzymes or proteins constituting a biosynthetic pathway. In some embodiments, the Metschnikowia species also can include other genetic modifications that facilitate or optimize biosynthesis of a particular compound or that confer other useful functions onto the host microbial organism. One such other functionality can include, for example, augmentation of the synthesis of one or more of the pathway precursors for a particular compound.

In some embodiments, a Metschnikowia species provided herein contains the enzymatic capability to synthesize compounds such as xylitol, arabitol, ethanol, n-butanol, isobutanol, isopropanol, ethyl acetate, or phenyl-ethyl alcohol from xylose. In this specific embodiment it can be useful to increase the synthesis or accumulation of a compound to, for example, drive the biosynthesis pathway reactions toward the production of the desired compound. Increased synthesis or accumulation can be accomplished by, for example, overexpression of nucleic acids encoding one or more of the biosynthesis pathway enzymes or proteins for producing compounds such as xylitol, arabitol, ethanol, n-butanol, isobutanol, isopropanol, ethyl acetate, or phenyl-ethyl alcohol from xylose. Overexpression of the enzyme or enzymes and/or protein or proteins of the biosynthesis pathways of desired pathway can occur, for example, through exogenous expression of the endogenous gene or genes, or through exogenous expression of the heterologous gene or genes. Therefore, the Metschnikowia species as provided herein can be readily modified for producing a desired compound, for example, through overexpression of one, two, three, four, five, and up to all nucleic acids encoding the biosynthetic pathway enzymes or proteins for the desired product. In addition, a Metschnikowia species can be generated by mutagenesis of an endogenous gene that results in an increase in activity of an enzyme in the biosynthetic pathway.

In particularly useful embodiments, exogenous expression of the encoding nucleic acids is employed. Exogenous expression confers the ability to custom tailor the expression and/or regulatory elements to the host and application to achieve a desired expression level that is controlled by the user. However, endogenous expression also can be utilized in other embodiments such as by removing a negative regulatory effector or induction of the gene's promoter when linked to an inducible promoter or other regulatory element. Thus, an endogenous gene having a naturally occurring inducible promoter can be up-regulated by providing the appropriate inducing agent, or the regulatory region of an endogenous gene can be engineered to incorporate an inducible regulatory element, thereby allowing the regulation of increased expression of an endogenous gene at a desired time. Similarly, an inducible promoter can be included as a regulatory element for an exogenous gene introduced into a Metschnikowia species.

It is understood that any of the one or more exogenous nucleic acids described herein can be introduced into a Metschnikowia species to produce a Metschnikowia species with increased production of a desired compound, such as xylitol, arabitol, ethanol, n-butanol, isobutanol, isopropanol, ethyl acetate, phenyl-ethyl alcohol, 2-methyl-butanol, or 3-methyl-butanol. The nucleic acids can be introduced so as to confer, for example, a biosynthetic pathway to produce ethanol from xylose onto the microbial organism. Alternatively, encoding nucleic acids can be introduced to produce an intermediate Metschnikowia species having the biosynthetic capability to catalyze some of the required reactions to confer biosynthetic capability. For example, a Metschnikowia species having a biosynthetic pathway can comprise at least two exogenous nucleic acids encoding desired enzymes or proteins. Thus, it is understood that any combination of two or more enzymes or proteins of a biosynthetic pathway can be included in a Metschnikowia species provided herein. Similarly, it is understood that any combination of three or more enzymes or proteins of a biosynthetic pathway can be included in a Metschnikowia species provided herein so long as the combination of enzymes and/or proteins of the desired biosynthetic pathway results in production of the corresponding desired compound. Similarly, any combination of four or more enzymes or proteins of a biosynthetic pathway as disclosed herein can be included in a Metschnikowia species provided herein, as desired, so long as the combination of enzymes and/or proteins of the desired biosynthetic pathway results in production of the corresponding desired compound.

In addition to the biosynthesis of a desired compound as described herein, the Metschnikowia species and methods provided herein also can be utilized in various combinations with each other and/or with other microbial organisms and methods well known in the art to achieve compound biosynthesis by other routes. For example, one alternative to produce ethanol other than use of the ethanol producers is through addition of a Metschnikowia species capable of converting an ethanol pathway intermediate to ethanol. One such procedure includes, for example, the fermentation by a Metschnikowia species that produces an ethanol pathway intermediate. The ethanol pathway intermediate can then be used as a substrate for a second microbial organism that converts the ethanol pathway intermediate to ethanol. The ethanol pathway intermediate can be added directly to another culture of the second organism or the original culture of the ethanol pathway intermediate producers can be depleted of these Metschnikowia species by, for example, cell separation, and then subsequent addition of the second organism to the fermentation broth can be utilized to produce the final compound without intermediate purification steps. Although ethanol is used as an example here, the same approach can be used for production of other desired compounds such as xylitol, arabitol, ethanol, n-butanol, isobutanol, isopropanol, ethyl acetate, phenyl-ethyl alcohol, 2-methyl-butanol, or 3-methyl-butanol.

In other embodiments, the Metschnikowia species and methods provided herein can be assembled in a wide variety of subpathways to achieve biosynthesis of a desired compound. In these embodiments, biosynthetic pathways for a desired compound described herein can be segregated into different Metschnikowia species, and the different Metschnikowia species can be co-cultured to produce the final compound. In such a biosynthetic scheme, the compound of one microbial organism is the substrate for a second microbial organism until the final compound is synthesized. For example, the biosynthesis of a desired compound can be accomplished by constructing a microbial organism that contains biosynthetic pathways for conversion of one pathway intermediate to another pathway intermediate or the compound. Alternatively, a desired compound also can be biosynthetically produced from Metschnikowia species through co-culture or co-fermentation using two organisms in the same vessel, where the first microbial organism produces an intermediate for the desired compound and the second microbial organism converts the intermediate to the desired compound. The desired compound can be xylitol, arabitol, ethanol, n-butanol, isobutanol, isopropanol, ethyl acetate, phenyl-ethyl alcohol, 2-methyl-butanol, or 3-methyl-butanol.

Given the teachings and guidance provided herein, those skilled in the art will understand that a wide variety of combinations and permutations exist for the Metschnikowia species and methods provided herein, together with other Metschnikowia species, with the co-culture of other Metschnikowia species having subpathways and with combinations of other chemical and/or biochemical procedures well known in the art to produce a desired compound.

Provided herein are methods of producing a bioderived compound as described herein. Such methods can include culturing an isolated Metschnikowia species having a metabolic pathway for producing the bioderived compound under conditions and for a sufficient period of time to produce the bioderived compound from xylose. Accordingly, in some embodiments, provided herein is a method for producing xylitol comprising culturing the isolated Metschnikowia species described herein under conditions and for a sufficient period of time to produce xylitol from xylose. In some embodiments, provided herein is a method for producing arabitol comprising culturing the isolated Metschnikowia species described herein under conditions and for a sufficient period of time to produce arabitol from xylose. In some embodiments, provided herein is a method for producing ethanol comprising culturing the isolated Metschnikowia species described herein under conditions and for a sufficient period of time to produce ethanol from xylose. In some embodiments, provided herein is a method for producing n-butanol comprising culturing the isolated Metschnikowia species described herein under conditions and for a sufficient period of time to produce n-butanol from xylose. In some embodiments, provided herein is a method for producing isobutanol comprising culturing the isolated Metschnikowia species described herein under conditions and for a sufficient period of time to produce isobutanol from xylose. In some embodiments, provided herein is a method for producing isopropanol comprising culturing the isolated Metschnikowia species described herein under conditions and for a sufficient period of time to produce isopropanol from xylose. In some embodiments, provided herein is a method for producing ethyl acetate comprising culturing the isolated Metschnikowia species described herein under conditions and for a sufficient period of time to produce ethyl acetate from xylose. In some embodiments, provided herein is a method for producing phenyl-ethyl alcohol comprising culturing the isolated Metschnikowia species described herein under conditions and for a sufficient period of time to produce phenyl-ethyl alcohol from xylose. In some embodiments, provided herein is a method for producing 2-methyl-butanol comprising culturing the isolated Metschnikowia species described herein under conditions and for a sufficient period of time to produce 2-methyl-butanol from xylose. In some embodiments, provided herein is a method for producing 3-methyl-butanol comprising culturing the isolated Metschnikowia species described herein under conditions and for a sufficient period of time to produce 3-methyl-butanol from xylose.

The methods provided herein include the production of the bioderived compound at a specified rate, conversion efficiency and/or concentration. Accordingly, in some embodiments, the method provided herein produces the bioderived compound (e.g., xylitol) from xylose at a rate of at least 0.1 g/L/h. In some embodiments, the method provided herein produces the bioderived compound (e.g., xylitol) from xylose at a rate of at least 0.2 g/L/h. In some embodiments, the method provided herein produces the bioderived compound (e.g., xylitol) from xylose at a rate of at least 0.3 g/L/h. In some embodiments, the method provided herein produces the bioderived compound (e.g., xylitol) from xylose at a rate of at least 0.4 g/L/h. In some embodiments, the method provided herein produces the bioderived compound (e.g., xylitol) from xylose at a rate of at least 0.50 g/L/h. In some embodiments, the method provided herein produces the bioderived compound (e.g., xylitol) from xylose at a rate of at least 0.60 g/L/h. In some embodiments, the method provided herein produces the bioderived compound (e.g., xylitol) from xylose at a rate of at least 0.70 g/L/h. In some embodiments, the method provided herein produces the bioderived compound (e.g., xylitol) from xylose at a rate of at least 0.80 g/L/h. In some embodiments, the method provided herein produces the bioderived compound (e.g., xylitol) from xylose at a rate of at least 0.90 g/L/h. In some embodiments, the method provided herein produces the bioderived compound (e.g., xylitol) from xylose at a rate of at least 1.00 g/L/h. In some embodiments, the method provided herein produces the bioderived compound (e.g., xylitol) from xylose at a rate of at least 1.50 g/L/h. In some embodiments, the method provided herein produces the bioderived compound (e.g., xylitol) from xylose at a rate of at least 2.00 g/L/h. In some embodiments, the method provided herein produces the bioderived compound (e.g., xylitol) from xylose at a rate of at least 2.50 g/L/h. In some embodiments, the method provided herein produces the bioderived compound (e.g., xylitol) from xylose at a rate of at least 3.00 g/L/h. In some embodiments, the method provided herein produces the bioderived compound (e.g., xylitol) from xylose at a rate of at least 3.50 g/L/h. In some embodiments, the method provided herein produces the bioderived compound (e.g., xylitol) from xylose at a rate of at least 4.00 g/L/h. In some embodiments, the method provided herein produces the bioderived compound (e.g., xylitol) from xylose at a rate of at least 5.00 g/L/h. In some embodiments, the method provided herein produces the bioderived compound (e.g., xylitol) from xylose at a rate of at least 6.00 g/L/h. In some embodiments, the method provided herein produces the bioderived compound (e.g., xylitol) from xylose at a rate of at least 7.00 g/L/h. In some embodiments, the method provided herein produces the bioderived compound (e.g., xylitol) from xylose at a rate of at least 8.00 g/L/h. In some embodiments, the method provided herein produces the bioderived compound (e.g., xylitol) from xylose at a rate of at least 9.00 g/L/h. In some embodiments, the method provided herein produces the bioderived compound (e.g., xylitol) from xylose at a rate of or at least 10.00 g/L/h.

In some embodiments, the method provided herein produces the bioderived compound (e.g., xylitol) from xylose at a conversion efficiency of at least 0.01 g bioderived compound per 1 g xylose. In some embodiments, the method provided herein produces the bioderived compound (e.g., xylitol) from xylose at a conversion efficiency of at least 0.02 g bioderived compound per 1 g xylose. In some embodiments, the method provided herein produces the bioderived compound (e.g., xylitol) from xylose at a conversion efficiency of at least 0.03 g bioderived compound per 1 g xylose. In some embodiments, the method provided herein produces the bioderived compound (e.g., xylitol) from xylose at a conversion efficiency of at least 0.04 g bioderived compound per 1 g xylose. In some embodiments, the method provided herein produces the bioderived compound (e.g., xylitol) from xylose at a conversion efficiency of at least 0.05 g bioderived compound per 1 g xylose. In some embodiments, the method provided herein produces the bioderived compound (e.g., xylitol) from xylose at a conversion efficiency of at least 0.06 g bioderived compound per 1 g xylose. In some embodiments, the method provided herein produces the bioderived compound (e.g., xylitol) from xylose at a conversion efficiency of at least 0.07 g bioderived compound per 1 g xylose. In some embodiments, the method provided herein produces the bioderived compound (e.g., xylitol) from xylose at a conversion efficiency of at least 0.08 g bioderived compound per 1 g xylose. In some embodiments, the method provided herein produces the bioderived compound (e.g., xylitol) from xylose at a conversion efficiency of at least 0.09 g bioderived compound per 1 g xylose. In some embodiments, the method provided herein produces the bioderived compound (e.g., xylitol) from xylose at a conversion efficiency of at least 0.1 g bioderived compound per 1 g xylose. In some embodiments, the method provided herein produces the bioderived compound (e.g., xylitol) from xylose at a conversion efficiency of at least 0.15 g bioderived compound per 1 g xylose. In some embodiments, the method provided herein produces the bioderived compound (e.g., xylitol) from xylose at a conversion efficiency of at least 0.2 g bioderived compound per 1 g xylose. In some embodiments, the method provided herein produces the bioderived compound (e.g., xylitol) from xylose at a conversion efficiency of at least 0.25 g bioderived compound per 1 g xylose. In some embodiments, the method provided herein produces the bioderived compound (e.g., xylitol) from xylose at a conversion efficiency of at least 0.3 g bioderived compound per 1 g xylose. In some embodiments, the method provided herein produces the bioderived compound (e.g., xylitol) from xylose at a conversion efficiency of at least 0.35 g bioderived compound per 1 g xylose. In some embodiments, the method provided herein produces the bioderived compound (e.g., xylitol) from xylose at a conversion efficiency of at least 0.4 g bioderived compound per 1 g xylose. In some embodiments, the method provided herein produces the bioderived compound (e.g., xylitol) from xylose at a conversion efficiency of at least 0.45 g bioderived compound per 1 g xylose. In some embodiments, the method provided herein produces the bioderived compound (e.g., xylitol) from xylose at a conversion efficiency of at least 0.5 g bioderived compound per 1 g xylose. In some embodiments, the method provided herein produces the bioderived compound (e.g., xylitol) from xylose at a conversion efficiency of at least 0.55 g bioderived compound per 1 g xylose. In some embodiments, the method provided herein produces the bioderived compound (e.g., xylitol) from xylose at a conversion efficiency of at least 0.6 g bioderived compound per 1 g xylose. In some embodiments, the method provided herein produces the bioderived compound (e.g., xylitol) from xylose at a conversion efficiency of at least 0.65 g bioderived compound per 1 g xylose. In some embodiments, the method provided herein produces the bioderived compound (e.g., xylitol) from xylose at a conversion efficiency of at least 0.7 g bioderived compound per 1 g xylose. In some embodiments, the method provided herein produces the bioderived compound (e.g., xylitol) from xylose at a conversion efficiency of at least 0.75 g bioderived compound per 1 g xylose. In some embodiments, the method provided herein produces the bioderived compound (e.g., xylitol) from xylose at a conversion efficiency of at least 0.8 g bioderived compound per 1 g xylose. In some embodiments, the method provided herein produces the bioderived compound (e.g., xylitol) from xylose at a conversion efficiency of at least 0.85 g bioderived compound per 1 g xylose. In some embodiments, the method provided herein produces the bioderived compound (e.g., xylitol) from xylose at a conversion efficiency of at least 0.9 g bioderived compound per 1 g xylose. In some embodiments, the method provided herein produces the bioderived compound (e.g., xylitol) from xylose at a conversion efficiency of at least 0.95 g bioderived compound per 1 g xylose. In some embodiments, the method provided herein produces the bioderived compound (e.g., xylitol) from xylose at a conversion efficiency of at least 1 g bioderived compound per 1 g xylose.

In some embodiments, the method provided herein produces the bioderived compound (e.g., xylitol) from xylose at a concentration of at least 1 g/L. In some embodiments, the method provided herein produces the bioderived compound (e.g., xylitol) from xylose at a concentration of at least 2 g/L. In some embodiments, the method provided herein produces the bioderived compound (e.g., xylitol) from xylose at a concentration of at least 3 g/L. In some embodiments, the method provided herein produces the bioderived compound (e.g., xylitol) from xylose at a concentration of at least 4 g/L. In some embodiments, the method provided herein produces the bioderived compound (e.g., xylitol) from xylose at a concentration of at least 5 g/L. In some embodiments, the method provided herein produces the bioderived compound (e.g., xylitol) from xylose at a concentration of at least 10 g/L. In some embodiments, the method provided herein produces the bioderived compound (e.g., xylitol) from xylose at a concentration of at least 20 g/L. In some embodiments, the method provided herein produces the bioderived compound (e.g., xylitol) from xylose at a concentration of at least 30 g/L. In some embodiments, the method provided herein produces the bioderived compound (e.g., xylitol) from xylose at a concentration of at least 40 g/L. In some embodiments, the method provided herein produces the bioderived compound (e.g., xylitol) from xylose at a concentration of at least 50 g/L. In some embodiments, the method provided herein produces the bioderived compound (e.g., xylitol) from xylose at a concentration of at least 60 g/L. In some embodiments, the method provided herein produces the bioderived compound (e.g., xylitol) from xylose at a concentration of at least 70 g/L. In some embodiments, the method provided herein produces the bioderived compound (e.g., xylitol) from xylose at a concentration of at least 80 g/L. In some embodiments, the method provided herein produces the bioderived compound (e.g., xylitol) from xylose at a concentration of at least 90 g/L. In some embodiments, the method provided herein produces the bioderived compound (e.g., xylitol) from xylose at a concentration of at least 100 g/L. In some embodiments, the method provided herein produces the bioderived compound (e.g., xylitol) from xylose at a concentration of at least 150 g/L. In some embodiments, the method provided herein produces the bioderived compound (e.g., xylitol) from xylose at a concentration of at least 200 g/L. In some embodiments, the method provided herein produces the bioderived compound (e.g., xylitol) from xylose at a concentration of at least 250 g/L. In some embodiments, the method provided herein produces the bioderived compound (e.g., xylitol) from xylose at a concentration of at least 300 g/L. In some embodiments, the method provided herein produces the bioderived compound (e.g., xylitol) from xylose at a concentration of at least 350 g/L. In some embodiments, the method provided herein produces the bioderived compound (e.g., xylitol) from xylose at a concentration of at least 400 g/L. In some embodiments, the method provided herein produces the bioderived compound (e.g., xylitol) from xylose at a concentration of at least 500 g/L.

Any of the Metschnikowia species described herein can be cultured to produce and/or secrete the desired bioderived compound including such as xylitol, arabitol, ethanol, n-butanol, isobutanol, isopropanol, ethyl acetate, phenyl-ethyl alcohol, 2-methyl-butanol, or 3-methyl-butanol. For example, the Metschnikowia species provided herein can be cultured for the biosynthetic production of a desired compound. Accordingly, in some embodiments, provided herein are culture media containing a desired bioderived compound described herein or intermediate thereof. In some aspects, the culture medium can also be separated from the Metschnikowia species that produced the desired bioderived compound or intermediate thereof. Methods for separating a microbial organism from culture medium are well known in the art. Exemplary methods include filtration, flocculation, precipitation, centrifugation, sedimentation, and the like.

For the production of the desired bioderived compound, including xylitol, arabitol, ethanol, n-butanol, isobutanol, isopropanol, ethyl acetate, phenyl-ethyl alcohol, 2-methyl-butanol, or 3-methyl-butanol, the Metschnikowia species provided herein are cultured in a medium with a carbon source and other essential nutrients. In some embodiments, the Metschnikowia species provided herein are cultured in an aerobic culture medium. The aerobic culturing can be batch, fed-bartch or continuous culturing, wherein the dissolved oxygen in the medium is above 50% of saturation. In some embodiments, the Metschnikowia species provided herein are cultured in a substantially anaerobic culture medium. As described herein, one exemplary growth condition for achieving biosynthesis of a desired compound such as xylitol, arabitol, ethanol, n-butanol, isobutanol, isopropanol, ethyl acetate, phenyl-ethyl alcohol, 2-methyl-butanol, or 3-methyl-butanol includes anaerobic culture or fermentation conditions. In certain embodiments, the Metschnikowia species provided herein can be sustained, cultured or fermented under anaerobic or substantially anaerobic conditions. Briefly, an anaerobic condition refers to an environment devoid of oxygen. Substantially anaerobic conditions include, for example, a culture, batch fermentation or continuous fermentation such that the dissolved oxygen concentration in the medium remains between 0 and 10% of saturation. Substantially anaerobic conditions also include growing or resting cells in liquid medium or on solid agar inside a sealed chamber maintained with an atmosphere of less than 1% oxygen. The percent of oxygen can be maintained by, for example, sparging the culture with an N₂/CO₂ mixture or other suitable non-oxygen gas or gases.

It is sometimes desirable to maintain anaerobic conditions in the fermenter to reduce the cost of the overall process. Such conditions can be obtained, for example, by first sparging the medium with nitrogen and then sealing the flasks with a septum and crimp-cap. For strains where growth is not observed anaerobically, microaerobic or substantially anaerobic conditions can be applied by perforating the septum with a small hole for limited aeration. Exemplary anaerobic conditions have been described previously and are well-known in the art. Exemplary aerobic and anaerobic conditions are described, for example, in United States publication 2009/0047719, filed Aug. 10, 2007. Fermentations can be performed in a batch, fed-batch or continuous manner, as disclosed herein. Fermentations can also be conducted in two phases, if desired. The first phase can be aerobic to allow for high growth and therefore high productivity, followed by an anaerobic phase of high yields.

If desired, the pH of the medium can be maintained at a desired pH, such as a pH of around 5.5-6.5 by addition of a base, such as NaOH or other bases, or acid, as needed to maintain the culture medium at a desirable pH. The growth rate can be determined by measuring optical density using a spectrophotometer (600 nm), and the xylose uptake rate by monitoring carbon source depletion over time.

The culture medium for the Metschnikowia species provided herein can include xylose, either as the sole source of carbon or in combination with one or more co-substrates described herein or known in the art. The culture medium can further include other supplements, such as yeast extract, and/or peptone. The culture medium can further include, for example, any other carbohydrate source which can supply a source of carbon to the Metschnikowia species. Such sources include, for example: other sugars such as cellobiose, galactose, glucose, ethanol, acetate, arabitol, sorbitol and glycerol. Thus, the culture medium can include xylose and the co-substrate glucose. The culture medium can include xylose and the co-substrate cellobiose. The culture medium can include xylose and the co-substrate galactose. The culture medium can include xylose and the co-substrate glycerol. The culture medium can include a combination of glucose, xylose and cellobiose. The culture medium can include a combination of glucose, xylose, and galactose. The culture medium can include a combination of glucose, xylose, and glycerol. The culture medium can include a combination of xylose, cellobiose, galactose and glycerol.

The culture medium can have 1%, 2%, 3%, 4%, 5%, 6%, 7%, 8%, 9%, 10%, 11%, 12%, 13%, 14%, 15%, 16%, 17%, 18%, 19%, 20%, or higher amount of a carbon source (w/v). In some embodiments, the culture medium can have 2% carbon source. In some embodiments, the culture medium can have 4% carbon source. In some embodiments, the culture medium can have 10% carbon source. In some embodiments, the culture medium can have 1%, 2%, 3%, 4%, 5%, 6%, 7%, 8%, 9%, 10%, 11%, 12%, 13%, 14%, 15%, 16%, 17%, 18%, 19%, 20%, or higher amount of xylose (w/v). The culture medium can have 1% xylose. The culture medium can have 2% xylose. The culture medium can have 3% xylose. The culture medium can have 4% xylose. The culture medium can have 5% xylose. The culture medium can have 6% xylose. The culture medium can have 7% xylose. The culture medium can have 8% xylose. The culture medium can have 9% xylose. The culture medium can have 10% xylose. The culture medium can have 11% xylose. The culture medium can have 12% xylose. The culture medium can have 13% xylose. The culture medium can have 14% xylose. The culture medium can have 15% xylose. The culture medium can have 16% xylose. The culture medium can have 17% xylose. The culture medium can have 18% xylose. The culture medium can have 19% xylose. The culture medium can have 20% xylose.

In some embodiments, xylose is not the only carbon source. For example, in some embodiments, the medium includes xylose and a C3 carbon source, a C4 carbon source, a C5 carbon source, a C6 carbon source, or a combination thereof. Accordingly, in some embodiments, the medium includes xylose and a C3 carbon source (e.g., glycerol). In some embodiments, the medium includes xylose and a C4 carbon source (e.g., erythrose or threose). In some embodiments, the medium includes xylose and a C5 carbon source (e.g., arabitol, ribose or lyxose). In some embodiments, the medium includes xylose and a C6 carbon source (e.g., glucose, galactose, mannose, allose, altrose, gulose, and idose). Alternatively or additionally, in some embodiments, the medium includes xylose and cellobiose, galactose, glucose, arabitol, sorbitol and glycerol, or a combination thereof. In a specific embodiment, the medium includes xylose and glucose. The amount of the two or more carbon sources in the medium can range independently from 1% to 20% (e.g., 1% to 20% xylose and 1% to 20% glucose), or alternatively 2% to 14% (e.g., 2% to 14% xylose and 2% to 14% glucose), or alternatively 4% to 10% (e.g., 4% to 10% xylose and 4% to 10%). In a specific embodiment, the amount of each of the carbon sources is 2% (e.g., 2% xylose and 2% glucose)

The culture medium can be a C5-rich medium, with a five carbon sugar (such as xylose) as the primary carbon source. The culture medium can also have a C6 sugar (six-carbon sugar). In some embodiments, the culture medium can have a C6 sugar as the primary carbon source. In some embodiments, the C6 sugar is glucose. The culture can have both a C6 sugar and a C5 sugar as the carbon source, and can have the C6 sugar and the C5 sugar present at different ratios. In some embodiment, the ratio of the amount of C6 sugar to that of the C5 sugar (the C6: C5 ratio) in the culture medium is between about 10:1 and about 1:20. For example, the C6: C5 ratio in the culture medium can be about 10:1, 9:1, 8:1, 7:1, 6:1, 5:1, 3:1, 2:1, 1:1, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19 or 1:20. In some embodiments, the C6: C5 ratio in the culture medium is about 3:1. In some embodiments, the C6: C5 ratio in the culture medium is about 1:1. In some embodiments, the C6: C5 ratio in the culture medium is about 1:5. In some embodiments, the C6: C5 ratio in the culture medium is about 1:10. The C5 sugar can be xylose, and the C6 sugar can be glucose. In some embodiments, the ratio of the amount of glucose to that of xylose (the glucose: xylose ratio) in the culture medium is between about 20:1 and about 1:10. For example, the glucose: xylose ratio in the culture medium can be about 20:1, 19:1, 18:1, 17:1, 16:1, 15:1, 14:1, 13:1, 12:1, 11:1, 10:1, 9:1, 8:1, 7:1, 6:1, 5:1, 3:1, 2:1, 1:1, 1:2, 1:3, 1:4, 1:5, 1:6, 1:7, 1:8, 1:9 or 1:10. In some embodiments, the glucose: xylose ratio in the culture medium is about 3:1. In some embodiments, the glucose: xylose ratio in the culture medium is about 1:1. In some embodiments, the glucose: xylose ratio in the culture medium is about 1:5. In some embodiments, the glucose: xylose ratio in the culture medium is about 1:10.

Other sources of carbohydrate include, for example, renewable feedstocks and biomass. Exemplary types of biomasses that can be used as feedstocks in the methods provided herein include cellulosic biomass and hemicellulosic biomass feedstocks or portions of feedstocks. Such biomass feedstocks contain, for example, carbohydrate substrates useful as carbon sources such as xylose, glucose, arabinose, galactose, mannose, fructose and starch. Given the teachings and guidance provided herein, those skilled in the art will understand that renewable feedstocks and biomass other than those exemplified above also can be used for culturing the Metschnikowia species provided herein for the production of the desired bioderived compound including such as xylitol, arabitol, ethanol, n-butanol, isobutanol, isopropanol, ethyl acetate, phenyl-ethyl alcohol, 2-methyl-butanol, or 3-methyl-butanol.

Accordingly, given the teachings and guidance provided herein, those skilled in the art will understand that a Metschnikowia species can be produced that secretes the biosynthesized compounds described herein when grown on xylose as a carbon source. Such compounds include, for example, xylitol, arabitol, ethanol, n-butanol, isobutanol, isopropanol, ethyl acetate, phenyl-ethyl alcohol, 2-methyl-butanol, or 3-methyl-butanol and any of the intermediate metabolites thereof. All that is required is to engineer in one or more of the required enzyme or protein activities to achieve biosynthesis of the desired compound or intermediate including, for example, inclusion of some or all of the biosynthetic pathways for producing the desired compound. Accordingly, provided herein is a Metschnikowia species that produces and/or secretes a desired compound such as xylitol, arabitol, ethanol, n-butanol, isobutanol, isopropanol, ethyl acetate, phenyl-ethyl alcohol, 2-methyl-butanol, or 3-methyl-butanol when grown on a carbohydrate or other carbon source and produces and/or secretes an intermediate metabolites shown in the biosynthesis pathway of the desired compound when grown on xylose and optionally other carbohydrate or carbon source.

The Metschnikowia species provided herein can be constructed using methods well known in the art as exemplified herein to exogenously express at least one nucleic acid encoding an enzyme or protein of a metabolic pathway in sufficient amounts to produce a desired compound from xylose. It is understood that the Metschnikowia species provided herein are cultured under conditions sufficient to produce a desired compound such as xylitol, arabitol, ethanol, n-butanol, isobutanol, isopropanol, ethyl acetate, phenyl-ethyl alcohol, 2-methyl-butanol, or 3-methyl-butanol. Following the teachings and guidance provided herein, the Metschnikowia species provided herein can achieve biosynthesis of the desired compound resulting in intracellular concentrations between about 0.1-200 mM or more. Generally, the intracellular concentration of the desired compound between about 3-150 mM, particularly between about 5-125 mM and more particularly between about 8-100 mM, including about 10 mM, 20 mM, 50 mM, 80 mM, or more. Intracellular concentrations between and above each of these exemplary ranges also can be achieved from the Metschnikowia species provided herein.

In some embodiments, culture conditions include anaerobic or substantially anaerobic growth or maintenance conditions. Exemplary anaerobic conditions have been described previously and are well known in the art. Exemplary anaerobic conditions for fermentation processes are described herein and are described, for example, in U.S. publication 2009/0047719. Any of these conditions can be employed with the Metschnikowia species as well as other anaerobic conditions well known in the art. Under such anaerobic or substantially anaerobic conditions, the producer strains can synthesize the desired compound at intracellular concentrations of 5-10 mM or more as well as all other concentrations exemplified herein. It is understood that, even though the above description refers to intracellular concentrations, the producing Metschnikowia species can produce the desired compound intracellularly and/or secrete the compound into the culture medium.

The methods provided herein can include any culturing process well known in the art, such as batch cultivation, fed-batch cultivation or continuous cultivation. Such process can include fermentation. Exemplary fermentation processes include, but are not limited to, fed-batch fermentation and batch separation; fed-batch fermentation and continuous separation; and continuous fermentation and continuous separation. In an exemplary batch fermentation protocol, the production organism is grown in a suitably sized bioreactor sparged with an appropriate gas. Under anaerobic conditions, the culture is sparged with an inert gas or combination of gases, for example, nitrogen, N₂/CO₂ mixture, argon, helium, and the like. As the cells grow and utilize the carbon source, additional carbon source(s) and/or other nutrients are fed into the bioreactor at a rate approximately balancing consumption of the carbon source and/or nutrients. The temperature of the bioreactor is maintained at a desired temperature, generally in the range of 22-37 degrees C., but the temperature can be maintained at a higher or lower temperature depending on the growth characteristics of the production organism and/or desired conditions for the fermentation process. Growth continues for a desired period of time to achieve desired characteristics of the culture in the fermenter, for example, cell density, compound concentration, and the like. In a batch fermentation process, the time period for the fermentation is generally in the range of several hours to several days, for example, 8 to 24 hours, or 1, 2, 3, 4 or 5 days, or up to a week, depending on the desired culture conditions. The pH can be controlled or not, as desired, in which case a culture in which pH is not controlled will typically decrease to pH 3-6 by the end of the run. Upon completion of the cultivation period, the fermenter contents can be passed through a cell separation unit, for example, a centrifuge, filtration unit, and the like, to remove cells and cell debris. In the case where the desired compound is expressed intracellularly, the cells can be lysed or disrupted enzymatically or chemically prior to or after separation of cells from the fermentation broth, as desired, in order to release additional compound. The fermentation broth can be transferred to a compound separations unit. Isolation of compound occurs by standard separations procedures employed in the art to separate a desired compound from dilute aqueous solutions. Such methods include, but are not limited to, liquid-liquid extraction using a water immiscible organic solvent (e.g., toluene or other suitable solvents, including but not limited to diethyl ether, ethyl acetate, tetrahydrofuran (THF), methylene chloride, chloroform, benzene, pentane, hexane, heptane, petroleum ether, methyl tertiary butyl ether (MTBE), dioxane, dimethylformamide (DMF), dimethyl sulfoxide (DMSO), and the like) to provide an organic solution of the compound, if appropriate, standard distillation methods, and the like, depending on the chemical characteristics of the compound of the fermentation process.

In an exemplary fully continuous fermentation protocol, the production organism is generally first grown up in batch mode in order to achieve a desired cell density. When the carbon source and/or other nutrients are exhausted, feed medium of the same composition is supplied continuously at a desired rate, and fermentation liquid is withdrawn at the same rate. Under such conditions, the compound concentration in the bioreactor generally remains constant, as well as the cell density. The temperature of the fermenter is maintained at a desired temperature, as discussed above. During the continuous fermentation phase, it is generally desirable to maintain a suitable pH range for optimized production. The pH can be monitored and maintained using routine methods, including the addition of suitable acids or bases to maintain a desired pH range. The bioreactor is operated continuously for extended periods of time, generally at least one week to several weeks and up to one month, or longer, as appropriate and desired. The fermentation liquid and/or culture is monitored periodically, including sampling up to every day, as desired, to assure consistency of compound concentration and/or cell density. In continuous mode, fermenter contents are constantly removed as new feed medium is supplied. The exit stream, containing cells, medium, and product, are generally subjected to a continuous compound separations procedure, with or without removing cells and cell debris, as desired. Continuous separations methods employed in the art can be used to separate the compound from dilute aqueous solutions, including but not limited to continuous liquid-liquid extraction using a water immiscible organic solvent (e.g., toluene or other suitable solvents, including but not limited to diethyl ether, ethyl acetate, tetrahydrofuran (THF), methylene chloride, chloroform, benzene, pentane, hexane, heptane, petroleum ether, methyl tertiary butyl ether (MTBE), dioxane, dimethylformamide (DMF), dimethyl sulfoxide (DMSO), and the like), standard continuous distillation methods, and the like, or other methods well known in the art.

In addition to the culturing and fermentation conditions disclosed herein, growth condition for achieving biosynthesis of the desired compound can include the addition of an osmoprotectant to the culturing conditions. In certain embodiments, the Metschnikowia species provided herein can be sustained, cultured or fermented as described herein in the presence of an osmoprotectant. Briefly, an osmoprotectant refers to a compound that acts as an osmolyte and helps a microbial organism as described herein survive osmotic stress. Osmoprotectants include, but are not limited to, betaines, amino acids, and the sugar trehalose. Non-limiting examples of such are glycine betaine, praline betaine, dimethylthetin, dimethylslfonioproprionate, 3-dimethyl sulfonio-2-methylproprionate, pipecolic acid, dimethyl sulfonioacetate, choline, L-carnitine and ectoine. In one aspect, the osmoprotectant is glycine betaine. It is understood to one of ordinary skill in the art that the amount and type of osmoprotectant suitable for protecting a microbial organism described herein from osmotic stress will depend on the microbial organism used. The amount of osmoprotectant in the culturing conditions can be, for example, no more than about 0.1 mM, no more than about 0.5 mM, no more than about 1.0 mM, no more than about 1.5 mM, no more than about 2.0 mM, no more than about 2.5 mM, no more than about 3.0 mM, no more than about 5.0 mM, no more than about 7.0 mM, no more than about 10 mM, no more than about 50 mM, no more than about 100 mM or no more than about 500 mM.

The culture conditions can include, for example, liquid culture procedures as well as fermentation and other large scale culture procedures. As described herein, particularly useful yields of the biosynthetic products can be obtained under aerobic, anaerobic or substantially anaerobic culture conditions.

The culture conditions described herein can be scaled up and grown continuously for manufacturing of a desired compound. Exemplary growth procedures include, for example, fed-batch fermentation and batch separation; fed-batch fermentation and continuous separation, or continuous fermentation and continuous separation. All of these processes are well known in the art. Fermentation procedures are particularly useful for the biosynthetic production of commercial quantities of a desired product. Generally, and as with non-continuous culture procedures, the continuous and/or near-continuous production includes culturing the Metschnikowia species provided herein in sufficient nutrients and medium to sustain and/or nearly sustain growth in an exponential phase. Continuous culture under such conditions can include, for example, growth or culturing for 1 day, 2, 3, 4, 5, 6 or 7 days or more. Additionally, continuous culture can include longer time periods of 1 week, 2, 3, 4 or 5 or more weeks and up to several months. Alternatively, organisms provided herein can be cultured for hours, if suitable for a particular application. It is to be understood that the continuous and/or near-continuous culture conditions also can include all time intervals in between these exemplary periods. It is further understood that the time of culturing the microbial organism provided herein is for a sufficient period of time to produce a sufficient amount of compound for a desired purpose.

In addition to the above fermentation procedures using Metschnikowia species provided herein using continuous production of substantial quantities of a desired compound, the bioderived compound also can be, for example, simultaneously subjected to chemical synthesis and/or enzymatic procedures to convert the compound to other compounds, or the bioderived compound can be separated from the fermentation culture and sequentially subjected to chemical and/or enzymatic conversion to convert the compound to other compounds, if desired.

To generate better producers, metabolic modeling can be utilized to optimize growth conditions. Modeling can also be used to design gene knockouts that additionally optimize utilization of the pathway (see, for example, U.S. patent publications US 2002/0012939, US 2003/0224363, US 2004/0029149, US 2004/0072723, US 2003/0059792, US 2002/0168654 and US 2004/0009466, and U.S. Pat. No. 7,127,379). Modeling analysis allows reliable predictions of the effects on cell growth of shifting the metabolism towards more efficient production of a desired product.

In some embodiments, the methods provided herein to produce a bioderived compound further include separating the bioderived compound from other components in the culture using a variety of methods well known in the art. The bioderived compound can be xylitol, arabitol, ethanol, n-butanol, isobutanol, isopropanol, ethyl acetate, phenyl-ethyl alcohol, 2-methyl-butanol, or 3-methyl-butanol. Such separation methods include, for example, extraction procedures as well as methods that include continuous liquid-liquid extraction, pervaporation, membrane filtration, membrane separation, reverse osmosis, electrodialysis, distillation, crystallization, centrifugation, extractive filtration, ion exchange chromatography, size exclusion chromatography, adsorption chromatography, ultrafiltration, activated charcoal adsorption, pH adjustment and precipitation, or a combination of one or more methods enumerated above. All of the above methods are well known in the art.

Also provided herein is a bioderived compound as described herein. In some embodiments, the bioderived compound, including xylitol, arabitol, ethanol, n-butanol, isobutanol, isopropanol, ethyl acetate, phenyl-ethyl alcohol, 2-methyl-butanol, or 3-methyl-butanol, is produced by the methods provided herein.

Provided herein are also compositions having a bioderived compound produced by the Metschnikowia species described herein, and an additional component. The component other than the bioderived compound can be a cellular portion, for example, a trace amount of a cellular portion of the culture medium, or can be fermentation broth or culture medium or a purified or partially purified fraction thereof produced in the presence of, a Metschnikowia species provided herein. Thus, in some embodiment, the composition is culture medium. In some embodiments, the culture medium can be culture medium from which the isolated Metschnikowia species provided herein has been removed. The composition can have, for example, a reduced level of a byproduct when produced by the Metschnikowia species provided herein. The composition can have, for example, one or more bioderived compound such as xylitol, arabitol, ethanol, n-butanol, isobutanol, isopropanol, ethyl acetate, phenyl-ethyl alcohol, 2-methyl-butanol, or 3-methyl-butanol, and a cell lysate or culture supernatant of a Metschnikowia species provided herein. The additional component can be a byproduct, or an impurity, such as glycerol, arabitol, a C7 sugar alcohol, or a combination thereof. The byproduct can be glycerol. The byproduct can be arabitol. The byproduct can be a C7 sugar alcohol (e.g., volemitol or an isomer thereof). In some embodiments, the byproduct or impurity (e.g., glycerol or arabitol, or both) is at least 10%, 20%, 30% or 40% greater than the amount of the respective byproduct or impurity produced by a microbial organism other than the isolated Metschnikowia species provided herein.

In some embodiments, the compositions provided herein can have a bioderived xylitol and an additional component. The additional component can be fermentation broth or culture medium. The additional component can be the supernatant of fermentation broth or culture medium. The additional component can be a cellular portion of fermentation broth or culture medium. The additional component can be the Metschnikowia species having an exogenous nucleic acid encoding a protein as described herein used to produce the bioderived xylitol. The additional component can be the cell lysate of the microbial organism provided herein. The additional component can be a byproduct, such as glycerol, arabitol, a C7 sugar alcohol, or a combination thereof.

In some embodiments, the compositions provided herein can have a bioderived arabitol and an additional component. The additional component can be fermentation broth or culture medium. The additional component can be the supernatant of fermentation broth or culture medium. The additional component can be a cellular portion of fermentation broth or culture medium. The additional component can be the Metschnikowia species having an exogenous nucleic acid encoding a protein as described herein used to produce the bioderived ethanol. The additional component can be the cell lysate of the microbial organism provided herein. The additional component can be a byproduct, such as glycerol, arabitol, a C7 sugar alcohol, or a combination thereof.

In some embodiments, the compositions provided herein can have a bioderived ethanol and an additional component. The additional component can be fermentation broth or culture medium. The additional component can be the supernatant of fermentation broth or culture medium. The additional component can be a cellular portion of fermentation broth or culture medium. The additional component can be the Metschnikowia species having an exogenous nucleic acid encoding a protein as described herein used to produce the bioderived ethanol. The additional component can be the cell lysate of the microbial organism provided herein. The additional component can be a byproduct, such as glycerol, arabitol, a C7 sugar alcohol, or a combination thereof.

In some embodiments, the compositions provided herein can have a bioderived n-butanol and an additional component. The additional component can be fermentation broth or culture medium. The additional component can be the supernatant of fermentation broth or culture medium. The additional component can be a cellular portion of fermentation broth or culture medium. The additional component can be the Metschnikowia species having an exogenous nucleic acid encoding a protein as described herein used to produce the bioderived n-butanol. The additional component can be the cell lysate of the microbial organism provided herein. The additional component can be a byproduct, such as glycerol, arabitol, a C7 sugar alcohol, or a combination thereof.

In some embodiments, the compositions provided herein can have a bioderived isobutanol and an additional component. The additional component can be fermentation broth or culture medium. The additional component can be the supernatant of fermentation broth or culture medium. The additional component can be a cellular portion of fermentation broth or culture medium. The additional component can be the Metschnikowia species having an exogenous nucleic acid encoding a protein as described herein used to produce the bioderived isobutanol. The additional component can be the cell lysate of the microbial organism provided herein. The additional component can be a byproduct, such as glycerol, arabitol, a C7 sugar alcohol, or a combination thereof.

In some embodiments, the compositions provided herein can have a bioderived isopropanol and an additional component. The additional component can be fermentation broth or culture medium. The additional component can be the supernatant of fermentation broth or culture medium. The additional component can be a cellular portion of fermentation broth or culture medium. The additional component can be the Metschnikowia species having an exogenous nucleic acid encoding a protein as described herein used to produce the bioderived isopropanol. The additional component can be the cell lysate of the microbial organism provided herein. The additional component can be a byproduct, such as glycerol, arabitol, a C7 sugar alcohol, or a combination thereof.

In some embodiments, the compositions provided herein can have a bioderived ethyl acetate and an additional component. The additional component can be fermentation broth or culture medium. The additional component can be the supernatant of fermentation broth or culture medium. The additional component can be a cellular portion of fermentation broth or culture medium. The additional component can be the Metschnikowia species having an exogenous nucleic acid encoding a protein as described herein used to produce the bioderived ethyl acetate. The additional component can be the cell lysate of the microbial organism provided herein. The additional component can be a byproduct, such as glycerol, arabitol, a C7 sugar alcohol, or a combination thereof.

In some embodiments, the compositions provided herein can have a bioderived phenyl-ethyl alcohol and an additional component. The additional component can be fermentation broth or culture medium. The additional component can be the supernatant of fermentation broth or culture medium. The additional component can be a cellular portion of fermentation broth or culture medium. The additional component can be the Metschnikowia species having an exogenous nucleic acid encoding a protein as described herein used to produce the bioderived phenyl-ethyl alcohol. The additional component can be the cell lysate of the microbial organism provided herein. The additional component can be a byproduct, such as glycerol, arabitol, a C7 sugar alcohol, or a combination thereof.

In some embodiments, the compositions provided herein can have a bioderived 2-methyl-butanol and an additional component. The additional component can be fermentation broth or culture medium. The additional component can be the supernatant of fermentation broth or culture medium. The additional component can be a cellular portion of fermentation broth or culture medium. The additional component can be the Metschnikowia species having an exogenous nucleic acid encoding a protein as described herein used to produce the bioderived 2-methyl-butanol. The additional component can be the cell lysate of the microbial organism provided herein. The additional component can be a byproduct, such as glycerol, arabitol, a C7 sugar alcohol, or a combination thereof.

In some embodiments, the compositions provided herein can have a bioderived 3-methyl-butanol and an additional component. The additional component can be fermentation broth or culture medium. The additional component can be the supernatant of fermentation broth or culture medium. The additional component can be a cellular portion of fermentation broth or culture medium. The additional component can be the microbial organisms having an exogenous nucleic acid encoding a protein as described herein used to produce the bioderived 3-methyl-butanol. The additional component can be the cell lysate of the microbial organism provided herein. The additional component can be a byproduct, such as glycerol, arabitol, a C7 sugar alcohol, or a combination thereof.

In some embodiments, the carbon feedstock and other cellular uptake sources such as phosphate, ammonia, sulfate, chloride and other halogens can be chosen to alter the isotopic distribution of the atoms present in the bioderived compound produced by Metschnikowia species provided herein. The various carbon feedstock and other uptake sources enumerated above will be referred to herein, collectively, as “uptake sources.” Uptake sources can provide isotopic enrichment for any atom present in the bioderived compound produced by Metschnikowia species provided herein, or in the byproducts or impurities. Isotopic enrichment can be achieved for any target atom including, for example, carbon, hydrogen, oxygen, nitrogen, sulfur, phosphorus, chloride or other halogens.

In some embodiments, the uptake sources can be selected to alter the carbon-12, carbon-13, and carbon-14 ratios. In some embodiments, the uptake sources can be selected to alter the oxygen-16, oxygen-17, and oxygen-18 ratios. In some embodiments, the uptake sources can be selected to alter the hydrogen, deuterium, and tritium ratios. In some embodiments, the uptake sources can be selected to alter the nitrogen-14 and nitrogen-15 ratios. In some embodiments, the uptake sources can be selected to alter the sulfur-32, sulfur-33, sulfur-34, and sulfur-35 ratios. In some embodiments, the uptake sources can be selected to alter the phosphorus-31, phosphorus-32, and phosphorus-33 ratios. In some embodiments, the uptake sources can be selected to alter the chlorine-35, chlorine-36, and chlorine-37 ratios.

In some embodiments, the isotopic ratio of a target atom can be varied to a desired ratio by selecting one or more uptake sources. An uptake source can be derived from a natural source, as found in nature, or from a man-made source, and one skilled in the art can select a natural source, a man-made source, or a combination thereof, to achieve a desired isotopic ratio of a target atom. An example of a man-made uptake source includes, for example, an uptake source that is at least partially derived from a chemical synthetic reaction. Such isotopically enriched uptake sources can be purchased commercially or prepared in the laboratory and/or optionally mixed with a natural source of the uptake source to achieve a desired isotopic ratio. In some embodiments, a target atom isotopic ratio of an uptake source can be achieved by selecting a desired origin of the uptake source as found in nature. For example, as discussed herein, a natural source can be a biobased derived from or synthesized by a biological organism or a source such as petroleum-based products or the atmosphere. In some such embodiments, a source of carbon, for example, can be selected from a fossil fuel-derived carbon source, which can be relatively depleted of carbon-14, or an environmental or atmospheric carbon source, such as CO₂, which can possess a larger amount of carbon-14 than its petroleum-derived counterpart.

The unstable carbon isotope carbon-14 or radiocarbon makes up for roughly 1 in 10¹² carbon atoms in the earth's atmosphere and has a half-life of about 5700 years. The stock of carbon is replenished in the upper atmosphere by a nuclear reaction involving cosmic rays and ordinary nitrogen (¹⁴N). Fossil fuels contain no carbon-14, as it decayed long ago. Burning of fossil fuels lowers the atmospheric carbon-14 fraction, the so-called “Suess effect”.

Methods of determining the isotopic ratios of atoms in a compound are well known to those skilled in the art. Isotopic enrichment is readily assessed by mass spectrometry using techniques known in the art such as accelerated mass spectrometry (AMS), Stable Isotope Ratio Mass Spectrometry (SIRMS) and Site-Specific Natural Isotopic Fractionation by Nuclear Magnetic Resonance (SNIF-NMR). Such mass spectral techniques can be integrated with separation techniques such as liquid chromatography (LC), high performance liquid chromatography (HPLC) and/or gas chromatography, and the like.

In the case of carbon, ASTM D6866 was developed in the United States as a standardized analytical method for determining the biobased content of solid, liquid, and gaseous samples using radiocarbon dating by the American Society for Testing and Materials (ASTM) International. The standard is based on the use of radiocarbon dating for the determination of a product's biobased content. ASTM D6866 was first published in 2004, and the current active version of the standard is ASTM D6866-11 (effective Apr. 1, 2011). Radiocarbon dating techniques are well known to those skilled in the art, including those described herein.

The biobased content of a compound is estimated by the ratio of carbon-14 (¹⁴C) to carbon-12 (¹²C). Specifically, the Fraction Modern (Fm) is computed from the expression: Fm=(S-B)/(M-B), where B, S and M represent the ¹⁴C/¹²C ratios of the blank, the sample and the modern reference, respectively. Fraction Modern is a measurement of the deviation of the ¹⁴C/¹²C ratio of a sample from “Modern.” Modern is defined as 95% of the radiocarbon concentration (in AD 1950) of National Bureau of Standards (NBS) Oxalic Acid I (i.e., standard reference materials (SRM) 4990b) normalized to δ¹³C_{CPDB}=−19 per mil (Olsson, The use of Oxalic acid as a Standard. in, Radiocarbon Variations and Absolute Chronology, Nobel Symposium, 12th Proc., John Wiley & Sons, New York (1970)). Mass spectrometry results, for example, measured by ASM, are calculated using the internationally agreed upon definition of 0.95 times the specific activity of NBS Oxalic Acid I (SRM 4990b) normalized to δ¹³C_{VPDB}=−19 per mil. This is equivalent to an absolute (AD 1950) ¹⁴C/¹²C ratio of 1.176±0.010×10⁻¹² (Karlen et al., Arkiv Geofysik, 4:465-471 (1968)). The standard calculations take into account the differential uptake of one isotope with respect to another, for example, the preferential uptake in biological systems of ¹²C over ¹³C over ¹⁴C, and these corrections are reflected as a Fm corrected for δ¹³.

An oxalic acid standard (SRM 4990b or HOx 1) was made from a crop of 1955 sugar beet. Although there were 1000 lbs made, this oxalic acid standard is no longer commercially available. The Oxalic Acid II standard (HOx 2; N.I.S.T designation SRM 4990 C) was made from a crop of 1977 French beet molasses. In the early 1980's, a group of 12 laboratories measured the ratios of the two standards. The ratio of the activity of Oxalic acid II to 1 is 1.2933±0.001 (the weighted mean). The isotopic ratio of HOx II is −17.8 per mil. ASTM D6866-11 suggests use of the available Oxalic Acid II standard SRM 4990 C (Hox2) for the modern standard (see discussion of original vs. currently available oxalic acid standards in Mann, Radiocarbon, 25(2):519-527 (1983)). A Fm=0% represents the entire lack of carbon-14 atoms in a material, thus indicating a fossil (for example, petroleum based) carbon source. A Fm=100%, after correction for the post-1950 injection of carbon-14 into the atmosphere from nuclear bomb testing, indicates an entirely modern carbon source. As described herein, such a “modern” source includes biobased sources.

As described in ASTM D6866, the percent modern carbon (pMC) can be greater than 100% because of the continuing but diminishing effects of the 1950s nuclear testing programs, which resulted in a considerable enrichment of carbon-14 in the atmosphere as described in ASTM D6866-11. Because all sample carbon-14 activities are referenced to a “pre-bomb” standard, and because nearly all new biobased products are produced in a post-bomb environment, all pMC values (after correction for isotopic fraction) must be multiplied by 0.95 (as of 2010) to better reflect the true biobased content of the sample. A biobased content that is greater than 103% suggests that either an analytical error has occurred, or that the source of biobased carbon is more than several years old.

ASTM D6866 quantifies the biobased content relative to the material's total organic content and does not consider the inorganic carbon and other non-carbon containing substances present. For example, a product that is 50% starch-based material and 50% water would be considered to have a Biobased Content=100% (50% organic content that is 100% biobased) based on ASTM D6866. In another example, a product that is 50% starch-based material, 25% petroleum-based, and 25% water would have a Biobased Content=66.7% (75% organic content but only 50% of the product is biobased). In another example, a product that is 50% organic carbon and is a petroleum-based product would be considered to have a Biobased Content=0% (50% organic carbon but from fossil sources). Thus, based on the well known methods and known standards for determining the biobased content of a compound or material, one skilled in the art can readily determine the biobased content and/or prepared downstream products that utilize provided herein having a desired biobased content.

Applications of carbon-14 dating techniques to quantify bio-based content of materials are known in the art (Currie et al., Nuclear Instruments and Methods in Physics Research B, 172:281-287 (2000)). For example, carbon-14 dating has been used to quantify bio-based content in terephthalate-containing materials (Colonna et al., Green Chemistry, 13:2543-2548 (2011)). Notably, polypropylene terephthalate (PPT) polymers derived from renewable 1,3-propanediol and petroleum-derived terephthalic acid resulted in Fm values near 30% (i.e., since 3/11 of the polymeric carbon derives from renewable 1,3-propanediol and 8/11 from the fossil end member terephthalic acid) (Currie et al., supra, 2000). In contrast, polybutylene terephthalate polymer derived from both renewable 1,4-butanediol and renewable terephthalic acid resulted in bio-based content exceeding 90% (Colonna et al., supra, 2011).

Accordingly, in some embodiments, provided herein are bioderived compounds that have a carbon-12, carbon-13, and carbon-14 ratio that reflects an atmospheric carbon, also referred to as environmental carbon, uptake source. The bioderived compounds include such as xylitol, arabitol, ethanol, n-butanol, isobutanol, isopropanol, ethyl acetate, phenyl-ethyl alcohol, 2-methyl-butanol, or 3-methyl-butanol. For example, in some aspects the bioderived compound can have an Fm value of at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 98% or as much as 100%. In some such embodiments, the uptake source is CO₂. In some embodiments, provided herein are bioderived compounds that have a carbon-12, carbon-13, and carbon-14 ratio that reflects petroleum-based carbon uptake source. In this aspect, the bioderived compounds provided herein can have an Fm value of less than 95%, less than 90%, less than 85%, less than 80%, less than 75%, less than 70%, less than 65%, less than 60%, less than 55%, less than 50%, less than 45%, less than 40%, less than 35%, less than 30%, less than 25%, less than 20%, less than 15%, less than 10%, less than 5%, less than 2% or less than 1%. In some embodiments, bioderived compounds provided herein can have a carbon-12, carbon-13, and carbon-14 ratio that are obtained by a combination of an atmospheric carbon uptake source with a petroleum-based uptake source. Using such a combination of uptake sources is one way by which the carbon-12, carbon-13, and carbon-14 ratio can be varied, and the respective ratios would reflect the proportions of the uptake sources.

Further, provided herein are also the products derived the bioderived compounds including such as xylitol, arabitol, ethanol, n-butanol, isobutanol, isopropanol, ethyl acetate, phenyl-ethyl alcohol, 2-methyl-butanol, or 3-methyl-butanol, wherein the bioderived compounds has a carbon-12, carbon-13, and carbon-14 isotope ratio of about the same value as the CO₂ that occurs in the environment. For example, in some aspects, provided herein are bioderived compounds having a carbon-12 versus carbon-13 versus carbon-14 isotope ratio of about the same value as the CO₂ that occurs in the environment, or any of the other ratios disclosed herein. It is understood, as disclosed herein, that a product can have a carbon-12 versus carbon-13 versus carbon-14 isotope ratio of about the same value as the CO₂ that occurs in the environment, or any of the ratios disclosed herein, wherein the product is generated from bioderived compounds as disclosed herein, wherein the bioderived compound is chemically modified to generate a final product. Methods of chemically modifying a bioderived compound to generate a desired product are well known to those skilled in the art, as described herein.

Provided herein are also biobased products having one or more bioderived compound produced by a Metschnikowia species described herein or produced using a method described herein. In some embodiments, provided herein are biobased products produced using a bioderived compound described herein, such as xylitol, arabitol, ethanol, n-butanol, isobutanol, isopropanol, ethyl acetate, phenyl-ethyl alcohol, 2-methyl-butanol, or 3-methyl-butanol. Such manufacturing can include chemically reacting the bioderived compound (e.g. chemical conversion, chemical functionalization, chemical coupling, oxidation, reduction, polymerization, copolymerization and the like) into the final product. In some embodiments, provided herein are biobased products having a bioderived compound described herein, such as xylitol, arabitol, ethanol, n-butanol, isobutanol, isopropanol, ethyl acetate, phenyl-ethyl alcohol, 2-methyl-butanol, or 3-methyl-butanol. In some embodiments, provided herein are biobased products having at least 2%, at least 3%, at least 5%, at least 10%, at least 15%, at least 20%, at least 25%, at least 30%, at least 35%, at least 40%, at least 50%, at least 60%, at least 70%, at least 80%, at least 90%, at least 95%, at least 98% or 100% bioderived compound as disclosed herein.

Provided herein are isolated polypeptides directed to the proteins of the H0 Metschnikowia sp. and isolated nucleic acids directed to the genes of the H0 Metschnikowia sp., as well as host cells comprising such nucleic acids. The presence of these nucleic acids in a Metschnikowia species can identify the Metschnikowia species as being the H0 Metschnikowia sp. or a variant thereof. Thus, provided herein is an isolated polypeptide that has the amino acid sequence of the proteins Aro10, Gxf2, Hgt19, Hxt5, Tef1, Xks1, Xyl1, Tal1 or Tkl1 or a variant thereof; an isolated nucleic acid that has a nucleic acid sequence that encodes the proteins Aro10, Gxf2, Hgt19, Hxt5, Tef1, Xks1, Xyl1, Tal1 or Tkl1 or a variant thereof an isolated nucleic acid that has the nucleic acid sequence of the gene for ACT1, ARO8, ARO10, GPD1, GXF1, GXF2, GXS1, HGT19, HXT2.6, HXT5, PGK1, QUP2, RPB1, RPB2, TEF1, TPI1, XKS1, XYL1, XYL2, XYT1, TAL1 or TKL1; as well as a host cell having such nucleic acid sequences and/or expressing such proteins.

Exemplary polypeptides of the H0 Metschnikowia sp. include Aro10 (SEQ ID NO: 37), Gxf2 (SEQ ID NO: 40), Hgt19 (SEQ ID NO: 42), Hxt5 (SEQ ID NO: 44), Tef1 (SEQ ID NO: 46), Xks1 (SEQ ID NO: 51), Xyl1 (SEQ ID NO: 52), Tall (SEQ ID NO: 55) and Tkl1 (SEQ ID NO: 56). Accordingly, in some embodiments, provided herein is an isolated polypeptide having the amino acid sequence of SEQ ID NO: 37. In some embodiments, provided herein is an isolated polypeptide having the amino acid sequence of SEQ ID NO: 40. In some embodiments, provided herein is an isolated polypeptide having the amino acid sequence of SEQ ID NO: 42. In some embodiments, provided herein is an isolated polypeptide having the amino acid sequence of SEQ ID NO: 44. In some embodiments, provided herein is an isolated polypeptide having the amino acid sequence of SEQ ID NO: 46. In some embodiments, provided herein is an isolated polypeptide having the amino acid sequence of SEQ ID NO: 51. In some embodiments, provided herein is an isolated polypeptide having the amino acid sequence of SEQ ID NO: 52. In some embodiments, provided herein is an isolated polypeptide having the amino acid sequence of SEQ ID NO: 55. In some embodiments, provided herein is an isolated polypeptide having the amino acid sequence of SEQ ID NO: 56.

Also provided herein are isolated polypeptides having an amino acid sequence that is a variant to a protein of the H0 Metschnikowia sp. described herein, but still retains the functional activity of the polypeptide. For example, in some embodiments, the isolated polypeptide has an amino acid sequence of any one of SEQ ID NOS: 37, 40, 42, 44, 46, 51, 52, 55 and 56, wherein the amino acid sequence includes 1, 2, 3, 4, 5, 6, 7, 8, 9, 10, 11, 12, 13, 14, 15, 16, 17, 18, 19, 20, 21, 22, 23, 24, or 25 amino acid substitutions, deletions or insertions. Variants of a protein provided herein also include, for example, deletions, fusions, or truncations when compared to the reference polypeptide sequence. Accordingly, in some embodiments, the isolated polypeptide provided herein has an amino acid sequence that is at least 95.0%, at least 95.1%, at least 95.2%, at least 95.3%, at least 95.4%, at least 95.5%, at least 95.6%, at least 95.7%, at least 95.8%, at least 95.9%, at least 96.0%, at least 96.1%, at least 96.2%, at least 96.3%, at least 96.4%, at least 96.5%, at least 96.6%, at least 96.7%, at least 96.8%, at least 96.9%, at least 97.0%, at least 97.1%, at least 97.2%, at least 97.3%, at least 97.4%, at least 97.5%, at least 97.6%, at least 97.7%, at least 97.8%, at least 97.9%, at least 98.0%, at least 98.1%, at least 98.2%, at least 98.3%, at least 98.4%, at least 98.5%, at least 98.6%, at least 98.7%, at least 98.8%, at least 98.9%, at least 99.0%, at least 99.1%, at least 99.2%, at least 99.3%, at least 99.4%, at least 99.5%, at least 99.6%, at least 99.7%, or at least 99.8% identical to any one of SEQ ID NOS: 37, 40, 42, 44, 46, 51, 52, 55 and 56.

Variants of the proteins described herein can also contain conservatively amino acids substitution, meaning that one or more amino acid can be replaced by an amino acid that does not alter the secondary and/or tertiary stricture of the protein. Such substitutions can include the replacement of an amino acid, by a residue having similar physicochemical properties, such as substituting one aliphatic residue (Ile, Val, Leu, or Ala) for another, or substitutions between basic residues Lys and Arg, acidic residues Glu and Asp, amide residues Gln and Asn, hydroxyl residues Ser and Tyr, or aromatic residues Phe and Tyr. Phenotypically silent amino acid exchanges are described more fully in Bowie et al., Science 247:1306-10 (1990). In addition, variants of a protein described herein include those having amino acid substitutions, deletions, or additions to the amino acid sequence outside functional regions of the protein so long as the substitution, deletion, or addition does not affect the function of the resulting polypeptide. Techniques for making these substitutions and deletions are well known in the art and include, for example, site-directed mutagenesis.

The isolated polypeptides provided herein also include functional fragments of the proteins described herein, which retain their function. In some embodiments, provided herein is an isolated polypeptide that is a functional fragment of a protein described herein. In some embodiments, provided herein is an isolated nucleic acid that encodes a polypeptide that is a functional fragment of a protein described herein. In some embodiments, the isolated polypeptide can be fragments of protein such as Aro10 (SEQ ID NO: 37), Gxf2 (SEQ ID NO: 40), Hgt19 (SEQ ID NO: 42), Hxt5 (SEQ ID NO: 44), Tef1 (SEQ ID NO: 46), Xks1 (SEQ ID NO: 51), Xyl1 (SEQ ID NO: 52), Tal1 (SEQ ID NO: 55), and Tkl1 (SEQ ID NO: 56), which retains the function of the protein.

In some embodiments, variants of the proteins described herein include covalent modification or aggregative conjugation with other chemical moieties, such as glycosyl groups, polyethylene glycol (PEG) groups, lipids, phosphate, acetyl groups, and the like. In some embodiments, variants of the proteins described herein further include, for example, fusion proteins formed of the protein described herein and another polypeptide. The added polypeptides for constructing the fusion protein include those that facilitate purification or oligomerization of the protein described herein, or those that enhance stability and/or function of the protein described herein.

The proteins described herein can be fused to heterologous polypeptides to facilitate purification. Many available heterologous peptides (peptide tags) allow selective binding of the fusion protein to a binding partner. Non-limiting examples of peptide tags include 6-His, thioredoxin, hemaglutinin, GST, and the OmpA signal sequence tag. A binding partner that recognizes and binds to the heterologous peptide tags can be any molecule or compound, including metal ions (for example, metal affinity columns), antibodies, antibody fragments, or any protein or peptide that selectively or specifically binds the heterologous peptide to permit purification of the fusion protein.

The proteins described herein can also be modified to facilitate formation of oligomers. For example, the protein described herein can be fused to peptide moieties that promote oligomerization, such as leucine zippers and certain antibody fragment polypeptides, such as Fc polypeptides. Techniques for preparing these fusion proteins are known, and are described, for example, in WO 99/31241 and in Cosman et al., Immunity 14:123-133 (2001). Fusion to an Fc polypeptide offers the additional advantage of facilitating purification by affinity chromatography over Protein A or Protein G columns. Fusion to a leucine-zipper (LZ), for example, a repetitive heptad repeat, often with four or five leucine residues interspersed with other amino acids, is described in Landschulz et al., Science 240:1759-64 (1988).

The protein described herein can be provided in an isolated form, or in a substantially purified form. The polypeptides can be recovered and purified from recombinant cell cultures by known methods, including, for example, ammonium sulfate or ethanol precipitation, anion or cation exchange chromatography, phosphocellulose chromatography, hydrophobic interaction chromatography, affinity chromatography, hydroxylapatite chromatography, and lectin chromatography. In some embodiments, protein chromatography is employed for purification.

In some embodiments, provided herein are recombinant Metschnikowia species having an exogenous nucleic acid encoding a protein described herein. In some embodiments, the recombinant Metschnikowia species has an exogenous nucleic acid encoding a protein described herein, wherein the protein has 1 to 25, 1 to 20, 1 to 15, 1 to 10, or 1 to 5, amino acid substitutions, deletions or insertions. In some embodiments, the protein is Aro10 (SEQ ID NO: 37), Gxf2 (SEQ ID NO: 40), Hgt19 (SEQ ID NO: 42), Hxt5 (SEQ ID NO: 44), Tef1 (SEQ ID NO: 46), Xks1 (SEQ ID NO: 51), and Xyl1 (SEQ ID NO: 52) and retains the function of the protein. In some embodiments, the protein has 1 to 10 amino acid substitutions, deletions or insertions of Aro10 (SEQ ID NO: 37), Gxf2 (SEQ ID NO: 40), Hgt19 (SEQ ID NO: 42), Hxt5 (SEQ ID NO: 44), Tef1 (SEQ ID NO: 46), Xks1 (SEQ ID NO: 51), and Xyl1 (SEQ ID NO: 52) and retains the function of the protein. In some embodiments, the protein has 1 to 5 amino acid substitutions, deletions or insertions of Aro10 (SEQ ID NO: 37), Gxf2 (SEQ ID NO: 40), Hgt19 (SEQ ID NO: 42), Hxt5 (SEQ ID NO: 44), Tef1 (SEQ ID NO: 46), Xks1 (SEQ ID NO: 51), and Xyl1 (SEQ ID NO: 52) and retains the function of the protein. The non-naturally occurring microbial organism can be a Metschnikowia species, including, but not limited to, the H0 Metschnikowia sp. described herein.

The proteins described herein can be recombinantly expressed by suitable hosts. When heterologous expression of the protein is desired, the coding sequences of specific genes can be modified in accordance with the codon usage of the host. The standard genetic code is well known in the art, as reviewed in, for example, Osawa et al., Microbiol Rev. 56(1):229-64 (1992). Yeast species, including but not limited to Saccharomyces cerevisiae, Candida azyma, Candida diversa, Candida magnoliae, Candida rugopelliculosa, Yarrowia lipolytica, and Zygoascus hellenicus, use the standard code. Certain yeast species use alternative codes. For example, “CUG,” standard codon for “Leu,” encodes “Ser” in species such as Candida albicans, Candida cylindracea, Candida melibiosica, Candida parapsilosis, Candida rugose, Pichia stipitis, and Metschnikowia species. The codon table for the H0 Metschnikowia sp. is provided herein.

Furthermore, the hosts can simultaneously produce other forms of the same category of proteins such that multiple forms of the same type of protein are expressed in the same cell. For example, the hosts can simultaneously produce different transporters, which can form oligomers to transport the same sugar. Alternatively, the different transporters can function independently to transport different sugars.

Variants of proteins described herein can be generated by conventional methods known in the art, such as by introducing mutations at particular locations by oligonucleotide-directed site-directed mutagenesis. Site-directed-mutagenesis is considered an informational approach to protein engineering and can rely on high-resolution crystallographic structures of target proteins for specific amino acid changes (Van Den Burg et al., PNAS 95:2056-60 (1998)). Computational methods for identifying site-specific changes for a variety of protein engineering objectives are also known in the art (Hellinga, Nature Structural Biology 5:525-27 (1998)).

Other techniques known in the art include, but are not limited to, non-informational mutagenesis techniques (referred to generically as “directed evolution”). Directed evolution, in conjunction with high-throughput screening, allows testing of statistically meaningful variations in protein conformation (Arnold, 1998). Directed evolution technology can include diversification methods similar to that described by Crameri et al., Nature 391:288-91 (1998), site-saturation mutagenesis, staggered extension process (StEP) (Zhao et al., Nature Biotechnology 16:258-61 (1998)), and DNA synthesis/reassembly (U.S. Pat. No. 5,965,408).

As disclosed herein, a nucleic acid encoding a protein described herein can be introduced into a host organism. In some cases, it can also be desirable to modify an activity of protein to increase production of a desired product. For example, known mutations that increase the activity of a protein can be introduced into an encoding nucleic acid molecule. Additionally, optimization methods can be applied to increase the activity of a protein and/or decrease an inhibitory activity, for example, decrease the activity of a negative regulator.

One such optimization method is directed evolution. Directed evolution is a powerful approach that involves the introduction of mutations targeted to a specific gene in order to improve and/or alter the properties of an enzyme. Improved and/or altered enzymes can be identified through the development and implementation of sensitive high-throughput screening assays that allow the automated screening of many enzyme variants (for example, >10⁴). Iterative rounds of mutagenesis and screening typically are performed to afford an enzyme with optimized properties. Computational algorithms that can help to identify areas of the gene for mutagenesis also have been developed and can significantly reduce the number of enzyme variants that need to be generated and screened. Numerous directed evolution technologies have been developed (for reviews, see Hibbert et al., Biomol. Eng 22:11-19 (2005); Huisman and Lalonde, In Biocatalysis in the pharmaceutical and biotec hnology industries pgs. 717-742 (2007), Patel (ed.), CRC Press; Often and Quax. Biomol. Eng 22:1-9 (2005).; and Sen et al., Appl Biochem. Biotechnol 143:212-223 (2007)) to be effective at creating diverse variant libraries, and these methods have been successfully applied to the improvement of a wide range of properties across many enzyme classes. Enzyme characteristics that have been improved and/or altered by directed evolution technologies include, for example: selectivity/specificity, for conversion of non-natural substrates; temperature stability, for robust high temperature processing; pH stability, for bioprocessing under lower or higher pH conditions; substrate or product tolerance, so that high product titers can be achieved; binding (Km), including broadening substrate binding to include non-natural substrates; inhibition (Kᵢ), to remove inhibition by products, substrates, or key intermediates; activity (kcat), to increases enzymatic reaction rates to achieve desired flux; expression levels, to increase protein yields and overall pathway flux; oxygen stability, for operation of air sensitive enzymes under aerobic conditions; and anaerobic activity, for operation of an aerobic enzyme in the absence of oxygen.

A number of exemplary methods have been developed for the mutagenesis and diversification of genes to target desired properties of specific enzymes. Such methods are well known to those skilled in the art. Any of these can be used to alter and/or optimize the activity of a protein described herein. Such methods include, but are not limited to EpPCR, which introduces random point mutations by reducing the fidelity of DNA polymerase in PCR reactions (Pritchard et al., J Theor. Biol. 234:497-509 (2005)); Error-prone Rolling Circle Amplification (epRCA), which is similar to epPCR except a whole circular plasmid is used as the template and random 6-mers with exonuclease resistant thiophosphate linkages on the last 2 nucleotides are used to amplify the plasmid followed by transformation into cells in which the plasmid is re-circularized at tandem repeats (Fujii et al., Nucleic Acids Res. 32:e145 (2004); and Fujii et al., Nat. Protoc. 1:2493-2497 (2006)); DNA or Family Shuffling, which typically involves digestion of two or more variant genes with nucleases such as Dnase I or EndoV to generate a pool of random fragments that are reassembled by cycles of annealing and extension in the presence of DNA polymerase to create a library of chimeric genes (Stemmer, Proc Natl Acad Sci USA 91:10747-10751 (1994); and Stemmer, Nature 370:389-391 (1994)); Staggered Extension (StEP), which entails template priming followed by repeated cycles of 2 step PCR with denaturation and very short duration of annealing/extension (as short as 5 sec) (Zhao et al., Nat. Biotechnol. 16:258-261 (1998)); Random Priming Recombination (RPR), in which random sequence primers are used to generate many short DNA fragments complementary to different segments of the template (Shao et al., Nucleic Acids Res 26:681-683 (1998)).

Additional methods include Heteroduplex Recombination, in which linearized plasmid DNA is used to form heteroduplexes that are repaired by mismatch repair (Volkov et al, Nucleic Acids Res. 27:e18 (1999); and Volkov et al., Methods Enzymol. 328:456-463 (2000)); Random Chimeragenesis on Transient Templates (RACHITT), which employs Dnase I fragmentation and size fractionation of single stranded DNA (ssDNA) (Coco et al., Nat. Biotechnol. 19:354-359 (2001)); Recombined Extension on Truncated templates (RETT), which entails template switching of unidirectionally growing strands from primers in the presence of unidirectional ssDNA fragments used as a pool of templates (Lee et al., J. Molec. Catalysis 26:119-129 (2003)); Degenerate Oligonucleotide Gene Shuffling (DOGS), in which degenerate primers are used to control recombination between molecules; (Bergquist and Gibbs, Methods Mol. Biol 352:191-204 (2007); Bergquist et al., Biomol. Eng 22:63-72 (2005); Gibbs et al., Gene 271:13-20 (2001)); Incremental Truncation for the Creation of Hybrid Enzymes (ITCHY), which creates a combinatorial library with 1 base pair deletions of a gene or gene fragment of interest (Ostermeier et al., Proc. Natl. Acad. Sci. USA 96:3562-3567 (1999); and Ostermeier et al., Nat. Biotechnol. 17:1205-1209 (1999)); Thio-Incremental Truncation for the Creation of Hybrid Enzymes (THIO-ITCHY), which is similar to ITCHY except that phosphothioate dNTPs are used to generate truncations (Lutz et al., Nucleic Acids Res 29:E16 (2001)); SCRATCHY, which combines two methods for recombining genes, ITCHY and DNA shuffling (Lutz et al., Proc. Natl. Acad. Sci. USA 98:11248-11253 (2001)); Random Drift Mutagenesis (RNDM), in which mutations made via epPCR are followed by screening/selection for those retaining usable activity (Bergquist et al., Biomol. Eng. 22:63-72 (2005)); Sequence Saturation Mutagenesis (SeSaM), a random mutagenesis method that generates a pool of random length fragments using random incorporation of a phosphothioate nucleotide and cleavage, which is used as a template to extend in the presence of “universal” bases such as inosine, and replication of an inosine-containing complement gives random base incorporation and, consequently, mutagenesis (Wong et al., Biotechnol. 1 3:74-82 (2008); Wong et al., Nucleic Acids Res. 32:e26 (2004); and Wong et al., Anal. Biochem. 341:187-189 (2005)); Synthetic Shuffling, which uses overlapping oligonucleotides designed to encode “all genetic diversity in targets” and allows a very high diversity for the shuffled progeny (Ness et al., Nat. Biotechnol. 20:1251-1255 (2002)); Nucleotide Exchange and Excision Technology NexT, which exploits a combination of dUTP incorporation followed by treatment with uracil DNA glycosylase and then piperidine to perform endpoint DNA fragmentation (Muller et al., Nucleic Acids Res. 33:e117 (2005)).

Further methods include Sequence Homology-Independent Protein Recombination (SHIPREC), in which a linker is used to facilitate fusion between two distantly related or unrelated genes, and a range of chimeras is generated between the two genes, resulting in libraries of single-crossover hybrids (Sieber et al., Nat. Biotechnol. 19:456-460 (2001)); Gene Site Saturation Mutagenesis™ (GSSM™), in which the starting materials include a supercoiled double stranded DNA (dsDNA) plasmid containing an insert and two primers which are degenerate at the desired site of mutations (Kretz et al., Methods Enzymol. 388:3-11 (2004)); Combinatorial Cassette Mutagenesis (CCM), which involves the use of short oligonucleotide cassettes to replace limited regions with a large number of possible amino acid sequence alterations (Reidhaar-Olson et al. Methods Enzymol. 208:564-586 (1991); and Reidhaar-Olson et al. Science 241:53-57 (1988)); Combinatorial Multiple Cassette Mutagenesis (CMCM), which is essentially similar to CCM and uses epPCR at high mutation rate to identify hot spots and hot regions and then extension by CMCM to cover a defined region of protein sequence space (Reetz et al., Angew. Chem. Int. Ed Engl. 40:3589-3591 (2001)); the Mutator Strains technique, in which conditional is mutator plasmids, utilizing the mutD5 gene, which encodes a mutant subunit of DNA polymerase III, to allow increases of 20 to 4000-X in random and natural mutation frequency during selection and block accumulation of deleterious mutations when selection is not required (Selifonova et al., Appl. Environ. Microbiol. 67:3645-3649 (2001)); Low et al., J. Mol. Biol. 260:359-3680 (1996)).

Additional exemplary methods include Look-Through Mutagenesis (LTM), which is a multidimensional mutagenesis method that assesses and optimizes combinatorial mutations of selected amino acids (Rajpal et al., Proc. Natl. Acad. Sci. USA 102:8466-8471 (2005)); Gene Reassembly, which is a DNA shuffling method that can be applied to multiple genes at one time or to create a large library of chimeras (multiple mutations) of a single gene (Tunable GeneReassembly™ (TGR™) Technology supplied by Verenium Corporation), in Silico Protein Design Automation (PDA), which is an optimization algorithm that anchors the structurally defined protein backbone possessing a particular fold, and searches sequence space for amino acid substitutions that can stabilize the fold and overall protein energetics, and generally works most effectively on proteins with known three-dimensional structures (Hayes et al., Proc. Natl. Acad. Sci. USA 99:15926-15931 (2002)); and Iterative Saturation Mutagenesis (ISM), which involves using knowledge of structure/function to choose a likely site for enzyme improvement, performing saturation mutagenesis at chosen site using a mutagenesis method such as Stratagene QuikChange (Stratagene; San Diego Calif.), screening/selecting for desired properties, and, using improved clone(s), starting over at another site and continue repeating until a desired activity is achieved (Reetz et al., Nat. Protoc. 2:891-903 (2007); and Reetz et al., Angew. Chem. Int. Ed Engl. 45:7745-7751 (2006)).

Any of the aforementioned methods for mutagenesis can be used alone or in any combination. Additionally, any one or combination of the directed evolution methods can be used in conjunction with adaptive evolution techniques, as described herein or otherwise known in the art.

Provided herein are isolated nucleic acids having nucleic acid sequences encoding the proteins described herein as well as the specific encoding nucleic acid sequences of the genes described herein. Nucleic acids provided herein include those having the nucleic acid sequence provided in the sequence listing; those that hybridize to the nucleic acid sequences provided in the sequence listing, under high stringency hybridization conditions (for example, 42°, 2.5 hr., 6×SCC, 0.1% SDS); and those having substantial nucleic acid sequence identity with the nucleic acid sequence provided in the sequence listing. The nucleic acids provided herein also encompass equivalent substitutions of codons that can be translated to produce the same amino acid sequences. Provided herein are also vectors including the nucleic acids described herein. The vector can be an expression vector suitable for expression in a host microbial organism. The vector can be a viral vector.

The nucleic acids provided herein include those encoding proteins having an amino acid sequence as described herein, as well as their variants that retain their function. The nucleic acids provided herein can be cDNA, chemically synthesized DNA, DNA amplified by PCR, RNA, or combinations thereof. Due to the degeneracy of the genetic code, two DNA sequences can differ and yet encode identical amino acid sequences.

Provided herein are also useful fragments of nucleic acids encoding the proteins described herein, include probes and primers. Such probes and primers can be used, for example, in PCR methods to amplify or detect the presence of nucleic acids encoding the proteins described herein in vitro, as well as in Southern and Northern blots for analysis. Cells expressing the proteins described herein can also be identified by the use of such probes. Methods for the production and use of such primers and probes are well known.

Provided herein are also fragments of nucleic acids encoding the proteins described herein that are antisense or sense oligonucleotides having a single-stranded nucleic acid capable of binding to a target mRNA or DNA sequence of the protein or nucleic acid sequence described herein.

A nucleic acid encoding a protein described herein can include nucleic acids that hybridize to a nucleic acid disclosed herein by SEQ ID NO or a nucleic acid molecule that hybridizes to a nucleic acid molecule that encodes an amino acid sequence disclosed herein by SEQ ID NO. Hybridization conditions can include highly stringent, moderately stringent, or low stringency hybridization conditions that are well known to one of skill in the art such as those described herein.

Stringent hybridization refers to conditions under which hybridized polynucleotides are stable. As known to those of skill in the art, the stability of hybridized polynucleotides is reflected in the melting temperature (Tm) of the hybrids. In general, the stability of hybridized polynucleotides is a function of the salt concentration, for example, the sodium ion concentration and temperature. A hybridization reaction can be performed under conditions of lower stringency, followed by washes of varying, but higher, stringency. Reference to hybridization stringency relates to such washing conditions. Highly stringent hybridization includes conditions that permit hybridization of only those nucleic acid sequences that form stable hybridized polynucleotides in 0.018M NaCl at 65° C., for example, if a hybrid is not stable in 0.018M NaCl at 65° C., it will not be stable under high stringency conditions, as contemplated herein. High stringency conditions can be provided, for example, by hybridization in 50% formamide, 5×Denhart's solution, 5×SSPE, 0.2% SDS at 42° C., followed by washing in 0.1×SSPE, and 0.1% SDS at 65° C. Hybridization conditions other than highly stringent hybridization conditions can also be used to describe the nucleic acid sequences disclosed herein. For example, the phrase moderately stringent hybridization refers to conditions equivalent to hybridization in 50% formamide, 5×Denhart's solution, 5×SSPE, 0.2% SDS at 42° C., followed by washing in 0.2×SSPE, 0.2% SDS, at 42° C. The phrase low stringency hybridization refers to conditions equivalent to hybridization in 10% formamide, 5×Denhart's solution, 6×SSPE, 0.2% SDS at 22° C., followed by washing in 1×SSPE, 0.2% SDS, at 37° C. Denhart's solution contains 1% Ficoll, 1% polyvinylpyrolidone, and 1% bovine serum albumin (BSA). 20×SSPE (sodium chloride, sodium phosphate, ethylene diamide tetraacetic acid (EDTA)) contains 3M sodium chloride, 0.2M sodium phosphate, and 0.025 M (EDTA). Other suitable low, moderate and high stringency hybridization buffers and conditions are well known to those of skill in the art and are described, for example, in Sambrook et al., Molecular Cloning: A Laboratory Manual, Third Ed., Cold Spring Harbor Laboratory, New York (2001); and Ausubel et al., Current Protocols in Molecular Biology, John Wiley and Sons, Baltimore, Md. (1999).

Nucleic acids encoding a protein provided herein include those having a certain percent sequence identity to a nucleic acid sequence disclosed herein by SEQ ID NO. For example, a nucleic acid molecule can have at least 95.0%, at least 95.1%, at least 95.2%, at least 95.3%, at least 95.4%, at least 95.5%, at least 95.6%, at least 95.7%, at least 95.8%, at least 95.9%, at least 96.0%, at least 96.1%, at least 96.2%, at least 96.3%, at least 96.4%, at least 96.5%, at least 96.6%, at least 96.7%, at least 96.8%, at least 96.9%, at least 97.0%, at least 97.1%, at least 97.2%, at least 97.3%, at least 97.4%, at least 97.5%, at least 97.6%, at least 97.7%, at least 97.8%, at least 97.9%, at least 98.0%, at least 98.1%, at least 98.2%, at least 98.3%, at least 98.4%, at least 98.5%, at least 98.6%, at least 98.7%, at least 98.8%, at least 98.9%, at least 99.0%, at least 99.1%, at least 99.2%, at least 99.3%, at least 99.4%, at least 99.5%, at least 99.6%, at least 99.7%, or at least 99.8% sequence identity, or be identical, to a sequence selected from SEQ ID NOS: 57-78.

Accordingly, in some embodiments, the isolated nucleic acid provided herein has a nucleic acid sequence of the genes of the H0 Metschnikowia sp. disclosed herein, including ACT1 (SEQ ID NO: 57), ARO8 (SEQ ID NO: 58), ARO10 (SEQ ID NO: 59), GPD1 (SEQ ID NO: 60), GXF1 (SEQ ID NO: 61), GXF2 (SEQ ID NO: 62), GXS1 (SEQ ID NO: 63), HXT19 (SEQ ID NO: 64), HXT2.6 (SEQ ID NO: 65), HXT5 (SEQ ID NO: 66), PGK1 (SEQ ID NO: 67), QUP2 (SEQ ID NO: 68), RPB1 (SEQ ID NO: 69), RPB2 (SEQ ID NO: 70), TEF1 (SEQ ID NO: 71), TPI1 (SEQ ID NO: 72), XKS1 (SEQ ID NO: 73), XYL1 (SEQ ID NO: 74), XYL2 (SEQ ID NO: 75), XYT1 (SEQ ID NO: 76), TAL1 (SEQ ID NO: 77), or TKL1 (SEQ ID NO: 78). Accordingly, in some embodiments, provided herein is an isolated nucleic acid having a nucleic acid sequence of ACT1 (SEQ ID NO: 57). In some embodiments, provided herein is an isolated nucleic acid having a nucleic acid sequence of ARO8 (SEQ ID NO: 58). In some embodiments, provided herein is an isolated nucleic acid having a nucleic acid sequence of ARO10 (SEQ ID NO: 59). In some embodiments, provided herein is an isolated nucleic acid having a nucleic acid sequence of GPD1 (SEQ ID NO: 60). In some embodiments, provided herein is an isolated nucleic acid having a nucleic acid sequence of GXF1 (SEQ ID NO: 61). In some embodiments, provided herein is an isolated nucleic acid having a nucleic acid sequence of GXF2 (SEQ ID NO: 62). In some embodiments, provided herein is an isolated nucleic acid having a nucleic acid sequence of GXS1 (SEQ ID NO: 63). In some embodiments, provided herein is an isolated nucleic acid having a nucleic acid sequence of HXT19 (SEQ ID NO: 64). In some embodiments, provided herein is an isolated nucleic acid having a nucleic acid sequence of HXT2.6 (SEQ ID NO: 65). In some embodiments, provided herein is an isolated nucleic acid having a nucleic acid sequence of HXT5 (SEQ ID NO: 66). In some embodiments, provided herein is an isolated nucleic acid having a nucleic acid sequence of PGK1 (SEQ ID NO: 67). In some embodiments, provided herein is an isolated nucleic acid having a nucleic acid sequence of QUP2 (SEQ ID NO: 68). In some embodiments, provided herein is an isolated nucleic acid having a nucleic acid sequence of RPB1 (SEQ ID NO: 69). In some embodiments, provided herein is an isolated nucleic acid having a nucleic acid sequence of RPB2 (SEQ ID NO: 70). In some embodiments, provided herein is an isolated nucleic acid having a nucleic acid sequence of TEF1 (SEQ ID NO: 71). In some embodiments, provided herein is an isolated nucleic acid having a nucleic acid sequence of TPI1 (SEQ ID NO: 72). In some embodiments, provided herein is an isolated nucleic acid having a nucleic acid sequence of XKS1 (SEQ ID NO: 73). In some embodiments, provided herein is an isolated nucleic acid having a nucleic acid sequence of XYL1 (SEQ ID NO: 74). In some embodiments, provided herein is an isolated nucleic acid having a nucleic acid sequence of XYL2 (SEQ ID NO: 75). In some embodiments, provided herein is an isolated nucleic acid having a nucleic acid sequence of or XYT1 (SEQ ID NO: 76). In some embodiments, provided herein is an isolated nucleic acid having a nucleic acid sequence of or TALI (SEQ ID NO: 77). In some embodiments, provided herein is an isolated nucleic acid having a nucleic acid sequence of or TKL1 (SEQ ID NO: 78).

It is understood that modifications which do not substantially affect the activity of the various embodiments of this invention are also provided within the definition of the invention provided herein. Accordingly, the following examples are intended to illustrate but not limit the present invention. Throughout this application various publications have been referenced. The disclosures of these publications in their entireties, including GenBank and GI number publications, are hereby incorporated by reference in this application in order to more fully describe the state of the art to which this invention pertains.

### EXAMPLE I

### Identification of H0 Metschnikowia sp.

This example demonstrates that the H0 Metschnikowia sp. belongs to the genus of Metschnikowia and has D1/D2 and ITS sequences that most closely relates to the Metschnikowia pulcherrima clade, but it has such high variability within its D1/D2 region that the generally applicable 1% threshold for species identification cannot be used. However, the high variability is mainly confined to two particular regions and a conserved D1/D2 region has been identified. Phylogenetic analysis using the RPB2 gene sequence shows that the H0 Metschnikowia sp. is a new species that is dusted with Metschnikowia zizyphicola as a sub group, as compared to other members of the Metschnikowia pulcherrima clade. Morphological and physiological characteristics, in particular the growth profile of H0 Metschnikowia sp. in medium having xylose, confirms that H0 Metschnikowia sp. is a new species that is closely related to Metschnikowia zizyphicola.

### D1/D2 Domain and ITS Sequence Analysis

Sequence analysis of the domains 1 and 2 (D1/D2 domain) of the large subunit (LSU) rRNA gene and internal transcribed spacer (ITS), which is located between the small subunit (SSU) and LSU rRNA genes, is a generally accepted tool for yeast species identification (Kurtzman and Robnett, 1998, Antonie Van Leeuwenkoek, 73:331-371). Previous studies of ascomycetous yeasts have demonstrated that strains with more than 1% substitution in the D1/D2 domain usually represent separate species (Kurtzman & Robnett, 1998). Exceptions have been found in Clavispora lusitaniae (Lachance et al., 2003, FEMS Yeast Res. 4:253-258), Metschnikowia andauensis and Metschnikowia fructicola (Sipiczki et al., 2013, PLoS One, 8:e67384), in which some strains show greater than 1% divergence or heterogeneity in the D1/D2 domain.

The D1/D2 domain of the H0 Metschnikowia sp. was amplified from its genomic DNA using primers NL1 (5′-GCATATCAATAAGCGGAGGAAAAG-3′; SEQ ID NO: 26) and NL4 (5′-GGTCCGTGTTTCAAGACGG -3′; SEQ ID NO: 27). The following exemplary 499 base sequence of D1/D2 domain (starting from immediately after primer NL1 and ending before primer NL4) was identified for H0 Metschnikowia sp.:

(SEQ ID NO: 1)

AAACCAACAGGGATTGCCTCAGTAACGGCGAGTGAAGCGGCAAAAGCTCA
AATTTGAAATCCCCCGGGAATTGTAATTTGAAGAGATTTGGGTCCGGCCG
GCAGGGGTTAAGTCCACTGGAAAGTGGCGCCACAGAGGGTGACAGCCCCG
TGAACCCCTTCAACGCCTTCATCCCAGATCTCCAAGAGTCGAGTTGTTTG
GGAATGCAGCTCTAAGTGGGTGGTAAATTCCATCTAAAGCTAAATACCGG
CGAGAGACCGATAGCGAACAAGTACAGTGATGGAAAGATGAAAAGCACTT
TGAAAAGAGAGTGAAAAAGTACGTGAAATTGTTGAAAGGGAAGGGCTTGC
AAGCAGACACTTAACTGGGCCAGCATCGGGGCGGCGGGAAACAAAACCAC
CGGGGAATGTACCTTTCGAGGATTATAACCCCGGTCCTTATTTCCTCGCC
ACCCCGAGGCCTGCAATCTAAGGATGCTGGCGTAATGGTTGCAAGTCGC

This exemplary D1/D2 sequence was a pool of multiple types of D1/D2 domains—a type of consensus sequence covering all types in a cell.

The above sequence was compared against the NCBI Nucleotide collection (nr/nt) database using the Nucleotide Basic Local Alignment Search Tool (BLASTN). A taxonomy report from the BLASTN search was generated (Table 1). The taxonomy report showed that among the total 105 hits, 104 hits are from the genus Metschnikowia with most species belonging to the Metschnikowia pulcherrima clade, including Metschnikowia pulcherrima, Metschnikowia fructicola, Metschnikowia andauensis, Metschnikowia chrysoperlae, Metschnikowia sinensis, Metschnikowia shanxiensis and Metschnikowia zizyphicola.

TABLE 1

| | Number of | Number of | |
|---|---|---|---|
| Taxonomy | hits | Organisms | Description |
| Saccharomycetes | 105 | 35 | |
| Metschnikowia | 104 | 34 | |
| Metschnikowia sp. | 45 | 1 | Metschnikowia sp. hits |
| Metschnikowia sp. 4 MS-2013 | 1 | 1 | Metschnikowia sp. 4 MS-2013 hits |
| Metschnikowia sp. 3 MS-2013 | 1 | 1 | Metschnikowia sp. 3 MS-2013 hits |
| Metschnikowia sp. 1 MS-2013 | 1 | 1 | Metschnikowia sp. 1 MS-2013 hits |
| Metschnikowia sp. 9 MS-2013 | 1 | 1 | Metschnikowia sp. 9 MS-2013 hits |
| Metschnikowia sp. 2 MS-2013 | 1 | 1 | Metschnikowia sp. 2 MS-2013 hits |
| Metschnikowia pulcherrima | 7 | 1 | Metschnikowia pulcherrima hits |
| Metschnikowia sp. MS-2013 | 6 | 1 | Metschnikowia sp. MS-2013 hits |
| Metschnikowia sp. 6 MS-2013 | 1 | 1 | Metschnikowia sp. 6 MS-2013 hits |
| Metschnikowia sp. 11-1090 | 5 | 1 | Metschnikowia sp. 11-1090 hits |
| Metschnikowia sp. 11-1088 | 9 | 1 | Metschnikowia sp. 11-1088 hits |
| Metschnikowia aff. fructicola HA 1634 | 1 | 1 | Metschnikowia aff. fructicola HA 1634 |
| | | | hits |
| Metschnikowia aff. fructicola HA 1656 | 1 | 1 | Metschnikowia aff. fructicola HA 1656 |
| | | | hits |
| Metschnikowia aff. fructicola HA 1648 | 1 | 1 | Metschnikowia aff. fructicola HA 1648 |
| | | | hits |
| Metschnikowia aff. fructicola HA 1651 | 1 | 1 | Metschnikowia aff. fructicola HA 1651 |
| | | | hits |
| Metschnikowia andauensis | 2 | 1 | Metschnikowia andauensis hits |
| Metschnikowia aff. fructicola BBS1-19a | 1 | 1 | Metschnikowia aff. fructicola BBS1-19a |
| | | | hits |
| Metschnikowia aff. chrysoperlae NRRL | 2 | 1 | Metschnikowia aff. chrysoperlae NRRL |
| Y-6259 | | | Y-6259 hits |
| Metschnikowia aff. chrysoperlae | 1 | 1 | Metschnikowia aff. chrysoperlae |
| P34A005 | | | P34A005 hits |
| Metschnikowia chrysoperlae | 1 | 1 | Metschnikowia chrysoperlae hits |
| Metschnikowia aff. fructicola KKS | 1 | 1 | Metschnikowia aff. fructicola KKS hits |
| Metschnikowia aff. fructicola D3896 | 1 | 1 | Metschnikowia aff. fructicola D3896 hits |
| Metschnikowia aff. fructicola D3895 | 1 | 1 | Metschnikowia aff. fructicola D3895 hits |
| Metschnikowia sp. YS W1 | 1 | 1 | Metschnikowia sp. YS W1 hits |
| Metschnikowia sp. 4.3.38 | 1 | 1 | Metschnikowia sp. 4.3.38 hits |
| Metschnikowia sp. NRRL Y-6148 | 1 | 1 | Metschnikowia sp. NRRL Y-6148 hits |
| Metschnikowia aff. chrysoperlae | 1 | 1 | Metschnikowia aff. chrysoperlae |
| P34A004 | | | P34A004 hits |
| Metschnikowia aff. fructicola HA 1652 | 1 | 1 | Metschnikowia aff. fructicola HA 1652 |
| | | | hits |
| Metschnikowia aff. fructicola HA 1627 | 1 | 1 | Metschnikowia aff. fructicola HA 1627 |
| | | | hits |
| Metschnikowia aff. fructicola HA 1647 | 1 | 1 | Metschnikowia aff. fructicola HA 1647 |
| | | | hits |
| Metschnikowia sp. 11-1089 | 2 | 1 | Metschnikowia sp. 11-1089 hits |
| Metschnikowia sp. 5 MS-2013 | 1 | 1 | Metschnikowia sp. 5 MS-2013 hits |
| Metschnikowia aff. chrysoperlae HA 1623 | 1 | 1 | Metschnikowia aff. chrysoperlae HA |
| | | | 1623 hits |
| Metschnikowia aff. chrysoperlae | 1 | 1 | Metschnikowia aff. chrysoperlae |
| P44A006 | | | P44A006 hits |

The above identified D1/D2 domain of the H0 Metschnikowia sp. (SEQ ID NO: 1) was further compared to the D1/D2 domain of specific species within the Metschnikowia pulcherrima clade (Table 2). Numerous differences were identified. For example, the number of nucleotide variations in the D1/D2 domain sequence between the H0 Metschnikowia sp. and the Metschnikowia pulcherrima clade species of Metschnikowia pulcherrima, Metschnikowia fructicola, Metschnikowia andauensis, Metschnikowia chrysoperlae, Metschnikowia sinensis, Metschnikowia shanxiensis and Metschnikowia zizyphicola were 11 (2.2%), 14 (2.8%), 11 (2.2%), 11 (2.2%), 11 (2.2%), 11 (2.2%) and 12 (2.4%), respectively.

TABLE 2

| | Strain | 26s rDNA |
|---|---|---|
| Taxon | designation | accession no. |
| M. andauensis | CBS 10809 | AJ745110 |
| M. chrysoperlae | CBS 9803 | AY452047 |
| M. fructicola | CBS 8853 | AF360542 |
| M. pulcherrima | CBS 5833 | U45736 |
| M. shanxiensis | CBS 10359 | DQ367883 |
| M. sinensis | CBS 10357 | DQ367881 |
| M. zizyphicola | CBS 10358 | DQ367882 |

Analysis of the D1/D2 domain and ITS sequence was also conducted by the CBS-KNAW Fungal Biodiversity Centre. The H0 Metschnikowia sp. was cultivated on the medium Malt Extact Agar (MEA, OXOID). DNA was extracted after an incubation period of 3-4 days in the dark at 25° C. using the MoBio—UltraClean Microbial DNA Isolation Kit. Fragments containing the D1/D2 domain were amplified using the primers LROR (5′-ACCCGCTGAACTTAAGC-3′; SEQ ID NO: 28) and LR5 (5′-TCCTGAGGGAAACTTCG-3′; SEQ ID NO: 29) (Vilgalys and Hester, 1990, J. Bacteriol., 172(8):4238-4246). Fragments containing the Internal Transcribed Spacer 1 and 2 and the 5.8S gene (ITS) was amplified using the primers LS266 (5′-GCATTCCCAAACAACTCGACTC-3′; SEQ ID NO: 30) and V9G (5′-TTACGTCCCTGCCCTTTGTA-3′; SEQ ID NO: 31) (Gerrits van den Ende & de Hoog 1999)). The PCR fragments were sequenced with the ABI Prism Big Dye™ Terminator v. 3.0 Ready Reaction Cycle sequencing Kit. Samples were analyzed on an ABI PRISM 3700 Genetic Analyzer and contigs were assembled using the forward and reverse sequences with the programme SeqMan from the LaserGene package. The following D1/D2 and ITS sequences were identified:

D1/D2 domain sequence:

(SEQ ID NO: 32)

GATTGCCTCAGTAACGGCGAGTGAAGCGGCAAAAGCTCAAATTTGAAATC
CCCCGGGAATTGTAATTTGAAGAGATTTGGGTCCGGCCGGCGGGGGTTAA
GTCCACTGGAAAGTGGCGCCACAGAGGGTGACAGCCCCGTGAACCCCTTT
AAAGCCTTCATCCCAGATCTCCAAGAGTCGAGTTGTTTGGGAATGCAGCT
CTAAGTGGGTGGTAAATTCCATCTAAAGCTAAATACCGGCGAGAGACCGA
TAGCGAACAAGTACAGTGATGGAAAGATGAAAAGCACTTTGAAAAGAGAG
TGAAAAAGTACGTGAAATTGTTGAAAGGGAAGGGCTTGCAAGCAGACACT
TAACTGGGCCAGCATCGGGGCGGCGGGAAACAAAACCACCGGGGAATGTA
CCTTTCGAGGATTATAACCCCGGTCTCTATTTCCATGCTGCCCCGAGGCC
TGCAATCTAAGGATGCTGGCGTAATGGTTGCAAGTCGCCCGTCTTGAAAC
ACGGACCAAGGAGTCTAACAATCATGCAAGTGTTTGGGCCCAAAACCCAT
ACGCGCAATGAAAGTAACCGGAGCGAACCTTCTGGTGCAGCTCCAGCCAC
ACCGAGACCCAAATCCCGGTGTGAGCAAGCATGGCTGTTGGGACCCGAAA
GATGGTGAACTATACCTGGATAGGGTGAAGCCAGAGGAAACTCTGGTGGA
GGCTCGTAGCGGTTCTGACGTGCAAATCGATCGTCGAATCTGGGTATAGG
GGCGAAAGAC
ITS sequence:

(SEQ ID NO: 33)

CTTAGTGAGGCCTCTGGATTGAATCTAGGGCCGGGGCGACCCGGCCGTGG
GTTGAGAAACTGGTCAAACTTGGTCATTTAGAGGAAGTAAAAGTCGTAAC
AAGGTTTCCGTAGGTGAACCTGCGGAAGGATCATTAAAAATATTATTACA
CACTTTTAGGAAAAACCTCTGAACCTTTTTTTTCATATACACTTTTAAAA
AACTTTCAACAACGGATCTCTTGGTTCTCGCATCGATGAAGAACGCAGCG
AATTGCGATACGTAATATGACTTGCAGACGTGAATCATTGAATCTTTGAA
CGCACATTGCGCCCCGGGGTATTCCCCAGGGCATGCGTGGGTGAGCGATA
TTTACTCTCAAACCTCCGGTTTGGTCCTGCTTCGGCCTAATATCAACGGC
GCTAGAATAAGTTTTAGCCCCATTCTTTTTCCTCACCCTCGTAAGACTAC
CCGCTGAACTTAAGCATATCAATAAGCGGAGGAAAAGAAACCAACAGGGA
TTGCCTCAGTAACGGCGAGTGAAGCGGCAAAAGCTCAAATTTGAAATCCC
CCGGGAATTGTAATTTGAAGAGATTTGGGTCCGGCCGGCGGGGGTTAAGT
CCACTGGAAAGTGGCGCCACAGAGGGTGACAGCCCCGTGA

These sequences were compared against the NCBI Nucleotide collection (nr/nt) database using the Nucleotide Basic Local Alignment Search Tool (BLASTN) and in a large fungal database of the CBS-KNAW Fungal Biodiversity Centre with sequences of most of the type strains. This comparison showed that the H0 Metschnikowia sp. is a new species within the genus Metschnikowia. The closest known species within this genus was identified as being Metschnikowia andauensis, which had 97% sequence identity for the D1/D2 sequence. Additionally, Metschnikowia pulcherrima was shown to have a 98% sequence identity for the D1/D2 sequence, but only a 94% sequence identity for the ITS sequence, and Metschnikowia shanxiensis was shown to have only a 96% sequence identity for the D1/D2 sequence and a 98% sequence identity for a short fragment of the ITS sequence.

However, as indicated above, the D1/D2 domain of the type strains of Metschnikowia andauensis and Metschnikowia fructicola were reported as being non-homogenous. For example, it has been reported that up to 18 (3.6%) substitutions within M andauensis clones and up to 25 (5%) substitutions within M. fructicola clones can be found (Sipiczki et al., 2013, PLoS One, 8:e67384). Thus, in order to see if the D1/D2 domain of the H0 Metschnikowia sp. is homogenous, DNA was extracted from 6 colonies streaked from the original H0 Metschnikowia sp. permanent stock, amplified by PCR using the primers ITS1 (5′-TCCGTAGGTGAACCTGCGG-3′; SEQ ID NO: 34) and NL4 (5′-GGTCCGTGTTTCAAGACGG-3′; SEQ ID NO: 27), which are flanked by 20 nt sequence identical to the plasmid pUC19 for assembly cloning. The PCR products were gel purified and cloned into the SacI and HindIII sites of pUC19. The cloned plasmids were sequenced from both ends and the sequences were analyzed using Geneious 7.1.9.

In the 32 total D1/D2 domain sequences cloned and analyzed, there are 23 types (Table 3) with variations of up to 23 bases (4.6%) exceeding the difference between H0 Metschnikowia sp. and the type strains of M. pulcherrima clade.

TABLE 3

| | | | Number of |
|---|---|---|---|
| | | | nucleotide |
| | | | substitutions vs |
| Type | Clone | Sequence | H1-7 |
| 1 | H01-1, | AAACCAACAGGGATTGCCTCAGTAACGGCGAGTGAAGCGGCAAAAG | 3 |
| | H02-2 | CTCAAATTTGAAATCCCCCGGGAATTGTAATTTGAAGAGATTTGGGT | |
| | | CCGGCCGGCGGGGGTTAAGTCCACTGGAAAGTGGCGCCACAGAGGG | |
| | | TGACAGCCCCGTGAGCCCCTCTAACGCCTCTACCCCAAATCTCCAAG | |
| | | AGTCGAGTTGTTTGGGAATGCAGCTCTAAGTGGGTGGTAAATTCCAT | |
| | | CTAAAGCTAAATACCGGCGAGAGACCGATAGCGAACAAGTACAGTG | |
| | | ATGGAAAGATGAAAAGCACTTTGAAAAGAGAGTGAAAAAGTACGTG | |
| | | AAATTGTTGAAAGGGAAGGGCTTGCAAGCAGACACTTAACTGGGCC | |
| | | AGCATCGGGGCGGCGGGAAACAAAACCACCGGGGAATGTACCTTTC | |
| | | GAGGATTATAACCCCGGTCTCAATTTCCTCACCACCCCGAGGCCTGC | |
| | | AATCTAAGGATGCTGGCGTAATGGTTGCAAGTCGC (SEQ ID NO: 3) | |
| 2 | H01-2, | AAACCAACAGGGATTGCCTCAGTAACGGCGAGTGAAGCGGCAAAAG | 20 |
| | H01-3, | CTCAAATTTGAAATCCCCCGGGAATTGTAATTTGAAGAGATTTGGGT | |
| | H03-2 | CCGGCCGGCGGGGGTTAAGTCCACTGGAAAGTGGCGCCACAGAGGG | |
| | | TGACAGCCCCGTGAACCCCTTCAAAGCCTTCATCCCAGATCTCCAAG | |
| | | AGTCGAGTTGTTTGGGAATGCAGCTCTAAGTGGGTGGTAAATTCCAT | |
| | | CTAAAGCTAAATACCGGCGAGAGACCGATAGCGAACAAGTACAGTG | |
| | | ATGGAAAGATGAAAAGCACTTTGAAAAGAGAGTGAAAAAGTACGTG | |
| | | AAATTGTTGAAAGGGAAGGGCTTGCAAGCAGACACTTAACTGGGCC | |
| | | AGCATCGGGGCGGCGGGAAACAAAACCACCGGGGAATGTACCTTTC | |
| | | GAGGATTATAACCCCGGTCTCTATTTCCATGTTGCCCCGAGGCCTGC | |
| | | ATTCTAAGGATGCTGGCGTAATGGTTGCAAGTCGC (SEQ ID NO: 4) | |
| 3 | H02-1 | AAACCAACAGGGATTGCCTCAGTAACGGCGAGTGAAGCGGCAAAAG | 11 |
| | | CTCAAATTTGAAATCCCCCGGGAATTGTAATTTGAAGAGATTTGGGT | |
| | | CCGGCCGGCAGGGGTTAAGTCCACTGGAAAGTGGCGCCACAGAGGG | |
| | | TGACAGCCCCGTGAGCCCCTCTAAAGCCTCTACCCCAGATCTCCAAG | |
| | | AGTCGAGTTGTTTGGGAATGCAGCTCTAAGTGGGTGGTAAATTCCAT | |
| | | CTAAAGCTAAATACCGGCGAGAGACCGATAGCGAACAAGTACAGTG | |
| | | ATGGAAAGATGAAAAGCACTTTGAAAAGAGAGTGAAAAAGTACGTG | |
| | | AAATTGTTGAAAGGGAAGGGCTTGCAAGCAGACACTTAACTGGGCC | |
| | | AGCATCGGGGCGGCGGGAAACAAAACCACCGGGGAATGTACCTTTC | |
| | | GAGGATTATACCCCTGGTCTCTATTTCCATGTTGCCCCGAGGCCTGCA | |
| | | ATCTAAGGATGCTGGCGTAATGGTTGCAAGTCGC (SEQ ID NO: 5) | |
| 4 | H02-3 | AAACCAACAGGGATTGCCTCAGTAACGGCGAGTGAAGCGGCAAAAG | 11 |
| | | CTCAAATTTGAAATCCCCCGGGAATTGTAATTTGAAGAGATTTGGGT | |
| | | CCGGCCGGCGGGGGTTAAGTCCACTGGAAAGTGGCGCCACAGAGGG | |
| | | TGACAGCCCCGTGAGCCCCTCTAACGCCTCTACCCCAAATCTCCAAG | |
| | | AGTCGAGTTGTTTGGGAATGCAGCTCTAAGTGGGTGGTAAATTCCAT | |
| | | CTAAAGCTAAATACCGGCGAGAGACCGATAGCGAACAAGTACAGTG | |
| | | ATGGAAAGATGAAAAGCACTTTGAAAAGAGAGTGAAAAAGTACGTG | |
| | | AAATTGTTGAAAGGGAAGGGCTTGCAAGCAGACACTTAACTGGGCC | |
| | | AGCATCGGGGCGGCGGGAAACAAAACCACCGGGGAATGTACCTTTC | |
| | | GAGGATTATAACCCCGGTCTCTATTTCCATGTTGCCCCGAGGCCTGC | |
| | | ATTCTAAGGATGCTGGCGTAATGGTTGCAAGTCGC (SEQ ID NO: 6) | |
| 5 | H03-1 | AAACCAACAGGGATTGCCTCAGTAACGGCGAGTGAAGCGGCAAAAG | 12 |
| | | CTCAAATTTGAAATCCCCCGGGAATTGTAATTTGAAGAGATTTGGGT | |
| | | CCGGCCGGCGGGGGTTAAGTCCACTGGAAAGTGGCGCCACAGAGGG | |
| | | TGACAGCCCCGTGAGCCCCTCTAACGCCTCTACCCCAGATCTCCAAG | |
| | | AGTCGAGTTGTTTGGGAATGCAGCTCTAAGTGGGTGGTAAATTCCAT | |
| | | CTAAAGCTAAATACCGGCGAGAGACCGATAGCGAACAAGTACAGTG | |
| | | ATGGAAAGATGAAAAGCACTTTGAAAAGAGAGTGAAAAAGTACGTG | |
| | | AAATTGTTGAAAGGGAAGGGCTTGCAAGCAGACACTTAACTGGGCC | |
| | | AGCATCGGGGCGGCGGGAAACAAAACCACCGGGGAATGTACCTTTC | |
| | | GAGGATTATAACCCCGGTCTCTATTTCCATGTTGCCCCGAGGCCTGC | |
| | | ATTCTAAGGATGCTGGCGTAATGGTTGCAAGTCGC (SEQ ID NO: 7) | |
| 6 | H1-1, | AAACCAACAGGGATTGCCTCAGTAACGGCGAGTGAAGCGGCAAAAG | 13 |
| | H1-3 | CTCAAATTTGAAATCCCCCGGGAATTGTAATTTGAAGAGATTTGGGT | |
| | | CCGGCCGGCAGGGGTTAAGTCCACTGGAAAGTGGCGCCACAGAGGG | |
| | | TGACAGCCCCGTGAACCCCTTTAACGCCCTCATCCCAGATCTCCAAG | |
| | | AGTCGAGTTGTTTGGGAATGCAGCTCTAAGTGGGTGGTAAATTCCAT | |
| | | CTAAAGCTAAATACCGGCGAGAGACCGATAGCGAACAAGTACAGTG | |
| | | ATGGAAAGATGAAAAGCACTTTGAAAAGAGAGTGAAAAAGTACGTG | |
| | | AAATTGTTGAAAGGGAAGGGCTTGCAAGCAGACACTTAACTGGGCC | |
| | | AGCATCGGGGCGGCGGGAAACAAAACCACCGGGGAATGTACCTCTC | |
| | | GAGGATTATAACCCCGGTCTCAATTTCCTTGTTGCCCCGAGGCCTGC | |
| | | AATCTAAGGATGCTGGCGTAATGGTTGCAAGTCGC (SEQ ID NO: 8) | |
| 7 | H1-2 | AAACCAACAGGGATTGCCTCAGTAACGGCGAGTGAAGCGGCAAAAG | 20 |
| | | CTCAAATTTGAAATCCCCCGGGAATTGTAATTTGAAGAGATTTGGGT | |
| | | CCGGCCGGCAGGGGTTAAGTCCACTGGAAAGTGGCGCCACAGAGGG | |
| | | TGACAGCCCCGTGAACCCCCTCAACGCCCTCATCCCAGATCTCCAAG | |
| | | AGTCGAGTTGTTTGGGAATGCAGCTCTAAGTGGGTGGTAAATTCCAT | |
| | | CTAAAGCTAAATACCGGCGAGAGACCGATAGCGAACAAGTACAGTG | |
| | | ATGGAAAGATGAAAAGCACTTTGAAAAGAGAGTGAAAAAGTACGTG | |
| | | AAATTGTTGAAAGGGAAGGGCTTGCAAGCAGACACTTAACTGGGCC | |
| | | AGCATCGGGGCGGCGGGAAACAAAACCACCGGGGAATGTACCTTTC | |
| | | GAGGATTATACCCCTGGTCTCTATTTCCATGTTGCCCCGAGGCCTGCA | |
| | | ATCTAAGGATGCTGGCGTAATGGTTGCAAGTCGC (SEQ ID NO: 9) | |
| 8 | H1-4 | AAACCAACAGGGATTGCCTCAGTAACGGCGAGTGAAGCGGCAAAAG | 23 |
| | | CTCAAATTTAAAATCCCCCGGGAATTGTAATTTGAAGAGATTTGGGT | |
| | | CCGGCCGGCAGGGGTTAAGTCCACTGGAAAGTGGCGCCACAGAGGG | |
| | | TGACAGCCCCGTGAACCCCTTTAACGCCCTCATCCCAGATCTCCAAG | |
| | | AGTCGAGTTGTTTGGGAATGCAGCTCTAAGTGGGTGGTAAATTCCAT | |
| | | CTAAAGCTAAATACCGGCGAGAGACCGATAGCGAACAAGTACAGTG | |
| | | ATGGAAAGATGAAAAGCACTTTGAAAAGAGAGTGAAAAAGTACGTG | |
| | | AAATCGTTGAAAGGGAAGGGCTTGCAAGCAGACACTTAACTGGGCC | |
| | | AGCATCGGGGCGGCGGGGAGCAAAACCACCGGGGAATGTACCTTTC | |
| | | GAGGATTATAACCCCGGCCCTTACTCCCATACTGCCCCGAGGCCTGC | |
| | | AATCTAAGGATGCTGGCGTAATGGTTGCAAGTCGC (SEQ ID NO: 10) | |
| 9 | H1-5, | AAACCAACAGGGATTGCCTCAGTAACGGCGAGTGAAGCGGCAAAAG | 18 |
| | H2-5, | CTCAAATTTGAAATCCCCCGGGAATTGTAATTTGAAGAGATTTGGGT | |
| | H2-7 | CCGGCCGGCAGGGGTTAAGTCCACTGGAAAGTGGCGCCACAGAGGG | |
| | | TGACAGCCCCGTGAACCCCTTTAACGCCCTCATCCCAGATCTCCAAG | |
| | | AGTCGAGTTGTTTGGGAATGCAGCTCTAAGTGGGTGGTAAATTCCAT | |
| | | CTAAAGCTAAATACCGGCGAGAGACCGATAGCGAACAAGTACAGTG | |
| | | ATGGAAAGATGAAAAGCACTTTGAAAAGAGAGTGAAAAAGTACGTG | |
| | | AAATTGTTGAAAGGGAAGGGCTTGCAAGCAGACACTTAACTGGGCC | |
| | | AGCATCGGGGCGGCGGGGAGCAAAACCACCGGGGAATGTACCTTTC | |
| | | GAGGATTATAACCCCGGCCCTTACTCCCACACCACCCCGAGGCCTGC | |
| | | AATCTAAGGATGCTGGCGTAATGGTTGCAAGTCGC (SEQ ID NO: 11) | |
| 10 | H1-6 | AAACCAACAGGGATTGCCTCAGTAACGGCGAGTGAAGCGGCAAAAG | 9 |
| | | CTCAAATTTGAAATCCCCCGGGAATTGTAATTTGAAGAGATTTGGGT | |
| | | CCGGCCGGCGGGGGTTAAGTCCACTGGAAAGTGGCGCCACAGAGGG | |
| | | TGACAGCCCCGTGAACCCCTTTAACGCCCTCATCCCAGATCTCCAAG | |
| | | AGTCGAGTTGTTTGGGAATGCAGCTCTAAGTGGGTGGTAAATTCCAT | |
| | | CTAAAGCTAAATACCGGCGAGAGACCGATAGCGAACAAGTACAGTG | |
| | | ATGGAAAGATGAAAAGCACTTTGAAAAGAGAGTGAAAAAGTACGTG | |
| | | AAATTGTTGAAAGGGAAGGGCTTGCAAGCAGACACTTAACTGGGCC | |
| | | AGCATCGGGGCGGCGGGAAACAAAACCACCGGGGAATGTACCTCTC | |
| | | GAGGATTATAACCCCGGTCTCAATTTCCTCACCACCCCGAGGCCTGC | |
| | | AATCTAAGGATGCTGGCGTAATGGTTGCAAGTCGC (SEQ ID NO: 12) | |
| 11 | H1-7 | AAACCAACAGGGATTGCCTCAGTAACGGCGAGTGAAGCGGCAAAAG | 0 |
| | | CTCAAATTTGAAATCCCCCGGGAATTGTAATTTGAAGAGATTTGGGT | |
| | | CCGGCCGGCAGGGGTTAAGTCCACTGGAAAGTGGCGCCACAGAGGG | |
| | | TGACAGCCCCGTGAGCCCCTCTAAAGCCTCTACCCCAAATCTCCAAG | |
| | | AGTCGAGTTGTTTGGGAATGCAGCTCTAAGTGGGTGGTAAATTCCAT | |
| | | CTAAAGCTAAATACCGGCGAGAGACCGATAGCGAACAAGTACAGTG | |
| | | ATGGAAAGATGAAAAGCACTTTGAAAAGAGAGTGAAAAAGTACGTG | |
| | | AAATTGTTGAAAGGGAAGGGCTTGCAAGCAGACACTTAACTGGGCC | |
| | | AGCATCGGGGCGGCGGGAAACAAAACCACCGGGGAATGTACCTCTC | |
| | | GAGGATTATAACCCCGGTCTCAATTTCCTCACCACCCCGAGGCCTGC | |
| | | AATCTAAGGATGCTGGCGTAATGGTTGCAAGTCGC (SEQ ID NO: 13) | |
| 12 | H1-8 | AAACCAACAGGGATTGCCTCAGTAACGGCGAGTGAAGCGGCAAAAG | 15 |
| | | CTCAAATTTGAAATCCCCCGGGAATTGTAATTTGAAGAGATTTGGGT | |
| | | CCGGCCGGCAGGGGTTAAGTCCACTGGAAAGTGGCGCCACAGAGGG | |
| | | TGACAGCCCCGTAAACCCCTTCAAAGCCTTCATCCCAGATCTCCAAG | |
| | | AGTCGAGTTGTTTGGGAATGCAGCTCTAAGTGGGTGGTAAATTCCAT | |
| | | CTAAAGCTAAATACCGGCGAGAGACCGATAGCGAACAAGTACAGTG | |
| | | ATGGAAAGATGAAAAGCACTTTGAAAAGAGAGTGAAAAAGTACGTG | |
| | | AAATTGTTGAAAGGGAAGGGCTTGCAAGCAGACACTTAACTGGGCC | |
| | | AGCATCGGGGCGGCGGGAAACAAAACCACCGGGGAATGTACCTTTC | |
| | | GAGGATTATAACCCCGGTCCTTACTCCCTCACCATCCCGAGGCCTGC | |
| | | AATCTAAGGATGCTGGCGTAATGGTTGCAAGTCGC (SEQ ID NO: 14) | |
| 13 | H2-1 | AAACCAACAGGGATTGCCTCAGTAACGGCGAGTGAAGCGGCAAAAG | 14 |
| | | CTCAAATTTGAAATCCCCCGGGAATTGTAATTTGAAGAGATTTGGGT | |
| | | CCGGCCGGCAGGGGTTAAGTCCACTGGAAAGTGGCGCCACAGAGGG | |
| | | TGACAGCCCCGTGAACCCCTTCAAAGCCTTCATCCCAGATCTCCAAG | |
| | | AGTCGAGTTGTTTGGGAATGCAGCTCTAAGTGGGTGGTAAATTCCAT | |
| | | CTAAAGCTAAATACCGGCGAGAGACCGATAGCGAACAAGTACAGTG | |
| | | ATGGAAAGATGAAAAGCACTTTGAAAAGAGAGTGAAAAAGTACGTG | |
| | | AAATTGTTGAAAGGGAAGGGCTTGCAAGCAGACACTTAACTGGGCC | |
| | | AGCATCGGGGCGGCGGGAAACAAAACCACCGGGGAATGTACCTTTC | |
| | | GAGGATTATAACCCCGGTCCTTACTCCCACACCACCCCGAGGCCTGC | |
| | | AATCTAAGGATGCTGGCGTAATGGTTGCAAGTCGC (SEQ ID NO: 15) | |
| 14 | H2-2 | AAACCAACAGGGATTGCCTCAGTAACGGCGAGTGAAGCGGCAAAAG | 14 |
| | | CTCAAATTTGAAATCCCCCGGGAATTGTAATTTGAAGAGATTTGGGT | |
| | | TCGGCCGGCAGGGGTTAAGTCCACTGGAAAGTGGCGCCACAGAGGG | |
| | | TGACAGCCCCGTGAACCCCTTCAACGCCTTCATCCCAGATCTCCAAG | |
| | | AGTCGAGTTGTTTGGGAATGCAGCTCTAAGTGGGTGGTAAATTCCAT | |
| | | CTAAAGCTAAATACCGGCGAGAGACCGATAGCGAACAAGTACAGTG | |
| | | ATGGAAAGATGAAAAGCACTTTGAAAAGAGAGTGAAAAAGTACGTG | |
| | | AAATTGTTGAAAGGGAAGGGCTTGCAAGCAGACACTTAACTGGGCC | |
| | | AGCATCGGGGCGGCGGGAAACAAAACCACCGGGGAATGTACCTCTC | |
| | | GAGGATTATAACCCCGGTCTCAATTTCCTTGTTGCCCCGAGGCCTGC | |
| | | AATCTAAGGATGCTGGCGTAATGGTTGCAAGTCGC (SEQ ID NO: 16) | |
| 15 | H2-3 | AAACCAACAGGGATTGCCTCAGTAACGGCGAGTGAAGCGGCAAAAG | 16 |
| | | CTCAAATTTGAAATCCCCCGGGAATTGTAATTTGAAGAGATTTGGGT | |
| | | CCGGCCGGCAGGGGTTAAGTCCACTGGAAAGTGGCGCCACAGAGGG | |
| | | TGACAGCCCCGTGAACCCCTTCAACGCCCTCATCCCAGATCTCCAAG | |
| | | AGTCGAGTTGTTTGGGAATGCAGCTCTAAGTGGGTGGTAAATTCCAT | |
| | | CTAAAGCTAAATACCGGCGAGAGACCGATAGCGAACAAGTACAGTG | |
| | | ATGGAAAGATGAAAAGCACTTTGAAAAGAGAGTGAAAAAGTACGTG | |
| | | AAATTGTTGAAAGGGAAGGGCTTGCAAGCAGACACTTAACTGGGCC | |
| | | AGCATCGGGGCGGCGGGAAACAAAACCACCGGGGAATGTACCTTTC | |
| | | GAGGATTATAACCCCGGTCCTTACTCCCTCACCATCCCGAGGCCTGC | |
| | | AATCTAAGGATGCTGGCGTAATGGTTGCAAGTCGC (SEQ ID NO: 17) | |
| 16 | H2-4 | AAACCAACAGGGATTGCCTCAGTAACGGCGAGTGAAGCGGCAAAAG | 20 |
| | | CTCAAATTTAAAATCCCCCGGGAATTGTAATTTGAAGAGATTTGGGT | |
| | | CCGGCCGGCGGGGGTTAAGTCCACTGGAAAGTGGCGCCACAGAGGG | |
| | | TGACAGCCCCGTGAACCCCTTTAACGCCCTCATCCCAGATCTCCAAG | |
| | | AGTCGAGTTGTTTGGGAATGCAGCTCTAAGTGGGTGGTAAATTCCAT | |
| | | CTAAAGCTAAATACCGGCGAGAGACCGATAGCGAACAAGTACAGTG | |
| | | ATGGAAAGATGAAAAGCACTTTGAAAAGAGAGTGAAAAAGTACGTG | |
| | | AAATTGTTGAAAGGGAAGGGCTTGCAAGCAGACACTTAACTGGGCC | |
| | | AGCATCGGGGCGGCGGGGAGCAAAACCACCGGGGAATGTACCTTTC | |
| | | GAGGATTATAACCCCGGCCCTTACTCCCACAcCACCCCGAGGCCTGC | |
| | | AATCTAAGGATGCTGGCGTAATGGTTGCAAGTCGC (SEQ ID NO: 18) | |
| 17 | H2-6, | AAACCAACAGGGATTGCCTCAGTAACGGCGAGTGAAGCGGCAAAAG | 12 |
| | H3-7 | CTCAAATTTGAAATCCCCCGGGAATTGTAATTTGAAGAGATTTGGGT | |
| | | CCGGCCGGCGGGGGTTAAGTCCACTGGAAAGTGGCGCCACAGAGGG | |
| | | TGACAGCCCCGTGAACCCCCTCAACGCCCTCATCCCAGATCTCCAAG | |
| | | AGTCGAGTTGTTTGGGAATGCAGCTCTAAGTGGGTGGTAAATTCCAT | |
| | | CTAAAGCTAAATACCGGCGAGAGACCGATAGCGAACAAGTACAGTG | |
| | | ATGGAAAGATGAAAAGCACTTTGAAAAGAGAGTGAAAAAGTACGTG | |
| | | AAATTGTTGAAAGGGAAGGGCTTGCAAGCAGACACTTAACTGGGCC | |
| | | AGCATCGGGGCGGCGGGAAACAAAACCACCGGGGAATGTACCTTTC | |
| | | GAGGATTATAACCCCGGTCTCAATTTCCTCACCACCCCGAGGCCTGC | |
| | | AATCTAAGGATGCTGGCGTAATGGTTGCAAGTCGC (SEQ ID NO: 19) | |
| 18 | H2-8 | AAACCAACAGGGATTGCCTCAGTAACGGCGAGTGAAGCGGCAAAAG | 18 |
| | | CTCAAATTTGAAATCCCCCGGGAATTGTAATTTGAAGAGATTTGGGT | |
| | | CCGGCCGGCAGGGGTTAAGTCCACTGGAAAGTGGCGCCACAGAGGG | |
| | | TGACAGCCCCGTGAACCCCCTCAACGCCCTCATCCCAGATCTCCAAG | |
| | | AGTCGAGTTGTTTGGGAATGCAGCTCTAAGTGGGTGGTAAATTCCAT | |
| | | CTAAAGCTAAATACCGGCGAGAGACCGATAGCGAACAAGTACAGTG | |
| | | ATGGAAAGATGAAAAGCACTTTGAAAAGAGAGTGAAAAAGTACGTG | |
| | | AAATTGTTGAAAGGGAAGGGCTTGCAAGCAGACACTTAACTGGGCC | |
| | | AGCATCGGGGCGGCGGGGAGCAAAACCACCGGGGAATGTACCTTTC | |
| | | GAGGATTATAACCCCGGTCCTTTTTTCCTTGTTGCCCCGAGGCCTGCA | |
| | | ATCTAAGGATGCTGGCGTAATGGTTGCAAGTCGC (SEQ ID NO: 20) | |
| 19 | H3-1, | AAACCAACAGGGATTGCCTCAGTAACGGCGAGTGAAGCGGCAAAAG | 20 |
| | H3-4, | CTCAAATTTGAAATCCCCCGGGAATTGTAATTTGAAGAGATTTGGGT | |
| | H3-6 | TCGGCCGGCAGGGGTTAAGTCCACTGGAAAGTGGCGCCACAGAGGG | |
| | | TGACAGCCCCGTGAACCCCTTCAACGCCCTCATCCCAGATCTCCAAG | |
| | | AGTCGAGTTGTTTGGGAATGCAGCTCTAAGTGGGTGGTAAATTCCAT | |
| | | CTAAAGCTAAATACCGGCGAGAGACCGATAGCGAACAAGTACAGTG | |
| | | ATGGAAAGATGAAAAGCACTTTGAAAAGAGAGTGAAAAAGTACGTG | |
| | | AAATTGTTGAAAGGGAAGGGCTTGCAAGCAGACACTTAACTGGGCC | |
| | | AGCATCGGGGCGGCGGGAAACAAAACCACCGGGGAATGTACCTTTC | |
| | | GAGGATTATAACCCCGGTCCTTTTTTCCTTGTTGCCCCGAGGCCTGCA | |
| | | ATCTAAGGATGCTGGCGTAATGGTTGCAAGTCGC (SEQ ID NO: 21) | |
| 20 | H3-2 | AAACCAACAGGGATTGCCTCAGTAACGGCGAGTGAAGCGGCAAAAG | 8 |
| | | CTCAAATTTGAAATCCCCCGGGAATTGTAATTTGAAGAGATTTGGGT | |
| | | CCGGCCGGCAGGGGTTAAGTCCACTGGAAAGTGGCGCCACAGAGGG | |
| | | TGACAGCCCCGTGAACCCCTTCAACGCCTTCATCCCAGATCTCCAAG | |
| | | AGTCGAGTTGTTTGGGAATGCAGCTCTAAGTGGGTGGTAAATTCCAT | |
| | | CTAAAGCTAAATACCGGCGAGAGACCGATAGCGAACAAGTACAGTG | |
| | | ATGGAAAGATGAAAAGCACTTTGAAAAGAGAGTGAAAAAGTACGTG | |
| | | AAATTGTTGAAAGGGAAGGGCTTGCAAGCAGACACTTAACTGGGCC | |
| | | AGCATCGGGGCGGCGGGAAACAAAACCACCGGGGAATGTACCTCTC | |
| | | GAGGATTATAACCCCGGTCTCAATTTCCTCACCACCCCGAGGCCTGC | |
| | | AATCTAAGGATGCTGGCGTAATGGTTGCAAGTCGC (SEQ ID NO: 22) | |
| 21 | H3-3 | AAACCAACAGGGATTGCCTCAGTAACGGCGAGTGAAGCGGCAAAAG | 15 |
| | | CTCAAATTTGAAATCCCCCGGGAATTGTAATTTGAAGAGATTTGGGT | |
| | | CCGGCCGGCAGGGGTTAAGTCCACTGGAAAGTGGCGCCACAGAGGG | |
| | | TGACAGCCCCGTGAACCCCTTCAAAGCCTTCATCCCAGATCTCCAAG | |
| | | AGTCGAGTTGTTTGGGAATGCAGCTCTAAGTGGGTGGTAAATTCCAT | |
| | | CTAAAGCTAAATACCGGCGAGAGACCGATAGCGAACAAGTACAGTG | |
| | | ATGGAAAGATGAAAAGCACTTTGAAAAGAGAGTGAAAAAGTACGTG | |
| | | AAATTGTTGAAAGGGAAGGGCTTGCAAGCAGACACTTAACTGGGCC | |
| | | AGCATCGGGGCGGCGGGAAACAAAACCACTGGGGAATGTACCTTTC | |
| | | GAGGATTATAACCCCGGTCCTTACTCCCTCACCATCCCGAGGCCTGC | |
| | | AATCTAAGGATGCTGGCGTAATGGTTGCAAGTCGC (SEQ ID NO: 23) | |
| 22 | H3-5 | AAACCAACAGGGATTGCCTCAGTAACGGCGAGTGAAGCGGCAAAAG | 12 |
| | | CTCAAATTTGAAATCCCCCGGGAATTGTAATTTGAAGAGATTTGGGT | |
| | | CCGGCCGGCAGGGGTTAAGTCCACTGGAAAGTGGCGCCACAGAGGG | |
| | | TGACAGCCCCGTGAACCCCTTCAAAGCTTTTACCCCAGATCTCCAAG | |
| | | AGTCGAGTTGTTTGGGAATGCAGCTCTAAGTGGGTGGTAAATTCCAT | |
| | | CTAAAGCTAAATACCGGCGAGAGACCGATAGCGAACAAGTACAGTG | |
| | | ATGGAAAGATGAAAAGCACTTTGAAAAGAGAGTGAAAAAGTACGTG | |
| | | AAATTGTTGAAAGGGAAGGGCTTGCAAGCAGACACTTAACTGGGCC | |
| | | AGCATCGGGGCGGCGGGAAACAAAACCACCGGGGAATGTACCTTTC | |
| | | GAGGATTATAACCCCGGTCTCAATTTCCTTGTTGCCCCGAGGCCTGC | |
| | | AATCTAAGGATGCTGGCGTAATGGTTGCAAGTCGC (SEQ ID NO: 24) | |
| 23 | H3-8 | AAACCAACAGGGATTGCCTCAGTAACGGCGAGTGAAGCGGCAAAAG | 17 |
| | | CTCAAATTTGAAATCCCCCGGGAATTGTAATTTGAAGAGATTTGGGT | |
| | | TCGGCCGGCAGGGGTTAAGTCCACTGGAAAGTGGCGCCACAGAGGG | |
| | | TGACAGCCCCGTGAACCCCTTCAACGCCCTCATCCCAGATCTCCAAG | |
| | | AGTCGAGTTGTTTGGGAATGCAGCTCTAAGTGGGTGGTAAATTCCAT | |
| | | CTAAAGCTAAATACCGGCGAGAGACCGATAGCGAACAAGTACAGTG | |
| | | ATGGAAAGATGAAAAGCACTTTGAAAAGAGAGTGAAAAAGTACGTG | |
| | | AAATTGTTGAAAGGGAAGGGCTTGCAAGCAGACACTTAACTGGGCC | |
| | | AGCATCGGGGCGGCGGGAAACAAAACCACCGGGGAATGTACCTTTC | |
| | | GAGGATTATAACCCCGGTCCTTACTCCCTCACCATCCCGAGGCCTGC | |
| | | AATCTAAGGATGCTGGCGTAATGGTTGCAAGTCGC (SEQ ID NO: 25) | |

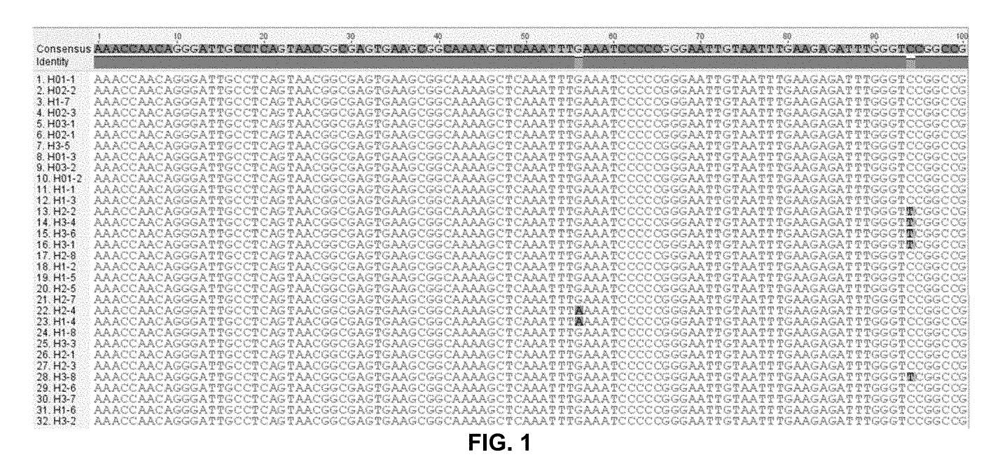

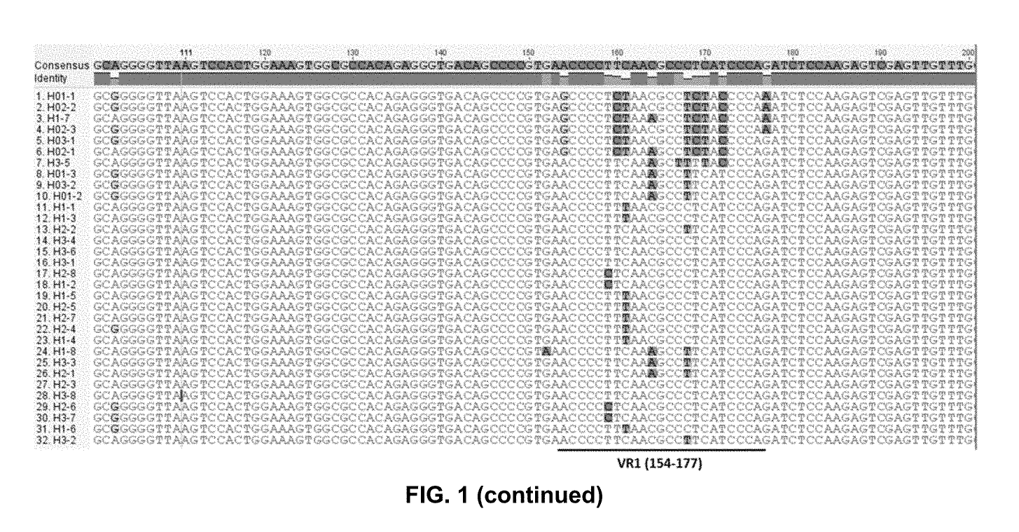

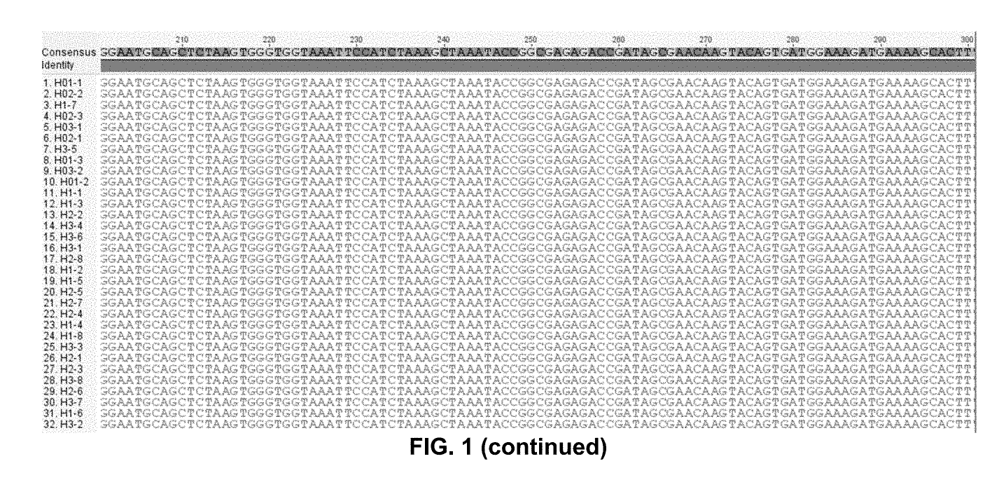

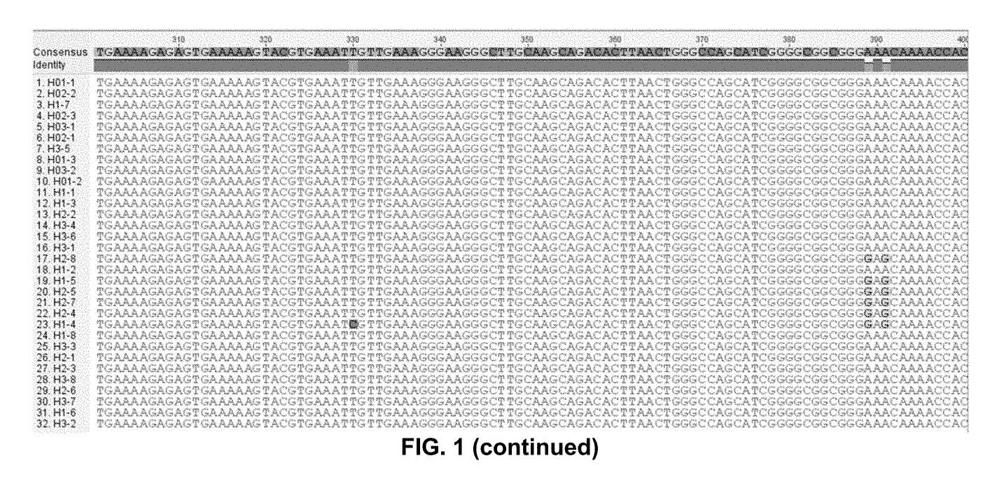

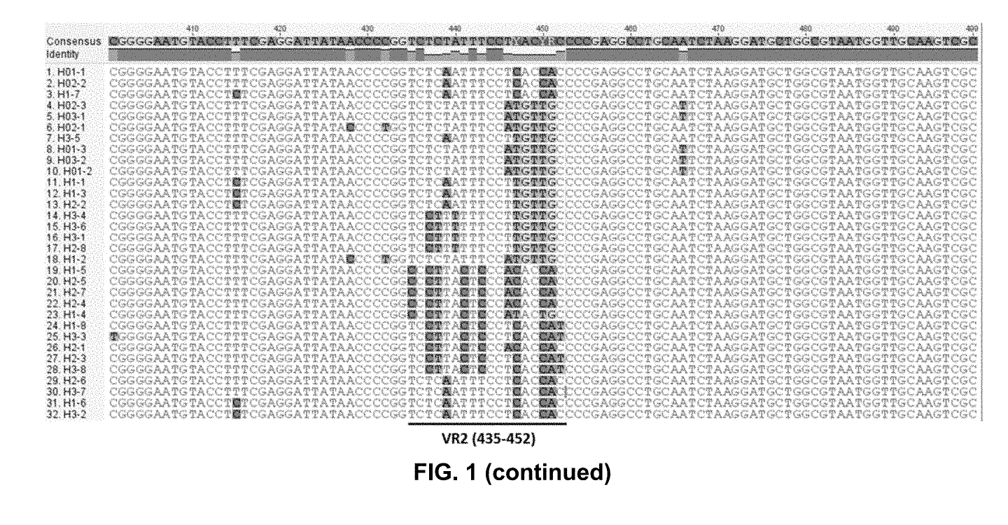

The variations in the D1/D2 regions were confined to two major areas that are located between nucleotides 154-177 and 435-452 of SEQ ID NO: 1 (FIG. 1). Outside of these two major variable regions, there were only 9 positions where a nucleotide difference was observed in at least two clones. In a single clone, the number of variable nucleotides outside the two highly variable regions was 0 (type 13, 15 and 22), or 1 (type 1, 6, 11, 12, 17, 19, 20, 21 and 23), or 2 (type 2, 3, 4, 5, 7, 9, 10, 14 and 18), or 3 (type 8), or 4 (type 16).

Additionally, the following consensus D1/D2 domain sequence was identified:

(SEQ ID NO: 2)

AAACCAACAGGGATTGCCTCAGTAACGGCGAGTGAAGCGGCAAAAGCTCA
AATTTGAAATCCCCCGGGAATTGTAATTTGAAGAGATTTGGGTCCGGCCG
GCAGGGGTTAAGTCCACTGGAAAGTGGCGCCACAGAGGGTGACAGCCCCG
TGAACCCCTTCAACGCCCTCATCCCAGATCTCCAAGAGTCGAGTTGTTTG
GGAATGCAGCTCTAAGTGGGTGGTAAATTCCATCTAAAGCTAAATACCGG
CGAGAGACCGATAGCGAACAAGTACAGTGATGGAAAGATGAAAAGCACTT
TGAAAAGAGAGTGAAAAAGTACGTGAAATTGTTGAAAGGGAAGGGCTTGC
AAGCAGACACTTAACTGGGCCAGCATCGGGGCGGCGGGAAACAAAACCAC
CGGGGAATGTACCTTTCGAGGATTATAACCCCGGTCTCTATTTCCTYACY
RCCCCGAGGCCTGCAATCTAAGGATGCTGGCGTAATGGTTGCAAGTCGC

All identified D1/D2 domain sequences for the H0 Metschnikowia sp. had at least a 97.1% sequence identity to the consensus D1/D2 sequence.

Based on these results, it was clear that the H0 Metschnikowia sp. is a member of the Metschnikowia genus and closely related to the species of the Metschnikowia pulcherrima clade, but it was apparent further characterization beyond the D1/D2 domain sequence was needed to differentiate the H0 Metschnikowia sp. from the other members of Metschnikowia pulcherrima clade.

### RNA Polymerase II (RPB2) Gene Sequence Analysis

The ACT1, 1st and 2nd codon positions of EF2 and RPB2 sequences have been used for phylogenetic analysis for all known species in the Metschnikowiaceae family (Guzman et al., 2013, Mol. Phylogenet. Evol., 68(2):161-175). Accordingly, analysis of the RPB2 sequence from the H0 Metschnikowia sp. was analyzed.

Partial RPB2 gene sequences were extracted from GeneBank for six Metschnikowia pulcherrima clade species and one outgroup species, Metschnikowia kunwiensis, which is close to but has separated from Metschnikowia pulcherrima (Table 4).

TABLE 4

| | Strain | RPB2 |
|---|---|---|
| Taxon | designation | accession no. |
| M. andauensis | CBS 10809 | KC859678 |
| M. chrysoperlae | CBS 9803 | KC859686 |
| M. fructicola | CBS 8853 | KC859693 |
| M. pulcherrima | CBS 5833 | KC859707 |
| M. shanxiensis | CBS 10359 | KC859710 |
| M. sinensis | CBS 10357 | KC859713 |
| M. zizyphicola | CBS 10358 | KC859716 |
| M. kunwiensis | CBS 9067 | KC859701 |

The RPB2 gene sequence from the H0 Metschnikowia sp. was extracted from H0 Metschnikowia sp. whole genome shotgun contigs, and is represented by:

(SEQ ID NO: 70)

ATGTCGCAGGAGCCGGTAGAAGACCCTTACGTCTACGACGAGGAGGACGC
GCACAGCATCACGCCCGAGGACTGCTGGACGGTGATTCTGTCGTTTTTCC
AGGAAAAAGGCCTTGTCTCACAGCAGTTGGACTCGTTCGACGAGTTCATC
GAGTCAAACATCCAGGAGTTGGTGTGGGAGGACTCGCACTTGATTCTCGA
CCAGCCGGCGCAACATACTTCCGAGGACCAGTATGAAAATAAGCGGTTTG
AAATCACGTTTGGCAAGATCTATATTTCGAAGCCAACGCAGACCGAGGGC
GACGGAACAACGCACCCGATGTTCCCACAGGAGGCACGCTTGCGTAACTT
GACCTACAGCTCGCCGCTTTACGTGGACATGCTGAAAAAGAAGTTTCTTT
CCGATGACAGAGTGAGAAAGGGTAACGAGCTAGAATGGGTGGAGGAGAAA
GTCGATGGCGAGGAGGCCCAGCTGAAGGTGTTCTTGGGTAAGGTGCCAAT
CATGCTAAGGTCGAAGTTTTGCATGTTGCGGGACTTGGGCGAGCACGAGT
TCTACGAGTTGAAAGAGTGCCCTTACGATATGGGTGGCTATTTCGTCATC
AACGGTTCCGAAAAAGTCTTGATCGCCCAGGAGCGCTCGGCGGCTAACAT
TGTCCAGGTGTTTAAGAAGGCAGCGCCCTCGCCCATCTCGCACGTGGCGG
AGATCCGTTCCGCGCTTGAAAAGGGTTCCCGTTTGATCTCCTCGATGCAG
ATCAAACTATATGGTCGTGACGACAAGGGCACCACTGGCAGAACAATCAA
GGCCACATTGCCCTACATCAAGGAAGACATCCCGATTGTGATTGTATTCA
GAGCCCTCGGCGTGGTCCCCGATGGAGACATTTTGGAACACATTTGTTAC
GATGCAAACGATTGGCAAATGTTAGAGATGTTGAAGCCATGTGTGGAGGA
AGGTTTCGTGATCCAGGAGCGCGAAGTCGCACTTGACTTTATCGGTAGAA
GAGGTGTCTTGGGTATCAGAAGGGAAAAGCGTATCCAGTACGCAAAGGAT
ATTTTACAGAAAGAGTTGTTGCCTAACATCACACAGGAGGCCGGTTTCGA
GTCAAGAAAGGCATTCTTCTTGGGTTACATGGTCAACCGTTTGTTGTTAT
GTGCATTAGAAAGAAAGGAGCCTGACGACAGAGATCATTTTGGCAAGAAG
AGATTGGATTTGGCCGGACCCTTGTTGGCATCCTTGTTCCGTCTCTTATT
CAAAAAGCTTACCAGGGATATCTATAACTACATGCAGCGGTGCGTGGAGA
ATGACAAGGAGTTTAATCTCACGTTGGCGGTCAAGTCACAGACCATCACT
GATGGTTTGCGGTACTCGTTGGCCACAGGTAATTGGGGTGAACAAAGAAA
GGCCATGAGTGCACGTGCCGGTGTGTCGCAGGTGTTGAACAGATACACAT
ACTCATCGACATTGTCGCATTTGAGAAGAACAAATACTCCAATTGGCCGT
GACGGTAAGATCGCCAAACCTAGACAGTTGCACAACACCCACTGGGGTCT
TGTATGTCCTGCAGAAACTCCTGAGGGTCAGGCGTGTGGTTTGGTGAAGA
ATTTGTCTTTGATGACGTGTATATCCGTTGGTACCTCTTCCGAGCCGATC
TTGTATTTCTTGGAAGAGTGGGGTATGGAACCCTTGGAGGACTATGTTCC
TTCGAACGCACCAGACTGCACAAGAGTCTTTGTCAACGGTGTATGGGTTG
GCACACACAGAGAACCGGCACAGCTTGTCGATACCATGAGGAGGTTGAGA
AGGAAGGGCGATATCTCTCCCGAGGTGTCGATCATCAGGGACATCAGAGA
AATGGAGTTCAAGATCTTCACCGATGCAGGCCGTGTCTACCGTCCGTTGT
TCATCGTGGACGACGACCCAGAGTCCGAAACCAAGGGTGAGTTGATGTTG
CAAAAAGAGCACGTGCACAAGTTGTTGAACTCGGCCTACGATGAATATGA
CGAGGATGACTCCAATGCGTACACATGGTCGTCGTTGGTGAATGATGGTG
TGGTAGAGTACGTTGACGCCGAGGAGGAGGAGACAATCATGATCGCCATG
ACCCCAGAGGATTTGGAGGCTTCCAAGAGTGCGTTGTCGGAGACTCAGCA
ACAGGATCTTCAAATGGAGGAACAAGAGCTTGATCCTGCAAAGCGAATCA
AACCAACTTATACCTCATCCACACACACCTTCACGCATTGTGAGATTCAT
CCTTCGATGATTTTGGGTGTCGCCGCCTCTATCATTCCGTTCCCCGACCA
TAACCAGTCGCCGCGTAACACATACCAGTCTGCTATGGGTAAACAAGCCA
TGGGTGTATTTTTGACTAACTATGCCGTTAGAATGGACACAATGGCAAAT
ATCTTATACTACCCACAGAAACCCTTGGCCACAACAAGAGCCATGGAGCA
CTTGAAGTTCCGTGAGTTGCCTGCTGGTCAGAATGCAGTGGTGGCCATTG
CTTGTTACTCCGGCTACAACCAAGAAGATTCCATGATCATGAACCAGTCG
TCGATTGATAGAGGATTGTTCCGGTCTTTGTTTTTCAGATCTTACATGGA
TCTAGAGAAGAGACAAGGTATGAAAGCCTTGGAGACGTTTGAAAAGCCAT
CCAGATCTGACACCTTGAGATTGAAGCATGGAACCTACGAAAAGTTAGAT
GACGATGGTTTGATCGCGCCTGGTGTCAGGGTCAGTGGTGAGGATATCAT
CATCGGTAAAACCACACCTATTCCACCTGACACCGAGGAGTTGGGTCAGA
GAACCCAGTATCATACCAAGAGAGATGCCTCGACGCCATTGAGAAGCACG
GAGTCTGGTATTGTTGACCAGGTTCTTTTGACCACAAATGGTGACGGCGC
CAAGTTCGTCAAGGTCAGAATGAGAACGACGAAGGTTCCACAAATCGGTG
ACAAGTTTGCCTCCAGACACGGACAAAAGGGTACAATCGGTGTCACATAT
AGACACGAGGATATGCCTTTCAGTGCACAGGGTATTGTGCCTGACTTGAT
CATAAACCCGCATGCTATTCCATCTCGTATGACAGTCGCTCACTTGATCG
AGTGTTTGTTGTCGAAAGTCTCTTCCTTGTCCGGATTGGAAGGTGACGCC
TCGCCATTCACGGACGTCACAGCCGAGGCTGTTTCCAAATTGTTGAGAGA
GCACGGATACCAATCTAGAGGTTTCGAGGTGATGTACAATGGTCACACCG
GTAAGAAGATGATGGCGCAAGTGTTCTTTGGCCCAACGTACTACCAGAGA
TTGAGGCATATGGTGGATGACAAGATCCACGCTAGAGCCAGAGGTCCAGT
TCAAGTTTTGACCAGGCAGCCTGTGGAAGGTAGATCCAGGGATGGTGGAT
TACGTTTCGGAGAGATGGAGAGAGATTGTATGATTGCGCACGGAGCTGCT
GGATTCTTAAAGGAAAGATTGATGGAGGCTTCGGATGCTTTCAGAGTTCA
CGTTTGTGGAATCTGTGGTTTGATGTCGGTGATTGCAAACTTGAAGAAGA
ACCAGTTCGAGTGTCGGTCGTGCAAAAACAAGACCAACATTTACCAGATC
CACATTCCATACGCAGCCAAATTGTTGTTCCAGGAGTTGATGGCCATGAA
CATTTCTCCTAGATTGTACACGGAGAGATCAGGAATCAGTGTGCGTGTCT
GA

Sequences were edited in Genieous 7.1.9 and aligned using ClustalW. A neighbor-joining tree was built using Genieous 7.1.9 tree builder.

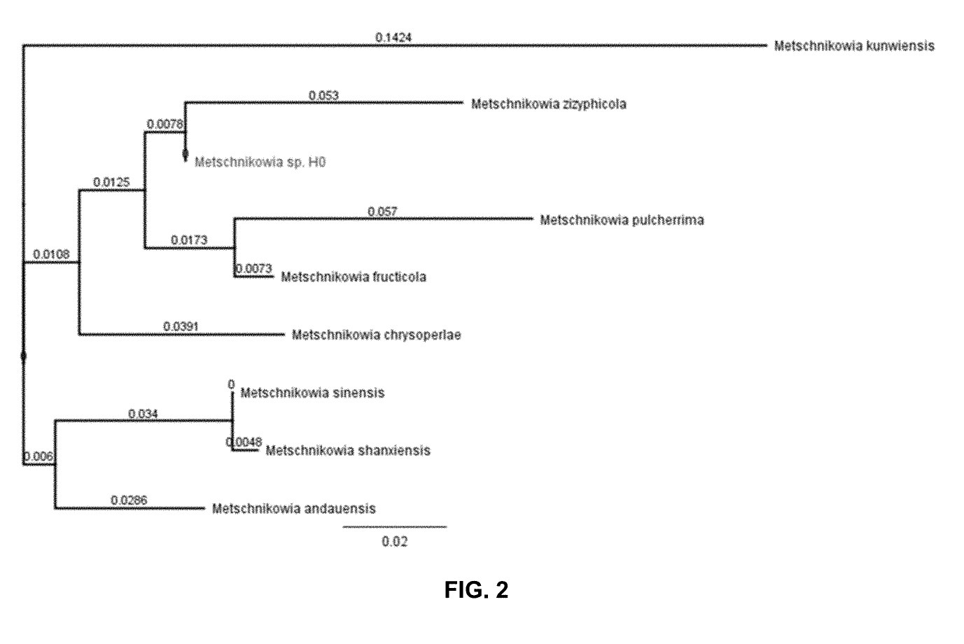

The phylogenetic distance between members of the Metschnikowia pulcherrima clade was closer than the distance between the Metschnikowia pulcherrima species and the Metschnikowia kunwiensis outgroup (FIG. 2). The H0 Metschnikowia sp. was clustered with Metschnikowia zizyphicola as a sub group (FIG. 2). The other sub groups are: (a) Metschnikowia pulcherrima and Metschnikowia fructicola; (b) M. andauensis, M. sinensis and M. shaxiensis; and (c) M. chrysoperlae (FIG. 2).

The above phylogenetic analysis shows that the H0 Metschnikowia sp. is a new species that is dusted with Metschnikowia zizyphicola as a sub group, as compared to other members of the Metschnikowia pulcherrima clade.

### Morphological and Physiological Characteristics

The H0 Metschnikowia sp. shares certain morphological and physiological characteristics with other Metschnikowia species, but it does have distinctive characteristics as well. For example, like other Metschnikowia pulcherrima clade species, H0 Metschnikowia sp. cells are globose to oval. Budding is multilateral. Abundant spherical chlamydospore-like ‘pulcherrima’ cells are present when H0 Metschnikowia sp. yeast cells are grown in YPD broth for 7 days at 30° C. The H0 Metschnikowia sp. can slowly grow at 4° C., it grows well at 20° C. to 33° C., and do not grow at 37° C. on YPD agar. The H0 Metschnikowia sp. secretes pink pigment to the medium. The H0 Metschnikowia sp. can assimilate D-glucose, D-galactose, D-xylose, sucrose, glycerol, ethanol, succinate and cellobiose and weakly ferment glucose.

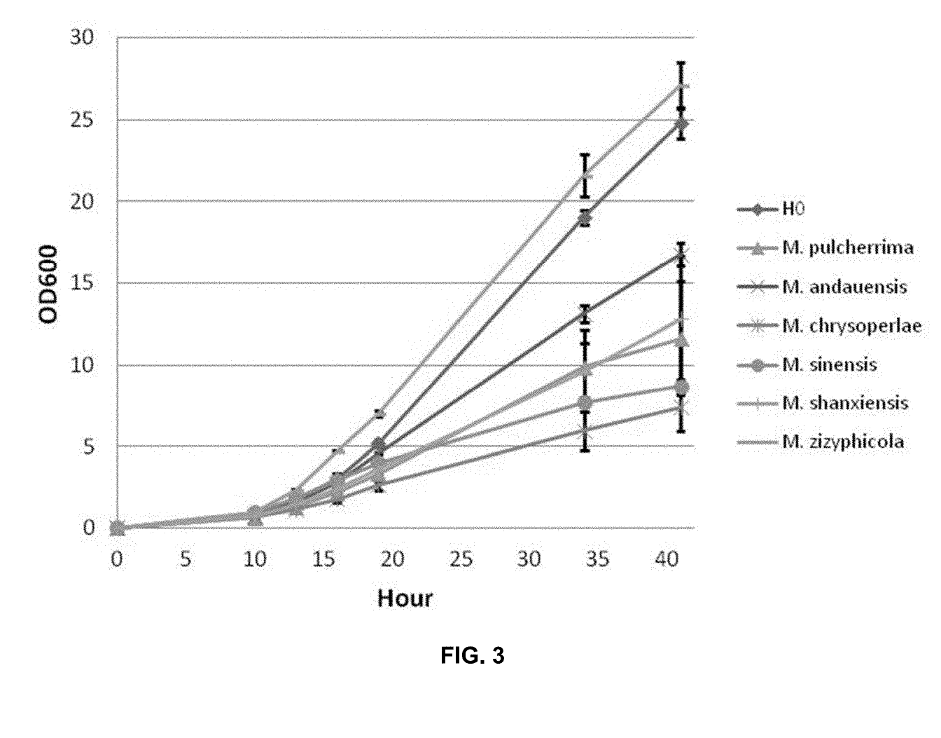

The H0 Metschnikowia sp. is distinguished from other members of Metschnikowia pulcherrima clade species by its growth in YP medium plus 2% xylose for extended time period. At the late stages of aerobic growth in YP plus 2% xylose medium for 41 hours with initial OD₆₀₀ at 0.03, the optical density at OD₆₀₀ of both H0 Metschnikowia sp. and Metschnikowia zizyphicola cultures were close and much higher than that of other strains (FIG. 3). The close relationship of H0 Metschnikowia sp. with Metschnikowia zizyphicola revealed by the xylose growth profile is consistent with the result for RPB2 sequence analyses discussed above.

Based on all of the above experiments, it is clear that the H0 Metschnikowia sp. is a novel Metschnikowia pulcherrima clade species and can be separated from other members by the RPB2 sequence and its xylose growth profile.

### EXAMPLE II

### Production of Xylitol from Xylose of H0 Metschnikowia sp.

This example demonstrates that the H0 Metschnikowia sp. produces xylitol from xylose when cultured in YEP medium containing xylose.

The production of xylitol from xylose was assayed for the H0 Metschnikowia sp. in yeast extract peptone (YEP) medium supplemented with 4% w/v or 10% w/v xylose. As a control, S. cerevisiae wine yeast M2 was also assayed.

H0 Metschnikowia sp. cells were inoculated into 50 ml of YEP+4% w/v or 10% w/v xylose medium in a 125 ml flask and grown at 30° C. incubater with shaking at 120 rpm. A 1 ml sample was taken from the culture and cells were removed by centrifugation. The supernatant was filtrated through a 0.22 μm nylon syringe filter into a HPLC sample vial. The xylitol content in the supernatant was analyzed by HPLC on Rezex RPM-monosaccharide Pb+2 column (Phenomenex) at 80° C. using water as a mobile phase at a rate of 0.6 ml/min. The peaks were detected with an Agilent G1362A refractive index detector (Agilent).

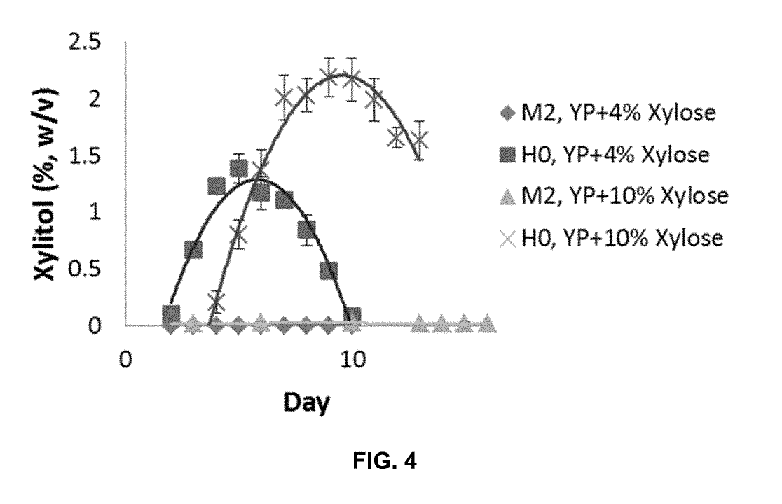

The H0 Metschnikowia sp. produced xylitol via a xylose dependent pathway. For example, in 4% xylose medium, the H0 Metschnikowia sp. produced approximately 13.8 g/L of xylitol from 40 g/L of xylose in 5 days, whereas in 10% xylose it produced approximately 23 g/L of xylitol from 100 g/L of xylose in 10 days (FIG. 4). When xylose was used up, the H0 Metschnikowia sp. started to consume the xylitol in the medium (FIG. 4). In both mediums, the S. cerevisiae M2 species produced no xylitol (FIG. 4).

### EXAMPLE III

### Production of Various Compounds by the H0 Metschnikowia sp.

This example demonstrates that the H0 Metschnikowia sp. produces several different compounds as well as xylitol when cultured in YEP medium containing xylose.

The H0 Metschnikowia sp. was grown in YEP medium containing 4% xylose at 30° C. Samples were taken on day 3 and day 6 post inoculation, and were analyzed by gas chromatography-mass spectrometry (GCMS) for volatile compounds as well as for xylitol.

This assay showed that xylitol, isopropanol, ethanol, isobutanol, n-butanol and 2-phenylethyl alcohol were produced by the H0 Metschnikowia sp. Table 5 shows the average concentration of these products measured on Days 3 and 6. The rate of production for each of these compounds was determined to be about 0.11 g/L/h of xylitol, about 6.8E-05 g/L/h of n-butanol, about 2.5E-04 g/L/h of isobutanol, about 2.4E-04 g/L/h of isopropanol, about 2.64E-04 g/L/h of ethanol and about 3.73E-06 g/L/h of 2-phenylethyl alcohol at a relative ratio of 99.26% xylitol, 0.061% n-butanol, 0.223% isobutanol, 0.217% isopropanol, 0.236% ethanol and 0.003% 2-phenylethyl alcohol when cultured under aerobic conditions for three days in liquid yeast extract peptone (YEP) medium comprising 4% xylose.

TABLE 5

| | Concentration | | Concentration | |
|---|---|---|---|---|
| | Day | | Day | |
| | 3 [μg/ml] | stdv | 6 [μg/ml] | stdv |
| Xylitol | 8000 | 0.01 | NT | NT |
| Isopropanol | 17.58 | 1.32 | 19.93 | 1.94 |
| Ethanol | 19.74 | 0.64 | 94.49 | 1.27 |
| Isobutanol | 18.1 | 0.1 | 20.95 | 0.21 |
| n-Butanol | 4.9 | 0.3 | 0.84 | 0.03 |
| 2-phenylethyl alcohol | 0.27 | 0.26 | 4.11 | 0.55 |

NT = not tested.

### EXAMPLE IV

### Growth and Production of Metabolites Specific to the H0 Metschnikowia sp.

This example demonstrates that the H0 Metschnikowia sp. grows differentially and produces different metabolites when compared to a close relative species (Metschnikowia pulcherrima flavia).

Three single colonies of H0 Metschnikowia sp. and Metschnikowia pulcherrima flavia (FL) were inoculated into 5 ml yeast extract peptone dextrose (YEPD) media respectively, grown at 30° C. overnight. Cultures were shifted to 100 ml YEPD and grown at 30° C. for 4 hours. Cells were collected and inoculated into 200 ml medium in a 500 ml flask with OD₆₀₀=1.0. Four different types of medium were used: 1) YNBG: yeast nitrogen base with 4% glucose, 2) YNBX: yeast nitrogen base with 4% xylose, 3) YNBGX: yeast nitrogen base with 2% glucose and 2% xylose, and 4) YPDX: YEP with 2% dextrose and 2% xylose. Cultures were grown at 30° C. with shaking at 180 rpm. Samples were taken daily to monitor growth, which was measured by OD_{600,} and the metabolite content, which was measured by High Performance Liquid Chromatography (HPLC). The volatile compounds produced by H0 Metschnikowia sp. and FL were measured by headspace GC-MS. The OD₆₀₀ and HPLC data are the averages of three biological replicates. Standard deviations were also calculated. GC-MS data was compared roughly by the peak height.

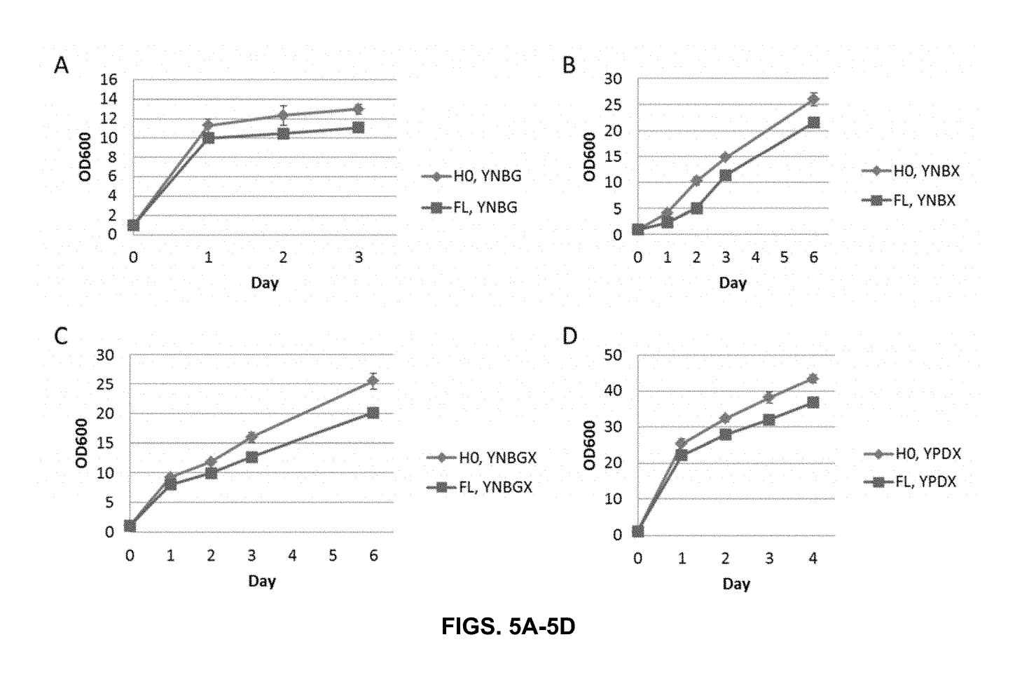

Differences were observed in the growth rate between H0 Metschnikowia sp. and FL strains in all media tested. Specifically, H0 grows faster than FL (FIGS. 5A-5D). For example, on day 3 the ratio of OD₆₀₀ with H0 Metschnikowia sp. versus FL was 1.17 in YNBG (FIG. 5A), 1.30 in YNBX (FIG. 5B), 1.26 in YNBGX (FIG. 5C), and 1.19 in YPDX (FIG. 5D).

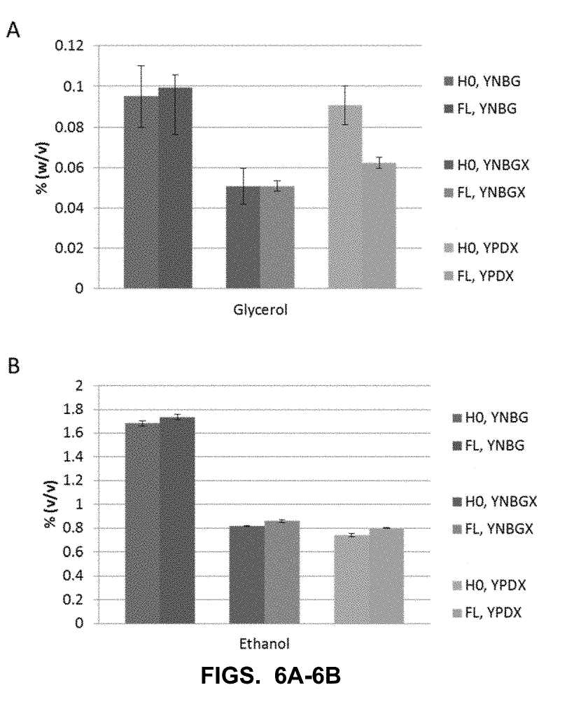

Glycerol and ethanol were detected on day 1 in the YNBG, YNBGX and YPDX media. The concentrations were similar between both strains in YNBG and YNBGX media (FIGS. 6A and 6B). However, in YPDX medium, H0 Metschnikowia sp. produced 45% more glycerol than FL (905 mg/L vs. 624 mg/L; FIG. 6A).

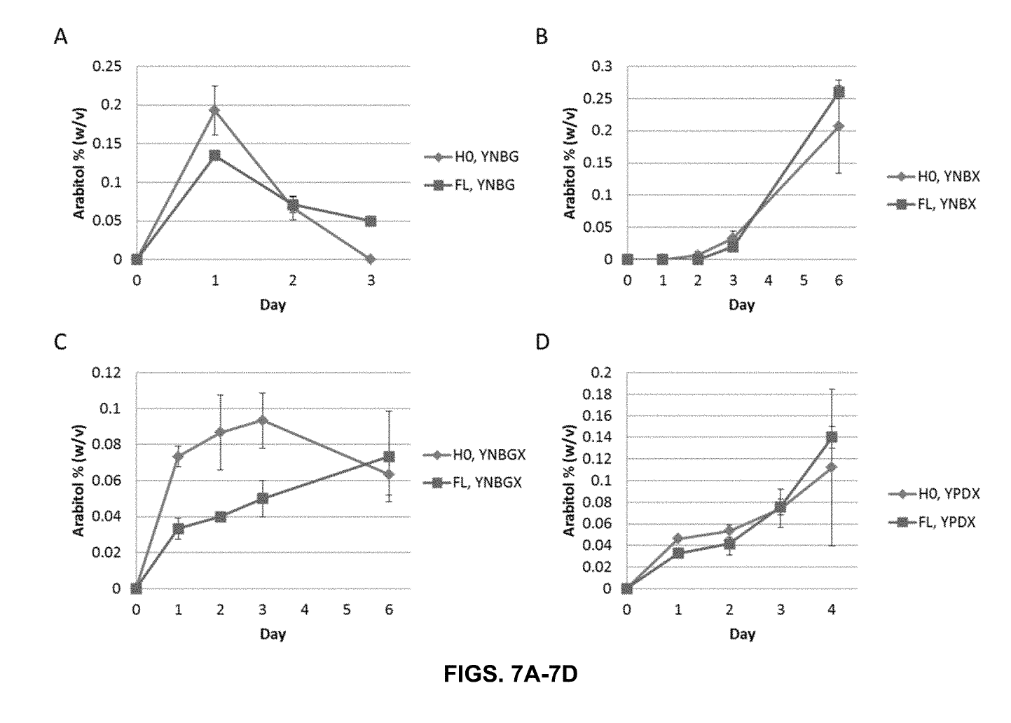

Both H0 Metschnikowia sp. and FL produced arabitol in all growth media (FIGS. 7A-7D). However, in YNBG medium, H0 Metschnikowia sp. produced 60 mg/L more arabitol than FL on day 1 (FIG. 7A). Most dramatically, in YNBGX medium, H0 Metschnikowia sp. produced a significantly higher amount of arabitol on day 1, day 2 and day 3—with H0 Metschnikowia sp. producing about 40 mg/L more arabitol than FL (FIG. 7C). In YNBX and YPDX media, the arabitol levels were similar between the two species (FIGS. 7B and 7D).

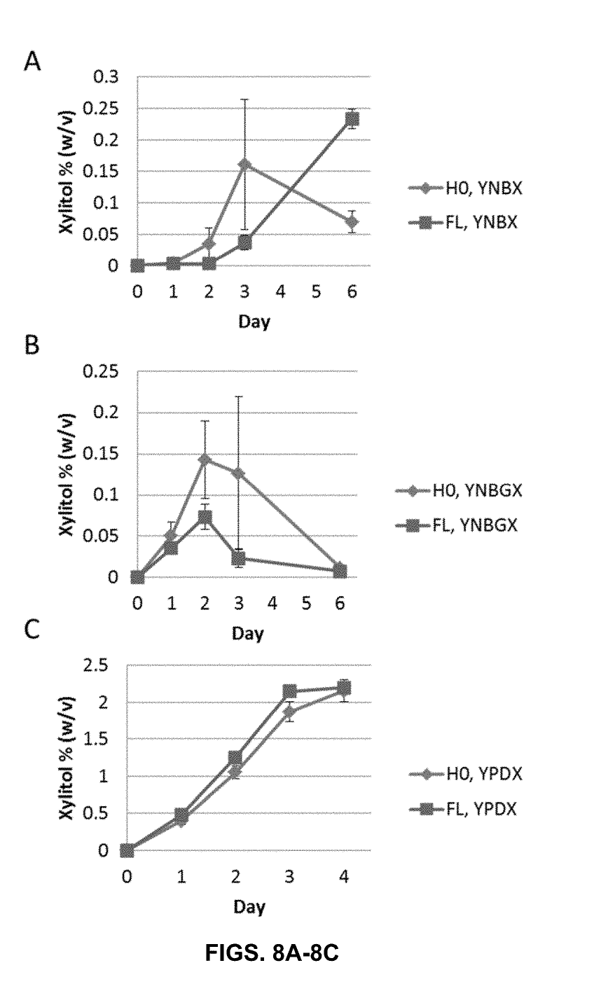

The H0 Metschnikowia sp. produced the maximum amount of xylitol on day 3 in YNBX (1.61 g/L), day 2 in YNBGX (1.43 g/L) and day 4 in YPDX (21.5 g/L) media, while FL produced maximum xylitol on day 6 in YNBX (2.33 g/L), day 2 in YNBGX (0.73 g/L) and day 4 in YPDX (21.9 g/L) (FIGS. 8A-8C). The ratio of xylitol content on day 3 between H0 Metschnikowia sp. and FL was 4.39 in YNBX, 5.43 in YNBGX and 0.87 in YPDX.

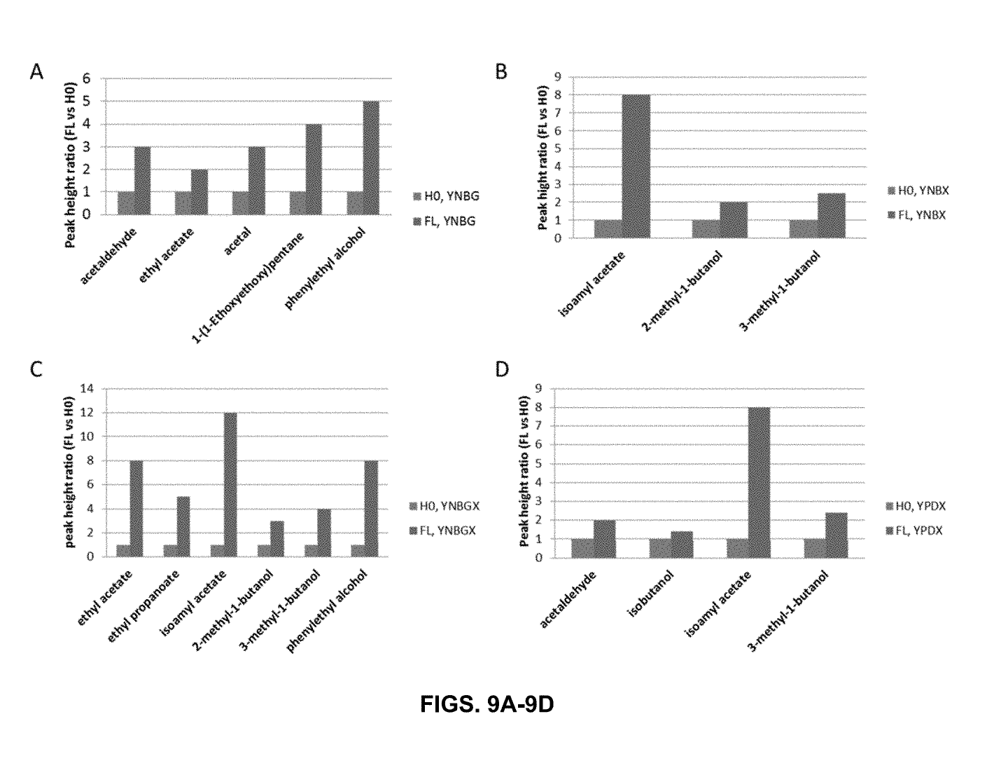

The volatile compounds in the media after growing for 1 day in YNBG and 3 days in YNBX, YNBGX, and YPDX, respectively, were measured by head space GC-MS. The peak height ratio was calculated and compared between the FL and H0 Metschnikowia sp. This analysis showed that FL produced more volatile compounds than H0 (FIGS. 9A-9D). Specifically, FL produced more acetaldehyde, ethyl acetate, acetal, 1-(1-Ethoxyethoxy) pentane, and phenylethyl alcohol in YNBG medium (FIG. 9A); more isoamyl acetate, 2-methyl-1-butanol, and 3-methyl-1-butanol in YNBX medium (FIG. 9B); more ethyl acetate, ethyl propanoate, isoamyl acetate, 2-methyl-1-butanol, 3-methyl-1-butanol, and phenylethyl alcohol in YNBGX medium (FIG. 9C) and more acetaldehyde, isobutanol, isoamyl acetate, 3-methyl-1-butanol, ethyl nonanoate, and phenylethyl alcohol in YPDX medium (FIG. 9D).

Based on the above results, the profile of growth and the secreted metabolites between H0 Metschnikowia sp. and FL species show differences in the growth rate and the content as well as the dynamics of some metabolites during the growth in different medium.

### EXAMPLE V

### Identification of H0 Metschnikowia sp. Specific Genes and Proteins

This example demonstrates that numerous genes and proteins that are unique to the H0 Metschnikowia sp. have been identified.

Homology searches were conducted using the following parameters: The genes ACT1, ARO8, ARO10, GPD1, PGK1, RPB1, RPB2, TEF1, TPI1 XKS1, TAL1 and TKL1 were identified by homology searches using corresponding protein sequences from Saccharomyces cerevisiae with program tblastn in Geneious 7.1.9 in a H0 Metschnikowia sp. whole genome comprised of shotgun contigs. The genes XYL1, XYL2,HXT2.6, QUP2, GXF1 and GXF2 were identified by homology searches of the Pichia stiptis Xyl 1, Xyl2, Hxt2.6, Qup2 and Sut1 proteins in H0 Metschnikowia sp. whole genome comprised of shotgun contigs. The genes GXS1 and XYT1 were identified by homology searches of the Candida intermedia Gxs1 and Gxf1 proteins in H0 Metschnikowia sp. whole genome comprised of shotgun contigs The HXT5 gene was identified by homology search of the Candida albicans Hxt5 protein in H0 Metschnikowia sp. whole genome comprised of shotgun contigs. The HGT19 gene was identified by searching the H0 Metschnikowia sp. transcriptome for xylose induced proteins with the gene ontology term category of “major facilitators.”

Based on the above experiments, several unique amino acid sequences corresponding to known proteins were identified. Additionally, several unique encoding nucleic acid sequences corresponding to known genes were identified. Table 6 provides a list of exemplary proteins and encoding nucleic acid sequences from the H0 Metschnikowia sp. of which all of the encoding nucleic acid sequences are unique and several of the corresponding proteins are unique.

TABLE 6

| Description | Sequence |
|---|---|
| Amino acid sequence | MCKAGFAGDDAPRAVFPSIVGRPRHQGIMVGMGQKDSYVGDEAQSKRGILTLR |
| of Act1 protein from | YPIEHGIVNNWDDMEKIWHHTFYNELRVAPEEHPVLLTEAPMNPKSNREKMTQI |
| H0 Metschnikowia sp. | MFETFNVPAFYVSIQAVLSLYSSGRTTGIVLDSGDGVTHLVPIYAGFSMPHGILRL |
| | NLAGRDLTDYLMKILSERGYTFSTTAEREIVRDIKEKLCYVALDFEQEMQTSSQS |
| | SAIEKSYELPDGQVITIGNERFRAAEALFRPTDLGLEAVGIDQTTYNSIIKCDVDV |
| | RKELYGNIVMSGGTTLFPGIAERMQKEITALAPSSMKVKIIAPPERKYSVWIGGSI |
| | LASLSTFQQMWISKQEYDESGPTIVHHKCF (SEQ ID NO: 35) |
| Amino acid sequence | MTKPLAKDLQHHLSTEAKSRKGSALKGAFKYYNQPGMTFLGGGLPLSDYFPFD |
| of Aro8 protein from | KITADVPSAPFPNGCGARVTESDKTVIEVHKRKQDNSDSGYADVELARSLQYGY |
| H0 Metschnikowia sp. | TEGHTELVQFLRDHTDTIHRVPYEDWDVITNVGNTQAWDAVLRTFTSRGDVILV |
| | EDHTFSSAMETAHAHGVTTYPVVMDTEGIVPSALEKLLDNWVGAKPRMLYTIC |
| | TGQNPTGSCLSGERRREVYSLAQKHDLIIIEDEPYYFLQMEPYTRDLALRSSKHV |
| | HGHEEFIKALVPSFISMDVDGRVLRLDSVSKTIAPGARLGWVVGQKRLLERFLRL |
| | HETSIQNASGFTQSLLNGLFQRWGQKGYLDWLIGIRAEYTHKRDVAIDALYKYF |
| | PQEVVTILPPVAGMFFVVNLDASKHPKFEELGSDPLAVENSLYEAGLAHGCLMIP |
| | GSWFKADGETTPPQAPVPVDESLKNSIFFRGTYAAVPLDELEVGLKKFGEAVKA |
| | EFGL (SEQ ID NO: 36) |
| Amino acid sequence | MAPIITRASSEETTPQITDDQIPLGEYLFLRICQANPKLRSVFGIPGDFSLALLEHLY |
| of Aro10 protein from | TKSVAKKVEFVGFCNELNAAYAADGYAKHIDGLSVLLTTFGVGELSTLNAIAGA |
| H0 Metschnikowia sp. | FTEYAPVLHIVGTTSTKQAEQSRAAGTRDVRNIHHLVQNKNPLCAPNHDVYKPM |
| | VESLSVCQESLDMNGDLNLEKIDNVLRMVTNERRPGYIFIPSDVSDIMVSAGRLN |
| | QPLTFSELTDESALKNMASRILAKLYNSKHPSVLGDALADRFGGQTALDNLVEK |
| | LPSNFVKLFSTLLARNIDETLPNYIGVYSGKLSSDKIVIDELERNTDFLLTLGHAN |
| | NEINSGVYSTDFSAITEYVEVHPDYILIDGEYVLIKNAETGKRLFSIVDLLTKLVSD |
| | FDASKMIHNNHAVNNIRARRETKQFSSLDTVSPGVITQNKLVDFFNDYLRPNDIL |
| | LCDTCSFLFGVFELKFPRGVKFIAQTLYESIGYALPATFGAARAERDLGTNRRVV |
| | LIQGDGSAQMTIQEWSTYLRYDISSPEIFLLNNEGYTVERMIKGPTRSYNDIQDT |
| | WKWTEFFKIFGDEDCEKHEAEKVNTTNELEALTRRKTSEKIRLYELKLSKLDIVD |
| | KFRILRE (SEQ ID NO: 37) |
| Amino acid sequence | MTATAPFKIESPFRIAIIGSGNWGTAVAKLVAENTAEKPEIFQKQVNMVVVFEEDI |
| of Gpd1 protein from | NGRKLTEIINTDHENVKYMPEVKLPENLVANPDIEATVKDADLLIFNIPHQFLPRV |
| H0 Metschnikowia sp. | CKQLVGKVSPTARAISCLKGLEVDASGCKLLSQSITDTLGIYCGVLSGANIANEV |
| | ARGRWSETSIAYNRPTDFRGEGKDICEFVLKEAFHRRYFHVRVIKDVIGASIAGA |
| | LKNVVAIAAGFVEGEGWGDNAKSAIMRIGLKETIHFASYWEKFGIQGLSAPEPTT |
| | FTEESAGVADLITTCSGGRNVKVARYMIEKNVDAWEAEKALLNGQSSQGIITAK |
| | EVHELLVNYKLQEEFPLFEATYAVIYENADVNTWPTILAE (SEQ ID NO: 38) |
| Amino acid sequence | MSQDELHTKSGVETPINDSLLEEKHDVTPLAALPEKSFKDYISISIFCLFVAFGGFV |
| of Gxf1 protein from | FGFDTGTISGFVNMSDFKTRFGEMNAQGEYYLSNVRTGLMVSIFNVGCAVGGIF |
| H0 Metschnikowia sp. | LCKIADVYGRRIGLMFSMVVYVVGIIIQIASTTKWYQYFIGRLIAGLAVGTVSVIS |
| | PLFISEVAPKQLRGTLVCCFQLCITLGIFLGYCTTYGTKTYTDSRQWRIPLGICFA |
| | WALFLVAGMLNMPESPRYLVEKSRIDDARKSIARSNKVSEEDPAVYTEVQLIQA |
| | GIDREALAGSATWMELVTGKPKIFRRVIMGVMLQSLQQLTGDNYFFYYGTTIFK |
| | AVGLQDSFQTSIILGIVNFASTFVGIYAIERMGRRLCLLTGSACMFVCFIIYSLIGTQ |
| | HLYKNGFSNEPSNTYKPSGNAMIFITCLYIFFFASTWAGGVYCIVSESYPLRIRSK |
| | AMSVATAANWMWGFLISFFTPFITSAIHFYYGFVFTGCLAFSFFYVYFFVVETKG |
| | LSLEEVDILYASGTLPWKSSGWVPPTADEMAHNAFDNKPTDEQV (SEQ ID NO: |
| | 39) |
| Amino acid sequence | MSAEQEQQVSGTSATIDGSASLKQEKTAEEEDAFKPKPATAYFFISFLCGLVAFG |
| of Gxf2 protein from | GYVFGFDTGTISGFVNMDDYLMRFGQQHADGTYYLSNVRTGLIVSIFNIGCAVG |
| H0 Metschnikowia sp. | GLALSKVGDIWGRRIGIMVAMIIYMVGIIIQIASQDKWYQYFIGRLITGLGVGTTS |
| | VLSPLFISESAPKHLRGTLVCCFQLMVTLGIFLGYCTTYGTKNYTDSRQWRIPLGL |
| | CFAWALLLISGMVFMPESPRFLIERQRFDEAKASVAKSNQVSTEDPAVYTEVELI |
| | QAGIDREALAGSAGWKELITGKPKMLQRVILGMMLQSIQQLTGNNYFFYYGTTI |
| | FKAVGMSDSFQTSIVLGIVNFASTFVGIWAIERMGRRSCLLVGSACMSVCFLIYSI |
| | LGSVNLYIDGYENTPSNTRKPTGNAMIFITCLFIFFFASTWAGGVYSIVSETYPLRI |
| | RSKGMAVATAANWMWGFLISFFTPFITSAIHFYYGFVFTGCLIFSFFYVFFFVRET |
| | KGLSLEEVDELYATDLPPWKTAGWTPPSAEDMAHTTGFAEAAKPTNKHV (SEQ |
| | ID NO: 40) |
| Amino acid sequence | MGLESNKLIRKYINVGEKRAGSSGMGIFVGVFAALGGVLFGYDTGTISGVMAMP |
| of Gxs1 protein from | WVKEHFPKDRVAFSASESSLIVSILSAGTFFGAILAPLLTDTLGRRWCIIISSLVVF |
| H0 Metschnikowia sp. | NLGAALQTAATDIPLLIVGRVIAGLGVGLISSTIPLYQSEALPKWIRGAVVSCYQW |
| | AITIGIFLAAVINQGTHKINSPASYRIPLGIQMAWGLILGVGMFFLPETPRFYISKG |
| | QNAKAAVSLARLRKLPQDHPELLEELEDIQAAYEFETVHGKSSWSQVFTNKNKQ |
| | LKKLATGVCLQAFQQLTGVNFIFYFGTTFFNSVGLDGFTTSLATNIVNVGSTIPGI |
| | LGVEIFGRRKVLLTGAAGMCLSQFIVAIVGVATDSKAANQVLIAFCCIFIAFFAAT |
| | WGPTAWVVCGEIFPLRTRAKSIAMCAASNWLLNWAIAYATPYLVDSDKGNLGT |
| | NVFFIWGSCNFFCLVFAYFMIYETKGLSLEQVDELYEKVASARKSPGFVPSEHAF |
| | REHADVETAMPDNFNLKAEAISVEDASV (SEQ ID NO: 41) |
| Amino acid sequence | MSEKPVVSHSIDTTSSTSSKQVYDGNSLLKTSNERDGERGNILSQYTEEQAMQM |
| of Hgt19 protein from | GRNYALKHNLDATLFGKAAAVARNPYEFNSMSFLTEEEKVALNTEQTKKWHIP |
| H0 Metschnikowia sp. | RKLVEVIALGSMAAAVQGMDESVVNGATLFYPTAMGITDIKNADLIEGLINGAP |
| | YLCCAIMCWTSDYWNRKLGRKWTIFWTCAISAITCIWQGLVNLKWYHLFIARFC |
| | LGFGIGVKSATVPAYAAETTPAKIRGSLVMLWQFFTAVGIMLGYVASLAFYYIG |
| | DNGISGGLNWRLMLGSACLPAIVVLVQVPFVPESPRWLMGKERHAEAYDSLRQ |
| | LRFSEIEAARDCFYQYVLLKEEGSYGTQPFFSRIKEMFTVRRNRNGALGAWIVMF |
| | MQQFCGINVIAYYSSSIFVESNLSEIKAMLASWGFGMINFLFAIPAFYTIDTFGRRN |
| | LLLTTFPLMAVFLLMAGFGFWIPFETNPHGRLAVITIGIYLFACVYSAGEGPVPFT |
| | YSAEAFPLYIRDLGMGFATATCWFFNFILAFSWPRMKNAFKPQGAFGWYAAWN |
| | IVGFFLVLWFLPETKGLTLEELDEVFDVPLRKHAHYRTKELVYNLRKYFLRQNP |
| | KPLPPLYAHQRMAVTNPEWLEKTEVTHEENI (SEQ ID NO: 42) |
| Amino acid sequence | MSSTTDTLEKRDTEPFTSDAPVTVHDYIAEERPWWKVPHLRVLTWSVFVITLTST |
| of Hxt2.6 protein from | NNGYDGSMLNGLQSLDIWQEDLGHPAGQKLGALANGVLFGNLAAVPFASYFCD |
| H0 Metschnikowia sp. | RFGRRPVICFGQILTIVGAVLQGLSNSYGFFLGSRIVLGFGAMIATIPSPTLISEIAY |
| | PTHRETSTFAYNVCWYLGAIIASWVTYGTRDLQSKACWSIPSYLQAALPFFQVC |
| | MIWFVPESPRFLVAKGKIDQARAVLSKYHTGDSTDPRDVALVDFELHEIESALEQ |
| | EKLNTRSSYFDFFKKRNFRKRGFLCVMVGVAMQLSGNGLVSYYLSKVLDSIGIT |
| | ETKRQLEINGCLMIYNFVICVSLMSVCRMFKRRVLFLTCFSGMTVCYTIWTILSA |
| | LNEQRHFEDKGLANGVLAMIFFYYFFYNVGINGLPFLYITEILPYSHRAKGLNLF |
| | QFSQFLTQIYNGYVNPIAMDAISWKYYIVYCCILFVELVIVFFTFPETSGYTLEEV |
| | AQVFGDEAPGLHNRQLDVAKESLEHVEHV (SEQ ID NO: 43) |
| Amino acid sequence | MSIFEGKDGKGVSSTESLSNDVRYDNMEKVDQDVLRHNFNFDKEFEELEIEAAQ |
| of Hxt5 protein from | VNDKPSFVDRILSLEYKLHFENKNHMVVVLLGAFAAAAGLLSGLDQSIISGASIGM |
| H0 Metschnikowia sp. | NKALNLTEREASLVSSLMPLGAMAGSMIMTPLNEWFGRKSSLIISCIWYTIGSAL |
| | CAGARDHHMMYAGRFILGVGVGIEGGCVGIYISESVPANVRGSIVSMYQFNIAL |
| | GEVLGYAVAAIFYTVHGGWRFMVGSSLVFSTILFAGLFFLPESPRWLVHKGRNG |
| | MAYDVVVKRLRDINDESAKLEFLEMRQAAYQERERRSQESLFSSWGELFTIARNR |
| | RALTYSVIMITLGQLTGVNAVMYYMSTLMGAIGFNEKDSVFMSLVGGGSLLIGT |
| | IPAILWMDRFGRRVVVGYNLVGFFVGLVLVGVGYRFNPVTQKAASEGVYLTGLI |
| | VYFLFFGSYSTLTWVIPSESFDLRTRSLGMTICSTFLYLWSFTVTYNFTKMSAAFT |
| | YTGLTLGFYGGIAFLGLIYQVCFMPETKDKTLEEIDDIFNRSAFSIARENISNLKKG |
| | IW (SEQ ID NO: 44) |
| Amino acid sequence | MSLSNKLSVKDLDLANKRVFIRVDFNVPLDGTTITNNQRIVAALPTIKYVLEQKP |
| of Pgk1 protein from | KAVILASHLGRPNGERVEKYSLAPVAKELQSLLSDQKVTFLNDSVGPEVEKAVN |
| H0 Metschnikowia sp. | SASQGEVFLLENLRYHIEEEGSKKVDGNKVKASKEDVEKFRQGLTALADVYVN |
| | DAFGTAHRAHSSMVGLELPQKAAGFLMAKELEYFAKALENPTRPFLAILGGAKV |
| | SDKIQLIDNLLDKVDILIVGGGMAFTFKKVLDNMPIGTSLFDEAGSKNVENLIAK |
| | AKKNNVEIVLPVDFVTADDFNKDANTGVATQEEGIPDGWMGLDAGPKSRELFA |
| | EAVAKAKTIVVVNGPPGVFEFEKFAQGTKSLLDAAVKSAEAGNTVIIGGGDTATV |
| | AKKFGVVEKLSHVSTGGGASLELLEGKELPGVVAISDKQ (SEQ ID NO: 45) |
| Amino acid sequence | MGFRNLKRRLSNVGDSMSVHSVKEEEDFSRVEIPDEIYNYKIVLVALTAASAAIII |
| of Qup2 protein from | GYDAGFIGGTVSLTAFKSEFGLDKMSATAASAIEANVVSVFQAGAYFGCLFFYPI |
| H0 Metschnikowia sp. | GEIWGRKIGLLLSGFLLTFGAAISLISNSSRGLGAIYAGRVLTGLGIGGCSSLAPIY |
| | VSEIAPAAIRGKLVGCWEVSWQVGGIVGYWINYGVLQTLPISSQQWIIPFAVQLIP |
| | SGLFWGLCLLIPESPRFLVSKGKIDKARKNLAYLRGLSEDHPYSVFELENISKAIE |
| | ENFEQTGRGFFDPLKALFFSKKMLYRLLLSTSMFMMQNGYGINAVTYYSPTIFKS |
| | LGVQGSNAGLLSTGIFGLLKGAASVFWVFFLVDTFGRRFCLCYLSLPCSICMWYI |
| | GAYIKIANPSAKLAAGDTATTPAGTAAKAMLYIWTIFYGITWNGTTWVICAEIFP |
| | QSVRTAAQAVNASSNWFWAFMIGHFTGQALENIGYGYYFLFAACSAIFPVVVW |
| | FVYPETKGVPLEAVEYLFEVRPWKAHSYALEKYQIEYNEGEFHQHKPEVLLQGS |
| | ENSDTSEKSLA (SEQ ID NO: 46) |
| Amino acid sequence | MDQTTKKPRDGGLNDPRLGSIDRNFKCQTCGEDMAECPGHFGHIELAKPVFHIG |
| of Rpb1 protein from | FIAKIKKVCECVCMHCGKLLVDDANPLMAQAIRIRDPKKRFNAVWNVSKTKMV |
| H0 Metschnikowia sp. | CEADTINEEGQVTAGRGGCGHTQPTVRRDGLKLWGTWKQNKTYDENEQPERR |
| | LLSPSEILSVFRHISPEDCHKLGFNEDYARPEWMLITVLPVPPPPVRPSIAFNDTAR |
| | GEDDLTFKLADILKANINVQRLEIDGSPQHVISEFEALLQFHVATYMDNDIAGQP |
| | QALQKTGRPIKSIRARLKGKEGRLRGNLMGKRVDFSARTVISGDPNLDLDQVGV |
| | PISIARTLTYPEVVTPYNIHKLTEYVRNGPNEHPGAKYVIRDTGDRIDLMYNKRA |
| | GDIALQYGWKVERHLMDDDPVLFNRQPSLHKMSMMAHRVKVMPYSTFRLNLS |
| | VTSPYNADFDGDEMNLHVPQSPETRAEMSQICAVPLQIVSPQSNKPVMGIVQDT |
| | LCGIRKMTLRDNFIEYEQVMNMLYWIPNWDGVIPPPAVLKPKPLWSGKQLLSM |
| | AIPKGIHLQRFDDGRDMLSPKDSGMLIVDGEIIFGVVDKKTVGATGGGLIHTVMR |
| | EKGPYVCAQLFSSIQKVVNYWLLHNGFSIGIGDTIADKDTMRDVTTTIQEAKQK |
| | VQEIIIDAQQNKLEPEPGMTLRESFEHNVSRILNQARDTAGRSAEMNLKDSNNVK |
| | QMVTSGSKGSFINISQMSACVGQQIVEGKRIPFGFGDRTLPHFTKDDYSPESKGFV |
| | ENSYLRGLTPQEFFFHAMAGREGLIDTAVKTAETGYIQRRLVKALEDIMVHYDG |
| | TTRNSLGDIIQFVYGEDGIDATSVEKQSVDTIPGSDSSFEKRYRIDVLDPAKSIPES |
| | LLESGKQIKGDVAVQKVLDEEYDQLLKDRKFLREVVFPNGDYNWPLPVNLRRII |
| | QNAQQIFHSGRQKASDLRLEEIVEGVQSLCTKLLVLRGKTELIKEAQENATLLFQ |
| | CLLRSRLAARRVIEEFKLNKVSFEWVCGEIESQFQKSIVHPGEMVGVVAAQSIGE |
| | PATQMTLNTFHYAGVSSKNVTLGVPRLKEILNVAKNIKTPALTVYLEPEIAVDIE |
| | KAKVVQSAIEHTTLKNVTSSTEIYYDPDPRSTVIEEDYDTVEAYFAIPDEKVEETI |
| | DNQSPWLLRLELDRAKMLDKQLTMAQVAEKISQNFGEDLFVIWSDDTADKLIIR |
| | CRVIRDPKLEEEGEHEEDQILKRVEAHMLETISLRGIPGITRVFMMQHKMSTPDA |
| | DGEFSQKQEWVLETDGVNLAEVITVPGVDASRTYSNNFIEILSVLGIEATRTALFK |
| | EILNVIAFDGSYVNYRHMALLVDVMTARGHLMAITRHGINRAETGALMRCSFEE |
| | TVEILLDAGAAAELDDCRGISENVILGQMPPLGTGAFDVMVDEKMLQDASVSSD |
| | IGVAGQTDGGATPYRDYEMEDDKIQFEEGAGFSPIHTANVSDASGSLTSYGGQPS |
| | MVSPTSPFSFGATSPGYGGVTSPAYGATSPTYSPTSPTYSPTSPSYSPTSPSYSPTSP |
| | SYSPTSPSYSPTSPSYSPTSPSYSPTSPSYSPTSPSYSPTSPSYSPTSPSYSPTSPSYSP |
| | TSPSYSPTSPSYSPTSPSYSPTSPSYSPTSPSYSPTSPSYSPTSPSYSPTSPQYSPTSPS |
| | YSPTSPQYSPTSPSYSPTSPQYSPTSPSYSPTSPQYSPTSPQYSPGSPAYSPGSPSYST |
| | EKKDEDKK (SEQ ID NO: 47) |
| Amino acid sequence | MSQEPVEDPYVYDEEDAHSITPEDCWTVISSFFQEKGLVSQQLDSFDEFIESNIQE |
| of Rpb2 protein from | LVWEDSHLILDQPAQHTSEDQYENKRFEITFGKIYISKPTQTEGDGTTHPMFPQEA |
| H0 Metschnikowia sp. | RLRNLTYSSPLYVDMSKKKFLSDDRVRKGNELEWVEEKVDGEEAQSKVFLGKV |
| | PIMLRSKFCMLRDLGEHEFYELKECPYDMGGYFVINGSEKVLIAQERSAANIVQV |
| | FKKAAPSPISHVAEIRSALEKGSRLISSMQIKLYGRDDKGTTGRTIKATLPYIKEDI |
| | PIVIVFRALGVVPDGDILEHICYDANDWQMLEMLKPCVEEGFVIQEREVALDFIG |
| | RRGVLGIRREKRIQYAKDILQKELLPNITQEAGFESRKAFFLGYMVNRLLLCALE |
| | RKEPDDRDHFGKKRLDLAGPLLASLFRLLFKKLTRDIYNYMQRCVENDKEFNLT |
| | LAVKSQTITDGLRYSLATGNWGEQRKAMSARAGVSQVLNRYTYSSTLSHLRRT |
| | NTPIGRDGKIAKPRQLHNTHWGLVCPAETPEGQACGLVKNLSLMTCISVGTSSEP |
| | ILYFLEEWGMEPLEDYVPSNAPDCTRVFVNGVVVVGTHREPAQLVDTMRRLRRK |
| | GDISPEVSIIRDIREMEFKIFTDAGRVYRPLFIVDDDPESETKGELMLQKEHVHKLL |
| | NSAYDEYDEDDSNAYTWSSLVNDGVVEYVDAEEEETIMIAMTPEDLEASKSALS |
| | ETQQQDLQMEEQELDPAKRIKPTYTSSTHTFTHCEIHPSMILGVAASIIPFPDHNQS |
| | PRNTYQSAMGKQAMGVFLTNYAVRMDTMANILYYPQKPLATTRAMEHLKFRE |
| | LPAGQNAVVAIACYSGYNQEDSMIMNQSSIDRGLFRSLFFRSYMDLEKRQGMKA |
| | LETFEKPSRSDTLRLKHGTYEKLDDDGLIAPGVRVSGEDIIIGKTTPIPPDTEELGQ |
| | RTQYHTKRDASTPLRSTESGIVDQVLLTTNGDGAKFVKVRMRTTKVPQIGDKFA |
| | SRHGQKGTIGVTYRHEDMPFSAQGIVPDLIINPHAIPSRMTVAHLIECLLSKVSSLS |
| | GLEGDASPFTDVTAEAVSKLLREHGYQSRGFEVMYNGHTGKKMMAQVFFGPT |
| | YYQRLRHMVDDKIHARARGPVQVLTRQPVEGRSRDGGLRFGEMERDCMIAHG |
| | AAGFLKERLMEASDAFRVHVCGICGLMSVIANLKKNQFECRSCKNKTNIYQIHIP |
| | YAAKLLFQELMAMNISPRLYTERSGISVRV (SEQ ID NO: 48) |
| Amino acid sequence | MGKEKSHVNVVVIGHVDSGKSTTTGHLIYKCGGIDKRTIEKFEKEAAELGKGSF |
| of Tef1 protein from | KYAWVLDKLKAERERGITIDIALWKFETPKYHVTVIDAPGHRDFIKNMITGTSQA |
| H0 Metschnikowia sp. | DCAILIIAGGVGEFEAGISKDGQTREHALLAYTLGVRQLIVAVNKMDSVKWDKN |
| | RFEEIIKETSNFVKKVGYNPKTVPFVPISGWNGDNMIEASTNCPWYKGWEKETK |
| | AGKSSGKTLLEAIDAIEPPTRPTDKALRLPLQDVYKIGGIGTVPVGRVETGVIKAG |
| | MVVTFAPAGVTTEVKSVEMHHEQLVEGLPGDNVGFNVKNVSVKEIRRGNVCG |
| | DSKQDPPKAAASFTAQVIVLNHPGQISSGYSPVLDCHTAHIACKFDTLLEKIDRRT |
| | GKSLESEPKFVKSGDAAIVKMVPTKPMCVEAFTDYPPLGRFAVRDMRQTVAVG |
| | VIKAVEKSDKAGKVTKAAQKAAKK (SEQ ID NO: 49) |
| Amino acid sequence | MARQFFVGGNFKMNGTKESLTAIVDTLNKADLPENVEVVIAPPAPYLSLVVEAN |
| of Tpi1 protein from | KQKTVEVAAQNVFSKASGAYTGEIAPQQLKDLGANWTLTGHSERRTIIKESDEFI |
| H0 Metschnikowia sp. | AEKTKFALESGVSVILCIGETLEEKKAGITLEVCARQLDAVSKIVSDWTNVVIAY |
| | EPVWAIGTGLAATAQDAQDIHKEIRAHLSKTIGAEQAEAVRILYGGSVNGKNAV |
| | DFKDKADVDGFLVGGASLKPEFIDIIKSRL (SEQ ID NO: 50) |
| Amino acid sequence | MTYSSSSGLFLGFDLSTQQLKIIVTNENLKALGTYHVEFDAQFKEKYAIKKGVLS |
| of Xks1 protein from | DEKTGEILSPVHMWLEAIDHVFGLMKKDNFPFGKVKGISGSGMQHGSVFWSKS |
| H0 Metschnikowia sp. | ASSSLKNMAEYSSLTEALADAFACDTSPNWQDHSTGKEIKDFEKVVGGPDKLAE |
| | ITGSRAHYRFTGLQIRKLAVRSENDVYQKTDRISLVSSFVASVLLGRITTIEEADA |
| | CGMNLYNVTESKLDEDLLAIAAGVHPKLDNKSKRETDEGVKELKRKIGEIKPVS |
| | YQTSGSIAPYFVEKYGFSPDSKIVSFTGDNLATIISLPLRKNDVLVSLGTSTTVLLV |
| | TESYAPSSQYHLFKHPTIKNAYMGMICYSNGALARERVRDAINEKYGVAGDSW |
| | DKFNEILDRSGDFNNKLGVYFPIGEIVPNAPAQTKRMEMNSHEDVKEIEKWDLE |
| | NDVTSIVESQTVSCRVRAGPMLSGSGDSNEGTPENENRKVKTLIDDLHSKFGEIY |
| | TDGKPQSYESLTSRPRNIYFVGGASRNKSIIHKMASIMGATEGNFQVEIPNACALG |
| | GAYKASWSLECESRQKWVHFNDYLNEKYDFDDVDEFKVDDKWLNYIPAIGLLS |
| | KLESNLDQN (SEQ ID NO: 51) |
| Amino acid sequence | MATIKLNSGYDMPQVGFGCWKVTNSTCADTIYNAIKVGYRLFDGAEDYGNEKE |
| of Xyl1 protein from | VGEGINRAIDEGLVARDELFVVSKLWNNFHHPDNVEKALDKTLGDLNVEYLDL |
| H0 Metschnikowia sp. | FLIHFPIAFKFVPFEEKYPPGFYCGEGDKFIYEDVPLLDTWRALEKFVKKGKIRSIG |
| | ISNFSGALIQDLLRGAEIPPAVLQIEHHPYLQQPRLIEYVQSKGIAITAYSSFGPQSF |
| | VELDHPKVKECVTLFEHEDIVSIAKAHDKSAGQVLLRWATQRGLAVIPKSNKTE |
| | RLLSNLNVNDFDLSEAELEQIAKLDVGLRFNNPWDWDKIPIFH (SEQ ID NO: 52) |
| Amino acid sequence | MPANPSLVLNKVNDITFENYEVPLLTDPNDVLVQVKKTGICGSDIHYYTHGRIGD |
| of Xyl2 protein from | FVLTKPMVLGHESAGVVVEVGKGVTDLKVGDKVAIEPGVPSRTSDEYKSGHYN |
| H0 Metschnikowia sp. | LCPHMCFAATPNSNPDEPNPPGTLCKYYKSPADFLVKLPEHVSLELGAMVEPLT |
| | VGVHASRLGRVTFGDHVVVFGAGPVGILAAAVARKFGAASVTIVDIFDSKLELA |
| | KSIGAATHTFNSMTEGVLSEALPAGVRPDVVLECTGAEICVQQGVLALKAGGRH |
| | VQVGNAGSYLKFPITEFVTKELTLFGSFRYGYNDYKTSVAILDENYKNGKENAL |
| | VDFEALITHRFPFKNAIEAYDAVRAGDGAVKCIIDGPE (SEQ ID NO: 53) |
| Amino acid sequence | MGYEEKLVAPALKFKNFLDKTPNIHNVYVIAAISCTSGMMFGFDISSMSVFVDQ |
| of Xyt1 protein from | QPYLKMFDNPSSVIQGFITASMSLGSFFGSLTSTFISEPFGRRASLFICGILWVIGAA |
| H0 Metschnikowia sp. | VQSSSQNRAQLICGRIIAGWGIGFGSSVAPVYGSEMAPRKIRGTIGGIFQFSVTVGI |
| | FIMFLIGYGCSFIQGKASFRIPWGVQMVPGLILLIGLFFIPESPRWLAKQGYWEDA |
| | EIIVANVQAKGNRNDANVQIEMSEIKDQLMLDEHLKEFTYADLFTKKYRQRTIT |
| | AIFAQIWQQLTGMNVMMYYIVYIFQMAGYSGNTNLVPSLIQYIINMAVTVPALF |
| | CLDLLGRRTILLAGAAFMMAWQFGVAGILATYSEPAYISDTVRITIPDDHKSAAK |
| | GVIACCYLFVCSFAFSWGVGIWVYCSEVVVGDSQSRQRGAALATSANWIFNFAIA |
| | MFTPSSFKNITWKTYIIYATFCACMFIHVFFFFPETKGKRLEEIGQLWDEGVPAWR |
| | SAKWQPTVPLASDAELAHKMDVAHAEHADLLATHSPSSDEKTGTV (SEQ ID |
| | NO: 54) |
| Amino acid sequence | MSNSLESLKATGTVIVTDTGEFDSIAKYTPQDATTNPSLILAASKKAEYAKVIDV |
| of Tal1 protein from | AIKYAEDKGSNPKEKAAIALDRLLVEFGKEILSIVPGRVSTEVDARLSFDKDATV |
| H0 Metschnikowia sp. | KKALEIIELYKSIGISKDRVLIKIASTWEGIQAAKELEAKHDIHCNLTLLFSFVQAV |
| | ACAEAKVTLISPFVGRILDWYKASTGKEYDAESDPGVVSVRQIYNYYKKYGYNT |
| | IVMGASFRNTGEIKALAGCDYLTVAPKLLEELMNSSEEVPKVLDAASASSASEEK |
| | VSYIDDESEFRFLLNEDAMATEKLAQGIRGFAKDAQTLLAELENRFK (SEQ ID |
| | NO: 55) |
| Amino acid sequence | MSDIDQLAISTIRLLAVDAVAKANSGHPGAPLGLAPAAHAVVVKEMKFNPKNPD |
| of Tkl1 protein from | WVNRDRFVLSNGHACALLYAMLHLYGFDMSLDDLKQFRQLNSKTPGHPEKFEI |
| H0 Metschnikowia sp. | PGAEVTTGPLGQGISNAVGLAIAQKQFAATFNKDDFAISDSYTYAFLGDGCLME |
| | GVASEASSLAGHLQLNNLIAFWDDNKISIDGSTEVAFTEDVLKRYEAYGWDTLTI |
| | EKGDTDLEGVAQAIKTAKASKKPTLIRLTTIIGYGSLQQGTHGVHGAPLKPDDIK |
| | QLKEKFGFDPTKSFVVPQEVYDYYGTLVKKNQELESEWNKTVESYIQKFPEEGA |
| | VLARRLKGELPEDWAKCLPTYTADDKPLATRKLSEMALIKILDVVPELIGGSADL |
| | TGSNLTRAPDMVDFQPPQTGLGNYAGRYIRYGVREHGMGAIMNGIAGFGAGFR |
| | NYGGTFLNFVSYAAGAVRLSALSHLPVIWVATHDSIGLGEDGPTHQPIETLAHFR |
| | ATPNISVVVRPADGNEVSAAYKSAIESTSTPHILALTRQNLPQLAGSSVEKASTGG |
| | YTVYQTTDKPAVIIVASGSEVAISIDAAKKLEGEGIKANVVSLVDFHTFDKQPLD |
| | YRLSVLPDGVPIMSVEVMSSFGWSKYSHEQFGLNRFGASGKAEDLYKFFDFTPE |
| | GVADRAAKTVQFYKGKDLLSPLNRAF (SEQ ID NO: 56) |
| Nucleotide sequence | ATGTGCAAAGCCGGTTTTGCCGGTGACGACGCACCTCGTGCTGTGTTCCCATC |
| of ACT1 gene from H0 | TATCGTGGGTAGACCAAGACACCAGGGTATCATGGTCGGCATGGGTCAAAAG |
| Metschnikowia sp. | GACTCTTATGTTGGTGACGAGGCCCAGTCCAAGAGAGGTATTTTGACTTTGA |
| | GATACCCCATTGAGCATGGTATCGTGAACAACTGGGACGACATGGAGAAGAT |
| | CTGGCATCACACCTTCTACAACGAGTTGAGAGTCGCCCCTGAGGAACACCCA |
| | GTCTTGTTGACCGAGGCTCCAATGAACCCTAAGTCCAACAGAGAGAAGATGA |
| | CTCAAATCATGTTCGAGACTTTCAACGTTCCGGCTTTCTACGTTTCCATCCAG |
| | GCCGTCTTGTCCTTGTACTCCTCCGGTAGAACCACTGGTATTGTTTTAGATTCT |
| | GGTGACGGTGTTACTCACTTGGTTCCTATCTATGCTGGATTCTCCATGCCTCA |
| | CGGTATTTTGAGATTGAACTTGGCTGGTAGAGACTTGACCGACTACTTGATG |
| | AAGATTTTGTCCGAGCGTGGTTACACTTTCTCCACCACTGCCGAGAGAGAAA |
| | TTGTCCGTGACATCAAGGAGAAATTGTGCTACGTCGCCTTGGACTTTGAGCA |
| | GGAGATGCAAACGTCTTCTCAATCTTCCGCTATCGAGAAATCGTACGAGTTG |
| | CCAGATGGACAAGTCATCACTATTGGTAACGAGAGATTTAGAGCTGCCGAGG |
| | CCTTGTTCCGTCCTACTGACTTGGGCTTGGAGGCTGTTGGTATCGACCAAACC |
| | ACTTACAACTCTATCATCAAGTGTGACGTCGACGTTAGAAAGGAGTTGTACG |
| | GTAACATTGTTATGTCCGGTGGTACTACTTTATTCCCAGGTATTGCTGAGCGT |
| | ATGCAAAAGGAGATTACCGCGTTGGCTCCTTCCTCCATGAAGGTCAAGATTA |
| | TTGCTCCACCTGAGAGAAAGTACTCTGTATGGATTGGTGGCTCCATCTTGGCT |
| | TCCTTGTCCACTTTCCAACAGATGTGGATCTCGAAGCAAGAGTACGACGAGT |
| | CTGGACCAACTATCGTTCACCACAAGTGTTTTTAA (SEQ ID NO: 57) |
| Nucleotide sequence | ATGACTAAACCACTTGCTAAGGATTTGCAGCACCACTTGAGCACGGAGGCCA |
| of AR08 gene from | AGTCACGCAAGGGCCTGGCGCTTAAGGGCGCATTCAAGTACTACAACCAGCC |
| H0 Metschnikowia sp. | CGGGATGACGTTTCTCGGCGGCGGATTGCCCCTTCTGGACTATTTCCCCTTTG |
| | ATAAAATCACTGCGGACGTGCCGCTGGCGCCGTTCCCAAACGGATGTGGTGC |
| | GAGAGTCACCGAATCAGACAAAACCGTGATTGAGGTGCATAAGCGGAAACA |
| | AGACAACAGTGACAGCGGCTACGCGGACGTTGAGTTGGCGCGTAGTTTGCAG |
| | TACGGATACACGGAGGGACACACTGAGCTTGTGCAGTTCTTACGTGACCACA |
| | CCGACACGATCCACCGCGTGCCATATGAAGATTGGGACGTGATCACCAATGT |
| | GGGCAACACGCAAGCGTGGGACGCCGTGTTGCGGACGTTTACGCTGCGTGGT |
| | GACGTGATCTTGGTGGAAGACCACACCTTTTCGCTGGCCATGGAGACCGCGC |
| | ACGCGCACGGCGTCACCACTTATCCCGTGGTGATGGACACCGAGGGAATCGT |
| | GCCATCGGCGTTGGAGAAACTCTTGGACAACTGGGTTGGCGCAAAGCCGCGC |
| | ATGCTCTACACGATCTGCACGGGACAGAACCCAACTGGATCGTGTCTCAGTG |
| | GGGAACGCCGCCGCGAGGTGTATCTGTTGGCACAGAAACATGATTTGATCAT |
| | CATCGAGGACGAGCCGTACTACTTCTTGCAGATGGAGCCATATACACGTGAT |
| | TTGGCGCTTCGCCTGCTGAAGCACGTGCACGGCCATGAGGAGTTCATCAAGG |
| | CGCTTGTTCCCTCGTTCATCTCGATGGACGTGGACGGACGTGTGCTCCGACTC |
| | GACTCCGTGTCGAAGACGATCGCTCCAGGCGCCCGTTTGGGCTGGGTCGTGG |
| | GGCAGAAACGCCTCTTGGAGCGATTCTTGCGTTTGCACGAAACGTCGATCCA |
| | GAACGCTTCGGGTTTCACGCAGCTGCTCTTGAACGGCTTGTTTCAAAGATGG |
| | GGCCAGAAGGGATACTTGGACTGGTTGATTGGTATCCGTGCTGAGTACACTC |
| | ACAAGAGGGACGTGGCAATTGATGCTTTATACAAGTACTTCCCGCAAGAAGT |
| | AGTGACGATTTTGCCGCCCGTGGCCGGTATGTTCTTTGTTGTCAACTTGGACG |
| | CCAGCAAGCACCCGAAATTTGAGGAGTTGGGCAGCGACCCGTTGGCTGTCGA |
| | GAACAGCCTCTACGAGGCTGGCTTGGCGCACGGGTGCTTGATGATTCCTGGC |
| | TCGTGGTTCAAGGCTGACGGCGAGACCACCCCGCCACAAGCGCCTGTGCCTG |
| | TGGACGAGCTGTTGAAGAACAGCATTTTCTTTAGGGGTACTTACGCGGCAGT |
| | ACCCTTGGACGAGTTGGAGGTTGGCTTGAAGAAGTTTGGCGAGGCTGTCAAG |
| | GCCGAGTTTGGTTTGTAA (SEQ ID NO: 58) |
| Nucleotide sequence | ATGGCACCAATCATCACCAGGGCTTCATCCGAAGAAACAACACCCCAAATTA |
| of ARO10 gene from | CAGACGACCAGATCCCTTTGGGGGAGTACCTTTTCCTCAGAATCTGCCAGGC |
| H0 Metschnikowia sp. | AAATCCAAAACTTCGCTCGGTGTTTGGCATTCCCGGAGACTTCAGTTTGGCGT |
| | TATTGGAGCATCTCTATACCAAGCTGGTGGCGAAAAAAGTTGAGTTTGTTGG |
| | TTTCTGTAACGAGCTCAATGCGGCATATGCAGCAGATGGATATGCAAAGCAT |
| | ATTGACGGCTTGAGTGTCTTGCTTACGACTTTTGGGGTGGGAGAACTATCCAC |
| | TTTGAACGCCATAGCCGGCGCATTCACAGAGTACGCTCCAGTATTGCATATT |
| | GTCGGCACCACATCTACGAAACAGGCGGAGCAGTCCAGGGCGGCAGGCACG |
| | AGAGATGTAAGAAACATCCATCACTTGGTGCAGAACAAAAACCCGCTTTGTG |
| | CGCCCAATCACGATGTATATAAGCCCATGGTGGAAAGTTTATCTGTATGCCA |
| | GGAATCCTTGGACATGAATGGCGACTTGAACTTGGAAAAGATCGATAACGTC |
| | TTGAGAATGGTCACAAATGAGAGGAGACCAGGGTACATTTTCATTCCGAGCG |
| | ATGTTTCCGATATCATGGTTTCCGCAGGCAGGTTGAATCAGCCGTTGACCTTT |
| | AGTGAATTGACAGATGAGTCTGCGTTGAAAAACATGGCCCTGAGAATTTTGG |
| | CAAAACTTTACAATTCAAAGCACCCTTCTGTACTTGGCGATGCATTAGCAGA |
| | CAGGTTTGGGGGGCAAACTGCTTTGGATAACCTTGTTGAAAAGTTACCATCG |
| | AATTTCGTCAAGTTGTTTTCCACGCTTTTGGCCAGAAACATCGACGAGACTTT |
| | ACCGAACTATATCGGGGTCTACAGCGGCAAATTGTCCTCCGATAAGATTGTC |
| | ATTGACGAATTGGAGAGAAACACCGACTTTTTGTTGACCCTCGGCCATGCTA |
| | ACAATGAGATCAATTCCGGGGTATACTCAACTGACTTTTCTGCAATCACCGA |
| | GTATGTGGAGGTGCATCCAGATTACATTCTCATTGATGGCGAGTACGTTCTCA |
| | TCAAAAACGCAGAAACCGGAAAGAGATTGTTTTCAATTGTTGATTTGCTTAC |
| | TAAGCTTGTCTCAGATTTCGATGCATCGAAGATGATTCACAACAATCATGCTG |
| | TTAACAACATTAGAGCGAGGCGCGAAACCAAGCAGTTTTCGTCATTGGATAC |
| | GGTTTCGCCTGGAGTGATCACGCAAAACAAGTTGGTTGATTTTTTCAATGACT |
| | ACTTGCGGCCAAACGATATCTTGTTGTGCGATACATGCAGTTTTCTTTTTGGT |
| | GTGTTCGAGCTTAAGTTCCCGAGGGGCGTCAAGTTTATTGCACAAACCTTATA |
| | CGAATCGATCGGGTATGCACTTCCCGCGACTTTTGGCGCTGCAAGGGCCGAA |
| | AGGGATTTGGGCACGAACAGAAGAGTGGTGTTGATACAGGGAGATGGTTCT |
| | GCCCAAATGACAATCCAGGAATGGTCCACATATTTGAGATACGACATTCTGT |
| | CGCCAGAAATCTTTTTGCTCAACAACGAGGGCTACACGGTTGAAAGGATGAT |
| | CAAAGGGCCCACTCGGTCCTATAACGATATTCAGGACACTTGGAAATGGACG |
| | GAATTTTTCAAGATTTTCGGCGACGAAGACTGCGAGAAGCATGAGGCTGAAA |
| | AAGTCAACACCACAAACGAATTGGAAGCTTTGACTAGGCGCAAAACAAGCG |
| | AGAAGATCCGCTTGTATGAACTCAAGTTGAGCAAATTAGACATTGTGGACAA |
| | ATTTCGGATCTTGCGTGAATAG (SEQ ID NO: 59) |
| Nucleotide sequence | ATGACCGCTACTGCTCCTTTCAAGATCGAATCCCCCTTCAGAATTGCCATCAT |
| of GPD1 gene from | CGGCTCCGGTAACTGGGGTACCGCCGTGGCCAAGCTTGTGGCTGAGAACACC |
| H0 Metschnikowia sp. | GCTGAGAAGCCGGAAATCTTCCAGAAACAGGTGAACATGTGGGTGTTTGAGG |
| | AGGACATCAACGGCCGCAAATTGACCGAGATCATCAACACTGACCATGAGA |
| | ACGTCAAGTACATGCCAGAGGTGAAGTTGCCAGAAAACTTGGTTGCAAACCC |
| | AGACATTGAGGCCACCGTCAAGGATGCTGACCTCCTTATTTTCAACATCCCCC |
| | ACCAGTTCTTGCCAAGAGTGTGCAAGCAATTGGTTGGCAAGGTTTCGCCTAC |
| | CGCCAGAGCCATTTCCTGTCTTAAGGGCTTGGAGGTGGATGCCTCTGGCTGC |
| | AAATTGTTGTCGCAGTCCATCACCGACACCTTGGGCATCTACTGTGGTGTCTT |
| | GTCCGGTGCCAACATCGCCAACGAGGTGGCTAGAGGCCGCTGGTCCGAGACC |
| | TCCATCGCCTACAACAGACCCACCGACTTCCGTGGCGAGGGCAAGGATATCT |
| | GTGAGTTTGTGTTGAAGGAGGCCTTCCACAGAAGATACTTCCACGTGCGCGT |
| | GATCAAGGACGTTATTGGCGCCTCGATCGCCGGTGCGTTGAAGAACGTTGTG |
| | GCCATTGCCGCCGGCTTCGTCGAAGGTGAGGGCTGGGGTGACAATGCCAAGT |
| | CTGCCATCATGAGAATCGGCCTCAAGGAGACCATTCACTTTGCCTCGTACTG |
| | GGAGAAGTTTGGCATCCAGGGTCTTTCTGCTCCTGAGCCTACCACCTTCACCG |
| | AGGAGTCTGCCGGTGTTGCCGACTTGATCACCACGTGTTCCGGTGGTAGAAA |
| | CGTCAAGGTTGCCAGATACATGATTGAGAAGAATGTCGACGCTTGGGAGGCT |
| | GAGAAGGCCTTGTTGAACGGCCAGTCCTCGCAAGGTATCATCACCGCCAAGG |
| | AGGTGCACGAGTTGTTGGTGAACTACAAGTTGCAAGAGGAGTTCCCATTGTT |
| | CGAGGCCACCTACGCTGTCATTTACGAGAACGCCGATGTCAACACCTGGCCT |
| | ACGATTTTGGCCGAGTAA (SEQ ID NO: 60) |
| Nucleotide sequence | ATGTCTCAAGACGAACTTCATACAAAGTCTGGTGTTGAAACACCAATCAACG |
| of GXF1 gene from | ATTCGCTTCTCGAGGAGAAGCACGATGTCACCCCACTCGCGGCATTGCCCGA |
| H0 Metschnikowia sp. | GAAGTCCTTCAAGGACTACATTTCCATTTCCATTTTCTGTTTGTTTGTGGCATT |
| | TGGTGGTTTTGTTTTCGGTTTCGACACCGGTACGATTTCCGGTTTCGTCAACA |
| | TGTCCGACTTCAAGACCAGATTTGGTGAGATGAATGCCCAGGGCGAATACTA |
| | CTTGTCCAATGTTAGAACTGGTTTGATGGTTTCTATTTTCAACGTCGGTTGCG |
| | CCGTTGGTGGTATCTTCCTTTGTAAGATTGCCGATGTTTATGGCAGAAGAATT |
| | GGTCTTATGTTTTCCATGGTGGTTTATGTCGTTGGTATCATTATTCAGATTGCC |
| | TCCACCACCAAATGGTACCAATACTTCATTGGCCGTCTTATTGCTGGCTTGGC |
| | TGTGGGTACTGTTTCCGTCATCTCGCCACTTTTCATTTCCGAGGTTGCTCCTAA |
| | ACAGCTCAGAGGTACGCTTGTGTGCTGCTTCCAGTTGTGTATCACCTTGGGTA |
| | TCTTTTTGGGTTACTGCACGACCTACGGTACAAAGACTTACACTGACTCCAGA |
| | CAGTGGAGAATCCCATTGGGTATCTGTTTCGCGTGGGCTTTGTTTTTGGTGGC |
| | CGGTATGTTGAACATGCCCGAGTCTCCTAGATACTTGGTTGAGAAATCGAGA |
| | ATCGACGATGCCAGAAAGTCCATTGCCAGATCCAACAAGGTTTCCGAGGAAG |
| | ACCCCGCCGTGTACACCGAGGTGCAGCTTATCCAGGCTGGTATTGACAGAGA |
| | GGCCCTTGCCGGCAGCGCCACATGGATGGAGCTTGTGACTGGTAAGCCCAAA |
| | ATCTTCAGAAGAGTCATCATGGGTGTCATGCTTCAGTCCTTGCAACAATTGAC |
| | TGGTGACAACTACTTTTTCTACTACGGAACCACGATTTTCAAGGCTGTTGGCT |
| | TGCAGGACTCTTTCCAGACGTCGATTATCTTGGGTATTGTCAACTTTGCCTCG |
| | ACTTTTGTCGGTATTTACGCCATTGAGAGAATGGGCAGAAGATTGTGTTTGTT |
| | GACCGGATCTGCGTGCATGTTTGTGTGTTTCATCATCTACTCGCTCATTGGTA |
| | CGCAGCACTTGTACAAGAACGGCTTCTCTAACGAACCTTCCAACACATACAA |
| | GCCTTCCGGTAACGCCATGATCTTCATCACGTGTCTTTACATTTTCTTCTTTGC |
| | CTCGACCTGGGCCGGTGGTGTTTACTGTATCGTGTCCGAGTCTTACCCATTGA |
| | GAATCAGATCCAAGGCCATGTCTGTCGCCACCGCCGCCAACTGGATGTGGGG |
| | TTTCTTGATCTCGTTCTTCACGCCTTTCATCACCTCCGCCATCCACTTTTACTA |
| | CGGTTTTGTTTTCACTGGCTGCTTGGCGTTCTCCTTCTTCTACGTCTACTTCTTT |
| | GTCGTGGAGACCAAGGGTCTTTCCTTGGAGGAGGTTGACATTTTGTACGCTTC |
| | CGGTACGCTTCCATGGAAGTCCTCTGGCTGGGTGCCTCCTACCGCGGACGAA |
| | ATGGCCCACAACGCCTTCGACAACAAGCCAACTGACGAACAAGTCTAA (SEQ |
| | ID NO: 61) |
| Nucleotide sequence | ATGAGTGCCGAACAGGAACAACAAGTATCGGGCACATCTGCCACGATAGAT |
| of GXF2 gene from | GGGCTGGCGTCCTTGAAGCAAGAAAAAACCGCCGAGGAGGAAGACGCCTTC |
| H0 Metschnikowia sp. | AAGCCTAAGCCCGCCACGGCGTACTTTTTCATTTCGTTCCTCTGTGGCTTGGT |
| | CGCCTTTGGCGGCTACGTTTTCGGTTTCGATACCGGCACGATTTCCGGGTTTG |
| | TTAACATGGACGACTATTTGATGAGATTCGGCCAGCAGCACGCTGATGGCAC |
| | GTATTACCTTTCCAACGTGAGAACCGGTTTGATCGTGTCGATCTTCAACATTG |
| | GCTGTGCCGTCGGTGGTCTTGCGCTTTCGAAAGTTGGTGACATCTGGGGCAG |
| | AAGAATTGGTATTATGGTTGCTATGATCATCTACATGGTGGGAATCATCATCC |
| | AGATCGCTTCACAGGATAAATGGTACCAGTACTTCATTGGCCGTTTGATCACC |
| | GGGTTGGGTGTCGGCACCACGTCCGTGCTCAGTCCTCTTTTCATCTCCGAGTC |
| | GGCTCCGAAGCATTTGAGAGGCACCCTTGTGTGTTGTTTCCAGCTCATGGTCA |
| | CCTTGGGTATCTTTTTGGGCTACTGCACGACCTACGGTACCAAGAACTACACT |
| | GACTCGCGCCAGTGGCGGATTCCCTTGGGTCTTTGCTTTGCATGGGCGCTTTT |
| | GTTGATCTCGGGAATGGTTTTCATGCCCGAATCCCCACGTTTCTTGATTGAAC |
| | GCCAGAGATTCGACGAGGCGAAGGCCTCCGTGGCCAAATCGAACCAGGTCTC |
| | GACCGAGGACCCCGCCGTGTACACTGAAGTGGAGTTGATCCAGGCCGGTATT |
| | GACCGTGAGGCATTGGCCGGATCCGCTGGCTGGAAAGAGCTTATCACGGGCA |
| | AGCCCAAGATGTTGCAGCGTGTGATTTTGGGAATGATGCTCCAGTCGATCCA |
| | GCAGCTCACCGGTAACAACTACTTTTTCTACTACGGTACCACGATCTTCAAGG |
| | CCGTGGGCATGTCGGACTCGTTCCAGACCTCGATTGTTTTGGGTATTGTCAAC |
| | TTCGCCTCCACTTTTGTCGGAATCTGGGCCATCGAGCGTATGGGCCGCAGATC |
| | TTGTTTGCTTGTTGGTTCCGCGTGCATGAGTGTGTGTTTCTTGATCTACTCCAT |
| | CTTGGGTTCCGTCAACCTTTACATCGACGGCTACGAGAACACGCCTTCGAAC |
| | ACGCGTAAGCCTACCGGTAACGCCATGATCTTCATCACGTGTTTGTTTATCTT |
| | CTTCTTCGCCTCCACCTGGGCCGGTGGTGTGTACAGTATTGTTTCTGAAACAT |
| | ACCCATTGAGAATCCGGTCTAAAGGTATGGCCGTGGCCACCGCTGCCAACTG |
| | GATGTGGGGTTTCTTGATTTCGTTCTTCACGCCTTTCATCACCTCGGCCATCCA |
| | CTTCTACTACGGGTTTGTGTTCACAGGGTGTCTTATTTTCTCCTTCTTCTACGT |
| | GTTCTTCTTTGTTAGGGAAACCAAGGGTCTCTCGTTGGAAGAGGTGGATGAG |
| | TTATATGCCACTGACCTCCCACCATGGAAGACCGCGGGCTGGACGCCTCCTT |
| | CTGCTGAGGATATGGCCCACACCACCGGGTTTGCCGAGGCCGCAAAGCCTAC |
| | GAACAAACACGTTTAA (SEQ ID NO: 62) |
| Nucleotide sequence | ATGAGCATCTTTGAAGGCAAAGACGGGAAGGGGGTATCCTCCACCGAGTCGC |
| of GXS1 gene from H0 | TTTCCAATGACGTCAGATATGACAACATGGAGAAAGTTGATCAGGATGTTCT |
| Metschnikowia sp. | TAGACACAACTTCAACTTTGACAAAGAATTCGAGGAGCTCGAAATCGAGGCG |
| | GCGCAAGTCAACGACAAACCTTCTTTTGTCGACAGGATTTTATCCCTCGAATA |
| | CAAGCTTCATTTCGAAAACAAGAACCACATGGTGTGGCTCTTGGGCGCTTTC |
| | GCAGCCGCCGCAGGCTTATTGTCTGGCTTGGATCAGTCCATTATTTCTGGTGC |
| | ATCCATTGGAATGAACAAAGCATTGAACTTGACTGAACGTGAAGCCTCATTG |
| | GTGTCTTCGCTTATGCCTTTAGGCGCCATGGCAGGCTCCATGATTATGACACC |
| | TCTTAATGAGTGGTTCGGAAGAAAATCATCGTTGATTATTTCTTGTATTTGGT |
| | ATACCATCGGATCCGCTTTGTGCGCTGGCGCCAGAGATCACCACATGATGTA |
| | CGCTGGCAGATTTATTCTTGGTGTCGGTGTGGGTATAGAAGGTGGGTGTGTG |
| | GGCATTTACATTTCCGAGTCTGTCCCAGCCAATGTGCGTGGTAGTATCGTGTC |
| | GATGTACCAGTTCAATATTGCTTTGGGTGAAGTTCTAGGGTATGCTGTTGCTG |
| | CCATTTTCTACACTGTTCATGGTGGATGGAGGTTCATGGTGGGGTCTTCTTTA |
| | GTATTCTCTACTATATTGTTTGCCGGATTGTTTTTCTTGCCCGAGTCACCTCGT |
| | TGGTTGGTGCACAAAGGCAGAAACGGAATGGCATACGATGTGTGGAAGAGA |
| | TTGAGAGACATAAACGATGAAAGCGCAAAGTTGGAATTTTTGGAGATGAGA |
| | CAGGCTGCTTATCAAGAGAGAGAAAGACGCTCGCAAGAGTCTTTGTTCTCCA |
| | GCTGGGGCGAATTATTCACCATCGCTAGAAACAGAAGAGCACTTACTTACTC |
| | TGTCATAATGATCACTTTGGGTCAATTGACTGGTGTCAATGCCGTCATGTACT |
| | ACATGTCGACTTTGATGGGTGCAATTGGTTTCAACGAGAAAGACTCTGTGTTC |
| | ATGTCCCTTGTGGGAGGCGGTTCTTTGCTTATAGGTACCATTCCTGCCATTTT |
| | GTGGATGGACCGTTTCGGCAGAAGAGTTTGGGGTTATAATCTTGTTGGTTTCT |
| | TCGTTGGTTTGGTGCTCGTTGGTGTTGGCTACCGTTTCAATCCCGTCACTCAA |
| | AAGGCGGCTTCAGAAGGTGTGTACTTGACGGGTCTCATTGTCTATTTCTTGTT |
| | CTTTGGTTCCTACTCGACCTTAACTTGGGTCATTCCATCCGAGTCTTTTGATTT |
| | GAGAACAAGATCTTTGGGTATGACAATCTGTTCCACTTTCCTTTACTTGTGGT |
| | CTTTCACCGTCACCTACAACTTCACCAAGATGTCCGCCGCCTTCACATACACT |
| | GGGTTGACACTTGGTTTCTACGGTGGCATTGCGTTCCTTGGTTTGATTTACCA |
| | GGTCTGCTTCATGCCCGAGACGAAGGACAAGACTTTGGAAGAAATTGACGAT |
| | ATCTTCAATCGTTCTGCGTTCTCTATCGCGCGCGAGAACATCTCCAACTTGAA |
| | GAAGGGTATTTGGTAA (SEQ ID NO: 63) |
| Nucleotide sequence | ATGTCAGAAAAGCCTGTTGTGTCGCACAGCATCGACACGACGCTGTCTACGT |
| of HGT19 gene from | CATCGAAACAAGTCTATGACGGTAACTCGCTTCTTAAGACCCTGAATGAGCG |
| H0 Metschnikowia sp. | CGATGGCGAACGCGGCAATATCTTGTCGCAGTACACTGAGGAACAGGCCATG |
| | CAAATGGGCCGCAACTATGCGTTGAAGCACAATTTAGATGCGACACTCTTTG |
| | GAAAGGCGGCCGCGGTCGCAAGAAACCCATACGAGTTCAATTCGATGAGTTT |
| | TTTGACCGAAGAGGAAAAAGTCGCGCTTAACACGGAGCAGACCAAGAAATG |
| | GCACATCCCAAGAAAGTTGGTGGAGGTGATTGCATTGGGGTCCATGGCCGCT |
| | GCGGTGCAGGGTATGGATGAGTCGGTGGTGAATGGTGCAACGCTTTTCTACC |
| | CCACGGCAATGGGTATCACAGATATCAAGAATGCCGATTTGATTGAAGGTTT |
| | GATCAACGGTGCGCCCTATCTTTGCTGCGCCATCATGTGCTGGACATCTGATT |
| | ACTGGAACAGGAAGTTGGGCCGTAAGTGGACCATTTTCTGGACATGTGCCAT |
| | TTCTGCAATCACATGTATCTGGCAAGGTCTCGTCAATTTGAAATGGTACCATT |
| | TGTTCATTGCGCGTTTCTGCTTGGGTTTCGGTATCGGTGTCAAGTCTGCCACC |
| | GTGCCTGCGTATGCTGCCGAAACCACCCCGGCCAAAATCAGAGGCTCGTTGG |
| | TCATGCTTTGGCAGTTCTTCACCGCTGTCGGAATCATGCTTGGTTACGTGGCG |
| | TCTTTGGCATTCTATTACATTGGTGACAATGGCATTTCTGGCGGCTTGAACTG |
| | GAGATTGATGCTAGGATCTGCATGTCTTCCAGCTATCGTTGTGTTAGTCCAAG |
| | TTCCGTTTGTTCCAGAATCCCCTCGTTGGCTCATGGGTAAGGAAAGACACGCT |
| | GAAGCATATGATTCGCTCCGGCAATTGCGGTTCAGTGAAATCGAGGCGGCCC |
| | GTGACTGTTTCTACCAGTACGTGTTGTTGAAAGAGGAGGGCTCTTATGGAAC |
| | GCAGCCATTCTTCAGCAGAATCAAGGAGATGTTCACCGTGAGAAGAAACAG |
| | AAATGGTGCATTGGGCGCGTGGATCGTCATGTTCATGCAGCAGTTCTGTGGA |
| | ATCAACGTCATTGCTTACTACTCGTCGTCGATCTTCGTGGAGTCGAATCTTTC |
| | TGAGATCAAGGCCATGTTGGCGTCTTGGGGGTTCGGTATGATCAATTTCTTGT |
| | TTGCAATTCCAGCGTTCTACACCATTGACACGTTTGGCCGACGCAACTTGTTG |
| | CTCACTACTTTCCCTCTTATGGCGGTATTCTTACTCATGGCCGGATTCGGGTTC |
| | TGGATCCCGTTCGAGACAAACCCACACGGCCGTTTGGCGGTGATCACTATTG |
| | GTATCTATTTGTTTGCATGTGTCTACTCTGCGGGCGAGGGACCAGTTCCCTTC |
| | ACATACTCTGCCGAAGCATTCCCGTTGTATATCCGTGACTTGGGTATGGGCTT |
| | TGCCACGGCCACGTGTTGGTTCTTCAACTTCATTTTGGCATTTTCCTGGCCTA |
| | GAATGAAGAATGCATTCAAGCCTCAAGGTGCCTTTGGCTGGTATGCCGCCTG |
| | GAACATTGTTGGCTTCTTCTTAGTGTTATGGTTCTTGCCCGAGACAAAGGGCT |
| | TGACGTTGGAGGAATTGGACGAAGTGTTTGATGTGCCTTTGAGAAAACACGC |
| | GCACTACCGTACCAAAGAATTAGTATACAACTTGCGCAAATACTTCTTGAGG |
| | CAGAACCCTAAGCCATTGCCGCCACTTTATGCACACCAAAGAATGGCTGTTA |
| | CCAACCCAGAATGGTTGGAAAAGACCGAGGTCACGCACGAGGAGAATATCT |
| | AG (SEQ ID NO: 64) |
| Nucleotide sequence | ATGCTGAGCACTACCGATACCCTCGAAAAAAGGGACACCGAGCCTTTCACTT |
| of HXT2.6 gene from | CAGATGCTCCTGTCACAGTCCATGACTATATCGCAGAGGAGCGTCCGTGGTG |
| H0 Metschnikowia sp. | GAAAGTGCCGCATTTGCGTGTATTGACTTGGTCTGTTTTCGTGATCACCCTCA |
| | CCTCCACCAACAACGGGTATGATGGCCTGATGTTGAATGGATTGCAATCCTT |
| | GGACATTTGGCAGGAGGATTTGGGTCACCCTGCGGGCCAGAAATTGGGTGCC |
| | TTGGCCAACGGTGTTTTGTTTGGTAACCTTGCTGCTGTGCCTTTTGCTTCGTAT |
| | TTCTGCGATCGTTTTGGTAGAAGGCCGGTCATTTGTTTCGGACAGATCTTGAC |
| | AATTGTTGGTGCTGTATTACAAGGTTTGTCCAACAGCTATGGATTTTTTTTGG |
| | GTTCGAGAATTGTGTTGGGTTTTGGTGCTATGATAGCCACTATTCCGCTGCCA |
| | ACATTGATTTCCGAAATCGCCTACCCTACGCATAGAGAAACTTCCACTTTCGC |
| | CTACAACGTGTGCTGGTATTTGGGAGCCATTATCGCCTCCTGGGTCACATACG |
| | GCACCAGAGATTTACAGAGCAAGGCTTGCTGGTCAATTCCTTCTTATCTCCAG |
| | GCCGCCTTACCTTTCTTTCAAGTGTGCATGATTTGGTTTGTGCCAGAGTCTCC |
| | CAGATTCCTCGTTGCCAAGGGCAAGATCGACCAAGCAAGGGCTGTTTTGTCT |
| | AAATACCATACAGGAGACTCGACTGACCCCAGAGACGTTGCGTTGGTTGACT |
| | TTGAGCTCCATGAGATTGAGAGTGCATTGGAGCAGGAAAAATTGAACACTCG |
| | CTCGTCATACTTTGACTTTTTCAAGAAGAGAAACTTTAGAAAGAGAGGCTTCT |
| | TGTGTGTCATGGTCGGTGTTGCAATGCAGCTTTCTGGAAACGGCTTAGTGTCC |
| | TATTACTTGTCGAAAGTGCTAGACTCGATTGGAATCACTGAAACCAAGAGAC |
| | AGCTCGAGATCAATGGCTGCTTGATGATCTATAACTTTGTCATCTGCGTCTCG |
| | TTGATGAGTGTTTGCCGTATGTTCAAAAGAAGAGTATTATTTCTCACGTGTTT |
| | CTCAGGAATGACGGTTTGCTACACGATATGGACGATTTTGTCAGCGCTTAAT |
| | GAACAGAGACACTTTGAGGATAAAGGCTTGGCCAATGGCGTGTTGGCAATGA |
| | TCTTCTTCTACTATTTTTTCTACAACGTTGGCATCAATGGATTGCCATTCCTAT |
| | ACATCACCGAGATCTTGCCTTACTCACACAGAGCAAAAGGCTTGAATTTATT |
| | CCAATTCTCGCAATTTCTCACGCAAATCTACAATGGCTATGTGAACCCAATCG |
| | CCATGGACGCAATCAGCTGGAAGTATTACATTGTGTACTGCTGTATTCTCTTC |
| | GTGGAGTTGGTGATTGTGTTTTTCACGTTCCCAGAAACTTCGGGATACACTTT |
| | GGAGGAGGTCGCCCAGGTATTTGGTGATGAGGCTCCCGGGCTCCACAACAGA |
| | CAATTGGATGTTGCGAAAGAATCACTCGAGCATGTTGAGCATGTTTGA (SEQ |
| | ID NO: 65) |
| Nucleotide sequence | ATGAGCATCTTTGAAGGCAAAGACGGGAAGGGGGTATCCTCCACCGAGTCGC |
| of HXT5 gene from | TTTCCAATGACGTCAGATATGACAACATGGAGAAAGTTGATCAGGATGTTCT |
| H0 Metschnikowia sp. | TAGACACAACTTCAACTTTGACAAAGAATTCGAGGAGCTCGAAATCGAGGCG |
| | GCGCAAGTCAACGACAAACCTTCTTTTGTCGACAGGATTTTATCCCTCGAATA |
| | CAAGCTTCATTTCGAAAACAAGAACCACATGGTGTGGCTCTTGGGCGCTTTC |
| | GCAGCCGCCGCAGGCTTATTGTCTGGCTTGGATCAGTCCATTATTTCTGGTGC |
| | ATCCATTGGAATGAACAAAGCATTGAACTTGACTGAACGTGAAGCCTCATTG |
| | GTGTCTTCGCTTATGCCTTTAGGCGCCATGGCAGGCTCCATGATTATGACACC |
| | TCTTAATGAGTGGTTCGGAAGAAAATCATCGTTGATTATTTCTTGTATTTGGT |
| | ATACCATCGGATCCGCTTTGTGCGCTGGCGCCAGAGATCACCACATGATGTA |
| | CGCTGGCAGATTTATTCTTGGTGTCGGTGTGGGTATAGAAGGTGGGTGTGTG |
| | GGCATTTACATTTCCGAGTCTGTCCCAGCCAATGTGCGTGGTAGTATCGTGTC |
| | GATGTACCAGTTCAATATTGCTTTGGGTGAAGTTCTAGGGTATGCTGTTGCTG |
| | CCATTTTCTACACTGTTCATGGTGGATGGAGGTTCATGGTGGGGTCTTCTTTA |
| | GTATTCTCTACTATATTGTTTGCCGGATTGTTTTTCTTGCCCGAGTCACCTCGT |
| | TGGTTGGTGCACAAAGGCAGAAACGGAATGGCATACGATGTGTGGAAGAGA |
| | TTGAGAGACATAAACGATGAAAGCGCAAAGTTGGAATTTTTGGAGATGAGA |
| | CAGGCTGCTTATCAAGAGAGAGAAAGACGCTCGCAAGAGTCTTTGTTCTCCA |
| | GCTGGGGCGAATTATTCACCATCGCTAGAAACAGAAGAGCACTTACTTACTC |
| | TGTCATAATGATCACTTTGGGTCAATTGACTGGTGTCAATGCCGTCATGTACT |
| | ACATGTCGACTTTGATGGGTGCAATTGGTTTCAACGAGAAAGACTCTGTGTTC |
| | ATGTCCCTTGTGGGAGGCGGTTCTTTGCTTATAGGTACCATTCCTGCCATTTT |
| | GTGGATGGACCGTTTCGGCAGAAGAGTTTGGGGTTATAATCTTGTTGGTTTCT |
| | TCGTTGGTTTGGTGCTCGTTGGTGTTGGCTACCGTTTCAATCCCGTCACTCAA |
| | AAGGCGGCTTCAGAAGGTGTGTACTTGACGGGTCTCATTGTCTATTTCTTGTT |
| | CTTTGGTTCCTACTCGACCTTAACTTGGGTCATTCCATCCGAGTCTTTTGATTT |
| | GAGAACAAGATCTTTGGGTATGACAATCTGTTCCACTTTCCTTTACTTGTGGT |
| | CTTTCACCGTCACCTACAACTTCACCAAGATGTCCGCCGCCTTCACATACACT |
| | GGGTTGACACTTGGTTTCTACGGTGGCATTGCGTTCCTTGGTTTGATTTACCA |
| | GGTCTGCTTCATGCCCGAGACGAAGGACAAGACTTTGGAAGAAATTGACGAT |
| | ATCTTCAATCGTTCTGCGTTCTCTATCGCGCGCGAGAACATCTCCAACTTGAA |
| | GAAGGGTATTTGGTAA (SEQ ID NO: 66) |
| Nucleotide sequence | ATGTCTTTATCTAACAAATTGTCTGTGAAAGACTTGGACCTCGCTAACAAGA |
| of PGK1 gene from | GAGTCTTCATCAGAGTCGACTTCAACGTTCCTCTTGACGGAACCACCATCACC |
| H0 Metschnikowia sp. | AACAACCAGAGAATTGTTGCTGCTTTGCCAACCATCAAATACGTCTTGGAGC |
| | AGAAGCCAAAGGCCGTCATCTTGGCTTCCCACTTGGGCAGACCAAACGGTGA |
| | GAGAGTTGAGAAGTACTCGTTGGCTCCAGTTGCCAAGGAATTGCAGTCCTTG |
| | TTGTCTGACCAGAAGGTCACATTCTTGAACGACAGCGTTGGACCTGAGGTCG |
| | AGAAGGCTGTCAACAGCGCCTCTCAGGGCGAGGTGTTCTTGTTGGAGAACTT |
| | GCGTTACCACATCGAGGAGGAAGGCTCCAAGAAGGTCGACGGCAACAAGGT |
| | CAAGGCTTCCAAGGAGGATGTCGAGAAGTTCAGACAAGGATTGACCGCCTTG |
| | GCCGACGTCTACGTCAACGACGCTTTCGGTACCGCCCACAGAGCCCACTCTT |
| | CTATGGTTGGTCTTGAATTGCCTCAGAAGGCTGCCGGTTTCTTGATGGCCAAG |
| | GAGTTGGAGTACTTCGCCAAGGCCTTGGAGAACCCTACCAGACCATTCTTGG |
| | CCATCTTGGGTGGTGCCAAGGTCTCCGACAAGATCCAGTTGATCGACAACTT |
| | GTTGGACAAGGTCGACATCTTGATTGTTGGTGGTGGTATGGCTTTCACCTTCA |
| | AGAAGGTTTTGGACAACATGCCAATTGGTACTTCTCTTTTCGACGAGGCCGG |
| | CTCCAAGAACGTCGAGAACTTGATTGCCAAGGCTAAGAAGAACAACGTCGA |
| | GATTGTCTTGCCCGTTGACTTTGTCACCGCTGACGACTTCAACAAGGATGCCA |
| | ACACTGGTGTTGCCACCCAAGAGGAGGGTATCCCAGACGGATGGATGGGTCT |
| | TGATGCCGGTCCAAAGTCCAGAGAACTCTTTGCTGAGGCTGTTGCTAAGGCC |
| | AAGACCATTGTCTGGAACGGCCCACCAGGTGTTTTCGAGTTTGAGAAATTCG |
| | CTCAGGGCACCAAGTCCTTGTTGGACGCTGCCGTCAAGTCCGCCGAGGCTGG |
| | CAACACCGTCATCATTGGCGGTGGTGACACTGCCACTGTTGCCAAGAAGTTC |
| | GGTGTCGTTGAGAAGTTGTCTCACGTCTCCACTGGTGGTGGTGCCTCCTTGGA |
| | GTTGTTGGAGGGTAAGGAGTTGCCAGGTGTCGTTGCCATTTCTGACAAGCAG |
| | TAA (SEQ ID NO: 67) |
| Nucleotide sequence | ATGGGCTTTCGCAACTTAAAGCGCAGGCTCTCAAATGTTGGCGACTCCATGT |
| of QUP2 gene from | CAGTGCACTCTGTGAAAGAGGAGGAAGACTTCTCCCGCGTGGAAATCCCGGA |
| H0 Metschnikowia sp. | TGAAATCTACAACTATAAGATCGTCCTTGTGGCTTTAACAGCGGCGTCGGCT |
| | GCCATCATCATCGGCTACGATGCAGGCTTCATTGGTGGCACGGTTTCGTTGAC |
| | GGCGTTCAAACTGGAATTTGGCTTGGACAAAATGTCTGCGACGGCGGCTTCT |
| | GCTATCGAAGCCAACGTTGTTTCCGTGTTCCAGGCCGGCGCCTACTTTGGGTG |
| | TCTTTTCTTCTATCCGATTGGCGAGATTTGGGGCCGTAAAATCGGTCTTCTTCT |
| | TTCCGGCTTTCTTTTGACGTTTGGTGCTGCTATTTCTTTGATTTCGAACTCGTC |
| | TCGTGGCCTTGGTGCCATATATGCTGGAAGAGTACTAACAGGTTTGGGGATT |
| | GGCGGATGTCTGAGTTTGGCCCCAATCTACGTTTCTGAAATCGCGCCTGCAGC |
| | AATCAGAGGCAAGCTTGTGGGCTGCTGGGAAGTGTCATGGCAGGTGGGCGG |
| | CATTGTTGGCTACTGGATCAATTACGGAGTCTTGCAGACTCTTCCGATTAGCT |
| | CACAACAATGGATCATCCCGTTTGCTGTACAATTGATCCCATCGGGGCTTTTC |
| | TGGGGCCTTTGTCTTTTGATTCCAGAGCTGCCACGTTTTCTTGTATCGAAGGG |
| | AAAGATCGATAAGGCGCGCAAAAACTTAGCGTACTTGCGTGGACTTAGCGAG |
| | GACCACCCCTATTCTGTTTTTGAGTTGGAGAACATTAGTAAGGCCATTGAAG |
| | AGAACTTCGAGCAAACAGGAAGGGGTTTTTTCGACCCATTGAAAGCTTTGTT |
| | TTTCAGCAAAAAAATGCTTTACCGCCTTCTCTTGTCCACGTCAATGTTCATGA |
| | TGCAGAATGGCTATGGAATCAATGCTGTGACATACTACTCGCCCACGATCTT |
| | CAAATCCTTAGGCGTTCAGGGCTCAAACGCCGGTTTGCTCTCAACAGGAATT |
| | TTCGGTCTTCTTAAAGGTGCCGCTTCGGTGTTCTGGGTCTTTTTCTTGGTTGAC |
| | ACATTCGGCCGCCGGTTTTGTCTTTGCTACCTCTCTCTCCCCTGCTCGATCTGC |
| | ATGTGGTATATTGGCGCATACATCAAGATTGCCAACCCTTCAGCGAAGCTTG |
| | CTGCAGGAGACACAGCCACCACCCCAGCAGGAACTGCAGCGAAAGCGATGC |
| | TTTACATATGGACGATTTTCTACGGCATTACGTGGAATGGTACGACCTGGGTG |
| | ATCTGCGCGGAGATTTTCCCCCAGTCGGTGAGAACAGCCGCGCAGGCCGTCA |
| | ACGCTTCTTCTAATTGGTTCTGGGCTTTCATGATCGGCCACTTCACTGGCCAG |
| | GCGCTCGAGAATATTGGGTACGGATACTACTTCTTGTTTGCGGCGTGCTCTGC |
| | AATCTTCCCTGTGGTAGTCTGGTTTGTGTACCCCGAAACAAAGGGTGTGCCTT |
| | TGGAGGCCGTGGAGTATTTGTTCGAGGTGCGTCCTTGGAAAGCGCACTCATA |
| | TGCTTTGGAGAAGTACCAGATTGAGTACAACGAGGGTGAATTCCACCAACAT |
| | AAGCCCGAAGTACTCTTACAAGGGTCTGAAAACTCGGACACGAGCGAGAAA |
| | AGCCTCGCCTGA (SEQ ID NO: 68) |
| Nucleotide sequence | ATGGACCAGACAACCAAGAAACCCAGAGATGGTGGCTTGAACGATCCACGT |
| of RPB1 gene from H0 | TTGGGCTCCATCGACCGTAACTTCAAGTGTCAAACCTGTGGCGAAGATATGG |
| Metschnikowia sp. | CTGAATGTCCGGGCCATTTTGGCCACATTGAGTTGGCCAAGCCCGTGTTTCAC |
| | ATCGGTTTTATTGCCAAGATCAAGAAAGTGTGCGAGTGTGTTTGTATGCACTG |
| | TGGAAAACTTCTTGTTGACGATGCTAACCCCTTGATGGCTCAGGCCATTCGGA |
| | TCAGGGATCCGAAGAAGCGCTTCAACGCCGTGTGGAACGTGTCCAAGACCAA |
| | GATGGTGTGTGAAGCAGACACTATCAATGAAGAAGGCCAGGTCACAGCCGG |
| | GAGAGGAGGATGTGGCCACACGCAGCCAACTGTGCGCAGAGACGGCTTGAA |
| | GTTGTGGGGTACTTGGAAACAGAACAAAACTTACGACGAGAACGAACAGCC |
| | AGAACGTCGTTTGTTAAGTCCATCAGAGATTTTGAGCGTTTTCAGACACATCA |
| | GCCCCGAGGACTGTCATAAGTTGGGCTTTAACGAGGACTATGCCAGACCTGA |
| | GTGGATGTTGATCACGGTTTTGCCTGTCCCACCACCACCAGTGAGGCCTTCCA |
| | TTGCCTTTAACGATACGGCTAGAGGTGAGGATGATTTGACGTTCAAGTTGGC |
| | TGACATTCTCAAAGCAAATATCAACGTACAGCGTCTTGAAATCGACGGTTCG |
| | CCACAGCACGTCATCAGTGAGTTCGAGGCTTTGTTACAGTTTCATGTGGCGAC |
| | TTACATGGATAATGATATCGCTGGCCAGCCTCAGGCGCTTCAAAAGACCGGT |
| | CGTCCTATCAAATCGATCAGAGCCAGATTGAAGGGTAAAGAGGGGAGATTG |
| | AGAGGTAACTTGATGGGCAAACGTGTGGACTTTTCTGCGCGTACTGTTATTTC |
| | TGGTGACCCCAATCTCGACCTTGACCAGGTCGGTGTGCCTATATCCATTGCTA |
| | GGACTTTGACTTATCCTGAGGTTGTCACCCCATACAACATTCACAAATTGACC |
| | GAGTATGTTCGCAATGGCCCTAATGAGCACCCTGGTGCGAAATATGTCATTC |
| | GTGACACCGGTGACCGTATTGATCTAATGTACAACAAAAGGGCGGGTGACAT |
| | TGCCTTGCAGTATGGGTGGAAGGTTGAACGTCATTTGATGGACGACGATCCA |
| | GTTTTGTTTAATCGTCAACCCTCCTTGCATAAGATGTCCATGATGGCACATCG |
| | AGTCAAAGTCATGCCCTACTCCACATTCAGATTGAATTTGTCCGTCACTTCTC |
| | CTTACAATGCTGATTTCGATGGTGATGAGATGAACTTACATGTTCCTCAGTCG |
| | CCTGAGACCAGAGCCGAGATGTCTCAAATTTGCGCGGTTCCGCTTCAAATCG |
| | TCTCTCCACAATCGAACAAACCTGTGATGGGTATTGTGCAAGACACATTGTG |
| | TGGTATCCGTAAAATGACATTACGCGACAATTTCATTGAATATGAGCAAGTC |
| | ATGAACATGTTGTACTGGATCCCTAACTGGGATGGTGTCATTCCTCCGCCGGC |
| | GGTACTCAAGCCCAAGCCATTGTGGTCGGGTAAACAGTTGTTGTCTATGGCC |
| | ATTCCCAAGGGTATTCACTTGCAGAGGTTCGATGACGGAAGGGACATGCTCA |
| | GTCCAAAAGATCTGGGGATGTTGATTGTTGACGGTGAGATCATCTTTGGTGTT |
| | GTTGACAAAAAAACCGTCGGCGCCACTGGAGGCGGATTGATCCACACGGTCA |
| | TGAGAGAGAAGGGTCCATACGTCTGTGCGCAGCTTTTCAGCTCGATCCAGAA |
| | GGTTGTCAATTATTGGCTTTTGCATAATGGTTTCTCTATCGGTATTGGTGACA |
| | CAATTGCCGACAAAGACACCATGCGTGATGTGACAACGACCATTCAAGAGGC |
| | CAAACAGAAGGTCCAGGAAATCATCATTGACGCCCAGCAAAACAAGTTGGA |
| | GCCTGAACCCGGTATGACTCTCAGAGAATCGTTCGAGCATAATGTTTCCCGT |
| | ATTCTCAATCAAGCTCGTGATACTGCTGGCCGTTCCGCTGAAATGAACTTGAA |
| | GGATCTGAACAACGTGAAACAGATGGTCACATCCGGATCGAAAGGTTCTTTC |
| | ATCAACATCTCTCAAATGTCTGCCTGTGTCGGTCAACAAATTGTTGAGGGTAA |
| | GCGTATTCCCTTCGGTTTTGGTGATCGTACGTTACCTCATTTTACCAAGGATG |
| | ACTACTCGCCTGAATCGAAGGGTTTTGTTGAGAACTCGTACCTCAGAGGCTT |
| | GACTCCCCAGGAGTTTTTCTTTCACGCTATGGCAGGAAGAGAAGGTCTTATTG |
| | ATACTGCCGTCAAGACTGCAGAAACAGGTTACATCCAGCGTCGTTTAGTCAA |
| | AGCTTTGGAAGATATTATGGTGCATTATGATGGCACAACCAGAAACTCTTTA |
| | GGCGACATCATCCAGTTTGTTTATGGTGAGGACGGAATTGATGCTACATCGG |
| | TTGAAAAGCAATCAGTTGATACTATACCCGGTTCAGACTCCTCGTTTGAGAA |
| | GCGCTACAGAATTGACGTTTTGGACCCAGCTAAATCCATTCCTGAGTCGTTGC |
| | TAGAGTCAGGCAAGCAAATCAAGGGAGATGTGGCAGTTCAGAAGGTGTTGG |
| | ATGAAGAGTACGACCAATTGCTCAAGGATCGTAAGTTCTTGAGAGAGGTTGT |
| | TTTCCCCAATGGTGACTACAACTGGCCATTACCCGTTAATTTGCGTCGTATTA |
| | TTCAAAATGCTCAGCAGATTTTCCACAGTGGCCGTCAAAAAGCTTCCGACTT |
| | AAGATTGGAAGAGATAGTCGAAGGCGTGCAGTCCCTTTGTACCAAGCTTCTT |
| | GTTCTCCGAGGAAAGACGGAGCTCATCAAGGAGGCGCAGGAAAATGCGACT |
| | TTGCTTTTCCAGTGCTTGTTGAGATCTAGGTTGGCTGCTCGTCGTGTCATTGA |
| | GGAGTTCAAGCTCAATAAGGTCTCTTTTGAATGGGTATGTGGTGAAATCGAG |
| | TCCCAGTTTCAGAAGTCTATTGTACACCCAGGTGAGATGGTTGGTGTTGTCGC |
| | TGCGCAGTCTATCGGTGAGCCTGCGACGCAGATGACTTTAAACACCTTCCATT |
| | ACGCCGGTGTCTCTTCCAAAAACGTTACCCTTGGTGTCCCTCGTCTTAAGGAA |
| | ATTTTGAATGTGGCGAAAAACATCAAAACGCCGGCTCTTACCGTGTACTTGG |
| | AGCCCGAGATCGCTGTTGACATTGAAAAGGCCAAGGTTGTTCAATCGGCTAT |
| | TGAACACACCACGTTGAAGAACGTGACCTCGTCCACAGAAATCTACTACGAT |
| | CCTGATCCTAGAAGCACCGTGATTGAGGAAGATTATGATACTGTTGAAGCTT |
| | ACTTTGCCATTCCCGACGAGAAGGTCGAGGAAACTATCGACAATCAGTCTCC |
| | ATGGTTGCTTCGTCTTGAATTGGACAGAGCCAAAATGTTGGATAAGCAACTT |
| | ACGATGGCTCAAGTGGCCGAGAAGATTTCGCAGAACTTTGGAGAAGACTTGT |
| | TCGTTATTTGGTCTGATGACACTGCAGACAAGTTGATCATCCGTTGTCGTGTT |
| | ATCCGCGATCCAAAATTGGAAGAGGAAGGCGAGCACGAGGAGGACCAAATT |
| | TTGAAGAGAGTGGAGGCCCACATGTTGGAGACAATCTCATTGCGTGGTATCC |
| | CTGGTATCACGAGAGTCTTTATGATGCAACATAAGATGAGCACGCCAGATGC |
| | GGATGGTGAATTTCTGCAAAAGCAAGAATGGGTTTTGGAAACTGATGGTGTA |
| | AACTTGGCCGAGGTCATCACTGTTCCTGGCGTCGATGCATCCCGAACCTATTC |
| | CAACAACTTCATCGAGATTCTTTCTGTGCTCGGTATTGAGGCGACTCGTACTG |
| | CTTTGTTCAAGGAAATTCTCAATGTCATTGCATTTGACGGTTCATACGTCAAC |
| | TACCGTCATATGGCTTTGCTTGTGGACGTCATGACTGCACGTGGTCATTTGAT |
| | GGCTATCACCCGTCATGGTATTAACAGAGCGGAAACTGGTGCTTTGATGCGT |
| | TGTTCTTTTGAAGAGACGGTTGAGATCTTGTTGGATGCTGGTGCCGCTGCTGA |
| | ACTAGATGACTGCCGTGGTATCTCCGAGAATGTCATATTAGGACAAATGCCA |
| | CCTTTGGGTACCGGTGCTTTTGATGTGATGGTCGACGAGAAGATGTTGCAGG |
| | ACGCAAGTGTGAGTTCTGATATTGGTGTTGCTGGTCAGACTGACGGAGGTGC |
| | GACGCCATATAGAGACTATGAGATGGAGGATGATAAGATTCAATTTGAGGA |
| | AGGTGCGGGATTCTCGCCAATTCATACCGCAAATGTATCTGATGCCTCTGGGT |
| | CTTTAACCTCGTACGGCGGGCAACCATCCATGGTATCACCTACCTCGCCATTC |
| | TCGTTTGGCGCCACGTCTCCTGGGTATGGCGGTGTGACCTCGCCTGCGTACGG |
| | CGCAACTTCGCCAACGTACTCACCAACGTCACCAACATACTCGCCAACTTCG |
| | CCCAGTTACTCACCGACGTCACCAAGTTACTCACCGACGTCACCAAGTTACTC |
| | ACCGACGTCACCAAGTTACTCACCGACGTCACCAAGTTACTCGCCAACATCG |
| | CCAAGTTATTCGCCAACTTCACCAAGTTATTCGCCAACTTCGCCAAGTTACTC |
| | GCCAACTTCGCCAAGTTATTCGCCTACTTCGCCAAGTTATTCGCCAACTTCGC |
| | CAAGTTACTCACCGACGTCACCAAGTTACTCACCGACGTCACCAAGTTACTC |
| | ACCGACGTCACCAAGTTACTCGCCTACTTCGCCAAGTTACTCGCCTACTTCGC |
| | CAAGTTACTCACCTACTTCGCCAAGTTATTCGCCTACTTCGCCTAGTTACTCA |
| | CCTACTTCGCCGCAGTATTCGCCAACTTCGCCTAGTTACTCTCCGACGTCGCC |
| | GCAGTATTCGCCAACTTCGCCAAGCTACTCGCCTACGTCACCGCAATACCTGC |
| | CAACGTCGCCAAGTTACTCGCCCACTTCGCCTCAATACTCTCCAACTTCGCCT |
| | CAATACTCGCCGGGCTCACCGGCATATTCACCAGGCTCACCACTGTACTCTAC |
| | TGAGAAGAAGGACGAGGACAAGAAGTGA (SEQ ID NO: 69) |
| Nucleotide sequence | ATGTCGCAGGAGCCGGTAGAAGACCCTTACGTCTACGACGAGGAGGACGCG |
| of RPB2 gene from H0 | CACAGCATCACGCCCGAGGACTGCTGGACGGTGATTCTGTCGTTTTTCCAGG |
| Metschnikowia sp. | AAAAAGGCCTTGTCTCACAGCAGTTGGACTCGTTCGACGAGTTCATCGAGTC |
| | AAACATCCAGGAGTTGGTGTGGGAGGACTCGCACTTGATTCTCGACCAGCCG |
| | GCGCAACATACTTCCGAGGACCAGTATGAAAATAAGCGGTTTGAAATCACGT |
| | TTGGCAAGATCTATATTTCGAAGCCAACGCAGACCGAGGGCGACGGAACAA |
| | CGCACCCGATGTTCCCACAGGAGGCACGCTTGCGTAACTTGACCTACAGCTC |
| | GCCGCTTTACGTGGACATGCTGAAAAAGAAGTTTCTTTCCGATGACAGAGTG |
| | AGAAAGGGTAACGAGCTAGAATGGGTGGAGGAGAAAGTCGATGGCGAGGA |
| | GGCCCAGCTGAAGGTGTTCTTGGGTAAGGTGCCAATCATGCTAAGGTCGAAG |
| | TTTTGCATGTTGCGGGACTTGGGCGAGCACGAGTTCTACGAGTTGAAAGAGT |
| | GCCCTTACGATATGGGTGGCTATTTCGTCATCAACGGTTCCGAAAAAGTCTTG |
| | ATCGCCCAGGAGCGCTCGGCGGCTAACATTGTCCAGGTGTTTAAGAAGGCAG |
| | CGCCCTCGCCCATCTCGCACGTGGCGGAGATCCGTTCCGCGCTTGAAAAGGG |
| | TTCCCGTTTGATCTCCTCGATGCAGATCAAACTATATGGTCGTGACGACAAGG |
| | GCACCACTGGCAGAACAATCAAGGCCACATTGCCCTACATCAAGGAAGACAT |
| | CCCGATTGTGATTGTATTCAGAGCCCTCGGCGTGGTCCCCGATGGAGACATTT |
| | TGGAACACATTTGTTACGATGCAAACGATTGGCAAATGTTAGAGATGTTGAA |
| | GCCATGTGTGGAGGAAGGTTTCGTGATCCAGGAGCGCGAAGTCGCACTTGAC |
| | TTTATCGGTAGAAGAGGTGTCTTGGGTATCAGAAGGGAAAAGCGTATCCAGT |
| | ACGCAAAGGATATTTTACAGAAAGAGTTGTTGCCTAACATCACACAGGAGGC |
| | CGGTTTCGAGTCAAGAAAGGCATTCTTCTTGGGTTACATGGTCAACCGTTTGT |
| | TGTTATGTGCATTAGAAAGAAAGGAGCCTGACGACAGAGATCATTTTGGCAA |
| | GAAGAGATTGGATTTGGCCGGACCCTTGTTGGCATCCTTGTTCCGTCTCTTAT |
| | TCAAAAAGCTTACCAGGGATATCTATAACTACATGCAGCGGTGCGTGGAGAA |
| | TGACAAGGAGTTTAATCTCACGTTGGCGGTCAAGTCACAGACCATCACTGAT |
| | GGTTTGCGGTACTCGTTGGCCACAGGTAATTGGGGTGAACAAAGAAAGGCCA |
| | TGAGTGCACGTGCCGGTGTGTCGCAGGTGTTGAACAGATACACATACTCATC |
| | GACATTGTCGCATTTGAGAAGAACAAATACTCCAATTGGCCGTGACGGTAAG |
| | ATCGCCAAACCTAGACAGTTGCACAACACCCACTGGGGTCTTGTATGTCCTG |
| | CAGAAACTCCTGAGGGTCAGGCGTGTGGTTTGGTGAAGAATTTGTCTTTGAT |
| | GACGTGTATATCCGTTGGTACCTCTTCCGAGCCGATCTTGTATTTCTTGGAAG |
| | AGTGGGGTATGGAACCCTTGGAGGACTATGTTCCTTCGAACGCACCAGACTG |
| | CACAAGAGTCTTTGTCAACGGTGTATGGGTTGGCACACACAGAGAACCGGCA |
| | CAGCTTGTCGATACCATGAGGAGGTTGAGAAGGAAGGGCGATATCTCTCCCG |
| | AGGTGTCGATCATCAGGGACATCAGAGAAATGGAGTTCAAGATCTTCACCGA |
| | TGCAGGCCGTGTCTACCGTCCGTTGTTCATCGTGGACGACGACCCAGAGTCC |
| | GAAACCAAGGGTGAGTTGATGTTGCAAAAAGAGCACGTGCACAAGTTGTTG |
| | AACTCGGCCTACGATGAATATGACGAGGATGACTCCAATGCGTACACATGGT |
| | CGTCGTTGGTGAATGATGGTGTGGTAGAGTACGTTGACGCCGAGGAGGAGGA |
| | GACAATCATGATCGCCATGACCCCAGAGGATTTGGAGGCTTCCAAGAGTGCG |
| | TTGTCGGAGACTCAGCAACAGGATCTTCAAATGGAGGAACAAGAGCTTGATC |
| | CTGCAAAGCGAATCAAACCAACTTATACCTCATCCACACACACCTTCACGCA |
| | TTGTGAGATTCATCCTTCGATGATTTTGGGTGTCGCCGCCTCTATCATTCCGTT |
| | CCCCGACCATAACCAGTCGCCGCGTAACACATACCAGTCTGCTATGGGTAAA |
| | CAAGCCATGGGTGTATTTTTGACTAACTATGCCGTTAGAATGGACACAATGG |
| | CAAATATCTTATACTACCCACAGAAACCCTTGGCCACAACAAGAGCCATGGA |
| | GCACTTGAAGTTCCGTGAGTTGCCTGCTGGTCAGAATGCAGTGGTGGCCATT |
| | GCTTGTTACTCCGGCTACAACCAAGAAGATTCCATGATCATGAACCAGTCGT |
| | CGATTGATAGAGGATTGTTCCGGTCTTTGTTTTTCAGATCTTACATGGATCTA |
| | GAGAAGAGACAAGGTATGAAAGCCTTGGAGACGTTTGAAAAGCCATCCAGA |
| | TCTGACACCTTGAGATTGAAGCATGGAACCTACGAAAAGTTAGATGACGATG |
| | GTTTGATCGCGCCTGGTGTCAGGGTCAGTGGTGAGGATATCATCATCGGTAA |
| | AACCACACCTATTCCACCTGACACCGAGGAGTTGGGTCAGAGAACCCAGTAT |
| | CATACCAAGAGAGATGCCTCGACGCCATTGAGAAGCACGGAGTCTGGTATTG |
| | TTGACCAGGTTCTTTTGACCACAAATGGTGACGGCGCCAAGTTCGTCAAGGT |
| | CAGAATGAGAACGACGAAGGTTCCACAAATCGGTGACAAGTTTGCCTCCAGA |
| | CACGGACAAAAGGGTACAATCGGTGTCACATATAGACACGAGGATATGCCTT |
| | TCAGTGCACAGGGTATTGTGCCTGACTTGATCATAAACCCGCATGCTATTCCA |
| | TCTCGTATGACAGTCGCTCACTTGATCGAGTGTTTGTTGTCGAAAGTCTCTTC |
| | CTTGTCCGGATTGGAAGGTGACGCCTCGCCATTCACGGACGTCACAGCCGAG |
| | GCTGTTTCCAAATTGTTGAGAGAGCACGGATACCAATCTAGAGGTTTCGAGG |
| | TGATGTACAATGGTCACACCGGTAAGAAGATGATGGCGCAAGTGTTCTTTGG |
| | CCCAACGTACTACCAGAGATTGAGGCATATGGTGGATGACAAGATCCACGCT |
| | AGAGCCAGAGGTCCAGTTCAAGTTTTGACCAGGCAGCCTGTGGAAGGTAGAT |
| | CCAGGGATGGTGGATTACGTTTCGGAGAGATGGAGAGAGATTGTATGATTGC |
| | GCACGGAGCTGCTGGATTCTTAAAGGAAAGATTGATGGAGGCTTCGGATGCT |
| | TTCAGAGTTCACGTTTGTGGAATCTGTGGTTTGATGTCGGTGATTGCAAACTT |
| | GAAGAAGAACCAGTTCGAGTGTCGGTCGTGCAAAAACAAGACCAACATTTA |
| | CCAGATCCACATTCCATACGCAGCCAAATTGTTGTTCCAGGAGTTGATGGCC |
| | ATGAACATTTCTCCTAGATTGTACACGGAGAGATCAGGAATCAGTGTGCGTG |
| | TCTGA (SEQ ID NO: 70) |
| Nucleotide sequence | ATGGGTAAAGAAAAGTCGCACGTCAACGTCGTTGTCATTGGACACGTCGATT |
| of TEF1 gene from H0 | CCGGTAAGTCTACTACCACCGGTCACTTGATCTACAAGTGTGGTGGTATTGAC |
| Metschnikowia sp. | AAGAGAACTATCGAGAAGTTCGAGAAGGAGGCCGCCGAGTTGGGTAAGGGT |
| | TCTTTCAAGTACGCTTGGGTGTTGGACAAGTTGAAGGCTGAGAGAGAGAGAG |
| | GTATCACTATCGACATTGCCTTGTGGAAGTTCGAGACTCCTAAGTACCACGTC |
| | ACCGTCATTGACGCCCCAGGTCACAGAGATTTCATCAAGAACATGATCACTG |
| | GTACTTCCCAGGCTGACTGTGCTATCTTGATCATCGCCGGTGGTGTTGGTGAG |
| | TTCGAGGCTGGTATCTCCAAGGATGGCCAGACCAGAGAGCACGCTTTGTTGG |
| | CTTACACCTTGGGTGTTAGACAATTGATTGTTGCCGTCAACAAGATGGACTCC |
| | GTCAAGTGGGACAAGAACAGATTTGAGGAGATCATCAAGGAGACCTCTAAC |
| | TTCGTCAAGAAGGTTGGTTACAACCCTAAGACTGTGCCATTCGTGCCAATCTC |
| | TGGTTGGAACGGTGACAACATGATTGAGGCTTCCACCAACTGCCCATGGTAC |
| | AAGGGTTGGGAGAAGGAGACCAAGGCCGGTAAGTCTTCCGGTAAGACCTTG |
| | TTGGAGGCCATTGACGCCATTGAGCCACCAACCAGACCTACCGACAAGGCCT |
| | TGAGATTGCCTTTGCAGGATGTCTACAAGATCGGTGGTATCGGAACGGTGCC |
| | AGTCGGCCGTGTCGAGACCGGTGTCATCAAGGCCGGTATGGTCGTCACCTTC |
| | GCCCCAGCTGGTGTCACCACTGAGGTCAAGTCCGTCGAGATGCACCACGAGC |
| | AGTTGGTTGAGGGTCTTCCAGGTGACAACGTTGGTTTCAACGTCAAGAACGT |
| | CTCTGTTAAGGAGATCAGAAGAGGTAACGTCTGTGGTGACTCCAAGCAGGAC |
| | CCACCAAAGGCTGCCGCTTCTTTCACCGCTCAGGTTATTGTGTTGAACCACCC |
| | TGGTCAGATCTCCTCTGGTTACTCTCCAGTGTTGGACTGTCACACCGCCCACA |
| | TTGCCTGTAAATTCGACACCTTGTTGGAGAAGATTGACAGAAGAACTGGTAA |
| | GTCCTTGGAGTCTGAGCCTAAGTTCGTCAAGTCTGGTGACGCCGCCATTGTCA |
| | AGATGGTGCCAACCAAGCCAATGTGTGTTGAGGCTTTCACCGACTACCCACC |
| | TTTGGGTAGATTCGCCGTCAGAGACATGAGACAGACTGTTGCTGTCGGTGTC |
| | ATCAAGGCCGTCGAGAAGTCCGACAAGGCTGGTAAGGTCACCAAGGCTGCTC |
| | AGAAGGCTGCCAAGAAGTAA (SEQ ID NO: 71) |
| Nucleotide sequence | ATGGCTCGTCAATTTTTCGTCGGAGGTAACTTCAAAATGAACGGCACTAAGG |
| of TPI1 gene from H0 | AGTCGCTCACCGCCATTGTCGACACCTTGAACAAGGCCGACTTGCCCGAGAA |
| Metschnikowia sp. | CGTCGAGGTGGTGATTGCTCCCCCAGCCCCATACCTTTCCCTCGTGGTCGAGG |
| | CCAACAAGCAGAAGACCGTGGAGGTCGCTGCTCAAAACGTGTTCAGCAAGG |
| | CCTCCGGTGCCTACACAGGTGAGATTGCTCCTCAGCAATTGAAGGACTTGGG |
| | CGCCAACTGGACCTTGACCGGCCACTCTGAGAGAAGAACGATCATCAAGGA |
| | GTCCGACGAGTTCATCGCCGAGAAGACCAAGTTTGCTTTGGAGTCTGGTGTT |
| | AGCGTCATCTTGTGTATCGGTGAGACCTTGGAGGAGAAGAAGGCTGGCATCA |
| | CGCTTGAGGTGTGCGCCAGACAATTGGACGCTGTGTCCAAGATTGTTTCCGA |
| | CTGGACCAACGTCGTCATTGCTTACGAGCCCGTCTGGGCTATTGGTACTGGCT |
| | TGGCCGCCACTGCCCAGGATGCTCAGGACATCCACAAGGAGATCAGAGCCCA |
| | CTTGTCTAAGACCATTGGCGCTGAACAAGCCGAGGCCGTCAGAATCTTGTAC |
| | GGTGGTTCCGTCAACGGCAAAAACGCTGTTGACTTCAAGGACAAGGCTGATG |
| | TTGACGGATTCTTGGTTGGCGGTGCCTCCTTGAAGCCAGAGTTCATTGACATC |
| | ATCAAGTCTAGATTGTAA (SEQ ID NO: 72) |
| Nucleotide sequence | ATGACTTATAGTTCCAGCTCTGGCCTCTTTTTGGGCTTCGACTTGTCGACGCA |
| of XKS1 gene from H0 | GCAGCTTAAAATCATTGTGACAAACGAGAACTTGAAGGCGCTTGGTACCTAC |
| Metschnikowia sp. | CATGTTGAGTTTGATGCTCAATTCAAAGAGAAATACGCGATCAAAAAGGGTG |
| | TTTTGTCAGATGAAAAAACGGGCGAGATTTTATCACCCGTGCACATGTGGCT |
| | AGAGGCAATTGACCATGTCTTTGGGTTGATGAAAAAAGACAATTTCCCCTTC |
| | GGAAAAGTGAAAGGCATAAGCGGTTCAGGGATGCAGCACGGATCGGTCTTTT |
| | GGTCGAAGTCTGCTTCTTCATCCTTAAAGAATATGGCCGAATATTCCTCTTTA |
| | ACAGAAGCCTTGGCTGATGCCTTTGCGTGTGATACTTCTCCCAACTGGCAGG |
| | ACCATTCGACAGGGAAAGAAATCAAAGACTTTGAGAAAGTCGTTGGAGGCC |
| | CGGACAAATTGGCGGAAATTACAGGCTCAAGAGCTCACTACAGGTTCACTGG |
| | GTTGCAGATTCGGAAGTTGGCAGTGAGATCTGAGAATGACGTTTACCAGAAA |
| | ACCGATAGAATATCTTTGGTGTCGAGTTTTGTTGCGTCCGTTCTTTTGGGCAG |
| | GATCACCACAATTGAGGAGGCGGACGCTTGCGGAATGAATTTATACAATGTG |
| | ACCGAGTCTAAGCTTGATGAAGATTTGTTAGCAATCGCTGCAGGGGTGCATC |
| | CAAAGCTCGATAACAAATCCAAAAGGGAAACAGACGAGGGTGTCAAAGAAC |
| | TAAAGCGAAAGATTGGTGAGATCAAACCCGTGAGTTATCAGACTTCGGGCTC |
| | AATCGCACCATATTTTGTCGAGAAATACGGCTTCTCTCCAGATTCGAAGATTG |
| | TTTCGTTTACGGGTGATAATCTTGCGACCATCATCTCTTTGCCTTTGAGAAAA |
| | AACGACGTCTTGGTGTCACTAGGCACATCCACCACCGTACTTTTGGTGACCG |
| | AGAGCTACGCGCCTTCTTCGCAGTATCATCTTTTCAAGCATCCTACAATTAAG |
| | AATGCTTACATGGGAATGATTTGCTACAGTAATGGCGCGCTAGCAAGAGAAA |
| | GAGTTCGTGACGCCATCAATGAGAAGTATGGTGTGGCAGGGGATTCTTGGGA |
| | CAAGTTCAATGAGATCTTGGATCGCTCAGGCGACTTCAACAATAAGTTGGGT |
| | GTTTACTTTCCCATCGGTGAAATTGTGCCCAATGCTCCGGCCCAGACAAAGA |
| | GAATGGAAATGAACTCGCATGAGGATGTGAAAGAGATCGAAAAGTGGGATT |
| | TGGAAAACGATGTCACTTCTATTGTTGAGTCACAAACCGTTAGTTGCCGAGT |
| | GAGAGCGGGCCCAATGCTTTCTGGATCGGGTGACTCGAATGAAGGAACGCCC |
| | GAAAATGAAAATAGGAAAGTCAAAACACTCATCGACGATTTACACTCTAAGT |
| | TCGGCGAAATTTACACAGACGGGAAACCTCAGAGCTACGAGTCTTTGACTTC |
| | GAGGCCGCGGAACATCTACTTTGTCGGAGGGGCTTCAAGAAACAAGAGTATC |
| | ATACACAAGATGGCTTCGATCATGGGTGCTACCGAAGGAAACTTTCAGGTTG |
| | AGATTCCGAATGCGTGTGCTCTTGGCGGCGCCTACAAGGCAAGCTGGAGCCT |
| | TGAGTGTGAGAGCAGACAAAAGTGGGTGCACTTCAATGATTACCTCAATGAG |
| | AAGTACGATTTCGATGATGTGGATGAGTTCAAAGTGGACGACAAATGGCTCA |
| | ACTATATTCCGGCGATTGGCTTGTTGTCGAAATTGGAAAGCAACCTTGACCA |
| | GAACTAA (SEQ ID NO: 73) |
| Nucleotide sequence | ATGGCTACTATCAAATTGAACTCTGGATACGACATGCCCCAAGTGGGTTTTG |
| of XYL1 gene from H0 | GGTGCTGGAAAGTAACTAACAGTACATGTGCTGATACGATCTACAACGCGAT |
| Metschnikowia sp. | CAAAGTTGGCTACAGATTATTTGATGGCGCTGAAGATTACGGGAACGAGAAA |
| | GAGGTGGGCGAAGGAATCAACAGGGCCATTGACGAAGGCTTGGTGGCACGT |
| | GACGAGTTGTTCGTGGTGTCCAAGCTCTGGAACAACTTCCATCATCCAGACA |
| | ACGTCGAGAAGGCGTTGGACAAGACTTTGGGCGACTTGAATGTCGAGTACTT |
| | GGACTTGTTCTTGATCCATTTCCCAATTGCGTTCAAATTCGTGCCCTTTGAGG |
| | AGAAATACCCGCCCGGCTTCTACTGTGGAGAAGGCGATAAGTTTATCTACGA |
| | GGATGTGCCTTTGCTTGACACGTGGCGGGCATTGGAGAAGTTTGTGAAGAAG |
| | GGTAAGATCAGATCCATCGGAATCTCGAACTTTTCCGGCGCGTTGATCCAGG |
| | ACTTGCTCAGGGGCGCCGAGATCCCCCCTGCCGTGTTGCAGATTGAGCACCA |
| | CCCATACTTGCAGCAGCCCAGATTGATTGAGTATGTGCAGTCCAAGGGTATT |
| | GCCATCACAGCCTACTCCTCTTTTGGCCCACAGTCGTTTGTGGAGTTGGACCA |
| | CCCCAAGGTCAAGGAGTGTGTCACGCTTTTCGAGCACGAAGACATTGTTTCC |
| | ATCGCTAAAGCTCACGACAAGTCCGCGGGCCAGGTATTATTGAGGTGGGCCA |
| | CGCAAAGGGGTCTTGCCGTGATTCCAAAGTCAAACAAAACCGAGCGTTTGTT |
| | GCTGAATTTGAATGTGAACGATTTTGATCTCTCTGAAGCAGAATTGGAGCAA |
| | ATCGCAAAGTTGGACGTGGGCTTGCGCTTCAACAACCCTTGGGACTGGGACA |
| | AGATTCCAATCTTCCATTAA (SEQ ID NO: 74) |
| Nucleotide sequence | ATGCCTGCTAACCCATCCTTGGTTTTGAACAAAGTGAACGACATCACGTTCG |
| of XYL2 gene from H0 | AGAACTACGAGGTTCCGTTACTCACAGACCCCAACGATGTATTGGTTCAGGT |
| Metschnikowia sp. | GAAAAAGACTGGAATCTGTGGATCTGACATCCACTACTACACCCACGGCAGA |
| | ATTGGCGACTTCGTGTTGACAAAGCCAATGGTTTTGGGCCACGAATCCGCCG |
| | GTGTGGTCGTGGAGGTCGGCAAAGGTGTCACTGACTTGAAGGTTGGTGATAA |
| | GGTTGCCATTGAGCCCGGAGTGCCTTCTCGCACCAGTGACGAGTACAAGAGT |
| | GGCCACTACAACTTGTGCCCACACATGTGTTTTGCCGCCACGCCCAACTCTAA |
| | CCCCGACGAGCCAAACCCGCCAGGGACTTTGTGCAAATATTACAAGTCCCCA |
| | GCGGACTTCTTGGTGAAATTGCCTGAGCACGTCTCCCTTGAGTTGGGCGCTAT |
| | GGTCGAGCCTTTGACTGTCGGTGTGCACGCCTCGCGTTTGGGCCGTGTCACTT |
| | TTGGTGACCACGTTGTGGTTTTCGGTGCTGGCCCAGTCGGTATCCTTGCGGCT |
| | GCCGTGGCCAGAAAGTTTGGCGCTGCCAGCGTGACTATCGTCGACATCTTCG |
| | ACAGCAAATTGGAATTGGCCAAGTCCATTGGCGCGGCCACTCACACATTCAA |
| | CTCAATGACTGAGGGTGTTCTTTCGGAGGCTTTGCCCGCGGGCGTGAGACCT |
| | GACGTTGTATTGGAGTGCACTGGAGCAGAGATCTGTGTGCAGCAAGGTGTAC |
| | TTGCGTTGAAGGCTGGTGGCCGCCACGTGCAAGTTGGAAATGCCGGCTCCTA |
| | TCTCAAATTCCCCATCACCGAATTTGTTACCAAGGAGTTGACTCTCTTTGGAT |
| | CCTTCCGTTACGGTTACAACGACTACAAGACGTCGGTCGCCATCTTGGACGA |
| | GAATTACAAGAACGGGAAGGAGAATGCGTTGGTGGACTTTGAAGCCTTGATT |
| | ACTCACCGTTTCCCCTTCAAGAATGCCATTGAGGCTTACGACGCGGTGCGCG |
| | CTGGCGACGGAGCTGTCAAGTGTATCATTGACGGCCCAGAGTAA (SEQ ID |
| | NO: 75) |
| Nucleotide sequence | ATGGGTTACGAGGAAAAGCTTGTAGCGCCCGCGTTGAAATTCAAAAACTTTC |
| of XYT1 gene from H0 | TTGACAAAACCCCCAATATTCACAATGTCTATGTCATTGCCGCCATCTCCTGT |
| Metschnikowia sp. | ACATCAGGTATGATGTTTGGATTTGATATCTCGTCGATGTCTGTCTTTGTCGA |
| | CCAGCAGCCATACTTGAAGATGTTTGACAACCCTAGTTCCGTGATTCAAGGTT |
| | TCATTACCGCGCTGATGAGTTTGGGCTCGTTTTTCGGCTCGCTCACATCCACG |
| | TTCATCTCTGAGCCTTTTGGTCGTCGTGCATCGTTGTTCATTTGTGGTATTCTT |
| | TGGGTAATTGGAGCAGCGGTTCAAAGTTCGTCGCAGAACAGGGCCCAATTGA |
| | TTTGTGGGCGTATCATTGCAGGATGGGGCATTGGCTTTGGGTCATCGGTGGCT |
| | CCTGTTTACGGGTCCGAGATGGCTCCGAGAAAGATCAGAGGCACGATTGGTG |
| | GAATCTTCCAGTTCTCCGTCACCGTGGGTATCTTTATCATGTTCTTGATTGGGT |
| | ACGGATGCTCTTTCATTCAAGGAAAGGCCTCTTTCCGGATCCCCTGGGGTGTG |
| | CAAATGGTTCCCGGCCTTATCCTCTTGATTGGACTTTTCTTTATTCCTGAATCT |
| | CCCCGTTGGTTGGCCAAACAGGGCTACTGGGAAGACGCCGAAATCATTGTGG |
| | CCAATGTGCAGGCCAAGGGTAACCGTAACGACGCCAACGTGCAGATTGAAA |
| | TGTCGGAGATTAAGGATCAATTGATGCTTGACGAGCACTTGAAGGAGTTTAC |
| | GTACGCTGACCTTTTCACGAAGAAGTACCGCCAGCGCACGATCACGGCGATC |
| | TTTGCCCAGATCTGGCAACAGTTGACCGGTATGAATGTGATGATGTACTACA |
| | TTGTGTACATTTTCCAGATGGCAGGCTACAGCGGCAACACGAACTTGGTGCC |
| | CAGTTTGATCCAGTACATCATCAACATGGCGGTCACGGTGCCGGCGCTTTTCT |
| | GCTTGGATCTCTTGGGCCGTCGTACCATTTTGCTCGCGGGTGCCGCGTTCATG |
| | ATGGCGTGGCAATTCGGCGTGGCGGGCATTTTGGCCACTTACTCAGAACCGG |
| | CATATATCTCTGACACTGTGCGTATCACGATCCCCGACGACCACAAGTCTGCT |
| | GCAAAAGGTGTGATTGCATGCTGCTATTTGTTTGTGTGCTCGTTTGCATTCTC |
| | GTGGGGTGTCGGTATTTGGGTGTACTGTTCCGAGGTTTGGGGTGACTCCCAGT |
| | CGAGACAAAGAGGCGCCGCTCTTGCGACGTCGGCCAACTGGATCTTCAACTT |
| | CGCCATTGCCATGTTCACGCCGTCCTCATTCAAGAATATCACGTGGAAGACG |
| | TATATCATCTACGCCACGTTCTGTGCGTGCATGTTCATACACGTGTTTTTCTTT |
| | TTCCCAGAAACAAAGGGCAAGCGTTTGGAGGAGATAGGCCAGCTTTGGGAC |
| | GAAGGAGTCCCAGCATGGAGGTCAGCCAAGTGGCAGCCAACAGTGCCGCTC |
| | GCGTCCGACGCAGAGCTTGCACACAAGATGGATGTTGCGCACGCGGAGCAC |
| | GCGGACTTATTGGCCACGCACTCGCCATCTTCAGACGAGAAGACGGGCACGG |
| | TCTAA (SEQ ID NO: 76 75) |
| Nucleotide sequence | ATGTCTAACTCTTTGGAATCCTTGAAAGCTACCGGCACCGTGATCGTCACCGA |
| of TAL1 gene from H0 | CACTGGTGAGTTCGACTCGATTGCCAAGTACACCCCACAAGATGCCACCACC |
| Metschnikowia sp. | AACCCTTCGTTGATTTTAGCCGCCTCGAAAAAGGCTGAGTACGCCAAGGTGA |
| | TTGATGTTGCTATTAAATACGCCGAGGACAAGGGCAGCAACCCTAAGGAGAA |
| | GGCCGCCATTGCCTTGGACAGATTGTTGGTGGAGTTCGGTAAGGAAATCTTG |
| | CTGATTGTGCCTGGCAGAGTGTCTACCGAGGTTGACGCCAGATTGTCGTTTGA |
| | CAAGGACGCCACCGTCAAGAAGGCGCTTGAGATCATCGAATTGTACAAGTCC |
| | ATTGGCATCTCGAAGGACAGAGTGTTGATCAAGATCGCTTCCACCTGGGAAG |
| | GTATCCAGGCCGCCAAGGAGTTGGAGGCCAAGCACGACATCCACTGTAACTT |
| | GACGCTTTTGTTCAGTTTCGTGCAGGCGGTGGCGTGTGCCGAGGCCAAGGTC |
| | ACTTTGATCTCGCCTTTCGTCGGCAGAATCTTGGACTGGTACAAGGCCTCCAC |
| | CGGCAAGGAGTACGATGCCGAGTCCGACCCTGGTGTTGTGTCTGTCAGACAG |
| | ATCTACAACTACTACAAGAAGTACGGCTACAACACGATTGTCATGGGCGCGT |
| | CTTTCAGAAACACTGGCGAGATCAAGGCCTTGGCTGGCTGCGACTACTTGAC |
| | TGTGGCCCCTAAGTTGTTGGAGGAGTTGATGAACTCTTCCGAGGAGGTGCCT |
| | AAGGTGTTGGACGCTGCCTCGGCCAGCTCCGCGTCTGAGGAGAAGGTTTCCT |
| | ACATTGACGACGAGAGCGAGTTCAGATTCTTGTTGAACGAGGACGCCATGGC |
| | CACCGAGAAGTTGGCCCAGGGTATCAGAGGCTTTGCCAAGGACGCCCAGACC |
| | TTGTTGGCCGAGTTGGAGAACAGATTCAAGTAG (SEQ ID NO: 77) |
| Nucleotide sequence | ATGTCCGACATCGATCAATTGGCTATTTCTACCATCCGTTTGTTGGCGGTCGA |
| of TKL1 gene from H0 | CGCCGTGGCCAAGGCCAACTCTGGTCACCCCGGTGCCCCATTGGGTCTCGCC |
| Metschnikowia sp. | CCTGCCGCCCACGCCGTTTGGAAGGAGATGAAATTCAACCCAAAGAACCCCG |
| | ACTGGGTCAACAGAGACCGTTTTGTGTTGTCGAACGGTCACGCTTGCGCTTTG |
| | TTATACGCCATGTTGCACCTTTACGGCTTCGACATGTCGCTTGACGACTTGAA |
| | GCAGTTCCGTCAGTTGAACTCGAAAACACCCGGACATCCCGAGAAGTTTGAA |
| | ATCCCAGGTGCCGAGGTCACCACGGGCCCCTTGGGTCAGGGTATCTCCAACG |
| | CCGTGGGTTTGGCCATTGCACAGAAGCAATTCGCTGCCACGTTCAACAAGGA |
| | CGATTTCGCCATCTCTGACTCGTACACCTACGCCTTCTTGGGTGACGGATGTT |
| | TGATGGAGGGTGTCGCCTCGGAAGCATCTTCTTTGGCTGGCCACCTCCAATTG |
| | AACAACTTGATTGCGTTCTGGGACGACAACAAGATCTCGATCGATGGATCCA |
| | CTGAAGTGGCCTTCACCGAGGACGTGTTGAAGCGTTACGAGGCTTACGGTTG |
| | GGACACGCTCACGATTGAGAAGGGTGACACTGACTTGGAGGGCGTCGCTCAG |
| | GCGATCAAGACTGCCAAGGCGCTGAAGAAGCCTACTTTGATCCGTTTGACCA |
| | CCATCATCGGCTACGGCTCGCTCCAGCAGGGTACCCACGGTGTTCACGGTGC |
| | TCCATTGAAGCCAGATGACATCAAGCAGTTGAAGGAGAAGTTTGGCTTCGAC |
| | CCAACCAAGTCGTTTGTCGTGCCTCAGGAAGTTTACGACTACTACGGCACAC |
| | TCGTAAAGAAGAACCAGGAGTTGGAGTCCGAGTGGAACAAGACCGTCGAGT |
| | CCTACATCCAGAAATTCCCAGAGGAGGGCGCTGTCTTGGCGCGCAGACTCAA |
| | GGGTGAGTTGCCTGAGGACTGGGCCAAGTGCTTGCCTACTTACACCGCTGAT |
| | GACAAGCCGTTGGCCACGAGAAAGTTGTCTGAGATGGCTCTCATCAAGATCT |
| | TGGATGTCGTTCCAGAGCTTATTGGTGGCTCTGCCGACTTGACCGGCTCGAAC |
| | TTGACCCGTGCCCCTGACATGGTTGACTTCCAGCCCCCTCAGACCGGCTTGGG |
| | TAACTACGCTGGTAGATACATCCGTTACGGTGTGCGTGAGCACGGTATGGGT |
| | GCCATCATGAACGGTATCGCCGGTTTTGGTGCTGGTTTCCGTAACTACGGCGG |
| | TACCTTCTTGAACTTCGTCTCGTACGCCGCCGGTGCTGTGCGTTTGTCGGCTC |
| | TTTCTCACTTGCCTGTGATCTGGGTTGCTACGCATGACTCGATTGGTTTGGGT |
| | GAGGACGGTCCTACCCACCAGCCTATTGAGACCTTGGCCCACTTCAGAGCTA |
| | CCCCTAACATCTCTGTGTGGAGACCTGCTGACGGTAACGAGGTGTCAGCTGC |
| | TTACAAGTCTGCCATTGAGTCTACCTCTACCCCACACATCTTGGCCTTGACCA |
| | GACAGAACTTGCCTCAATTGGCTGGTTCTTCTGTGGAGAAGGCCTCTACCGGT |
| | GGTTACACCGTGTACCAGACCACTGACAAGCCTGCCGTCATCATCGTGGCTT |
| | CTGGTTCCGAGGTGGCCATCTCTATTGACGCCGCCAAGAAGTTGGAGGGTGA |
| | GGGCATCAAGGCCAACGTTGTTTCCTTGGTTGACTTCCACACTTTCGACAAGC |
| | AGCCTTTGGACTACCGTTTATCTGTTTTGCCAGATGGCGTGCCAATCATGTCC |
| | GTTGAGGTGATGTCCTCGTTCGGCTGGTCCAAGTATTCTCACGAGCAGTTCGG |
| | CTTGAACAGATTCGGTGCCTCCGGCAAGGCCGAAGACCTTTACAAGTTCTTC |
| | GACTTCACGCCAGAAGGCGTTGCTGACAGAGCCGCCAAGACCGTGCAGTTCT |
| | ACAAGGGCAAGGACCTCCTTTCGCCTTTGAACAGAGCCTTCTAA (SEQ ID |
| | NO: 78) |

The above identified amino acid and nucleic acid sequences were compared to their corresponding homologs in Metschnikowia fructicola 277 (FR) and Metschnikowia pulcherrima flavia (FL). Table 7 shows the percentage of nucleotide bases and amino acid residues that are identical to the H0 Metschnikowia sp. genes and proteins when compared to the FR and FL species.

TABLE 7

| | % identity of | | % identity of | |
|---|---|---|---|---|
| | nucleotide bases | | amino acid residues | |
| ORF name | FR homolog | FL homolog | FR homolog | FL homolog |
| H0_ACT1 | 99.6 | 99.7 | 100 | 100 |
| H0_ARO8 | 96.2 | 96.3 | 100 | 100 |
| H0_ARO10 | 97.4 | 97.6 | 95.6 | 96.7 |
| H0_GPD1 | 98.6 | 98.7 | 99.8 | 100 |
| H0_GXF1 | 98.7 | 98.7 | 100 | 99.8 |
| H0_GXF2 | 98.2 | 98.1 | 99.6 | 99.5 |
| H0_GXS1 | 98.5 | 98.2 | 100 | 99.8 |
| H0_HGT19 | 97.1 | 97.8 | 98.7 | 99 |
| H0_HXT2.6 | 98.2 | 98.3 | 100 | 99.2 |
| H0_HXT5 | 98.2 | 98.1 | 99.6 | 99.8 |
| H0_PGK1 | 99.3 | 99.8 | 100 | 100 |
| H0_QUP2 | 98.3 | 98 | 100 | 99.8 |
| H0_RPB1 | 97.9 | 97.6 | 100 | 99.9 |
| H0_RPB2 | 98.2 | 98.5 | 100 | 100 |
| H0_TEF1 | 98.8 | 99.2 | 99.8 | 99.8 |
| H0_TPI1 | 98.9 | 99.3 | 100 | 100 |
| H0_XKS1 | 97.1 | 96.6 | 98.2 | 97 |
| H0_XYL1 | 97.6 | 97.4 | 99.7 | 99.4 |
| H0_XYL2 | 98.3 | 98.3 | 99.7 | 100 |
| H0_XYT1 | 97.9 | 97.6 | 100 | 97.6 |
| H0_TAL1 | 98.6 | 98.8 | 99.7 | 99.4 |
| H0_TKL1 | 99.0 | 98.5 | 99.9 | 99.9 |

Accordingly, the H0 Metschnikowia sp. has unique nucleic acid sequences for the following genes: ACT1, ARO8, ARO10, GPD1, GXF1, GXF2, GXS1, HXT19, HXT2.6, HXT5, PGK1, QUP2, RPB1, RPB2, TEF1, TPI1, XKS1, XYL1, XYL2, XYT1, TAL1 and TKL1, as well as unique amino acid sequences for the following proteins: Aro10, Gxf2, Hgt19, Hxt5, Tef1, Xks1, Xyl1, Tal1 and Tkl1.

1. An isolated Metschnikowia species that produces:
(a) at least 0.1 g/L/h of xylitol from xylose when cultured under aerobic conditions and at 30° C. for three days in liquid yeast extract peptone (YEP) medium comprising 4% xylose;
(b) at least 1 g/L of xylitol from xylose when cultured under aerobic conditions and at 30° C. for three days in liquid yeast nitrogen base (YNB) medium comprising 4% xylose; or
(c) at least 1 g/L of xylitol from xylose when cultured under aerobic conditions and at 30° C. for two days in liquid yeast nitrogen base (YNB) medium comprising 2% xylose and 2% glucose.

2-3. (canceled)

4. An isolated Metschnikowia species that produces:
(a) about 0.11 g/L/h of xylitol, about 6.8E-05 g/L/h of n-butanol, about 2.5E-04 g/L/h of isobutanol, about 2.4E-04 g/L/h of isopropanol, about 2.64E-04 g/L/h of ethanol and about 3.73E-06 g/L/h of 2-phenylethyl alcohol when cultured under aerobic conditions for three days in liquid yeast extract peptone (YEP) medium comprising 4% xylose;
(b) compounds xylitol, n-butanol, isobutanol, isopropanol, ethanol and 2-phenylethyl alcohol at a concentration of about 8,000 mg/L xylitol, about 4.85 mg/L n-butanol, about 18.06 mg/L isobutanol, about 17.5 mg/L isopropanol, about 19.7 mg/L ethanol and about 0.269 mg/L 2-phenylethyl alcohol when cultured under aerobic conditions for three days in liquid yeast extract peptone (YEP) medium comprising 4% xylose; or
(c) compounds xylitol, n-butanol, isobutanol, isopropanol, ethanol and 2-phenylethyl alcohol at a relative ratio of 99.26% xylitol, 0.061% n-butanol, 0.223% isobutanol, 0.217% isopropanol, 0.236% ethanol and 0.003% 2-phenylethyl alcohol when cultured under aerobic conditions for three days in liquid yeast extract peptone (YEP) medium comprising 4% xylose.

5-6. (canceled)

7. An isolated Metschnikowia species comprising:
(a) a D1/D2 domain sequence that comprises: (1) a nucleic acid sequence that is at least 96.8% identical to SEQ ID NO: 1; (2) a nucleic acid sequence within the consensus sequence of SEQ ID NO: 2; or (3) a nucleic acid sequence comprising residues 1-153, 178 to 434 and 453 to 499 of SEQ ID NO: 2 with no more than 4 nucleotide substitutions therein, and at least one nucleic acid sequence encoding an amino acid sequence selected from the group consisting of SEQ ID NOS: 37, 40, 42, 44, 49, 51, 52, 55 and 56;
(b) a D1/D2 domain sequence that comprises: (1) a nucleic acid sequence that is at least 96.8% identical to SEQ ID NO: 1; or (2) a nucleic acid sequence within the consensus sequence of SEQ ID NO: 2; or (3) a nucleic acid sequence comprising residues 1-153, 178 to 434 and 453 to 499 of SEQ ID NO: 2 with no more than 4 nucleotide substitutions therein, and at least one encoding nucleic acid sequence selected from the group consisting of SEQ ID NOS: 57-78;
(c) (1) a D1/D2 domain sequence that is at least 96.8% identical to SEQ ID NO: 1; and (2) an encoding nucleic acid sequence of SEQ ID NO: 68, and wherein said isolated Metschnikowia species grows to an OD₆₀₀ of about 25 within 41 hours of culturing in yeast extract peptone (YEP) medium comprising 2% xylose as the sole carbon source; or
(d) (1) a nucleic acid sequence that is at least 97.1% identical to the D1/D2 domain consensus sequence of SEQ ID NO: 2; and (2) an encoding nucleic acid sequence of SEQ ID NO: 70.

8.-10. (canceled)

11. The isolated Metschnikowia species of claim 7, wherein the D1/D2 domain sequence comprises a nucleic acid sequence selected from the group consisting of SEQ ID NOS: 1 and 3-25.

12. The isolated Metschnikowia species of claim 7, wherein the D1/D2 domain sequence does not comprise the D1/D2 domain sequence of a Metschnikowia species selected from the group consisting of Metschnikowia andauensis, Metschnikowia chrysoperlae, Metschnikowia fructicola, Metschnikowia pulcherrima, Metschnikowia shanxiensis, Metschnikowia sinensis, and Metschnikowia zizyphicola.

13. An isolated Metschnikowia species designated Accession No. 081116-01, deposited at the International Depositary Authority of Canada, an International Depositary Authority, on Nov. 8, 2016, under the terms of the Budapest Treaty.

14. A method for producing xylitol comprising culturing the isolated Metschnikowia species of claim 1 under conditions and for a sufficient period of time to produce xylitol from xylose.

15. The method of claim 14, wherein the isolated Metschnikowia species produces at least 0.1 g/L/h, at least 0.2 g/L/h, at least 0.3 g/L/h, at least 0.4 g/L/h, at least 0.50 g/L/h, at least 0.60 g/L/h, at least 0.70 g/L/h, at least 0.80 g/L/h, at least 0.90 g/L/h, at least 1.00 g/L/h, at least 1.50 g/L/h, at least 2.00 g/L/h, at least 2.50 g/L/h, at least 3.00 g/L/h, at least 3.50 g/L/h, at least 4.00 g/L/h, at least 5.00 g/L/h, at least 6.00 g/L/h, at least 7.00 g/L/h, at least 8.00 g/L/h, at least 9.00 g/L/h, or at least 10.00 g/L/h of xylitol from xylose.

16. The method of claim 14, wherein the conditions comprise culturing the isolated Metschnikowia species in medium comprising xylose and a C3 carbon source, a C4 carbon source, a C5 carbon source, a C6 carbon source, or a combination thereof.

17. The method of claim 14, wherein the conditions comprise culturing the isolated Metschnikowia species in medium comprising xylose and a co-substrate selected from the group consisting of cellobiose, galactose, glucose, ethanol, acetate, arabinose, arabitol, sorbitol and glycerol, or a combination thereof

18-21. (canceled)

22. The method of claim 14, wherein the culturing comprises aerobic culturing conditions.

23. The method of claim 14, wherein the culturing comprises batch cultivation, fed-batch cultivation or continuous cultivation.

24. The method of claim 14, wherein the method further comprises separating the xylitol from other components in the culture.

25-26. (canceled)

27. A composition comprising the isolated Metschnikowia species of claim 1 or the bioderived xylitol of claim 26, or both.

28. The composition of claim 27, wherein the composition is culture medium comprising xylose.

29-32. (canceled)

33. An isolated Metschnikowia species designated Accession No. 081116-01, deposited at the International Depositary Authority of Canada, an International Depositary Authority, on Nov. 8, 2016, under the terms of the Budapest Treaty, wherein the Metschnikowia species further comprises a metabolic pathway capable of producing a bioderived compound from xylose or a genetic modification, or both.

34. The isolated Metschnikowia species of claim 33, wherein the metabolic pathway comprises at least one exogenous nucleic acid sequence encoding at least one enzyme of the metabolic pathway.

35. The isolated Metschnikowia species of claim 33, wherein the bioderived compound is selected from the group consisting of phenyl-ethyl alcohol, 2-methyl-butanol, and 3-methyl-butanol.

36. A method of producing a bioderived compound comprising culturing the isolated Metschnikowia species of claim 33 under conditions and for a sufficient period of time to produce the bioderived compound.

37. (canceled)

38. The method of claim 36, wherein the conditions comprise culturing the microbial organism in medium comprising xylose and a co-substrate selected from the group consisting of cellobiose, galactose, glucose, arabitol, sorbitol and glycerol, or a combination thereof.

39-47. (canceled)

48. A composition comprising the Metschnikowia species of claim 33.

49. The composition of claim 48, wherein the composition is culture medium comprising xylose.

50-53. (canceled)

54. An isolated polypeptide comprising an amino acid sequence selected from the group consisting of SEQ ID NOS: 37, 40, 42, 44, 49, 51, 52, 55 and 56.

55. An isolated nucleic acid comprising a nucleic acid sequence selected from the group consisting of SEQ ID NOS: 57-78.

56. A vector comprising the isolated nucleic acid sequence of claim 55.

57. A host cell comprising the vector of claim 56.
